(19) United States
(12) Patent Application Publication
(10) Pub. No.: US 2024/0218069 A1
(43) Pub. Date: Jul. 4, 2024

(54) BI-SPECIFIC ANTIBODIES COMPRISING ANTI-B7H3 BINDING MOLECULES

(71) Applicant: LYVGEN BIOPHARMA HOLDINGS LIMITED, Grand Cayman (KY)

(72) Inventors: Jieyi WANG, Belmont, CA (US); Yi WU, Shanghai (CN); Xiang XU, Shanghai (CN)

(73) Assignee: LYVGEN BIOPHARMA HOLDINGS LIMITED, Grand Cayman (KY)

(21) Appl. No.: 18/556,596

(22) PCT Filed: Apr. 28, 2022
(86) PCT No.: PCT/US2022/026731

§ 371 Date: Oct. 20, 2023

(30) Foreign Application Priority Data
Apr. 28, 2021 (WO) PCT/CN2021/090537

(51) Int. Cl. C07K 16/28; A61K 39/00; A61P 35/00

(52) U.S. Cl. CPC C07K 16/2827; A61P 35/00; C07K 16/2803; C07K 16/2809; C07K 16/2818; C07K 16/2878; A61K 39/00; C07K 2317/24; C07K 2317/31

(57) ABSTRACT

A multi-specific antibody (e.g., bi-specific or tri-specific) comprising one antigen binding moiety specific to human B7H3 and one or more antigen binding moieties specific to one or more antigens of interest, for example, CD40, CD137, Glucocorticoid-Induced TNFR-Related protein (GITR), OX40, CD3, CD28, and CD47. Also provided herein are methods for preparing such antibodies and methods of using such for modulating immune responses.

### CROSS REFERENCE TO RELATED APPLICATIONS

This application claims the benefit of the filing date of International Patent Application No. PCT/CN2021/090537, filed Apr. 28, 2021, the entire contents of which are incorporated by reference herein.

### SEQUENCE LISTING

The instant application contains a Sequence Listing which has been filed electronically in ASCII format and is hereby incorporated by reference in its entirety. Said ASCII copy, created on Apr. 27, 2022, is named 112238-0098-70010W02_SEQ.txt and is 2,650,405 bytes in size.

### BACKGROUND OF THE INVENTION

B7H3 (also known as CD276, an immune checkpoint molecule) is aberrantly overexpressed in many types of cancer, and such upregulation is generally associated with a poor clinical prognosis. Recent discoveries indicate a crucial role for B7H3 in promoting carcinogenesis and metastasis.

Considering the significant roles of B7H3 in cancer immunity and progression, the value of B7H3 in cancer diagnosis and treatment warrants further detailed study. Here, we develop new B7H3-targeting immune therapies that are effective and safe use in the treatment of cancer.

### SUMMARY OF THE INVENTION

The present disclosure is based, at least in part, on the development of bi-specific and multi-specific antibodies targeting both human B7H3 and other desired antigen, such as CD40, CD137, GITR, OX40, CD47, CD3 or CD28. Such bi-specific and multi-specific antibodies exhibit substantially similar antigen-binding affinity and specificity as the parent antibodies and show one or more superior features, for example, simultaneous binding to both target antigens, enhanced antagonistic activity of B7H3 and optionally of the other desired antigen, superior anti-tumor activities, or a combination thereof. For example, the B7H3/CD40 bsAb bispecific antibodies including Ly1581, Ly1579, Ly1663 and Ly1585 showed much stronger efficacy and better safety compared to the CD40 parental mAb Ly253-G2 (corresponding to CD40 Ab1 in Table 1).

Accordingly, the present disclosure features, in one aspect, a multi-specific antibody, comprising:
(a) a first antigen binding moiety binding to a first antigen, wherein the first antigen binding moiety comprising a first heavy chain variable region (V_{H}) and a first light chain variable region (V_{L});
(b) a second antigen binding moiety binding to a second antigen, wherein the second antigen binding moiety comprising a second V_{H} and a second V_{L}; and optionally
(c) a third antigen binding moiety binding to a third antigen, wherein the third antigen binding moiety comprising a third V_{H} and a third V_{L}.

One of the first antigen and the second antigen is human B7H3 and the other one is human CD40, human CD137, human Glucocorticoid-Induced TNFR-Related protein (GITR), human OX40, human CD3, human CD28, or human CD47. The optional third antigen is selected from the group consisting of human CD40, human CD137, human Glucocorticoid-Induced TNFR-Related protein (GITR), human OX40, human CD3, human CD28, and human CD47. The optional third antigen being different from the first antigen and the second antigen.

In some embodiments, the antigen binding moiety that binds B7H3 comprises (i) a heavy chain variable region (V_{H}) that comprises the same heavy chain complementary determining regions (CDRs) as antibody B7H3 Ab1 or B7H3 Ab2, and (ii) a light chain variable region (V_{L}) that comprises the same light chain CDRs as antibody B7H3 Ab1 or B7H3 Ab2.

In some embodiments, the antigen binding moiety that binds B7H3 comprises (i) a heavy chain variable region (V_{H}) that comprises heavy chain complementary determining regions (CDRs), which collectively contain up to 5 amino acid residue variations relative to the heavy chain CDR3 of antibody B7H3 Ab1 or B7H3 Ab2, and (ii) a light chain variable region (V_{L}) that comprises light chain CDRs, which collectively contain up to 5 amino acid residue variations relative to the light chain CDRs of antibody B7H3 Ab1 or B7H3 Ab2.

In some examples, the antigen binding moiety that binds B7H3 comprises (i) the V_{H} that comprises an amino acid sequence at least 80% identical to that of antibody B7H3 Ab1 or B7H3 Ab2; and (ii) the V_{L} that comprises an amino acid sequence at least 80% identical to that of antibody B7H3 Ab1 or B7H3 Ab2. In specific examples, the antigen binding moiety that binds B7H3 comprises the same V_{H} and same V_{L} as antibody B7H3 Ab1 or B7H3 Ab2.

Alternatively or in addition, the antigen binding moieties that bind human CD40, human CD137, human Glucocorticoid-Induced TNFR-Related protein (GITR), human OX40, human CD3, human CD28, or human CD47 each comprise (i) a heavy chain variable region (V_{H}) that comprises the same heavy chain complementary determining regions (CDRs) as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and (ii) a light chain variable region (V_{L}) that comprises the same light chain CDRs as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

In some embodiments, the antigen binding moieties that bind human CD40, human CD137, human Glucocorticoid-Induced TNFR-Related protein (GITR), human OX40, human CD3, human CD28, or human CD47 each comprise (i) a heavy chain variable region (V_{H}) that comprises heavy chain complementary determining regions (CDRs), which collectively contain up to 5 amino acid residue variations relative to the heavy chain CDR3 of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and (ii) a light chain variable region (V_{L}) that comprises light chain CDRs, which collectively contain up to 5 amino acid residue variations relative to the light chain CDRs of CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

In some examples, the antigen binding moieties that bind human CD40, human CD137, human Glucocorticoid-Induced TNFR-Related protein (GITR), human OX40, human CD3, human CD28, or human CD47 each comprise (i) the V_{H} that comprises an amino acid sequence at least 80% identical to that of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and (ii) the V_{L} that comprises an amino acid sequence at least 80% identical to that of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2. In specific examples, the antigen binding moieties that bind human CD40, human CD137, human Glucocorticoid-Induced TNFR-Related protein (GITR), human OX40, human CD3, human CD28, or human CD47 each comprise the same V_{H} and same V_{L} as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

In some embodiments, the multi-specific antibody disclosed herein can be a bi-specific antibody that comprises the first antigen binding moiety and the second antigen binding moiety. In some instances, the bi-specific antibody is of a two-chain format. In some instances, the bi-specific antibody is of a three-chain format. Alternatively, the bi-specific antibody is of a four-chain format.

In some examples, the bi-specific antibody disclosed herein may comprise (ii) a first antigen binding moiety that comprises a heavy chain that comprises the first V_{H} and a heavy chain constant region or a fragment thereof and a light chain that comprises the first V_{L} and a light chain constant region; and (ii) a second antigen binding moiety, which is a single chain variable fragment (scFv); and wherein the scFv is linked to either the heavy chain or the light chain of (i). The scFv may be linked to the N-terminus of the heavy chain of (i) or the C-terminus of the heavy chain. Such a bi-specific antibody may comprise a first polypeptide that comprises the heavy chain of (i) fused to the scFv, and a second polypeptide that comprises the light chain of (i). Alternatively, the bi-specific antibody may comprise a first polypeptide that comprise the heavy chain of (i); and a second polypeptide that comprises the light chain of (i) fused to the scFv.

In some instances, the first antigen binding moiety binds B7H3 and the second antigen binding moiety may bind one of CD40, CD137, GITR, OX40, CD3, CD28, and CD47. In some instances, the multi-specific antibody is a multi-chain complex comprising two copies of each of the first polypeptide and the second polypeptide.

In some instances, the bi-specific antibody is of a three-chain format, which may comprise (i) a first polypeptide that comprise a first heavy chain of the first antigen binding moiety, wherein the first heavy chain comprises the first V_{H} and a first heavy chain constant region or a fragment comprising the CH3 domain therein; (ii) a second polypeptide that comprises a second heavy chain of the first antigen binding moiety and the second antigen binding moiety, which is a single chain variable fragment (scFv) comprising the second V_{H} and second V_{L}, wherein the second heavy chain comprises the first V_{H} and a second heavy chain constant region or a fragment comprising the CH3 domain therein; and (iii) a third polypeptide that comprises a light chain of the first antigen binding moiety, which comprises the first V_{L} and a light chain constant region. In some examples, the first and second heavy chain constant regions comprise mutations in the CH3 domains that enhances heterodimeration over homodimeration as relative to the wild-type counterpart and/or reduces protein A binding. In some examples, the scFv is located between the first V_{H} and the second Fc fragment or the CH3 domain thereof in the second polypeptide. Alternatively, the scFv is located at the C-terminus of the second polypeptide.

In some instances, the bi-specific antibody disclosed herein may comprise: (i) a first polypeptide that comprise a first heavy chain of the first antigen binding moiety and one of the second V_{H} and the second V_{L} of the second antigen binding moiety; wherein the first heavy chain comprises the first V_{H} and a first heavy chain constant region or a fragment comprising the CH3 domain therein; (ii) a second polypeptide that comprises a second heavy chain of the first antigen binding moiety and the other one of the second V_{H} and second V_{L} of the second antigen binding moiety, wherein the second heavy chain comprises the first V_{H} and a second heavy chain constant region or a fragment comprising the CH3 domain therein; and (iii) a third polypeptide that comprises a light chain of the first antigen binding moiety, which comprises the first V_{L} and a light chain constant region. In some examples, the first and second heavy chain constant regions comprise mutations in the CH3 domains that enhances heterodimeration over homodimeration as relative to the wild-type counterpart. In some examples, the first and second heavy chain constant regions comprise the mutations, which are knob-in-hole mutations, charged mutations, or ZW1 mutations. Alternatively or in addition, one of the first and second heavy chain constant regions comprises a mutation that reduces protein A binding activity relative to the wild-type counterpart.

In some instances, the bi-specific antibody is of a four-chain format, which may comprise: (i) a first polypeptide comprising a first heavy chain of the first antigen binding moiety, the first heavy chain comprising the first V_{H} and a first heavy chain constant region or a fragment comprising the CH3 domain therein; (ii) a second polypeptide comprising a second heavy chain of the second antigen binding moiety, the second heavy chain comprising the second V_{H}, a light chain constant region, and a second heavy chain constant fragment comprising a CH3 domain; (iii) a third polypeptide comprising a light chain of the first antigen moiety, which comprises the first V_{L} and a light chain constant region; and (iv) a fourth polypeptide comprising a light chain of the second antigen moiety, the light chain comprising the second V_{L} linked to a CH1 domain of a heavy chain constant region. In some examples, the first and second heavy chain constant regions comprise mutations in the CH3 domains that enhances heterodimeration over homodimeration as relative to the wild-type counterpart.

In some instances, the bi-specific antibody disclosed herein may comprise: (i) a first polypeptide comprising a first heavy chain of the first antigen binding moiety, wherein the first heavy chain comprises the first V_{H} and a first heavy chain constant region or a fragment comprising the CH3 domain therein; (ii) a second polypeptide comprising a second heavy chain, which comprises the first V_{H}, the second antibody binding moiety that is a scFv fragment comprising the second V_{H} and the second V_{L}, and a second heavy chain constant region or a fragment comprising the CH3 domain therein; (iii) a third polypeptide comprising a light chain of the first antigen binding moiety, which comprises the first V_{L} and a light chain constant region; and (iv) a fourth polypeptide comprising the scFv. In some examples, the first and second heavy chain constant regions comprise mutations in the CH3 domains that enhances heterodimeration over homodimeration as relative to the wild-type counterpart.

In some instances, the bi-specific antibody disclosed herein may comprise: (i) a first polypeptide comprising a first heavy chain of the first antigen binding moiety, wherein the first heavy chain comprises the first V_{H} and a first heavy chain constant region or a fragment comprising the CH3 domain therein; (ii) a second polypeptide comprising a light chain of the first antigen binding moiety, which comprises the first V_{L} and a light chain constant region; (iii) a third polypeptide comprising the second V_{H}, a first TCR fragment, and a second heavy chain constant fragment comprising a CH3 domain; and (iv) a fourth polypeptide comprising the second V_{L} and a second TCR fragment. The first and second TCR fragments collectively are a TCR alpha chain fragment and a TCR beta chain fragment, which form a dimer. In some instances, the first and second heavy chain constant regions comprise mutations in the CH3 domains that enhances heterodimeration over homodimeration as relative to the wild-type counterpart. In some examples, the third polypeptide further comprises the first V_{H} linked to a CH1 domain of a heavy chain constant region.

In some examples, a bi-specific antibody disclosed herein comprises the first antigen binding moiety that binds B7H3 and the second antigen binding moiety that binds CD40. Examples include Ly1581, Ly1660, Ly1661, Ly1662, Ly1663, Ly1679, Ly1935, and Ly1936.

In some examples, a bi-specific antibody disclosed herein comprises the first antigen binding moiety that binds B7H3 and the second antigen binding moiety that binds CD137. Examples include Ly1937, Ly1938, Ly1939, Ly1940, Ly1941, Ly1942, Ly1943, and Ly1944.

In some examples, a bi-specific antibody disclosed herein comprises the first antigen binding moiety that binds B7H3 and the second antigen binding moiety that binds GITR. Examples include Ly1945, Ly1946, Ly1947, Ly1948, Ly1049, Ly1950, Ly1951, and Ly1952.

In some examples, a bi-specific antibody disclosed herein comprises the first antigen binding moiety that binds B7H3 and the second antigen binding moiety that binds OX40. Examples include Ly1953, Ly1954, Ly1955, Ly1956, Ly1957, Ly1958, Ly1959, and Ly1960.

In some examples, a bi-specific antibody disclosed herein comprises the first antigen binding moiety that binds B7H3 and the second antigen binding moiety that binds CD47. Examples include Ly2043, Ly2044, Ly2045, Ly2046, Ly2047, Ly2048, Ly2049, Ly2050, Ly2051, Ly2052, Ly2053, Ly2054, Ly2055, Ly2056, Ly2057, Ly2058, Ly2059, Ly2060, Ly2061, Ly2062, Ly2063, and Ly2064.

In some examples, a bi-specific antibody disclosed herein comprises the first antigen binding moiety that binds B7H3 and the second antigen binding moiety that binds CD3. Examples include Ly1900, Ly1901, Ly1902, Ly1903, and Ly1904.

In some embodiments, the multi-specific antibody disclosed herein is a tri-specific antibody that comprises (i) the first antigen binding moiety, (ii) the second antigen binding moiety, and (iii) the third antigen binding moiety.

In some examples, the tri-specific antibody disclosed herein may comprise: (i) a first polypeptide comprising a first heavy chain that comprises the first V_{H} of the first antigen binding moiety and first heavy chain constant region or a fragment comprising the CH3 domain therein; (ii) a second polypeptide comprising the first V_{H}, a second heavy chain comprising the second V_{H} of the second antigen binding moiety and a light chain constant region, and a second heavy chain constant region or a fragment comprising the CH3 domain therein; (iii) a third polypeptide comprising a light chain of the first antigen binding moiety, which comprises the first V_{L} and a light chain constant region; and (iv) a fourth polypeptide comprising the second V_{L} of the second antigen binding moiety and a CH1 fragment of a heavy chain constant region. The first polypeptide, the second polypeptide or both may further comprise the third antigen binding moiety, which is a single chain variable fragment (scFv). In some instances, the first and second heavy chain constant regions comprise mutations in the CH3 domains that enhances heterodimeration over homodimeration as relative to the wild-type counterpart.

In some examples, the tri-specific antibody disclosed here may comprise: (i) a first polypeptide comprising a first heavy chain that comprises the first V_{H} of the first antigen binding moiety and a first heavy chain constant region or a fragment comprising the CH3 domain therein; (ii) a second polypeptide comprising the first V_{H}, a second heavy chain comprising the second V_{L} of the second antigen binding moiety and a CH1 domain of a heavy chain constant region, and a second heavy chain constant region or a fragment comprising the CH3 domain therein; (iii) a third polypeptide comprising a light chain of the first antigen binding moiety; and (iv) a fourth polypeptide comprising the second V_{H} of the second antigen binding moiety and a light chain constant region. The first polypeptide, the second polypeptide or both may further comprise the third antigen binding moiety, which is a single chain variable fragment (scFv). In some examples, the first and second heavy chain constant regions comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety, first heavy chain constant region or a fragment comprising the CH3 domain therein, and one of the third V_{H} and third V_{L} of the third antigen binding moiety; (ii) a second polypeptide comprising the first V_{H}, a second heavy chain comprising the second V_{H} of the second antigen binding moiety and a light chain constant region, a second heavy chain constant region or a fragment comprising the CH3 domain therein, and the other one of the third V_{H} and third V_{L}; (iii) a third polypeptide comprising a light chain of the first antigen binding moiety, which comprises the first V_{L} and a light chain constant region; and (iv) a fourth polypeptide comprising the second V_{L} of the second antigen binding moiety and a CH1 domain of a heavy chain constant region. In some instances, the first and second heavy chain constant regions may comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety, a first heavy chain constant region or a fragment comprising the CH3 domain therein, and one of the third V_{H} and third V_{L} of the third antigen binding moiety; (ii) a second polypeptide comprising the first V_{H}, a second heavy chain comprising the second V_{L} of the second antigen binding moiety and a CH1 domain of a heavy chain constant region, a second heavy chain constant region or a fragment comprising the CH3 domain therein, and the other one of the third V_{H} and third V_{L}; (iii) a third polypeptide comprising a light chain of the first antigen binding moiety, which comprises the first V_{L} and a light chain constant region; and (iv) a fourth polypeptide comprising the second V_{H} of the second antigen binding moiety and a light chain constant region. In some instances, the first and second heavy chain constant regions may comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety and a first heavy chain constant region or a fragment comprising the CH3 domain therein; (ii) a second polypeptide comprising the first V_{H}, the second antigen binding moiety, which is a scFv, and a second heavy chain constant region or a fragment comprising the CH3 domain therein; and (iii) a third polypeptide comprising a light chain of the first antigen binding moiety, which comprises the first V_{L} and a light chain constant region. The first polypeptide, the second polypeptide, or both may further comprise the third antigen binding moiety, which is a scFv. In some instances, the first and second heavy chain constant regions may comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety, one of the second V_{H} and second V_{L} of the second antigen binding moiety, and a first heavy chain constant region or a fragment comprising the CH3 domain therein; (ii) a second polypeptide comprising the first V_{H}, the other one of the second V_{H} and second V_{L}, and a second heavy chain constant region or a fragment comprising the CH3 domain therein; and (iii) a third polypeptide comprising a light chain of the first antigen binding moiety, which comprises the first V_{L} and a light chain constant region. The first polypeptide, the second polypeptide, or both may further comprise the third antigen binding moiety, which is a scFv, or wherein the third polypeptide further comprises the third antigen binding moiety. In some instances, the first and second heavy chain constant regions may comprise mutations in the CH3 domains that enhances heterodimeration over homodimeration as relative to the wild-type counterpart.

In some examples, the tri-specific antibodies disclosed herein may comprise: (i) a first polypeptide comprising a first heavy chain that comprises the first V_{H} of the first antigen binding moiety, one of the second V_{H} and second V_{L} of the second antigen binding moiety, a first heavy chain constant region or a fragment comprising the CH3 domain therein, and one of the third V_{H} and third V_{L} of the third antigen binding moiety; (ii) a second polypeptide comprising the first V_{H}, the other one of the second V_{H} and second V_{L}, and a second heavy chain constant region or a fragment comprising the CH3 domain therein, and the other one of the third V_{H} and third V_{L}; and (iii) a third polypeptide comprising a light chain of the first antigen binding moiety, which comprises the first V_{L} and a light chain constant region. The first and second heavy chain constant regions may comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety, a first heavy chain constant region or a fragment comprising the CH3 domain therein, and the second antigen binding moiety, which is a scFv; (ii) a second polypeptide comprising the first V_{H}, a second heavy chain constant region or a fragment comprising the CH3 domain therein, and the third antigen binding moiety, which is a scFv; and (iii) a third polypeptide comprising a light chain of the first antigen binding moiety, which comprises the first V_{L} and a light chain constant region. In some instances, the first and second heavy chain constant regions comprise mutations in the CH3 domains that enhances heterodimeration over homodimeration as relative to the wild-type counterpart.

In some examples, the tri-specific antibody disclosed herein may comprise: (i) a first polypeptide comprising a first heavy chain that comprises the first V_{H} of the first antigen binding moiety and a first heavy chain constant region or a fragment comprising the CH3 domain therein; (ii) a second polypeptide comprising a second heavy chain that comprises the second V_{H} of the second antigen binding moiety and one of the third V_{H} and third V_{L} of the third antigen binding moiety, wherein the third V_{H} is fused with a light chain constant region or the third V_{L} is fused with a CH1 domain of a heavy chain constant region; (iii) a third polypeptide comprising a light chain of the second antigen binding moiety, which comprises the second V_{L} and a light chain constant region, and the other one of the third V_{H} and third V_{L} of the third antigen binding moiety, wherein the third V_{H} is fused with a light chain constant region or the third V_{L} is fused with a CH1 domain of a heavy chain constant region; and (iv) a fourth polypeptide comprising a light chain of the first antigen binding moiety, which comprises the first V_{L} and a light chain constant region. Either the second polypeptide or the third polypeptide may further comprise a second heavy chain constant region or a fragment comprising the CH3 domain therein. In some instances, the first and second heavy chain constant regions comprise mutations in the CH3 domains that enhances heterodimeration over homodimeration as relative to the wild-type counterpart.

In some examples, the tri-specific antibody disclosed herein may comprise: (i) a first polypeptide comprising the first V_{H} of the first antigen binding moiety, the second V_{H} of the second antigen binding moiety, and a first heavy chain constant region or a fragment comprising the CH3 domain therein; (ii) a second polypeptide comprising the third V_{H} of the third antigen binding moiety and a light chain constant domain; (iii) a third polypeptide comprising the third V_{L} of the third antigen binding moiety and a CH1 domain of a heavy chain constant region; and (iv) a fourth polypeptide comprising a light chain of the first antigen binding moiety, which comprises the first V_{L} and a light chain constant region. Either the second polypeptide or the third polypeptide may further comprise the second V_{L} of the second antigen binding moiety and a second heavy chain constant region or a fragment comprising the CH3 domain therein. In some instances, the first and second heavy chain constant regions comprise mutations in the CH3 domains that enhances heterodimeration over homodimeration as relative to the wild-type counterpart. In some instances, either the second polypeptide or the third polypeptide may further comprises the first V_{H} fused to a CH1 of a heavy chain constant region.

In some examples, the tri-specific antibody disclosed herein may comprise: (i) a first polypeptide comprising the first V_{H} of the first antigen binding moiety, the second V_{L} of the second antigen binding moiety, and a first heavy chain constant region or a fragment comprising the CH3 domain therein; (ii) a second polypeptide comprising the third V_{H} of the third antigen binding moiety and a light chain constant domain; (iii) a third polypeptide comprising the third V_{L} of the third antigen binding moiety and a CH1 domain of a heavy chain constant region; and (iv) a fourth polypeptide comprising a light chain of the first antigen binding moiety, which comprises the first V_{L} and a light chain constant region. Either the second polypeptide or the third polypeptide may further comprise the second V_{H} of the second antigen binding moiety and a second heavy chain constant region or a fragment comprising the CH3 domain therein. In some instances, the first and second heavy chain constant regions may comprise mutations in the CH3 domains that enhances heterodimeration over homodimeration as relative to the wild-type counterpart. In some instances, either the second polypeptide or the third polypeptide may further comprises the first V_{H} fused to a CH1 of a heavy chain constant region.

In some examples, tri-specific antibody disclosed herein may comprise: (i) a first polypeptide comprising a heavy chain of the 1^{st} antigen binding moiety and the second antigen binding moiety, wherein the heavy chain of the 1^{st} antigen binding moiety comprises the first V_{H} and a heavy chain constant region, and wherein the second antigen binding moiety is an scFv fragment; and (ii) a second polypeptide comprising a light chain of the 1S antigen binding moiety and the third antigen binding moiety, wherein the light chain comprises the first V_{L} and a light chain constant region, and wherein the third antigen binding moiety is an scFv fragment.

In some examples, the tri-specific antibody disclosed herein may comprise: (i) a first polypeptide comprising a heavy chain of the 1^{st} antigen binding moiety and the second antigen binding moiety, wherein the heavy chain of the 1^{st} antigen binding moiety comprises the first V_{H} and a first heavy chain constant region, and wherein the second antigen binding moiety is an scFv fragment; (ii) a second polypeptide comprising a heavy chain of the 1^{st} antigen binding moiety and the third antigen binding moiety, wherein the heavy chain comprises the first V_{H} and a second heavy chain constant region, and wherein the third antigen binding moiety is an scFv fragment; and (iii) a third polypeptide comprising a light chain of the first antigen binding moiety, the light chain comprising the first V_{L} and a light chain constant region. In some instances, the first and second heavy chain constant regions comprise mutations in the CH3 domains that enhances heterodimeration over homodimeration as relative to the wild-type counterpart.

In any of the multi-specific antibodies disclosed herein, the first and second heavy chain constant regions comprise the mutations, which are knob-in-hole mutations, charged mutations, or ZW1 mutations. Alternatively or in addition, one of the first and second heavy chain constant regions comprises a mutation that reduces protein A binding activity relative to the wild-type counterpart.

In some examples, the tri-specific antibody disclosed herein may bind to B7H3, CD3, and CD137. Examples include Ly1785, Ly1793, Ly1794, Ly1795, Ly1796, Ly1797, Ly1798, Ly1799, Ly1800, Ly1801, Ly1802, Ly1803, Ly1804, Ly1805, and Ly1849.

In some examples, the tri-specific antibody disclosed herein may bind to B7H3, CD3, and GITR. Examples include Ly1905, Ly1906, Ly1907, Ly1908, Ly1909, Ly1910, Ly1911, Ly1912, Ly1913, Ly1914, Ly1915, Ly1916, Ly1917, Ly1918, and Ly1933.

In some examples, the tri-specific antibody disclosed herein may bind to B7H3, CD3, and OX40. Examples include Ly1919, Ly1920, Ly1921, Ly1922, Ly1923, Ly1924, Ly1925, Ly1926, Ly1927, Ly1928, Ly1929, Ly1930, Ly1931, Ly1932, and Ly1934.

In some examples, the tri-specific antibody disclosed herein may bind to B7H3, CD137, and OX40. Examples include Ly2076, Ly2077, and Ly2078.

In some examples, the tri-specific antibody disclosed herein may bind to B7H3, CD137, and GITR. Examples include Ly2079, Ly2080, and Ly2081.

In some examples, the tri-specific antibody disclosed herein may bind to B7H3, CD3, and CD28. Examples include any one of Ly1968 to Ly2042.

In another aspect, the present disclosure features a humanized antibody specific to human B7H3. The humanized antibody comprises a heavy chain variable region (V_{H}) and a light chain variable region (V_{L}). In some examples, the V_{H} Comprises a framework of IGHV1-46*01 and heavy chain complementary determining regions (CDRs) 1, 2, and 3, which are either identical to those of parent murine antibody Ly383 or collectively contain no more than 5 amino acid residue variations relative to the parent murine antibody Ly383. In other examples, the V_{H} comprises a framework of IGHV1-2*02 and heavy chain CDRs 1, 2, and 3, which are either identical to those of parent murine antibody Ly387 or collectively contain no more than 5 amino acid residue variations relative to the parent murine antibody Ly387.

Alternatively or in addition, the V_{L} comprises a framework of IGKV3-11*01 and light chain CDRs 1, 2, and 3, which are either identical to those of the parent murine antibody Ly383 or Ly387 or collectively contain no more than 5 amino acid residue variations relative to the parent murine antibody Ly383 or Ly387.

In some examples, the antibody comprises the same heavy chain CDRs 1, 2, and 3 as antibody Ly383, and/or the same light chain CDRs 1, 2, and 3 of antibody Ly383. In some examples, the antibody comprises the same heavy chain CDRs 1, 2, and 3 as antibody Ly387, and/or the same light chain CDRs 1, 2, and 3 of antibody Ly387.

Any of the humanized antibodies disclosed herein may comprise a V_{H} that comprises one or more mutations in the V_{H} framework. The mutations in the V_{H} framework can be back mutations based on amino acid residues in the parent murine antibody at corresponding positions.

Any of the anti-B7H3 antibodies listed in Table 2 is within the scope of the present disclosure. In specific examples, the V_{H} comprises the amino acid sequence of SEQ ID NO: 35, 39, 47 or 49; and/or wherein the V_{L} comprises the amino acid sequence of SEQ ID NO: 37 or 41. Alternatively, the V_{H} comprises the amino acid sequence of SEQ ID NO: 43; and/or wherein the V_{L} comprises the amino acid sequence of SEQ ID NO: 45.

Any of the humanized antibodies disclosed herein may be a full-length antibody. In some examples, the full-length antibody is an IgG/kappa molecule. In some specific examples, the full-length antibody comprises a heavy chain that is an IgG1, IgG2, or IgG4 chain. In some instances, the heavy chain comprises a mutated Fc region, which exhibits altered binding affinity or selectivity to an Fc receptor as relative to the wild-type counterpart. Exemplary humanized anti-B7H3 antibodies include Ly1426, Ly1562, Ly1612, Ly1614, Ly1616, Ly1618, and Ly1442.

The present disclosure also features a pharmaceutical composition comprising any of the multi-specific antibodies or humanized anti-B7H3 antibodies disclosed herein and a pharmaceutically acceptable carrier.

In another aspect, the present disclosure features a nucleic acid or a nucleic acid set, which collectively encodes an antibody of any one of the preceding claims. In some instances, the nucleic acid or nucleic acid set can be an expression vector or an expression vector set.

Further, the present disclosure features a host cell, comprising the nucleic acid or nucleic acid set disclosed herein. In some examples, the host cell is a mammalian host cell.

In addition, the present disclosure features a method for producing any of the multi-specific antibodies or the humanized anti-B7H3 antibodies disclosed herein. The method may comprise: (i) culturing any of the host cells disclosed herein under conditions allowing for expression of the antibody; and (ii) harvesting the antibody thus produced.

In other aspects, the present disclosure also features a method for modulating immune responses, comprising administering an effective amount of any of the multi-specific and/or humanized anti-B7H3 antibodies, or the pharmaceutical composition comprising such, to a subject in need thereof. In some examples, the subject is a human patient having or suspected of having cancer.

Also within the scope of the present disclosure are any of the multi-specific and/or humanized anti-B7H3 antibodies for use in modulating immune responses or treating cancer, as well as using such antibodies for manufacturing a medicament for treatment of the target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1D are charts showing binding activity of anti-B7H3 antibodies as indicated to human B7H3 expressed on CHO cells. Binding of these anti-B7H3 antibodies are indicated by the mean fluorescence intensity (MFI). FIG. 1A: Clones Ly1426 and Ly383 at the concentrations as indicated. FIG. 1B: Clones Ly1562 and Ly383 at the concentrations as indicated. FIG. 1C: Clones Ly1442 and Ly387 at the concentrations as indicated. FIG. 1D: Clones Ly1612, Ly1614, Ly1616 and Ly1618 at the concentrations as indicated.

FIGS. 2A-2D are charts showing B7H3 or CD40 binding activity of anti-B7H3/CD40 bispecific antibodies as indicated to human B7H3 expressed on CHO cells. Binding of these anti-B7H3/CD40 bispecific antibodies are indicated by the mean fluorescence intensity (MFI).

FIG. 2A: B7H3 binding activity of Clones Ly1581, Ly1660, Ly1661, Ly1662, Ly1663, and Ly383 at the concentrations as indicated. FIG. 2B: B7H3 binding activity of Clones Ly1578, Ly1579, Ly1581, Ly1583, Ly1585, and Ly383 at the concentrations as indicated. FIG. 2C: CD40 binding activity of Clones Ly1581, Ly1660, Ly1661, Ly1662, Ly1663, and Ly253-G2 (CD40 Ab1) at the concentrations as indicated. FIG. 2D: CD40 binding activity of Clones Ly1578, Ly1579, Ly1581, Ly1583, Ly1585, and Ly253-G2 (CD40 Ab1) at the concentrations as indicated.

FIGS. 3A-3F are charts showing stimulation of human CD40 activation as indicated by IL8 secretion in a reporter assay by a number of anti-B7H3/CD40 antibodies. The agonistic activity of these bispecific antibodies was evaluated either without or with co-cultured B7H4 overexpressing CHO cells. The various antibodies are indicated on the x-axis, and the CD40 activation signal are indicated on the y-axis. FIG. 3A: Clones Ly1578, Ly1579, Ly1581, Ly1583, Ly253-G2, Ly383 and Ly387 at the concentrations as indicated for activating CD40 without co-cultured B7H3 overexpressing CHO cells. FIG. 3B: Clones Ly1578, Ly1579, Ly1581, Ly1583, Ly253-G2, Ly383 and Ly387 at the various concentrations as indicated for activating CD40 with co-cultured B7H3 overexpressing CHO cells. FIG. 3C: Clones Ly1585, Ly1587, Ly253-G2, Ly383 and Ly387 at the various concentrations as indicated for activating CD40 without co-cultured B7H3 overexpressing CHO cells. FIG. 3D: Clones Ly1585, Ly1587, Ly253-G2, Ly383 and Ly387 at the various concentrations as indicated for activating CD40 with co-cultured B7H3 overexpressing CHO cells. FIG. 3E: Clones Ly1581, Ly1660, Ly1661, Ly1662, Ly1663, Ly1679, Ly253-G2, Ly1612 and Ly383 at the various concentrations as indicated for activating CD40 without co-cultured B7H3 overexpressing CHO cells. FIG. 3F: Clones Ly1581, Ly1660, Ly1661, Ly1662, Ly1663, Ly1679, Ly253-G2, Ly1612 and Ly383 at the various concentrations as indicated for activating CD40 with co-cultured B7H3 overexpressing CHO cells.

FIGS. 4A-4C are a set of graphs showing the anti-tumor activity of anti-B7H3/CD40 antibodies in a human CD40 knock-in mouse syngeneic model with human B7H3-expressing tumor cells. FIG. 4A: anti-tumor effects in MC38-hB7H3 model of clones Ly1581, Ly1585, Ly1579, and Ly253-G2 at 10 mg/kg administered on day 0, 20 and 27 by intraperitoneal injection. FIG. 4B: anti-tumor effects in MC38-hB7H3 model of clones Ly1581, Ly1662, Ly1663, and Ly253-G2 at 10 mg/kg as shown administered on day 0, 20 and 27 by intraperitoneal injection. FIG. 4C: Clones Ly1581, Ly1585, Ly1579, and Ly253-G2 at 10 mg/kg administered on day 0, 20 and 27 by intraperitoneal injection.

FIGS. 5A-5C include diagrams showing representative bispecific antibody 2-chain formats. FIG. 5A: 2^{nd} antibody in scFv format fused to the C-terminus of the heavy chain of the 1^{st} antibody. FIG. 5B: 2^{nd} antibody in scFv format fused to the N-terminus of the heavy chain of the 1S antibody. FIG. 5C: 2^{nd} antibody in scFv format fused to the light chain of the 1^{st} antibody.

FIGS. 6A-6D include diagrams showing representative bispecific antibody 3-chain formats. FIG. 6A: 2^{nd} antibody in scFv format fused to a heavy chain of the 1^{st} antibody, in the middle of CH1 and Fc regions. FIG. 6B: 2^{nd} antibody in scFv format fused to the C-terminus of the Fc region of the 1^{st} antibody. FIG. 6C: V_{H} and V_{L} of 2^{nd} antibody each fused with a heavy chain of the 1^{st} antibody, between CH1 and CH3 regions. FIG. 6D: V_{H} and V_{L} of 2^{nd} antibody each fused with a heavy chain of the 1^{st} antibody, in middle of CH1 and Fc regions. In each of these representative formats, the Fc regions may contain mutations enhancing heterodimerization (e.g., KiH, Charge, or ZW) and/or reducing binding affinity to protein A (pA mut).

FIGS. 7A-7D include diagrams showing representative bispecific antibody 4-chain formats. FIG. 7A: 1^{st} and 2^{nd} antibodies in heterodimer format with knob-in-hole (KiH) and pA mut mutations. FIG. 7B: 1^{st} and 2^{nd} antibodies in heterodimer format with Charge and pA mut mutations. FIG. 7C: 1^{st} and 2^{nd} antibodies in heterodimer format with ZW1/ZW2 and pA mut mutations. FIG. 7D: 2^{nd} antibody in Fab format with one chain fused to one heavy chain of the 1^{st} antibody (between CH1 and Fc). In any of these formats, the V_{H} and V_{L} fragments of the 2^{nd} antibody may be in CrossMab form.

FIGS. 8A-8B include diagrams showing representative bispecific antibodies using TCRα and TCRβ fragments. FIG. 8A: V_{H} and V_{L} of 2^{nd} antibody each fused to one of a TCRα and a TCRβ fragment for dimerization. The V_{H} chain is further fused to an Fc region, which form a dimer with the heavy chain of the 1S antibody. FIG. 8B: V_{H} and V_{L} of 2^{nd} antibody each fused to one of a TCRα and a TCRβ fragment for dimerization. One of the two chains is further fused to a heavy chain of the 1^{st} antibody (between CH1 and Fc regions).

FIGS. 9A-9D include diagrams showing representative tri-specific antibody formats (4-chain). FIG. 9A: 2^{nd} antibody in Fab form and 3^{rd} antibody in scFv form. One scFv chain is fused with one heavy chain of the 1^{st} antibody and one scFv chain is fused with another heavy chain of the 1^{st} antibody, which further includes one of the Fab chain. FIG. 9B: 2^{nd} antibody in Fab form and 3^{rd} antibody in scFv form. One scFv chain is fused with a heavy chain of the 1^{st} antibody, which further includes one of the Fab chain. FIG. 9C: 2^{nd} antibody in Fab form and 3^{rd} antibody in scFv form. One scFv chain is fused with a heavy chain of the 1^{st} antibody and one Fab chain is fused with another heavy chain of the 1^{st} antibody. FIG. 9D: 2^{nd} antibody in Fab form and 3^{rd} antibody in V_{H}/V_{L} format (separate chains). One of the V_{H} and V_{L} fragments is fused with one heavy chain of the 1^{st} antibody and the other one is fused with another heavy chain of the 1^{st} antibody, which further includes one of the Fab chain. In any of these formats, the V_{H} and V_{L} fragments of the 2^{nd} antibody may be in CrossMab form. In each of these representative formats, the Fc regions may contain mutations enhancing heterodimerization (e.g., KiH, Charge, or ZW) and/or reducing binding affinity to protein A (pA mut).

FIGS. 10A-10C include diagrams showing representative tri-specific antibody formats (3-chain). FIG. 10A: 2^{nd} and 3^{rd} antibodies both in scFv form, one being fused with a heavy chain of the 1^{st} antibody and the other being fused with the other heavy chain of the 1^{st} antibody (between CH1 and Fc). FIG. 10B: 2^{nd} and 3^{rd} antibodies both in scFv form, one being fused with a heavy chain of the 1^{st} antibody and the other being fused with the other heavy chain of the 1^{st} antibody (between CH1 and Fc), which further includes a copy of the 3^{rd} antibody scFv. FIG. 10C: 2^{nd} and 3^{rd} antibodies both in scFv form, both being fused with a heavy chain of the 1^{st} antibody (one between CH1 and Fc and the other one at the C-terminus). In each of these representative formats, the Fc regions may contain mutations enhancing heterodimerization (e.g., KiH, Charge, or ZW) and/or reducing binding affinity to protein A (pA mut).

FIGS. 11A-11E include diagrams showing representative tri-specific antibody formats (3-chain). FIG. 11A: 2^{nd} antibody in V_{H}/V_{L} form (separate chains) and 3^{rd} antibody in scFv form. Each of the V_{H} and V_{L} fragments is fused with one heavy chain of the 1^{st} antibody, which further includes a scFv chain. FIGS. 11B and 11C: 2^{nd} antibody in V_{H}/V_{L} form (separate chains) and 3^{rd} antibody in scFv form. Each of the V_{H} and V_{L} fragments is fused with one heavy chain of the 1^{st} antibody. One of the heavy chains further includes a scFv chain. In each of these representative formats, the Fc regions may contain mutations enhancing heterodimerization (e.g., KiH, Charge, or ZW) and/or reducing binding affinity to protein A (pA mut). FIG. 11D: both 2^{nd} antibody and 3^{rd} antibody in V_{H}/V_{L} form (separate chains), each fused with one heavy chain of the 1S antibody. FIG. 11E: 2^{nd} antibody in V_{H}/V_{L} form (separate chains) and 3^{rd} antibody in scFv form. Each of V_{H} and V_{L} fragments is fused with one heavy chain of the 1^{st} antibody and a scFv chain is fused with each of the light chains of the 1^{st} antibody.

FIGS. 12A-12D include diagrams showing representative tri-specific antibody formats (3-chain or 4-chain). FIG. 12A: both 2^{nd} and 3^{rd} antibodies are in scFv form, each being fused with one heavy chain of the 1^{st} antibody. FIG. 12B: both 2^{nd} and 3^{rd} antibodies are in Fab form, one chain of each Fab is fused with one chain of the other Fab. FIG. 12C: 2^{nd} antibody in V_{H}/V_{L} form (separate chains) and 3^{rd} antibodies in Fab form. One of the V_{H} and V_{L} is fused with one heavy chain of the 1^{st} antibody and the other is fused with one chain of the Fab. FIG. 12D: 2^{nd} antibody in V_{H}/V_{L} form (separate chains) and 3^{rd} antibodies in Fab form. One of the V_{H} and V_{L} is fused with one heavy chain of the 1^{st} antibody and the other is fused with one chain of the Fab. The fusion chain further includes a V_{H}-CH1 fragment of the 1 antibody. In any of these formats, the V_{H} and V_{L} fragments of the 2^{nd} antibody may be in CrossMab form. In each of these representative formats, the Fc regions may contain mutations enhancing heterodimerization (e.g., KiH, Charge, or ZW) and/or reducing binding affinity to protein A (pA mut).

FIGS. 13A-13C include diagrams showing representative tri-specific antibody formats (2-chain or 3-chain format). FIGS. 13A-13B: both 2^{nd} and 3^{rd} antibodies are in scFv format, eaching being fused with the heavy chains and the light chains of the 1V antibody. FIG. 13C: both 2^{nd} and 3^{rd} antibodies are in scFv format, each being fused with one heavy chain of the 1^{st} antibody.

### DETAILED DESCRIPTION OF THE INVENTION

Provided herein are antibodies (e.g., humanized antibodies) specific to human B7H3 (i.e., anti-B7H3 antibodies). Also provided herein are multi-specific antibodies (e.g., bi-specific and tri-specific antibodies) comprising a first antigen binding moiety specific to B7H3 and one or more (e.g., a second and optionally a third) antigen binding moieties specific to an immune modulator, for example, CD40, CD137, GITR, OX40, CD47, CD3, or CD28. Such anti-B7H3 antibodies and multi-specific antibodies have various therapeutic, diagnostic, or research applications. For example, the antibodies may be used in modulating immune responses, such as anti-tumor immune responses, in subjects in need of such treatment. Such antibodies may also be used in cancer treatment or cancer diagnosis.

As used herein, an antibody (interchangeably used in plural form) refers to an immunoglobulin molecule capable of specific binding to a target, e.g., any of the target antigens disclosed herein, through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

The antibodies described herein can be murine, rat, human, or any other origin (including chimeric or humanized antibodies). Such antibodies are non-naturally occurring, i.e., would not be produced in an animal without human act (e.g., immunizing such an animal with a desired antigen or fragment thereof or isolated from antibody libraries).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

### I. Humanized Anti-B7H3 Antibodies

In some aspects, the present disclosure provides antibodies specific to a glucocorticoid induced TNFR-related (B7H3) polypeptide (“anti-B7H3 antibodies), which may be of any source, for example, human and/or monkey B7H3. Such anti-B7H3 antibodies may specifically bind B7H3 of a particular species (e.g., human B7H3). Alternatively, the anti-B7H3 antibodies described herein may cross-react with B7H3 antigens of different species (e.g., binding to both human and monkey B7H3). In some instances, the anti-B7H3 antibodies described herein can bind cell surface B7H3, for example, B7H3 expressed on cells (e.g., immune cells) that naturally express B7H3 on the surface.

B7H3, also known as CD276, is expressed on immune cells (such as antigen-presenting cells or macrophages) and tumor cells and has inhibitory roles on T cells, contributing to tumor cell immune evasion. Recent studies have shown that B7H3 is a crucial player in tumor growth and metastasis beyond the immune regulatory roles. Inhibition of B7H3 is a potential therapeutic strategy for B7H3 overexpressing tumors. B7H3 is a protein well known in the art. For example, the structural information of human B7H3 can be find under Gene ID: 80381.

In some embodiments, the anti-B7H3 antibodies disclosed herein are humanized antibodies derived from a non-human parent antibody clone, for example, a murine antibody binding to B7H3 such as human B7H3. Humanized antibodies refer to forms of non-human (e.g., murine) antibodies that are specific chimeric immunoglobulins, immunoglobulin chains, or antigen-binding fragments thereof that contain minimal sequence derived from the non-human immunoglobulin parent. For the most part, humanized antibodies are human immunoglobulins (recipient antibody), in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence.

The humanized antibody may also comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody. This is also termed one or more CDRs “derived from” one or more CDRs from the original antibody. Humanized antibodies may also involve affinity maturation.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

In some embodiments, the anti-B7H3 antibodies disclosed herein are humanized antibodies derived from murine parent clone Ly383, which are disclosed in Example 1 below. Such a humanized antibody may comprise a heavy chain framework of IGHV1-2*02 and/or a light chain framework of IGKV3-11*01. In addition, such a humanized antibody may comprise the same heavy chain and/or light chain complementary determining regions (CDRs) as the murine parent clone. Alternatively, the humanized anti-B7H3 antibodies, which may comprise the heavy chain framework of IGHV1-2*02 and/or a light chain framework of IGKV3-11*01, may comprise one or more amino acid residue variations in one or more CDR regions as relative to the corresponding CDR regions of the murine parent Ly383. For example, the humanized antibody may comprise up to 5 (e.g., up to 4, 3, 2, or 1) amino acid residues in the three heavy chain CDRs collectively. In other examples, the humanized antibody may comprise up to 5 (e.g., up to 4, 3, 2, or 1) amino acid residues in the three light chain CDRs collectively. In yet other examples, the humanized antibody may comprise up to 8 (e.g., up to 7, 6, 5, 4, 3, 2, or 1) amino acid residues in the three heavy chain CDRs and the three light chain CDRs collectively.

In some embodiments, the anti-B7H3 antibodies disclosed herein are humanized antibodies derived from murine parent clone Ly387, which are disclosed in Example 1 below. Such a humanized antibody may comprise a heavy chain framework of IGHV4-59*01 and/or a light chain framework of IGKV3-11*01. In addition, such a humanized antibody may comprise the same heavy chain and/or light chain complementary determining regions (CDRs) as the murine parent clone. Alternatively, the humanized anti-B7H3 antibodies, which may comprise the heavy chain framework of IGHV1-2*02 and/or a light chain framework of IGKV3-11*01, may comprise one or more amino acid residue variations in one or more CDR regions as relative to the corresponding CDR regions of the murine parent Lyv396. For example, the humanized antibody may comprise up to 5 (e.g., up to 4, 3, 2, or 1) amino acid residues in the three heavy chain CDRs collectively. In other examples, the humanized antibody may comprise up to 5 (e.g., up to 4, 3, 2, or 1) amino acid residues in the three light chain CDRs collectively. In yet other examples, the humanized antibody may comprise up to 8 (e.g., up to 7, 6, 5, 4, 3, 2, or 1) amino acid residues in the three heavy chain CDRs and the three light chain CDRs collectively.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, any of the humanized anti-B7H3 antibodies may comprise the same framework as those encoded by the human acceptor germline V_{H} and/or V_{L} gene. In other embodiments, the framework region of the humanized antibodies may comprise one or more mutations relative to those encoded by the human acceptor germline V_{H} and/or V_{L} gene. For example, one or more positions in the framework region of the V_{H} and/or V_{L} chain of a humanized antibody may contain one or more back mutations, which refer to changing a residue in the human acceptor germline gene back to the residue at the corresponding position of the murine parent. For example, humanized antibodies derived from murine parent clone Ly383 may comprise mutations (e.g., back mutations) at one or more of positions A40 (e.g., A40K), M48 (e.g., M48I), V68 (e.g., V68A), R72 (e.g., R72S), and/or T74 (e.g., T74K) in the heavy chain framework regions, and mutations (e.g., back mutations) at one or more of positions P45 (e.g., P45L), W46 (e.g., W46L), R65 (e.g., R65G), and/or Y70 (e.g., Y70F) in the heavy chain framework regions. In some examples, the humanized anti-B7H3 antibodies disclosed herein may comprise any of the heavy chain and light chain CDRs disclosed herein (e.g., any of the CDR combinations provided in Example 3 below). In addition, such a humanized anti-B7H3 antibody may comprise a heavy chain framework at least 80% (e.g., at least 85%, 90%, 95% or above) identical to the heavy chain framework region of IGHV1-2*02. Alternatively or in addition, the humanized anti-B7H3 antibody may comprise a light chain framework at least 80% (e.g., at least 85%, 90%, 95% or above) identical to the light chain framework region of IGKV3-11*01.

Any of the anti-B7H3 antibodies described herein may be a full-length antibody, which contains two heavy chains and two light chains, each including a variable domain and a constant domain. Alternatively, the heavy chain constant region of the antibodies described herein may comprise a single domain (e.g., CH1, CH2, or CH3) or a combination of any of the single domains. Antibody heavy and light chain constant regions are well known in the art, e.g., those provided in the IMGT database (www.imgt.org) or at www.vbase2.org/vbstat.php., both of which are incorporated by reference herein.

Alternatively, the antibodies disclosed herein can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

In some embodiments, the anti-B7H3 antibody is Ly383 disclosed in Example 1 below or a functional variant derived therefrom. Ly383 or a functional variant thereof may comprise V_{H} and V_{L} chains fused to a human heavy chain constant region and a human light chain constant region, respectively. The human heavy chain constant region may be from an IgG molecule and/or the human light chain constant region may be from a kappa chain. The heavy chain constant domain may be derived from a suitable Ig isoform, for example, a human IgG1, IgG2, or IgG4 molecule. In some embodiments, the constant domain may comprise one or more mutations in the Fc region to enhance or reduce binding affinity and/or binding specificity to an Fc receptor. Examples are provided herein or disclosed in WO/2018/183520 and PCT/US2019/053505 (filed on Sep. 27, 2019), the relevant disclosures of each of which are incorporated by reference for the purpose and subject matter referenced herein. Such a recombinant antibody may further comprise the same light chain variable region of TM676 fused to a human light chain constant region, for example, a kappa chain constant region.

In some embodiments, the anti-B7H3 antibody is Ly387 disclosed in Example 1 below or a functional variant derived therefrom. Ly387 or a functional variant thereof may comprise V_{H} and V_{L} chains fused to a human heavy chain constant region and a human light chain constant region, respectively. The human heavy chain constant region may be from an IgG molecule and/or the human light chain constant region may be from a kappa chain. The heavy chain constant domain may be derived from a suitable Ig isoform, for example, a human IgG1, IgG2, or IgG4 molecule. In some embodiments, the constant domain may comprise one or more mutations in the Fc region to enhance or reduce binding affinity and/or binding specificity to an Fc receptor. Examples are provided herein or disclosed in WO/2018/183520 and PCT/US2019/053505 (filed on Sep. 27, 2019), the relevant disclosures of each of which are incorporated by reference for the purpose and subject matter referenced herein. Such a recombinant antibody may further comprise the same light chain variable region of TM677 fused to a human light chain constant region, for example, a kappa chain constant region.

Exemplary anti-B7H3 antibodies and humanized versions thereof are provided in Example 1 and Table 2 below, which are also within the scope of the present disclosure.

### II. Multi-Specific Antibodies Comprising Anti-B7H3 Binding Moieties

In some aspects, the present disclosure also provides multi-specific antibodies comprising one antigen binding moiety specific to B7H3 and one or more additional antigen binding moieties specific to one or more additional antigens of interest, for example, an immune checkpoint or modulator molecule. Examples include, but are not limited to CD40, CD137, GITR, OX40, CD47, CD3, or CD28. In some examples, the multi-specific antibody discloses herein is a bi-specific antibody comprising one antigen binding moiety specific to B7H3 and one antigen binding moiety specific to one of antigens CD40, CD137, GITR, OX40, CD47, CD3, or CD28. In other examples, the multi-specific antibody disclosed herein is a tri-specific antibody comprising one antigen binding moiety specific to B7H3 and two additional antigen binding moieties specific to two antigens selected from CD40, CD137, GITR, OX40, CD47, CD3, or CD28.

### A. Antigen-Binding Moieties

Each antigen binding moiety in any of the multi-specific antibodies disclosed herein can be an antigen binding moiety in any form, including, but not limited to, intact (i.e., full-length) antibodies, antigen-binding fragments thereof (such as Fab, Fab′, F(ab′).sub.2, Fv, tribody, triFabs, tandem linked Fabs, a Fab-Fv, tandem linked V domains, tandem linked scFvs, and among other formats), single chain antibodies (scFv antibodies), and tetravalent antibodies. Any scFv fragment in a bi-specific or multi-specific antibody may be in V_{H}→V_{L} orientation. Alternatively, it can be in the V_{L}→V_{H} orientation.

An antigen binding moiety in any of the multi-specific antibodies disclosed herein may specifically bind to the corresponding target antigen(s) (e.g., B7H3, CD40, CD137, GITR, OX40, CD47, CD3, or CD28) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (e.g., those listed above) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second or third target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method). Alternatively, or in addition, the antibodies described herein may specifically binds the human antigen or a fragment thereof as relative to the monkey counterpart, or vice versa (e.g., having a binding affinity at least 10-fold higher to one antigen than the other as determined in the same assay under the same assay conditions). In other instances, the antibodies described herein may cross-react to human and a non-human antigen (e.g., monkey), e.g., the difference in binding affinity to the human and the non-human antigen is less than 5-fold, e.g., less than 2-fold, or substantially similar.

In some embodiments, an antigen binding moiety in any of the bi-specific or multi-specific antibodies as described herein has a suitable binding affinity for the target antigen(s) (e.g., B7H3, CD40, CD137, GITR, OX40, CD47, CD3, or CD28) or antigenic epitopes thereof. As used herein, “binding affinity” refers to the apparent association constant or K_{A}. The K_{A} is the reciprocal of the dissociation constant (K_{D}). The antibody described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The antigen binding moieties of a multi-specific antibody as disclosed herein may be derived from the parent antibody specific to B7H3 and the parent antibodies specific to the other antigens of interest listed in Table 1 below (heavy chain and light chain CDRs based on the Kabat scheme are identified in boldface).

TABLE 1
Parent Antibodies for Constructing Multi-Specific Antibodies

| Antibody | | | SED ID |
|---|---|---|---|
| Clones | | Amino acid sequence | NO: |
| B7H3 | VH | QVQLVQSGAEVKKPGASVKVSCKASGYTFTSYVMHWVRQKPGQGLEWIGYINP | 1 |
| Ab1 | | YNEGTESTDKFKGRATMTSDKSTSTVYMELSSLRSEDTAVYYCASIYYGYE | |
| | | GTYFGVWGQGTLVTVSS | |
| | VL | EIVLTQSPATLSLSPGERATLSCRASSSVSYMHWYQQKPGQAPRPWIYATSN | 2 |
| | | LASGIPARFSGSRSGTDYTLTISSLEPEDFAVYYCQQWSSNTLTFGGGTKVEI | |
| | | K | |
| B7H3 | VH | QVQLVQSGAEVKKPGASVKVSCKASGYTFTSYWMHWVRQAPGQGLEWMGMIHP | 3 |
| Ab2 | | NSGGTNYNEKFKGRVTMTRDTSISTAYMELSRLRSDDTAVYYCARSQATWF | |
| | | AYWGQGTLVTVSS | |
| | VL | EIVLTQSPATLSLSPGERATLSCRASSSVSSSYLHWYQQKPGQAPRLLIYST | 4 |
| | | SNLASGIPARFSGSGSGTDFTLTISSLEPEDFAVYYCQHYSGYPLTFGGGTKV | |
| | | EIK | |
| CD40 | VH | QVQLVQSGAEVKKPGASVKVSCKASGYTFTGYYMHWVRQAPGQGLEWMGWINP | 5 |
| Ab1 | | DSGGTNYAQKFQGRVTMTRDTSISTAYMELNRLRSDDTAVYYCARDQPLGY | |
| | | CTNGVCSYFDYWGQGTLVTVSS | |
| | VL | DIQMTQSPSSVSASVGDRVTITCRASQGIYSWLAWYQQKPGKAPNLLIYTAS | 6 |
| | | TLQSGVPSRFSGSGSGTDFTLTISSLQPEDFATYYCQQANIFPLTFGGGTKVE | |
| | | IK | |
| CD137 | VH | QVQLVQSGAEVKKPGASVKVSCKASGYTFAGFEMHWVRQAPGQGLEWMGAIDP | 7 |
| Ab1 | | KTGGTDYNQKFKDRVTMTRDTSISTAYMELSRLRSDDTAVYYCARDLGYFD | |
| | | VWGQGTLVTVSS | |
| | VL | DIQMTQSPSSLSASVGDRVTITCRASQDIRSNLNWYQQKPGGAVKLLIYYTS | 8 |
| | | RLHSGVPSRFSGSGSGTDYTLTISSLQPEDFATYFCQQSEKLPRTFGGGTKVE | |
| | | IR | |
| GITR | VH | QVQLQESGPGLVKPSETLSLTCTVSGFSLTSYNVHWIRQPPGKGLEWIGVIWS | 9 |
| Ab1 | | GVRTDYNSVLKPRVTISVDTSKNQFSLKLSSVTAADTAVYYCARGTYDDNY | |
| | | HDVMDAWGQGTLVTVSS | |
| | VL | EIVLTQSPATLSLSPGERATLSCRASKSVRTGMHWYQQKPGQAPRLLIYGAS | 10 |
| | | NLESGIPARFSGSGSGTDFTLTISSLEPEDFAVYYCQQSWNHFTFGQGTKLEI | |
| | | K | |
| OX40 | VH | EVQLVQSGAEVKKPGASVKVSCKASGYTFTDSYMSWVRQAPGQGLEWIGDMYP | 11 |
| Ab1 | | DNGDSSYNQKFRERVTITRDTSTSTAYLELSSLRSEDTAVYYCVLAPRWYF | |
| | | SVWGQGTLVTVSS | |
| | VL | DIQMTQSPSSLSASVGDRVTITCRASQDISNYLNWYQQKPGKAPKLLIYYTS | 12 |
| | | RLRSGVPSRFSGSGSGTDFTLTISSLQPEDFATYYCQQGHTLPPTFGQGTKVE | |
| | | IK | |
| CD28 | VH | QVQLVQSGAEVVKPGASVKVSCKASGYTFTSYYIHWVRQAPGQGLEWIGSIYP | 13 |
| Ab1 | | GNVNTNYAQKFQGRATLTVDTSISTAYMELSRLRSDDTAVYYCTRSHYGLD | |
| | | WNFDVWGKGTTVTVSS | |
| | VL | DIQMTQSPSSLSASVGDRVTITCQASQNIYVWLNWYQQKPGKAPKLLIYKAS | 14 |
| | | NLHTGVPSRFSGSGSGTDFTLTISSLQPEDIATYYCQQGQTYPYTFGQGTKLE | |
| | | IK | |
| CD28 | VH | QVQLVQSGAEVKKPGASVKVSCKASGYTFTSYYIHWVRQAPGQGLEWIGCIYP | 15 |
| Ab2 | | GNVNTNYNEKFKDRATLTVDTSISTAYMELSRLRSDDTAVYFCTRSHYGLD | |
| | | WNFDVWGQGTTVTVSS | |
| | VL | DIQMTQSPSSLSASVGDRVTITCHASQNIYVWLNWYQQKPGKAPKLLIYKAS | 16 |
| | | NLHTGVPSRFSGSGSGTDFTLTISSLQPEDFATYYCQQGQTYPYTFGGGTKVE | |
| | | IK | |
| CD28 | VH | QVQLQESGPGLVKPSETLSLTCTVSGGSISSYYWSWIRQPPGKGLEWIGYIYY | 17 |
| Ab3 | | SGITHYNPSLKSRVTISVDTSKIQFSLKLSSVTAADTAVYYCARWGVRRDY | |
| | | YYYGMDVWGQGTTVTVSS | |
| | VL | EIVLTQSPGTLSLSPGERATLSCRASQSVSSSYLAWYQQKPGQAPRLLIYGA | 18 |
| | | SSRATGIPDRFSGSGSGTDFTLTISRLEPEDFAVYYCQQYGSSPWTFGQGTKV | |
| | | EIK | |
| CD47 | VH | QVQLVQSGAEVKKPGASVKVSCKASGYTFTNYNMHWVRQAPGQRLEWMGTIYP | 19 |
| Ab1 | | GNDDTSYNQKFKDRVTITADTSASTAYMELSSLRSEDTAVYYCARGGYRAM | |
| | | DYWGQGTLVTVSS | |
| | VL | DIVMTQSPLSLPVTPGEPASISCRSSQSIVYSNGNTYLGWYLQKPGQSPQLL | 20 |
| | | IYKVSNRFSGVPDRFSGSGSGTDFTLKISRVEAEDVGVYYCFQGSHVPYTFG | |
| | | QGTKLEIK | |
| CD47 | VH | KVQLVESGGGLVKPGGSLRLSCAASGFTFSNAWMNWVRQAPGKGLEWVGRIKR | 21 |
| Ab2 | | KTDGETTDYAAPVKGRFSISRDDSKNTLYLQMNSLKTEDTAVYYCAGSNRA | |
| | | FDIWGQGTMVTVSA | |
| | VL | DIVMTQSPDSLAVSLGERATINCKSSQSVLYSSNNRNYLAWYQQKPGQPPKL | 22 |
| | | LINQASTRASGVPDRFSGSGSGTEFTLIISSLHAEDVAIYYCQQYYTPPLAF | |
| | | GGGTKLEIK | |
| CD3 | VH | EVQLVESGGGLVQPGGSLRLSCAASGFTFSTYAMNWVRQAPGKGLEWVGRIRS | 23 |
| Ab1 | | KYNNYATYYADSVKGRFTISRDDSKNSLYLQMNSLKTEDTAVYYCVRHGNF | |
| | | GNSYVSWFAYWGQGTLVTVSS | |
| | VL | QAVVTQEPSLTVSPGGTVTLTCRSSTGAVTTSNYANWVQQKPGQAPRGLIGG | 24 |
| | | TNKRAPWTPARFSGSLLGGKAALTITGAQAEDEADYYCALWYSNLWVFGGGT | |
| | | KLTVL | |
| CD3 | VH | QVQLVQSGAEVKKPGASVKVSCKASGYTFTRYTMHWVRQAPGQGLEWMGYINP | 25 |
| Ab2 | | SRGYTNYNQKFKDRVTMTRDTSTSTVYMELSSLRSEDTAVYYCARYYDDHY | |
| | | CLDYWGQGTLVTVSS | |
| | VL | DIQMTQSPSTLSASVGDRVTITCSASSSVSYMNWYQQKPGKAPKLLIYDTSK | 26 |
| | | LASGVPSRFSGSGSGTEFTLTISSLQPDDFATYYCQQWSSNPFTFGQGTKLEI | |
| | | K | |

As used herein, an antigen binding moiety in a multi-specific antibody “derived from” a parent antibody means that the parent antibody is used as a starting material for making one antigen binding moiety in the multi-specific antibody. The antigen binding moiety may comprise the same heavy chain and/or light chain CDRs as those of the parent antibody. Two antibodies having the same V_{H} and/or V_{L} CDRs means that their CDRs are identical when determined by the same approach (e.g., the Kabat definition, the Chothia definition, the AbM definition, and/or the contact definition as known in the art).

In some instances, an antigen binding moiety derived from a parent antibody may be a functional variant of the parent antibody. Such functional variants are substantially similar to the reference antibody, both structurally and functionally. A functional variant comprises substantially the same V_{H} and V_{L} CDRs as the reference antibody. For example, it may comprise only up to 5 (e.g., 4, 3, 2, or 1) amino acid residue variations in the total heavy chain CDR regions of the reference antibody and/or comprise only up to 5 (e.g., 4, 3, 2, or 1) amino acid residue variations in the total light chain CDR regions of the reference antibody. In some examples, the functional variant may comprise up to 8 (e.g., 7, 6, 5, 4, 3, 2, or 1) amino acid residue variations in the total heavy and light chain CDRs relative to those of the reference antibody. Such functional variants may bind the same epitope of B7H3 with substantially similar affinity (e.g., having a K_{D} value in the same order). Alternatively or in addition, the amino acid residue variations are conservative amino acid residue substitutions as disclosed herein.

In some embodiments, an antigen binding moiety in a multi-specific antibody as disclosed herein may comprise heavy chain CDRs that are at least 80% (e.g., 85%, 90%, 95%, or 98%) sequence identity, individually or collectively, as compared with the V_{H} CDRs of the corresponding parent antibody. Alternatively or in addition, the antigen binding moiety may comprise light chain CDRs that are at least 80% (e.g., 85%, 90%, 95%, or 98%) sequence identity, individually or collectively, as compared with the V_{L} CDRs as the parent antibody.

In other embodiments, the antigen binding moiety may comprise heavy chain CDRs that are at least 80% (e.g., 85%, 90%, 95%, or 98%) sequence identity, individually or collectively, as compared with the V_{H} CDRs of the corresponding parent antibody. Alternatively or in addition, the antigen binding moiety may comprise light chain CDRs that are at least 80% (e.g., 85%, 90%, 95%, or 98%) sequence identity, individually or collectively, as compared with the V_{L} CDRs as the parent antibody.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the multi-specific antibodies disclosed herein may be trivalent, tetravalent, pentavelnt, or hexavalent, which comprises one or two binding sites for B7H3 and the other antigen(s) (e.g., CD40, CD137, GITR, OX40, CD47, CD3, or CD28).

### B. Exemplary Formats for Multi-Specific Antibodies

Any of the multi-specific antibodies disclosed herein (e.g., bi-specific or tri-specific antibodies) may be in any bi-specific or multi-specific antibody format known in the art, for example, BsIgG, BsAb fragment, Bispecific fusion proteins, or BsAb conjugate. See, e.g., Mol. Immunol. 67(2):95-106 (2015), Trispecific IgG, Trispecific Ab fragment, Trispecific fusion proteins, or TsAb conjugate. See, e.g., Methods, Volume 154, 1 Feb. 2019, Pages 3-9.

### (i) Bi-Specific Antibodies

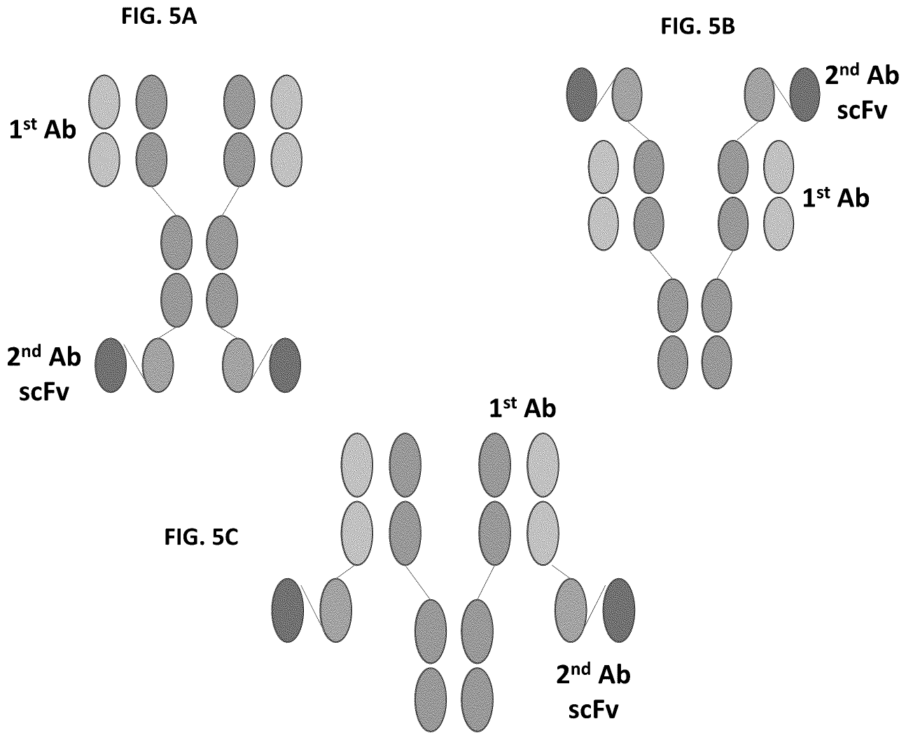

In some embodiments, the multi-specific antibodies disclosed herein are bi-specific antibodies comprising one antigen binding moiety specific to B7H3 and another antigen binding moiety specific to CD40, CD137, GITR, OX40, CD47, CD3, or CD28. Such a bi-specific antibody may be of any format known in the art. Exemplary bi-specific antibody formats are illustrated in FIGS. 5A-5C, FIGS. 6A-6D, FIGS. 7A-7D, and FIGS. 8A-8B, all of which are within the scope of the present disclosure.

In some examples, one antigen binding moiety in the bi-specific antibody (e.g., the anti-B7H3 moiety) is in a multi-chain antibody format as disclosed herein, and the other antigen binding moiety (e.g., specific to any of the other antigens of interest) can be in an scFv format. For example, the multi-chain antibody format comprises a light chain that comprises a V_{L} domain and a light chain constant region, and a heavy chain that comprises a V_{H} and a heavy chain constant domain or a fragment thereof (which optionally may comprise the CH3 domain).

In other examples, both antigen binding moieties in the bi-specific antibody may be in a a multi-chain antibody format as disclosed herein. For example, one antigen binding moiety (e.g., the anti-B7H3 moiety) may comprises a light chain that comprises a V_{L} domain and a light chain constant region, and a heavy chain that comprises a V_{H} and a heavy chain constant domain or a fragment thereof (which optionally may comprise the CH3 domain). The other antigen binding moiety (e.g., specific to the other antigen) may comprise a V_{H} fragment and a V_{L} fragment as separate chains (V_{H}/V_{L} format).

In some instances, the bi-specific antibody disclosed herein may be in a 2-chain format: comprising two different polypeptides, which collectively form the two antigen binding moieties. Such a 2-chain format bi-specific antibody may comprise multiple copies of one or both polypeptides, forming trivalent, tetravalent, pentavelnt, or hexavalent antibodies. See, e.g., FIGS. 5A-5C.

In some examples, the bi-specific antibody disclosed herein may comprise two chains: a first chain being a fusion protein of the scFv fragment of one antigen binding moiety and the heavy chain or the light chain of the other antigen binding moiety, and the second chain being the other chain of the other antigen binding moiety. For example, the bi-specific antibody may comprise a first chain that is a fusion protein of a scFv fragment of a first antigen binding moiety binding to a first antigen (e.g., CD40, CD137, GITR, OX40, CD47, CD3, or CD28) fused to the heavy chain of a second antigen binding moiety, which binds to a second antigen (e.g., B7H3), and a second chain which is the light chain of the second antigen binding moiety. In any of the fusion chains, the scFv fragment and the heavy or light chain may be in any order. In some instances, the scFv can be located at the N-terminus. In other instances, the heavy or light chain may be located at the N-terminus.

In some examples, the bi-specific antibody may be comprise two chains: (i) a first polypeptide comprising the V_{L} fragment of a first antigen binding moiety and a heavy chain comprising the V_{H} fragment of a second antigen binding moiety and an Fc fragment (e.g., a whole Fc fragment or a portion thereof such as CH2-CH3); and (ii) a second polypeptide comprising the V_{H} fragment of the first antigen binding moiety and the V_{L} fragment of the second antigen binding moiety. In the first polypeptide, the V_{L} fragment may be located at the N-terminus and the heavy chain may be located at the C-terminus. Alternatively, the V_{L} fragment may be located at the C-terminus and the heavy chain may be located at the N-terminus of the first polypeptide. Similarly, the second polypeptide may have the V_{H} fragment at the N-terminus and the V_{L} fragment at the C-terminus. Alternatively, the second polypeptide may have the V_{H} fragment at the C-terminus and the V_{L} fragment at the N-terminus.

For example, the bi-specific antibody may comprise: (i) a first polypeptide comprising the V_{L} fragment of a first antigen binding moiety that binds B7H3 and a heavy chain comprising the V_{H} fragment of a second antibody that binds CD40, CD137, GITR, OX40, CD47, CD3, or CD28 and an Fc fragment; and (ii) a second polypeptide comprising the V_{H} fragment of the first antigen binding moiety and the V_{L} fragment of the second antigen binding moiety. Alternatively, the bi-specific antibody may comprise (i) a first polypeptide comprising the V_{L} fragment of a first antigen binding moiety that binds CD40, CD137, GITR, OX40, CD47, CD3, or CD28 and a heavy chain comprising the V_{H} fragment of a second antibody that binds B7H3 and an Fc fragment; and (ii) a second polypeptide comprising the V_{H} fragment of the first antigen binding moiety and the V_{L} fragment of the second antigen binding moiety.

In other examples, the bi-specific antibody may comprise two chains: (i) a first polypeptide comprising the V_{H} fragment of a first antigen binding moiety and a heavy chain of a second antigen binding moiety (comprising the V_{H} fragment and an Fc fragment), and (ii) a second polypeptide comprising the V_{L} fragment of the first antigen binding moiety and the light chain of the second antigen binding moiety (e.g., comprising a light chain variable region and a light chain constant region). In the first polypeptide, the V_{H} fragment of the first antigen binding moiety may be located at the N-terminus. Alternatively, it may be located at the C-terminus. In the second polypeptide, the V_{L} fragment of the first antigen binding moiety may be located at the N-terminus. Alternatively, it may be located at the C-terminus. In some instances, the first antigen binding moiety binds B7H3 and the second antigen binding moiety binds CD40, CD137, GITR, OX40, CD47, CD3, or CD28. In other instances, the first antigen binding moiety binds CD40, CD137, GITR, OX40, CD47, CD3, or CD28 and the second antigen binding moiety binds B7H3.

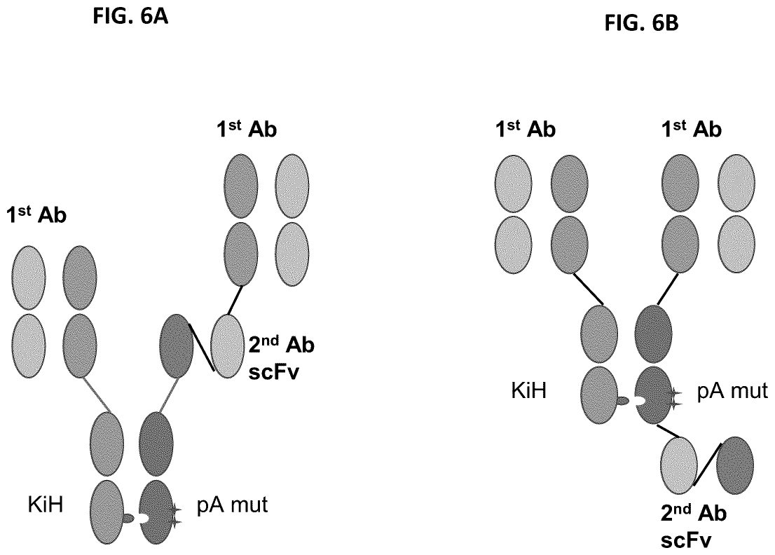

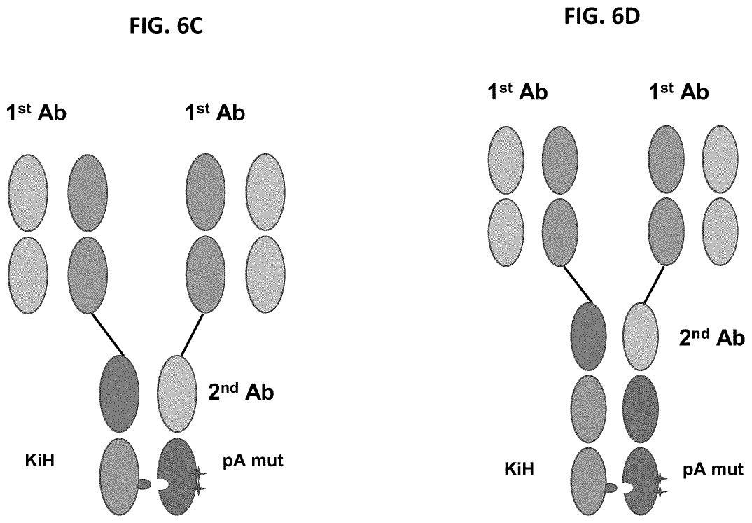

In some instances, the bi-specific antibody disclosed herein may be in a 3-chain format: comprising three different polypeptides, which collectively form the two antigen binding moieties. Such a 3-chain format bi-specific antibody may comprise multiple copies of one or more of the polypeptides, forming trivalent, tetravalent, pentavelnt, or hexavalent antibodies. See, e.g., FIGS. 6A-6D.

In some examples, a bi-specific antibody disclosed herein comprises three polypeptides. The first polypeptide comprises the heavy chain of the first antigen binding moiety (e.g., binding to B7H3) in the bi-specific antibody fused to the light chain of the second antigen binding moiety (e.g., binding to the second antigen such as CD40, CD137, GITR, OX40, CD47, CD3, or CD28). The second and third polypeptides comprise the light chain of the first antigen binding moiety and the heavy chain of the second antigen binding moiety, respectively. In some instances, the heavy chain of the second antigen binding moiety may comprise a V_{H} fragment and a heavy chain constant region such as CH1. Alternatively, the first polypeptide comprises the heavy chain of the second antigen binding moiety (e.g., binding to the second antigen such as CD40, CD137, GITR, OX40, CD47, CD3, or CD28) fused to the light chain of the first antigen binding moiety (e.g., binding to B7H3). The second and third polypeptides comprise the light chain of the second antigen binding moiety and the heavy chain of the first antigen binding moiety, respectively. In some instances, the heavy chain of the first antigen binding moiety may comprise a V_{H} fragment and a heavy chain constant region such as CH1. In some instances, the light chain fragment in the first polypeptide can be located at the N-terminus. Alternatively, it may be located at the C-terminus.

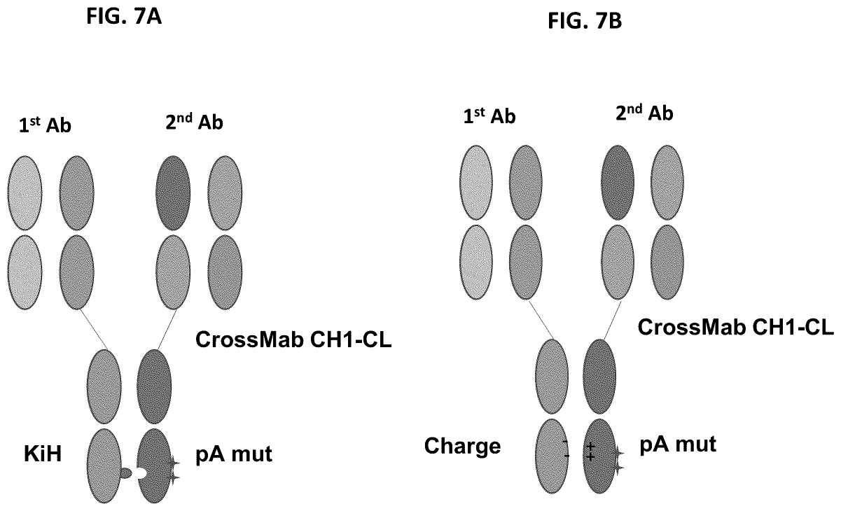

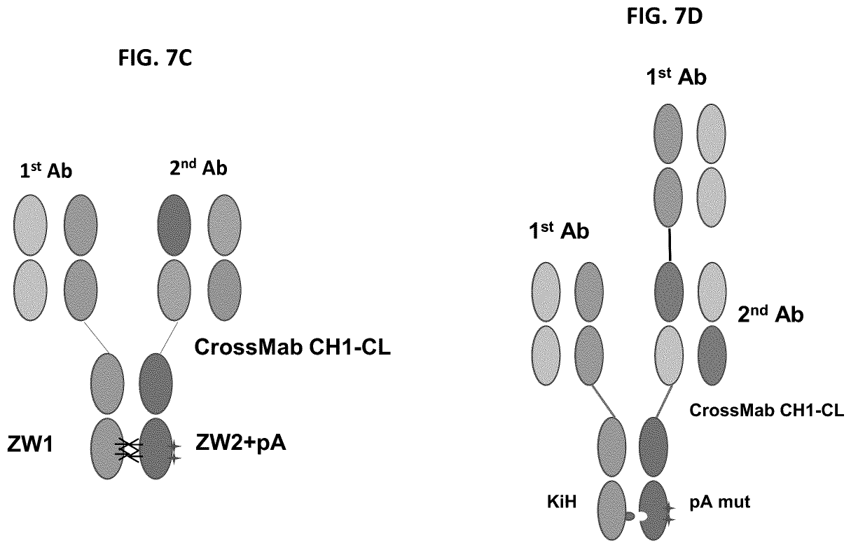

In some embodiments, any of the bi-specific antibodies disclosed herein may be in a IgG-like format (4-chain format): one arm binding to human B7H3 and another arm binding to CD40, CD137, GITR, OX40, CD47, CD3, or CD28. Each arm comprises a heavy chain and a light chain. Structurally it is made from half of anti-B7H3 antibody and half of antibody against CD40, CD137, GITR, OX40, CD47, CD3, or CD28, with the similar size and shape as natural IgG. See, e.g., FIGS. 7A-7D.

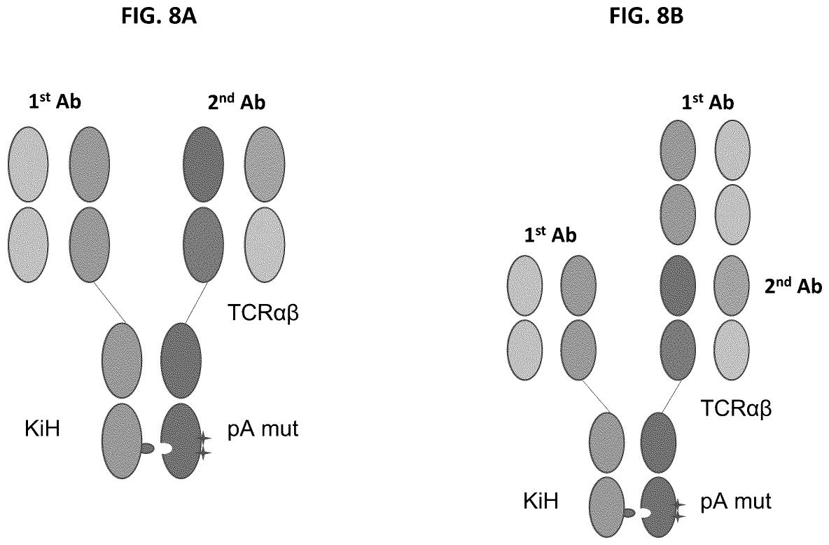

In some embodiments, a bi-specific antibody disclosed herein may comprise V_{H} and V_{L} antibody variable regions fused to TCR constant regions, respectively. See, e.g., WO2014014796A1, 23 Jan. 2014, CN1561343A, 5 Jan. 2005, PCT/CN2018/106766, 20 Sep. 2018. See, e.g., FIGS. 8A-8B.

### (ii) Tri-Specific Antibodies

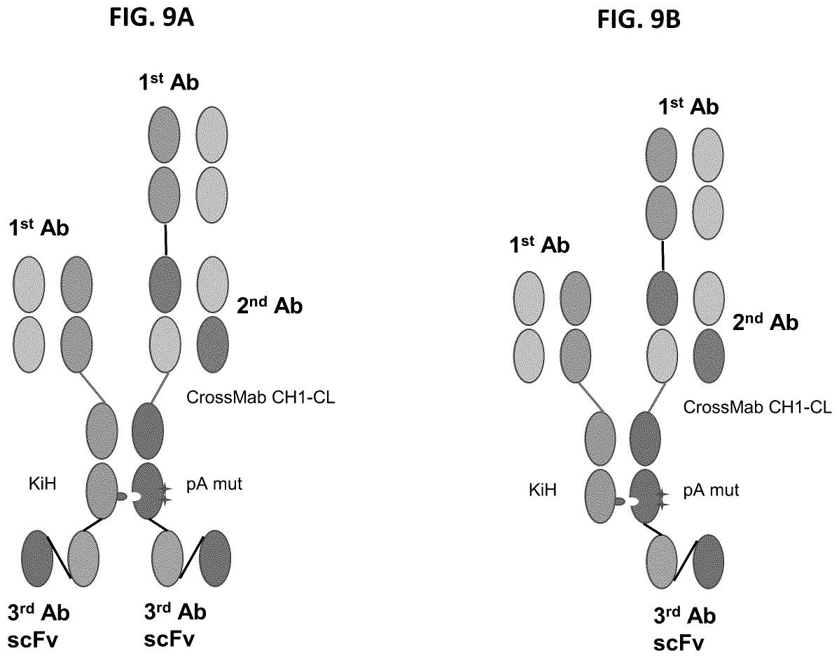

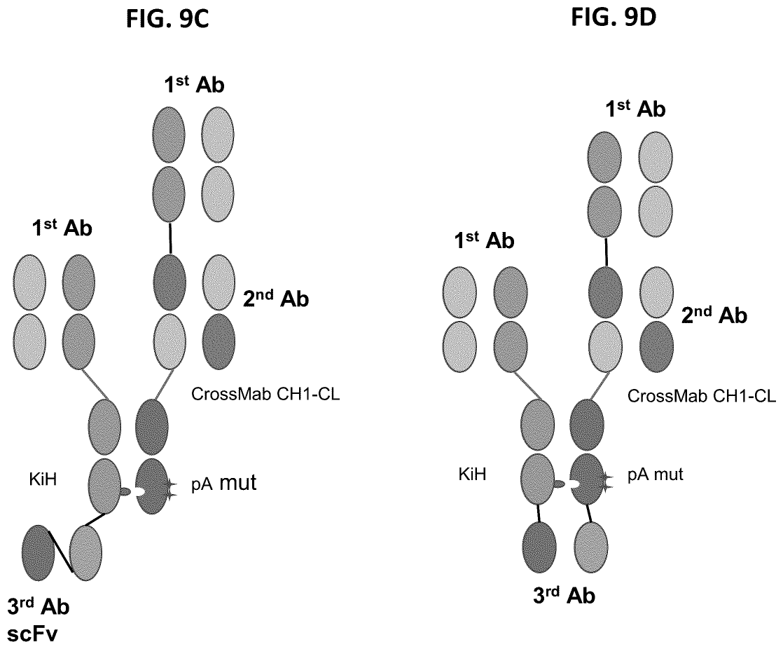

In some embodiments, the multi-specific antibodies disclosed herein are tri-specific antibodies comprising one antigen binding moiety specific to B7H3 and two additional antigen binding moieties specific to two different antigens selected from CD40, CD137, GITR, OX40, CD47, CD3, and CD28. Examples include, but are not limited to, anti-B7H3/CD3/CD137 tri-specific antibodies, anti-B7H3/CD3/GITR tri-specific antibodies, anti-B7H3/CD3/OX40 tri-specific antibodies, anti-B7H3/CD3/CD28 tri-specific antibodies, anti-B7H3/CD137/OX40 tri-specific antibodies, and anti-B7H3/CD137/GITR tri-specific antibodies. Such a tri-specific antibody may be of any format known in the art. Exemplary tri-specific antibody formats are illustrated in FIGS. 9A-9D, FIGS. 10A-10C, FIGS. 11A-11E, FIGS. 12A-12D, and FIGS. 13A-13C, all of which are within the scope of the present disclosure.

In some examples, one antigen binding moiety in the tri-specific antibody (e.g., the anti-B7H3 moiety) is in a multi-chain antibody format as disclosed herein, and the other two antigen binding moieties (e.g., specific to the other two antigens of interest) can be in an scFv format, in a Fab format, and/or in V_{H}/V_{L} format. For example, the multi-chain antibody format comprises a light chain that comprises a V_{L} domain and a light chain constant region, and a heavy chain that comprises a V_{H} and a heavy chain constant domain or a fragment thereof (which optionally may comprise the CH3 domain).

In other examples, two antigen binding moieties in the tri-specific antibody may be in a multi-chain antibody format as disclosed herein and the other antigen binding moiety may be in scFv or V_{H}/V_{L} format. For example, one antigen binding moiety (e.g., the anti-B7H3 moiety) may comprises a light chain that comprises a V_{L} domain and a light chain constant region, and a heavy chain that comprises a V_{H} and a heavy chain constant domain or a fragment thereof (which optionally may comprise the CH3 domain). Another antigen binding moiety (e.g., specific to one of the other antigen) may comprise a V_{H} fragment and a V_{L} fragment as separate chains (V_{H}/V_{L} format) or in scFv format. The third antigen binding moiety (e.g., specific to the other antigen of interest) may be in Fab format.

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in scFv form, and (iii) a third antigen binding moiety that is in Fab format. See, e.g., FIGS. 9A to 9D. In some examples, the first antigen binding moiety binds B7H3 and the second and third antigen binding moieties bind the two other antigens of interest (e.g., CD3 and one of CD137, GITR, OX40, and CD28). The three antigen binding moieties may be assembled to form the tri-specific antibody as disclosed herein in any suitable manner. Some non-limiting examples are provided in FIGS. 9A to 9D.

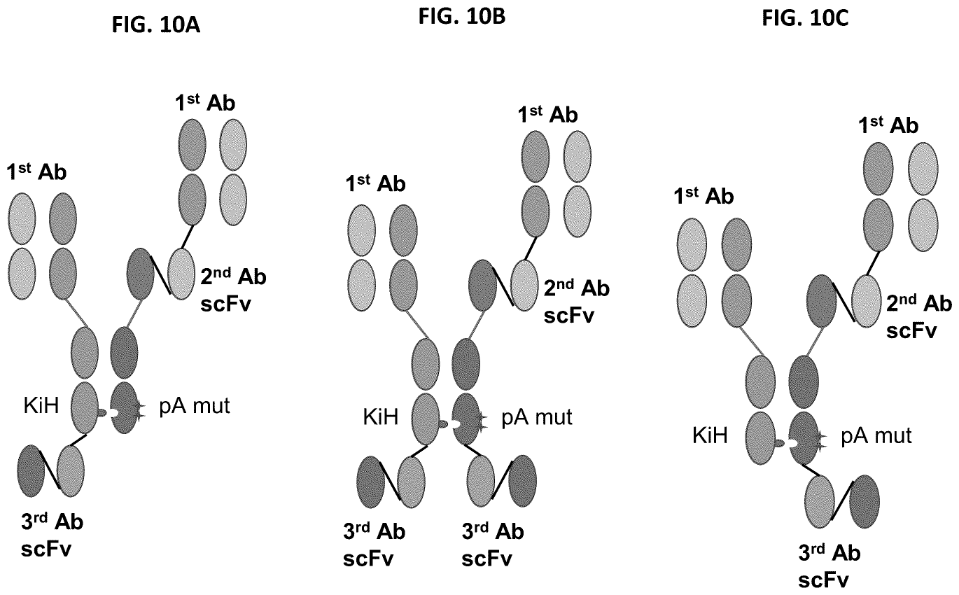

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in scFv format, and (iii) a third antigen binding moiety that is in scFv format. See, e.g., FIG. 10A to FIG. 10C. In some examples, the first antigen binding moiety binds B7H3 and the second and third antigen binding moieties bind the two other antigens of interest (e.g., C D3 and one of CD137, GITR, OX40, and CD28). The three antigen binding moieties may be assembled to form the tri-specific antibody as disclosed herein in any suitable manner. Some non-limiting examples are provided in FIGS. 10A-10C.

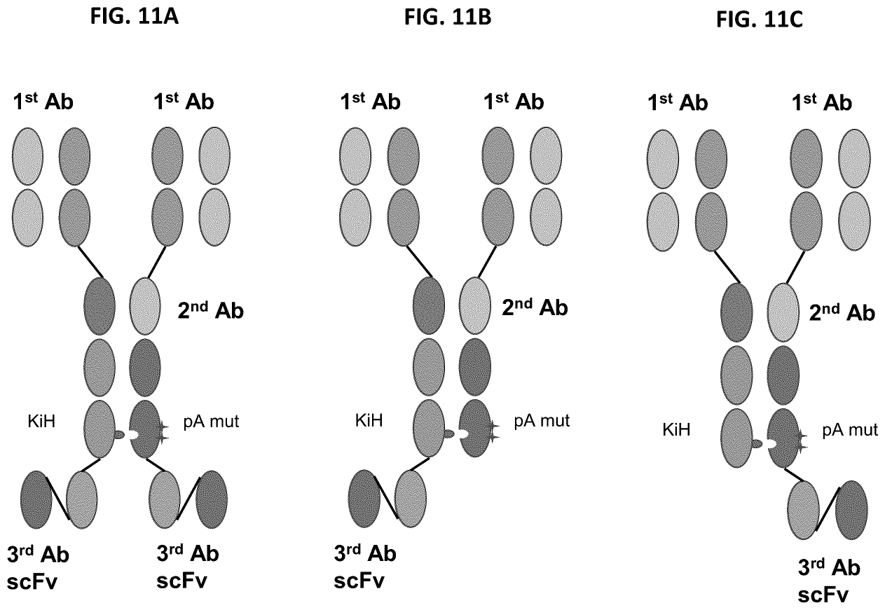

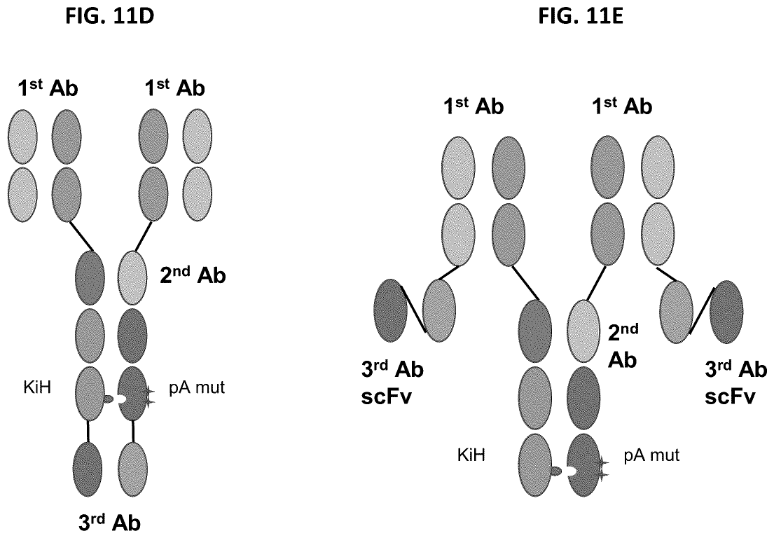

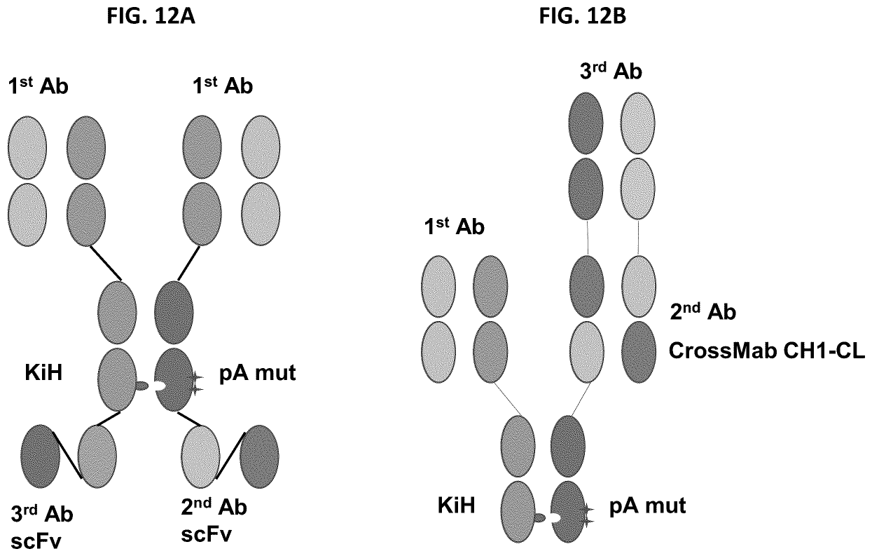

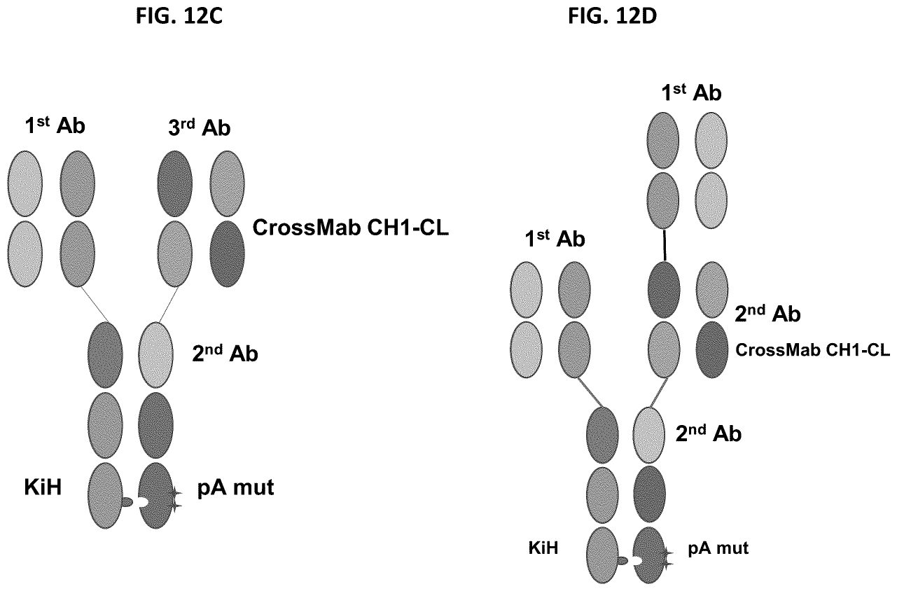

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in scFv format, and (iii) a third antigen binding moiety that is in V_{H}/V_{L} format. See, e.g., FIGS. 11A to 11C, and 11E, and FIG. 12A. In some examples, the first antigen binding moiety binds B7H3 and the second and third antigen binding moieties bind the two other antigens of interest (e.g., CD3 and one of CD137, GITR, OX40, and CD28). The three antigen binding moieties may be assembled to form the tri-specific antibody as disclosed herein in any suitable manner. Some non-limiting examples are provided in FIGS. 11A-11C and 11E, and FIG. 12A.

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in V_{H}/V_{L} format, and (iii) a third antigen binding moiety that is in V_{H}/V_{L} format. See, e.g., FIG. 11D. In some examples, the first antigen binding moiety binds B7H3 and the second and third antigen binding moieties bind the two other antigens of interest (e.g., CD3 and one of CD137, GITR, OX40, and CD28). The three antigen binding moieties may be assembled to form the tri-specific antibody as disclosed herein in any suitable manner. Some non-limiting examples are provided in FIG. 11D.

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in Fab format, and (iii) a third antigen binding moiety that is in Fab format. See, e.g., FIG. 12B. In some examples, the first antigen binding moiety binds B7H3 and the second and third antigen binding moieties bind the two other antigens of interest (e.g., CD3 and one of CD137, GITR, OX40, and CD28). The three antigen binding moieties may be assembled to form the tri-specific antibody as disclosed herein in any suitable manner. Some non-limiting examples are provided in FIG. 12B.

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in V_{H}/V_{L} format, and (iii) a third antigen binding moiety that is in Fab format. See, e.g., FIG. 12D. In some examples, the first antigen binding moiety binds B7H3 and the second and third antigen binding moieties bind the two other antigens of interest (e.g., CD3 and one of CD137, GITR, OX40, and CD28). The three antigen binding moieties may be assembled to form the tri-specific antibody as disclosed herein in any suitable manner. Some non-limiting examples are provided in FIG. 12D.

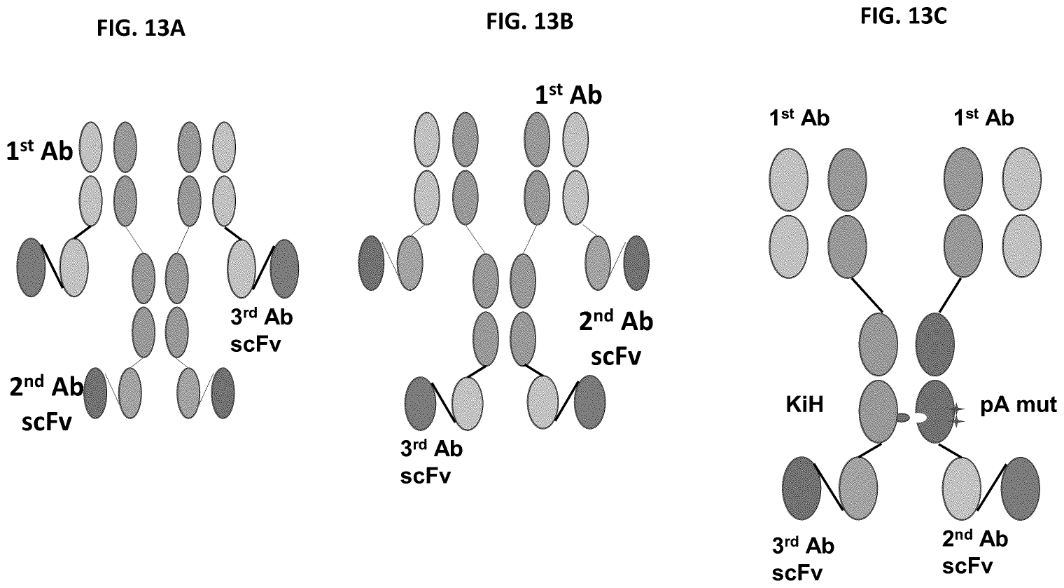

In some embodiments, the tri-specific antibody disclosed herein may comprise (i) a first antigen binding moiety in a multi-chain IgG like format (comprising a heavy chain that comprises a first V_{H} and a heavy chain constant region or a fragment thereof and a light chain that comprise a first V_{L} and a light chain constant region), (ii) a second antigen binding moiety that is in scFv format, and (iii) a third antigen binding moiety that is also in scFv format. See, e.g., FIGS. 13A-13C. In some examples, the scFv of the second antigen binding moiety is fused to a heavy chain of the first antigen binding moiety (e.g., to the C-terminus) and the scFv of the third antigen binding moiety is fused to a light chain of the first antigen binding moiety (e.g., to the C-terminus). FIG. 13A. Alternatively, the scFv of the third antigen binding moiety is fused to a heavy chain of the first antigen binding moiety (e.g., to the C-terminus) and the scFv of the second antigen binding moiety is fused to a light chain of the first antigen binding moiety (e.g., to the C-terminus). FIG. 13B. In another example, the scFv of the second antigen binding moiety is fused to one heavy chain of the first antigen binding moiety (e.g., to the C-terminus) and the scFv of the third antigen binding moiety is fused to the other heavy chain of the first antigen binding moiety (e.g., to the C-terminus). FIG. 13C.

(iii) Heterodimer Formation

In some embodiments, any of the multi-specific antibodies disclosed herein (e.g., bi-specific or tri-specific) are heterodimers formed by dimerization between two heavy chains. To facilitate heterodimeric assembly, mutations that enhance heterodimer formation may be introduced into the Fc regions of two heavy chains in a multi-specific antibodies. Examples include “knobs-into-holes” (Ridgway et al., Protein Engineering, 9 (7), pp. 617-21 (1996); Merchant et al., Nature Biotechnology, 16 (7), pp. 677-681 (1998)), electrostatics (Gunasekaran et al., Journal of Biological Chemistry, 285 (25), pp. 19637-19646 (2010)) or negative state designs (Kreudenstein et al., mAbs, 5 (5), pp. 646-654 (2013); Leaver-Fay et al., Structure, 24 (4), pp. 641-651 (2016)) (charged mutations). Other examples can be found in, e.g., Brinkmann et al., MABS (2017), 9(2):182-212, the relevant disclosures are incorporated by reference for the subject matter and purpose referenced herein.

In some examples, several strategies have been applied into designing orthogonal interfaces to facilitate cognate pairing, by swapping the domains of CH1 and CL, for example, CrossMab format (Schaefer et al., Proceedings of the National Academy of Sciences of the United States of America, 108 (27), pp. 11187-11192 (2011)), introducing alternatively disulphide bond (Mazor et al., mAbs, 7 (2), pp. 377-389 (2015)), mading further electrostatics in the CH1-CL region (Liu et al., Journal of Biological Chemistry, 290 (12), pp. 7535-7562 (2015)), and introducing mutations in both variable and constant domains (Lewis et al., Nature Biotechnology, 32 (2), pp. 191-198 (2014), Dillon et al., mAbs, 9 (2), pp. 213-230 (2017)). See also FIGS. 7A-7D, 9A-9D, 12B-12D, and 13C.

In some instances, mutations that reduce binding affinity to Protein A may be introduced into one or both of the heavy chain Fc regions in a multi-specific antibody to facilitate purification of the multi-specific antibodies. Such mutations are known in the art. See, e.g., Tustian et al., mAbs 8:828-838 (2016), the relevant disclosures of which are incorporated by reference for the purpose and subject matter referenced herein.

### (iv) Peptide Linkers

A peptide linker may be located between two fragments in a multi-specific antibody as disclosed herein, for example, between the V_{H} and V_{L} portions in a scFv fragment, between the scFv fragment and the heavy or light chain in a fusion chain, or between the heavy chain and light chain in a fusion polypeptide. Exemplary peptide linker includes the linker of (GGGGS)ₙ (SEQ ID NOs:665-670), in which n can be an integer between 1-6, for example, 1, 2, 3, 4, 5, or 6. Any of the peptide linkers described herein, e.g., the SGGGS (SEQ ID NO:671) linker or the (GGGGS)₄ (SEQ ID NO:668) linker, can comprise naturally occurring amino acids and/or non-naturally occurring amino acids. Naturally occurring amino acids include alanine (Ala), arginine (Arg), asparagine (Asn), aspartic acid (Asp), cysteine (Cys), glutamic acid (Glu), glutamine (Gin), glycine (Gly), histidine (His), isoleucine (He), leucine (Leu), lysine (Lys) methionine (Met), ornithine (Orn), phenylalanine (Phe), proline (Pro), serine (Ser), threonine (Thr), tryptophan (Trp), tyrosine (Tyr), and valine (Val). Non-naturally occurring amino acids can include protected amino acids such as naturally occurring amino acids protected with groups such as acetyl, formyl, tosyl, nitro and the like. Non-limiting examples of non-naturally occurring amino acids include azidohomoalanine, homopropargylglycine, homoallylglycine, p-bromophenylalanine, p-iodophenylalanine, azidophenylalanine, acetylphenylalanine or ethynylephenylalanine, amino acids containing an internal alkene such as trans-crotylalkene, serine allyl ether, allyl glycine, propargyl glycine, vinyl glycine, pyrrolysine, N-sigma-o-azidobenzyloxycarbonyl-L-Lysine (AzZLys), N-sigma-propargyloxycarbonyl-L-Lysine, N-sigma-2-azidoethoxycarbonyl-L-Lysine, N-sigma-tert-butyloxycarbonyl-L-Lysine (BocLys), N-sigma-allyloxycarbonyl-L-Lysine (AlocLys), N-sigma-acetyl-L-Lysine (AcLys), N-sigma-benzyloxycarbonyl-L-Lysine (ZLys), N-sigma-cyclopentyloxycarbonyl-L-Lysine (CycLys), N-sigma-D-prolyl-L-Lysine, N-sigma-nicotinoyl-L-Lysine (NicLys), N-sigma-N-Me-anthraniloyl-L-Lysine (NmaLys), N-sigma-biotinyl-L-Lysine, N-sigma-9-fluorenylmethoxycarbonyl-L-Lysine, N-sigma-methyl-L-Lysine, N-sigma-dimethyl-L-Lysine, N-sigma-multimethyl-L-Lysine, N-sigma-isopropyl-L-Lysine, N-sigma-dansyl-L-Lysine, N-sigma-o,p-dinitrophenyl-L-Lysine, N-sigma-p-toluenesulfonyl-L-Lysine, N-sigma-DL-2-amino-2carboxyethyl-L-Lysine, N-sigma-phenylpyruvamide-L-Lysine, N-sigma-pyruvamide-L-Lysine, azidohomoalanine, homopropargylglycine, homoallylglycine, p-bromophenylalanine, p-iodophenylalanine, azidophenylalanine, acetylphenylalanine or ethynylephenylalanine, amino acids containing and an internal alkene such as trans-crotylalkene, serine allyl ether, allyl glycine, propargyl glycine, and vinyl glycine.

### C. Exemplary Multi-Specific Antibodies

In some embodiments, the present disclosure provides bi-specific antibodies binding to B7H3 and one of CD40, CD137, GITR, OX40, CD47, CD3, and CD28. In addition, provided herein are tri-specific antibodies binding to B7H3 and two of the CD40, CD137, GITR, OX40, CD47, CD3, and CD28 antigens. Such bi-specific and tri-specific antibodies can comprise two or more antigen binding moieties derived from any of the parent antibodies provided herein (e.g., those listed in Table 1). Non-limiting examples are provided below.

### (i) Anti-B7H31CD40 Bi-Specific Antibodies

In some embodiments, the second antigen binding moiety in the bi-specific antibodies disclosed herein binds B7H3 and CD40, for example, human B7H3 and human CD40. Any antibody capable of binding to CD40 can be used in constructing the bi-specific antibodies disclosed herein. In some examples, the anti-CD40 portion of the bi-specific antibody described herein may be derived from the anti-CD40 antibodies provided herein (e.g., the anti-CD40 parent antibody provided in Table 1 above). The anti-CD40 antigen binding moiety may comprise the same heavy chain and/or light chain CDRs as a parent antibody. Alternatively, the antigen binding moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-CD40 antigen binding moiety in the bi-specific antibody may have the same heavy chain variable region and/or the same light chain variable region as the parent antibody. For example, the antigen binding moiety in the bi-specific antibody may have the same heavy chain and/or the same light chain as the parent antibody.

In some examples, the anti-B7H3/CD40 bi-specific antibodies may comprise an anti-CD40 moiety in scFv format and an anti-B7H3 moiety in multi-chain format. The anti-CD40 scFv fragment may be derived from any of the anti-CD40 antibodies disclosed herein, for example, the anti-CD40 parent antibody provided in Table 1 above the anti-CD40 parent antibody provided in Table 1 above. For example, the bi-specific antibody may comprise a first chain comprising the scFv fragment fused with the heavy chain of the anti-B7H3 antibody such as that of B7H3 Ab1 or B7H3 Ab2 shown in Table 1 above, and a second chain that is the light chain of the anti-B7H3 antibody. Alternatively, the bi-specific antibody may comprise a first chain comprising the scFv fragment may be fused with the light chain of the anti-B7H3 antibody such as that of B7H3 Ab1 or B7H3 Ab2 shown in Table 1 above, and a second chain that is the heavy chain of the anti-B7H3 antibody. In some instances, the heavy chain of the anti-B7H3 antibody may comprise a mutated Fc region having altered binding affinity and/or binding specificity to an Fc receptor such as those described herein.

In some embodiments, the anti-B7H3/CD40 bi-specific antibody disclosed herein may be in a three-chain format as disclosed herein. Such a bi-specific antibody may comprise a first polypeptide comprises the heavy chain of the first antigen binding moiety (e.g., binding to B7H3) fused to the light chain of second antigen binding moiety (e.g., binding to CD40), a second polypeptide comprising the light chain of the first antigen binding moiety, and a third polypeptide comprising the heavy chain of the second antigen binding moiety. In some instances, the heavy chain of the second antigen binding moiety may comprise a V_{H} fragment and a heavy chain constant region such as CH1. Alternatively, the bi-specific antibody may comprise a first polypeptide comprising the heavy chain of the second antigen binding moiety (e.g., binding to CD40) fused to the light chain of the first antigen binding moiety (e.g., binding to B7H3), a second polypeptide comprising the light chain of the second antigen binding moiety, and a third polypeptide comprising the heavy chain of the first antigen binding moiety. In some instances, the heavy chain of the first antigen binding moiety may comprise a V_{H} fragment and a heavy chain constant region such as CH1. In some instances, the light chain fragment in the first polypeptide can be located at the N-terminus. Alternatively, it can be located at the C-terminus.

In some examples, the bi-specific antibody may comprise two chains: (i) a first polypeptide comprising the V_{H} fragment of the first antigen binding moiety and the heavy chain of the second antigen binding moiety, and (ii) a second chain comprising the V_{L} fragment of the first antigen binding moiety and the light chain of the second antigen binding moiety. In some instances, the first antigen binding moiety binds B7H3 and the second antigen binding moiety binds CD40. In other instances, the first antigen binding moiety binds CD40 and the second antigen binding moiety binds B7H3

In other examples, the bi-specific antibody may comprise two chains: (i) a first polypeptide comprising the V_{L} fragment of a first antigen binding moiety and a heavy chain comprising the V_{H} fragment of a second antigen binding moiety and an Fc fragment (e.g., a whole Fc fragment or a portion thereof such as CH2-CH3); and (ii) a second polypeptide comprising the V_{H} fragment of the first antigen binding moiety and the V_{L} fragment of the second antigen binding moiety. In some instances, the first antigen binding moiety binds B7H3 and the second antigen binding moiety binds CD40. In other instances, the first antigen binding moiety binds CD40 and the second antigen binding moiety binds B7H3.

In some embodiments, any of the bi-specific antibodies disclosed herein may be in an IgG-like format (comprising 4-chains): one arm binding to human B7H3 and another arm binding to CD40. Structurally it is made from half of anti-B7H3 antibody and half of anti-CD40 antibody, with the similar size and shape as natural IgG.

In some aspect, the present disclosure provides a polypeptide complex comprising a first polypeptide comprising, from N-terminus to C-terminus, a first heavy chain variable domain (V_{H}) of a first antibody operably linked to a first T cell receptor (TCR) constant region (C1), and a second polypeptide comprising, from N-terminus to C-terminus, a first light chain variable domain (V_{L}) of the first antibody operably linked to a second TCR constant region (C2), wherein C1 and C2 are capable of forming a dimer comprising at least one non-native interchain bond between C1 and C2, and the non-native interchain bond is capable of stabilizing the dimer, and the first antibody has a first antigenic specificity.

In some aspect, the present disclosure provides a bispecific polypeptide complex, comprising a first antigen-binding moiety associated with a second antigen-binding moiety, wherein the first antigen-binding moiety comprising a first polypeptide comprising, from N-terminal to C-terminal, a first heavy chain variable domain (V_{H}) of a first antibody operably linked to a first T cell receptor (TCR) constant region (C1), and a second polypeptide comprising, from N-terminal to C-terminal, a first light chain variable domain (V_{L}) of the first antibody operably linked to a second TCR constant region (C2), wherein C1 and C2 are capable of forming a dimer comprising at least one non-native interchain bond between a first mutated residue comprised in C1 and a second mutated residue comprised in C2, and the non-native interchain bond is capable of stabilizing the dimer, and the first antibody has a first antigenic specificity, a second antigen-binding moiety has a second antigenic specificity which is different from the first antigenic specificity, and the first antigen-binding moiety and the second antigen-binding moiety are less prone to mispair than otherwise would have been if both the first and the second antigen-binding moieties are counterparts of natural Fab.

In some examples, any of the anti-B7H3/CD40 bispecific antibodies may comprise mutations for enhancing heterodimerization and/or reducing protein A binding such as those disclosed herein. Alternatively or in addition, the anti-B7H3/CD40 bispecific antibodies may be in CrossMab format.

Exemplary anti-B7H3/CD40 bi-specific antibodies are provided in Example 3 and Table 3, which are within the scope of the present disclosure.

### (ii) Anti-B7H31CD137, Anti-B7H3/GITR, and Anti-B7H31OX40 Bi-Specific Antibodies

In some embodiments, the bi-specific antibodies disclosed herein binds B7H3 and a second antigen, which is CD137, GITR or OX40, (e.g., human B7H3, human CD137, GITR or OX40. Any antibody capable of binding to B7H3, CD137, GITR or OX40 can be used in constructing the bi-specific antibodies disclosed herein, e.g., the parent antibodies listed in Table 1 above. In some examples, the anti-CD137, ant-GITR or anti-OX40 portion of the bi-specific antibody described herein may be derived from any of the anti-CD137, anti-GITR or anti-OX40 antibodies provided herein (e.g., the corresponding parent antibodies listed in Table 1 above). The anti-CD137, anti-GITR or anti-OX40 antigen binding moiety may comprise the same heavy chain and/or light chain CDRs as a parent antibody. Alternatively, the antigen binding moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-CD137, anti-GITR or anti-OX40 antigen binding moiety in the bi-specific antibody may have the same heavy chain variable region and/or the same light chain variable region as the parent antibody. For example, the antigen binding moiety in the bi-specific antibody may have the same heavy chain and/or the same light chain as the parent antibody.

In some examples, the anti-B7H3/CD137, anti-B7H3/GITR or anti-B7H3/OX40 bi-specific antibodies may comprise an anti-CD137, anti-GITR or anti-OX40 moiety in scFv format and an anti-B7H3 moiety in multi-chain format. The anti-CD137, anti-GITR or anti-OX40 scFv fragment may be derived from any of the anti-CD137, anti-GITR or anti-OX40 antibodies disclosed herein, for example, the corresponding parent antibodies listed in Table 1 above.

For example, the bi-specific antibody may comprise a first chain comprising the scFv fragment fused with the heavy chain of the anti-B7H3 antibody such as that of B7H3 Ab1 or B7H3 Ab2, and a second chain that is the light chain of the anti-B7H3 antibody. Alternatively, the bi-specific antibody may comprise a first chain comprising the scFv fragment may be fused with the light chain of the anti-B7H3 antibody such as that of B7H3 Ab1 or B7H3 Ab2, and a second chain that is the heavy chain of the anti-B7H3 antibody. In some instances, the heavy chain of the anti-B7H3 antibody may comprise a mutated Fc region having altered binding affinity and/or binding specificity to an Fc receptor such as those described herein.

In some embodiments, the anti-B7H3/CD137, anti-B7H3/GITR or anti-B7H3/OX40 bi-specific antibody disclosed herein may be in a three-chain format as disclosed herein. Such a bi-specific antibody may comprise a first polypeptide comprises the heavy chain of the first antigen binding moiety (e.g., binding to B7H3) fused to the light chain of second antigen binding moiety (e.g., binding to CD137, GITR or OX40), a second polypeptide comprising the light chain of the first antigen binding moiety, and a third polypeptide comprising the heavy chain of the second antigen binding moiety. In some instances, the heavy chain of the second antigen binding moiety may comprise a V_{H} fragment and a heavy chain constant region such as CH1. Alternatively, the bi-specific antibody may comprise a first polypeptide comprising the heavy chain of the second antigen binding moiety (e.g., binding to CD137, GITR or OX40) fused to the light chain of the first antigen binding moiety (e.g., binding to B7H3), a second polypeptide comprising the light chain of the second antigen binding moiety, and a third polypeptide comprising the heavy chain of the first antigen binding moiety. In some instances, the heavy chain of the first antigen binding moiety may comprise a V_{H} fragment and a heavy chain constant region such as CH1. In some instances, the light chain fragment in the first polypeptide can be located at the N-terminus. Alternatively, it can be located at the C-terminus.

In some examples, the bi-specific antibody may comprise two chains: (i) a first polypeptide comprising the V_{H} fragment of the first antigen binding moiety and the heavy chain of the second antigen binding moiety, and (ii) a second chain comprising the V_{L} fragment of the first antigen binding moiety and the light chain of the second antigen binding moiety. In some instances, the first antigen binding moiety binds B7H3 and the second antigen binding moiety binds CD137, GITR or OX40. In other instances, the first antigen binding moiety binds CD137, GITR or OX40 and the second antigen binding moiety binds B7H3

In other examples, the bi-specific antibody may comprise two chains: (i) a first polypeptide comprising the V_{L} fragment of a first antigen binding moiety and a heavy chain comprising the V_{H} fragment of a second antigen binding moiety and an Fc fragment (e.g., a whole Fc fragment or a portion thereof such as CH2-CH3); and (ii) a second polypeptide comprising the V_{H} fragment of the first antigen binding moiety and the V_{L} fragment of the second antigen binding moiety. In some instances, the first antigen binding moiety binds B7H3 and the second antigen binding moiety binds CD137, GITR or OX40. In other instances, the first antigen binding moiety binds CD137, GITR or OX40 and the second antigen binding moiety binds B7H3.

In some embodiments, any of the bi-specific antibodies disclosed herein may be in an IgG-like format (4-chain format): one arm binding to human B7H3 and another arm binding to CD137, GITR or OX40. Structurally it is made from half of anti-B7H3 antibody and half of anti-CD137, anti-GITR or anti-OX40 antibody, with the similar size and shape as natural IgG.

In some aspect, the present disclosure provides a polypeptide complex comprising a first polypeptide comprising, from N-terminus to C-terminus, a first heavy chain variable domain (V_{H}) of a first antibody operably linked to a first T cell receptor (TCR) constant region (C1), and a second polypeptide comprising, from N-terminus to C-terminus, a first light chain variable domain (V_{L}) of the first antibody operably linked to a second TCR constant region (C2), wherein C1 and C2 are capable of forming a dimer comprising at least one non-native interchain bond between C1 and C2, and the non-native interchain bond is capable of stabilizing the dimer, and the first antibody has a first antigenic specificity.

In some aspect, the present disclosure provides a bispecific polypeptide complex, comprising a first antigen-binding moiety associated with a second antigen-binding moiety, wherein the first antigen-binding moiety comprising a first polypeptide comprising, from N-terminal to C-terminal, a first heavy chain variable domain (V_{H}) of a first antibody operably linked to a first T cell receptor (TCR) constant region (C1), and a second polypeptide comprising, from N-terminal to C-terminal, a first light chain variable domain (V_{L}) of the first antibody operably linked to a second TCR constant region (C2), wherein C1 and C2 are capable of forming a dimer comprising at least one non-native interchain bond between a first mutated residue comprised in C1 and a second mutated residue comprised in C2, and the non-native interchain bond is capable of stabilizing the dimer, and the first antibody has a first antigenic specificity, a second antigen-binding moiety has a second antigenic specificity which is different from the first antigenic specificity, and the first antigen-binding moiety and the second antigen-binding moiety are less prone to mispair than otherwise would have been if both the first and the second antigen-binding moieties are counterparts of natural Fab.

In some examples, any of the anti-B7H3/CD40 bispecific antibodies may comprise mutations for enhancing heterodimerization and/or reducing protein A binding such as those disclosed herein. Alternatively or in addition, the anti-B7H3/CD40 bispecific antibodies may be in CrossMab format as also disclosed herein.

Exemplary anti-B7H3/CD137, anti-B7H3/GITR or anti-B7H3/OX40 bi-specific antibodies are provided in Example 4-6 and Table 4-6 below, which are within the scope of the present disclosure.

(iii) Anti-B7H31CD47 Bi-Specific Antibodies

In some embodiments, the bi-specific antibodies disclosed herein binds B7H3 and CD47, for example, human B7H3 and human CD47. Any antibody capable of binding to B7H3 and CD47 can be used in constructing the bi-specific antibodies disclosed herein. In some examples, the anti-CD47 portion of the bi-specific antibody described herein may be derived from any of the anti-CD47 antibodies provided herein (e.g., the corresponding parent antibodies listed in Table 1 above). The anti-CD47 antigen binding moiety may comprise the same heavy chain and/or light chain CDRs as the parent antibody. Alternatively, the antigen binding moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-CD47 antigen binding moiety in the bi-specific antibody may have the same heavy chain variable region and/or the same light chain variable region as the parent antibody. For example, the antigen binding moiety in the bi-specific antibody may have the same heavy chain and/or the same light chain as the parent antibody.

In some examples, the anti-B7H3/CD47 bi-specific antibodies may comprise an anti-CD47 moiety in scFv format and an anti-B7H3 moiety in multi-chain format. The anti-CD47 scFv fragment may be derived from any of the anti-CD47 antibodies disclosed herein, for example, the corresponding parent antibodies listed in Table 1 above.

For example, the bi-specific antibody may comprise a first chain comprising the scFv fragment fused with the heavy chain of the anti-B7H3 antibody such as that of Ly1562, and a second chain that is the light chain of the anti-B7H3 antibody. Alternatively, the bi-specific antibody may comprise a first chain comprising the scFv fragment may be fused with the light chain of the anti-B7H3 antibody such as that of B7H3 Ab1 or B7H3 Ab2, and a second chain that is the heavy chain of the anti-B7H3 antibody. In some instances, the heavy chain of the anti-B7H3 antibody may comprise a mutated Fc region having altered binding affinity and/or binding specificity to an Fc receptor such as those described herein.

In some embodiments, the anti-B7H3/CD47 bi-specific antibody disclosed herein may be in a three-chain format as disclosed herein. Such a bi-specific antibody may comprise a first polypeptide comprises the heavy chain of the first antigen binding moiety (e.g., binding to B7H3) fused to the light chain of second antigen binding moiety (e.g., binding to CD47), a second polypeptide comprising the light chain of the first antigen binding moiety, and a third polypeptide comprising the heavy chain of the second antigen binding moiety. In some instances, the heavy chain of the second antigen binding moiety may comprise a V_{H} fragment and a heavy chain constant region such as CH1. Alternatively, the bi-specific antibody may comprise a first polypeptide comprising the heavy chain of the second antigen binding moiety (e.g., binding to CD47) fused to the light chain of the first antigen binding moiety (e.g., binding to B7H3), a second polypeptide comprising the light chain of the second antigen binding moiety, and a third polypeptide comprising the heavy chain of the first antigen binding moiety. In some instances, the heavy chain of the first antigen binding moiety may comprise a V_{H} fragment and a heavy chain constant region such as CH1. In some instances, the light chain fragment in the first polypeptide can be located at the N-terminus. Alternatively, it can be located at the C-terminus.

In some examples, the bi-specific antibody may comprise two chains: (i) a first polypeptide comprising the V_{H} fragment of the first antigen binding moiety and the heavy chain of the second antigen binding moiety, and (ii) a second chain comprising the V_{L} fragment of the first antigen binding moiety and the light chain of the second antigen binding moiety. In some instances, the first antigen binding moiety binds B7H3 and the second antigen binding moiety binds CD47. In other instances, the first antigen binding moiety binds CD47 and the second antigen binding moiety binds B7H3

In other examples, the bi-specific antibody may comprise two chains: (i) a first polypeptide comprising the V_{L} fragment of a first antigen binding moiety and a heavy chain comprising the V_{H} fragment of a second antigen binding moiety and an Fc fragment (e.g., a whole Fc fragment or a portion thereof such as CH2-CH3); and (ii) a second polypeptide comprising the V_{H} fragment of the first antigen binding moiety and the V_{L} fragment of the second antigen binding moiety. In some instances, the first antigen binding moiety binds B7H3 and the second antigen binding moiety binds CD47. In other instances, the first antigen binding moiety binds CD47 and the second antigen binding moiety binds B7H3.

In some embodiments, any of the bi-specific antibodies disclosed herein may be in an IgG-like format: one arm binding to human B7H3 and another arm binding to CD47. Structurally it is made from half of anti-B7H3 antibody and half of anti-CD47 antibody, with the similar size and shape as natural IgG.

In some aspect, the present disclosure provides a polypeptide complex comprising a first polypeptide comprising, from N-terminus to C-terminus, a first heavy chain variable domain (V_{H}) of a first antibody operably linked to a first T cell receptor (TCR) constant region (C1), and a second polypeptide comprising, from N-terminus to C-terminus, a first light chain variable domain (V_{L}) of the first antibody operably linked to a second TCR constant region (C2), wherein C1 and C2 are capable of forming a dimer comprising at least one non-native interchain bond between C1 and C2, and the non-native interchain bond is capable of stabilizing the dimer, and the first antibody has a first antigenic specificity.

In some aspect, the present disclosure provides a bispecific polypeptide complex, comprising a first antigen-binding moiety associated with a second antigen-binding moiety, wherein the first antigen-binding moiety comprising a first polypeptide comprising, from N-terminal to C-terminal, a first heavy chain variable domain (V_{H}) of a first antibody operably linked to a first T cell receptor (TCR) constant region (C1), and a second polypeptide comprising, from N-terminal to C-terminal, a first light chain variable domain (V_{L}) of the first antibody operably linked to a second TCR constant region (C2), wherein C1 and C2 are capable of forming a dimer comprising at least one non-native interchain bond between a first mutated residue comprised in C1 and a second mutated residue comprised in C2, and the non-native interchain bond is capable of stabilizing the dimer, and the first antibody has a first antigenic specificity, a second antigen-binding moiety has a second antigenic specificity which is different from the first antigenic specificity, and the first antigen-binding moiety and the second antigen-binding moiety are less prone to mispair than otherwise would have been if both the first and the second antigen-binding moieties are counterparts of natural Fab.

In some examples, any of the anti-B7H3/CD47 bispecific antibodies may comprise mutations for enhancing heterodimerization and/or reducing protein A binding such as those disclosed herein. Alternatively or in addition, the anti-B7H3/CD47 bispecific antibodies may be in CrossMab format as also disclosed herein.

Exemplary anti-B7H3/CD47 bi-specific antibodies are provided in Example 5 and Table 5 below, which are within the scope of the present disclosure.

### (iv) Anti-B7H31CD3 Bi-Specific Antibodies

In some embodiments, the bi-specific antibodies disclosed herein binds B7H4 and CD3, for example, human B7H4 and human CD3. Any antibody capable of binding to CD3 can be used in constructing the bi-specific antibodies disclosed herein. In some examples, the anti-CD3 portion of the bi-specific antibody described herein may be derived from any of the anti-CD3 antibodies provided herein (e.g., the corresponding parent antibodies listed in Table 1 above). The anti-CD3 antigen binding moiety may comprise the same heavy chain and/or light chain CDRs as a parent antibody. Alternatively, the antigen binding moiety may comprise substantially similar heavy chain and/or light chain CDRs as those of the parent antibody (e.g., comprising no more than 5, 4, 3, 2, or 1 amino acid residue variations as compared with the parent antibody). In some instances, the anti-CD3 antigen binding moiety in the bi-specific antibody may have the same heavy chain variable region and/or the same light chain variable region as the parent antibody. For example, the antigen binding moiety in the bi-specific antibody may have the same heavy chain and/or the same light chain as the parent antibody.

In some examples, the anti-B7H3/CD3 bi-specific antibodies may comprise an anti-CD3 moiety in V_{H} and V_{L} fragment format and an anti-B7H3 moiety in multi-chain format. The anti-CD3 V_{H} and V_{L} fragment may be derived from any of the anti-CD3 antibodies disclosed herein, for example, each of the corresponding parent antibodies listed in Table 1 above.

For example, the bi-specific antibody may comprise a first chain comprising the Fab fragment of anti-B7H3 HC and anti-CD3 V_{H} and CH1 of anti-CD3 LC and an Fc fragment (e.g., a whole Fc fragment or a portion thereof such as CH2-CH3), a second chain that is the light chain of the anti-B7H3 antibody, a third chain that is the anti-CD3 V_{L} fused with CH1 of anti-CD3 HC, and a fourth chain that is the heavy chain of anti-B7H3 antibody. Two Fc fragments with engineered CH3 domain to create a “knob” in one and a “hole” in the other to promote heterodimerization.

In some examples, any of the anti-B7H3/CD3 bispecific antibodies may comprise mutations for enhancing heterodimerization and/or reducing protein A binding such as those disclosed herein. Alternatively or in addition, the anti-B7H3/CD3 bispecific antibodies may be in CrossMab format also disclosed herein

Exemplary anti-B7H3/CD3 bi-specific antibodies are provided in Example 8 and Table 8, which are within the scope of the present disclosure.

### (v) Anti-B7H3/CD31CD137 Tri-Specific Antibodies

In some embodiments, the tri-specific antibodies disclosed herein binds B7H3, CD3 and CD137, for example, human B7H3, human CD3 and CD137. Any antibody capable of binding to B7H3, CD3 and CD137 can be used in constructing the tri-specific antibodies disclosed herein. In some examples, the anti-B7H3 portion, the anti-CD3 and anti-CD137 portion of the tri-specific antibody described herein may be derived from any of the anti-B7H3, anti-CD3 and anti-CD137 parent antibodies provided in Table 1 above. The anti-B7H3, anti-CD3 and anti-CD137 antigen binding moieties may comprise the same heavy chain and/or light chain CDRs as a parent antibody. Alternatively, one or more of the antigen binding moieties may comprise substantially similar heavy chain and/or light chain CDRs as those of the parent antibody (e.g., comprising no more than 5, 4, 3, 2, or 1 amino acid residue variations as compared with the parent antibody). In some instances, the anti-B7H3, anti-CD3 and anti-CD137antigen binding moiety in the tri-specific antibody may have the same heavy chain variable region and/or the same light chain variable region as the parent antibody. For example, one or more of the antigen binding moieties in the tri-specific antibody may have the same heavy chain and/or the same light chain as the parent antibody.

In some examples, the anti-B7H3/CD3/CD137 tri-specific antibodies may be a multi-chain complex comprising an anti-CD137 moiety in scFv format and an anti-B7H3 moiety and anti-CD3 in V_{H}/V_{L} and/or Fab format. For example, the tri-specific antibody may comprise a first chain comprising the anti-CD137 scFv fragment fused with the heavy chain of the anti-B7H3 antibody such as that of B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-CD137 scFv fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In some examples, the anti-B7H3/CD3/CD137 tri-specific antibodies may comprise an anti-CD3 moiety in fused V_{H} and V_{L} (scFv) format and an anti-B7H3 moiety in multi-chain format. For example, the tri-specific antibody may comprise a first chain comprising anti-CD137 scFv fragment fused with the heavy chain of the anti-B7H3 antibody such as that of B7H3 Ab1 or B7H3 Ab2; a second chain comprising the Fab fragment of anti-B7H3 HC and anti-CD3 scFv fragment and an Fc fragment (e.g., a whole Fc fragment or a portion thereof such as CH2-CH3); a third chain that is the light chain of the anti-B7H3 antibody. Two Fc fragments with engineered CH3 domain to create a “knob” in one and a “hole” in the other to promote heterodimerization.

In some examples, the anti-B7H3/CD3/CD137 tri-specific antibodies may comprise an anti-CD3 moiety in fused V_{H} and V_{L} fragment format and an anti-B7H3 moiety in multi-chain format. The anti-CD3 fused V_{H} and V_{L} fragment may be derived from any of the anti-CD3 antibodies disclosed herein, for example, any of those provided in Table 1 above. For example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3) and anti-CD137 scFv fragment; a second chain comprising the V_{H} fragment of anti-B7H3 moiety and anti-CD3 V_{L} fragment and an Fc fragment (e.g., a whole Fc fragment or a portion thereof such as CH2-CH3); and a third chain that is the light chain of anti-B7H3 antibody. Two Fc fragments with engineered CH3 domain to create a “knob” in one and a “hole” in the other to promote heterodimerization.

In some examples, the anti-B7H3/CD3/CD137 tri-specific antibodies may comprise an anti-CD3 moiety in fused V_{H} and V_{L} fragment format (scFv) and an anti-B7H3 moiety in multi-chain format. For example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3); a second chain comprising the V_{H} fragment of anti-B7H3 moiety and anti-CD3 V_{L} fragment and an Fc fragment (e.g., a whole Fc fragment or a portion thereof such as CH2-CH3); and a third chain comprising the light chain of anti-B7H3 antibody and anti-CD137 scFv fragment. Two Fc fragments with engineered CH3 domain to create a “knob” in one and a “hole” in the other to promote heterodimerization.

In some examples, any of the anti-B7H3/CD3/CD137 trispecific antibodies may comprise mutations for enhancing heterodimerization and/or reducing protein A binding such as those disclosed herein. Alternatively or in addition, any of the antigen binding moieties in the anti-B7H3/CD3/CD137 trispecific antibodies may be in CrossMab format.

Exemplary anti-B7H3/CD3/CD137 tri-specific antibodies are provided in Example 9 and Table 9 below, which are within the scope of the present disclosure.

### (vi) Anti-B7H31CD31CD28 Tri-Specific Antibodies

In some embodiments, the tri-specific antibodies disclosed herein binds B7H3, CD3 and CD28, for example, human B7H3, human CD3 and human CD28. Any antibody capable of binding to B7H3, CD3 and CD28 can be used in constructing the tri-specific antibodies disclosed herein. In some examples, the anti-B7H3, anti-CD3 and anti-CD28 portions of the tri-specific antibody described herein may be derived from any of the corresponding parent anti-B7H3, anti-CD3 and anti-CD28 antibodies provided in Table 1 above. For example, one or more of the anti-B7H3, anti-CD3 and anti-CD28 antigen binding moieties may comprise the same heavy chain and/or light chain CDRs as a parent antibody, e.g., those listed in Table 1 above. Alternatively, the one or more antigen binding moieties may comprise substantially similar heavy chain and/or light chain CDRs as those of the corresponding parent antibody (e.g., comprising no more than 5, 4, 3, 2, or 1 amino acid residue variations as compared with the parent antibody). In some instances, one or more of the anti-B7H3, the anti-CD3 and anti-CD28 antigen binding moieties in the tri-specific antibody may have the same heavy chain variable region and/or the same light chain variable region as the corresponding parent antibodies. For example, the antigen binding moieties in the tri-specific antibody may have the same heavy chain and/or the same light chain as the corresponding parent antibodies.

In some examples, the anti-B7H3/CD3/CD28 tri-specific antibodies may comprise an anti-B7H3 binding moiety in multi-chain format (IgG like), an anti-CD3 binding moiety in Fab format, and an anti-CD28 binding moiety in scFv format. In some instances, the anti-CD28 scFv may be fused to the heavy chains of the anti-B7H3 binding moiety (either one or both). Alternatively or in addition, one chain of the anti-CD3 Fab may be fused with one heavy chain of the anti-B7H3 binding moiety (e.g., located in the constant region, for example, between CH1 and CH2 regions). Two Fc fragments with engineered CH3 domain to create a “knob” in one and a “hole” in the other to promote heterodimerization.

In some examples, the anti-B7H3/CD3/CD28 tri-specific antibodies may comprise an anti-B7H3 binding moiety in multi-chain format (IgG like), an anti-CD3 binding moiety in scFv format, and an anti-CD28 binding moiety also in scFv format. In some instances, the anti-CD28 scFv may be fused to the heavy chains of the anti-B7H3 binding moiety (either one or both). Alternatively or in addition, the anti-CD3 scFv o may be fused with one heavy chain of the anti-B7H3 binding moiety (e.g., located in the constant region, for example, between CH1 and CH2 regions). Two Fc fragments with engineered CH3 domain to create a “knob” in one and a “hole” in the other to promote heterodimerization.

In some examples, the anti-B7H3/CD3/CD28 tri-specific antibodies may comprise an anti-B7H3 binding moiety in multi-chain format (IgG like), an anti-CD3 binding moiety in V_{H}/V_{L} format, and an anti-CD28 binding moiety in scFv format. In some instances, the anti-CD28 scFv may be fused to the heavy chains of the anti-B7H3 binding moiety (either one or both). In other instances, the anti-CD28 scFv may be fused to the light chains of the anti-B7H3 binding moiety (either one or both). Alternatively or in addition, each of the V_{H} and V_{L} of the anti-CD3 moiety may be fused with one of the heavy chains of the anti-B7H3 binding moiety (e.g., located in the constant region, for example, between CH1 and CH2 regions). Two Fc fragments with engineered CH3 domain to create a “knob” in one and a “hole” in the other to promote heterodimerization.

In some examples, the anti-B7H3/CD3/CD28 tri-specific antibodies may comprise an anti-B7H3 binding moiety in multi-chain format (IgG like), an anti-CD3 binding moiety in V_{H}/V_{L} format, and an anti-CD28 binding moiety also in V_{H}/V_{L} format. In some instances, each of the V_{H} and V_{L} of the anti-CD28 binding moiety may be fused to one of the heavy chains of the anti-B7H3 binding moiety (e.g., located at the C-terminus). Alternatively or in addition, each of the V_{H} and V_{L} of the anti-CD3 moiety may also be fused with one of the heavy chains of the anti-B7H3 binding moiety (e.g., located in the constant region, for example, between CH1 and CH2 regions). Two Fc fragments with engineered CH3 domain to create a “knob” in one and a “hole” in the other to promote heterodimerization.

In some examples, any of the anti-B7H3/CD3/CD28 trispecific antibodies may comprise mutations for enhancing heterodimerization and/or reducing protein A binding such as those disclosed herein. Alternatively or in addition, the anti-B7H3/CD3/CD28 trispecific antibodies may be in CrossMab format.

Exemplary anti-B7H3/CD3/CD28 tri-specific antibodies are provided in Example 10 and Table 10, which are within the scope of the present disclosure.

(vii) Anti-B7H31CD31OX40 Tri-Specific Antibodies

In some embodiments, the tri-specific antibodies disclosed herein binds B7H3, CD3 and OX40, for example, human B7H3, human CD3 and human OX40. Any antibody capable of binding to B7H3, CD3 and OX40 can be used in constructing the tri-specific antibodies disclosed herein. In some examples, the anti-B7H3, anti-CD3 and anti-OX40 portions of the tri-specific antibody described herein may be derived from any of the corresponding parent anti-B7H3, anti-CD3 and anti-OX40 antibodies provided in Table 1 above. For example, one or more of the anti-B7H3, anti-CD3 and anti-OX40 antigen binding moieties may comprise the same heavy chain and/or light chain CDRs as a parent antibody, e.g., those listed in Table 1 above. Alternatively, the one or more antigen binding moieties may comprise substantially similar heavy chain and/or light chain CDRs as those of the corresponding parent antibody (e.g., comprising no more than 5, 4, 3, 2, or 1 amino acid residue variations as compared with the parent antibody). In some instances, one or more of the anti-B7H3, the anti-CD3 and anti-OX40 antigen binding moieties in the tri-specific antibody may have the same heavy chain variable region and/or the same light chain variable region as the corresponding parent antibodies. For example, the antigen binding moieties in the tri-specific antibody may have the same heavy chain and/or the same light chain as the corresponding parent antibodies.

In some examples, the anti-B7H3/CD3/OX40 tri-specific antibodies may be a multi-chain molecule comprising an anti-OX40 moiety in scFv format and an anti-B7H3 moiety and anti-CD3 in V_{H}/V_{L} and/or Fab fragment format. For example, the tri-specific antibody may comprise a first chain comprising the anti-OX40 scFv fragment fused with the heavy chain of the parent anti-B7H3 antibody such as B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-OX40 scFv fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In another example, the tri-specific antibody may comprise a first chain comprising the anti-OX40 scFv fragment fused with the heavy chain of the parent anti-B7H3 antibody such as B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3); and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In yet another example, the tri-specific antibody may comprise a first chain comprising the anti-OX40 V_{H} fragment fused with the heavy chain of the anti-B7H3 antibody such as that of B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-OX40 V_{L} fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In some examples, the anti-B7H3/CD3/OX40 tri-specific antibodies may comprise an anti-CD3 moiety in fused V_{H} and V_{L} fragment format (scFv) and an anti-B7H3 moiety in multi-chain format. For example, the tri-specific antibody may comprise a first chain comprising anti-OX40 scFv fragment fused with the heavy chain of the parent anti-B7H3 antibody such as that of B7H3 Ab1 or B7H3 Ab2; a second chain comprising the Fab fragment of anti-B7H3 HC and anti-CD3 scFv fragment and an Fc fragment (e.g., a whole Fc fragment or a portion thereof such as CH2-CH3); a third chain that is the light chain of the anti-B7H3 antibody. Two Fc fragments with engineered CH3 domain to create a “knob” in one and a “hole” in the other to promote heterodimerization.

In another example, the tri-specific antibody may comprise a first chain comprising anti-OX40 scFv fragment fused with the heavy chain of the parent anti-B7H3 antibody; a second chain comprising the Fab fragment of anti-B7H3 HC and anti-CD3 scFv fragment and an Fc fragment (e.g., a whole Fc fragment or a portion thereof such as CH2-CH3) and anti-OX40 scFv fragment; a third chain that is the light chain of the anti-B7H3 antibody. Two Fc fragments with engineered CH3 domain to create a “knob” in one and a “hole” in the other to promote heterodimerization.

In yet another example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3) and anti-OX40 scFv fragment; a second chain comprising the V_{H} fragment of anti-B7H3 moiety and anti-CD3 V_{L} fragment and an Fc fragment (e.g., a whole Fc fragment or a portion thereof such as CH2-CH3) and anti-OX40 scFv fragment; and a third chain that is the light chain of anti-B7H3 antibody. Two Fc fragments with engineered CH3 domain to create a “knob” in one and a “hole” in the other to promote heterodimerization.

For example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3) and anti-OX40 scFv fragment; a second chain comprising the V_{H} fragment of anti-B7H3 moiety and anti-CD3 V_{L} fragment and an Fc fragment (e.g., a whole Fc fragment or a portion thereof such as CH2-CH3); and a third chain that is the light chain of anti-B7H3 antibody. Two Fc fragments with engineered CH3 domain to create a “knob” in one and a “hole” in the other to promote heterodimerization.

Alternatively, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3); a second chain comprising the V_{H} fragment of anti-B7H3 moiety and anti-CD3 V_{L} fragment and an Fc fragment (e.g., a whole Fc fragment or a portion thereof such as CH2-CH3) and anti-OX40 scFv fragment; and a third chain that is the light chain of anti-B7H3 antibody. Two Fc fragments with engineered CH3 domain to create a “knob” in one and a “hole” in the other to promote heterodimerization.

In addition, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3) and anti-OX40 V_{H} fragment; a second chain comprising the V_{H} fragment of anti-B7H3 moiety and anti-CD3 V_{L} fragment and an Fc fragment (e.g., a whole Fc fragment or a portion thereof such as CH2-CH3) and anti-OX40 V_{L} fragment; and a third chain that is the light chain of anti-B7H3 antibody. Two Fc fragments with engineered CH3 domain to create a “knob” in one and a “hole” in the other to promote heterodimerization.

More over, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3); a second chain comprising the V_{H} fragment of anti-B7H3 moiety and anti-CD3 V_{L} fragment and an Fc fragment (e.g., a whole Fc fragment or a portion thereof such as CH2-CH3); and a third chain comprising the light chain of anti-B7H3 antibody and anti-OX40 scFv fragment. Two Fc fragments with engineered CH3 domain to create a “knob” in one and a “hole” in the other to promote heterodimerization. Further, the tri-specific antibody may comprise a first chain comprising the heavy chain of anti-B7H3 and anti-OX40 scFv fragment; a second chain comprising the heavy chain of anti-B7H3 and anti-CD3 scFv fragment; and a third chain that is the light chain of anti-B7H3 antibody. Two Fc fragments with engineered CH3 domain to create a “knob” in one and a “hole” in the other to promote heterodimerization.

In another example, the tri-specific antibody may comprise a first chain that the heavy chain of anti-B7H3 antibody; a second chain comprising the V_{H} fragment of anti-OX40 moiety and anti-CD3 V_{H} and CH1 of anti-CD3 LC and an Fc fragment (e.g., a whole Fc fragment or a portion thereof such as CH2-CH3); a third chain comprising the light chain of anti-B7H3 and anti-CD3 V_{L} and CH1 of anti-CD3 HC; and a fourth chain that is the light chain of anti-B7H3 antibody. Two Fc fragments with engineered CH3 domain to create a “knob” in one and a “hole” in the other to promote heterodimerization.

In some examples, any of the anti-B7H3/CD3/OX40 trispecific antibodies may comprise mutations for enhancing heterodimerization and/or reducing protein A binding such as those disclosed herein. Alternatively or in addition, the anti-B7H3/CD3/OX40 trispecific antibodies may be in CrossMab format.

Exemplary anti-B7H3/CD3/OX40 tri-specific antibodies are provided in Example 11 and Table 11, which are within the scope of the present disclosure.

(viii) Anti-B7H3/CD3/GITR Tri-Specific Antibodies

In some embodiments, the tri-specific antibodies disclosed herein binds B7H3, CD3 and GITR, for example, human B7H3, human CD3 and GITR. Any antibody capable of binding to B7H3, CD3 and GITR can be used in constructing the tri-specific antibodies disclosed herein. In some examples, the anti-B7H3, anti-CD3 and anti-GITR portion of the tri-specific antibody described herein may be derived from any of the anti-CD3 and anti-GITR parent antibodies provided in Table 1 above. One or more of the anti-B7H3, anti-CD3 and anti-GITR antigen binding moieties may comprise the same heavy chain and/or light chain CDRs as a parent antibody. Alternatively, one or more of the antigen binding moieties may comprise substantially similar heavy chain and/or light chain CDRs as those of the parent antibody (e.g., comprising no more than 5, 4, 3, 2, or 1 amino acid residue variations as compared with the parent antibody). In some instances, one or more of the anti-B7H3, anti-CD3 and anti-GITRantigen binding moiety in the tri-specific antibody may have the same heavy chain variable region and/or the same light chain variable region as the parent antibody. For example, one or more of the antigen binding moiety in the tri-specific antibody may have the same heavy chain and/or the same light chain as the parent antibody.

In some examples, the anti-B7H3/CD3/GITR tri-specific antibodies may be a multi-chain molecule comprising an anti-GITR moiety in scFv format and an anti-B7H3 moiety and anti-CD3 in V_{H}/V_{L} and/or Fab format. For example, the tri-specific antibody may comprise a first chain comprising the anti-GITR scFv fragment fused with the heavy chain of the anti-B7H3 antibody such as that of B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-GITR scFv fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In some examples, the anti-B7H3/CD3/GITR tri-specific antibodies may be a multi-chain molecule comprising an anti-GITR moiety in scFv format and an anti-B7H3 moiety and anti-CD3 V_{H}/V_{L} and/or Fab fragment format. For example, the tri-specific antibody may comprise a first chain that is the heavy chain of an anti-B7H3 parent antibody such as B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-GITR scFv fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In some examples, the anti-B7H3/CD3/GITR tri-specific antibodies may be a multi-chain molecule comprising an anti-GITR moiety in scFv format and an anti-B7H3 moiety and anti-CD3 in V_{H}/V_{L} and/or Fab format. For example, the tri-specific antibody may comprise a first chain comprising the anti-GITR V_{H} fragment fused with the heavy chain of the anti-B7H3 parent antibody such as B7H3 Ab1 or B7H3 Ab2, and a second chain comprising the Fab fragment of anti-B7H3 HC and anti-CD3 V_{H} and CH1 fragment of anti-CD3 LC and an Fc fragment (e.g., a whole Fc fragment or a portion thereof such as CH2-CH3) and anti-GITR V_{L} fragment; and a third chain that is the light chain of the anti-B7H3 antibody; and a fourth chain that is anti-CD3 V_{L} with CH1 fragment of anti-CD3 HC. Two Fc fragments with engineered CH3 domain to create a “knob” in one and a “hole” in the other to promote heterodimerization.

In some examples, the anti-B7H3/CD3/GITR tri-specific antibodies may comprise an anti-CD3 moiety in fused V_{H} and V_{L} fragment format (scFv) and an anti-B7H3 moiety in multi-chain format. For example, the tri-specific antibody may comprise a first chain comprising anti-GITR scFv fragment fused with the heavy chain of the parent anti-B7H3 antibody such as B7H3 Ab1 or B7H3 Ab2; a second chain comprising the Fab fragment of anti-B7H3 HC and anti-CD3 scFv fragment and an Fc fragment (e.g., a whole Fc fragment or a portion thereof such as CH2-CH3); a third chain that is the light chain of the anti-B7H3 antibody. Two Fc fragments with engineered CH3 domain to create a “knob” in one and a “hole” in the other to promote heterodimerization.

In some examples, the anti-B7H3/CD3/GITR tri-specific antibodies may be a multi-chain molecule comprising an anti-CD3 moiety in fused V_{H} and V_{L} fragment format (scFv) and an anti-B7H3 moiety in multi-chain format. For example, the tri-specific antibody may comprise a first chain is that the heavy chain of the parent anti-B7H3 antibody, such as B7H3 Ab1 or B7H3 Ab2; a second chain comprising the Fab fragment of anti-B7H3 HC and anti-CD3 scFv fragment and an Fc fragment (e.g., a whole Fc fragment or a portion thereof such as CH2-CH3) and anti-GITR scFv fragment; a third chain that is the light chain of the anti-B7H3 antibody. Two Fc fragments with engineered CH3 domain to create a “knob” in one and a “hole” in the other to promote heterodimerization.

In some examples, the anti-B7H3/CD3/GITR tri-specific antibodies may comprise an anti-CD3 moiety in fused V_{H} and V_{L} fragment format and an anti-B7H3 moiety in multi-chain format. For example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3) and anti-GITR scFv fragment; a second chain comprising the V_{H} fragment of anti-B7H3 moiety and anti-CD3 V_{L} fragment and an Fc fragment (e.g., a whole Fc fragment or a portion thereof such as CH2-CH3) and anti-GITR scFv fragment; and a third chain that is the light chain of anti-B7H3 antibody. Two Fc fragments with engineered CH3 domain to create a “knob” in one and a “hole” in the other to promote heterodimerization.

In some examples, the anti-B7H3/CD3/GITR tri-specific antibodies may be a multi-chain molecule comprising an anti-CD3 moiety in fused V_{H} and V_{L} fragment format (scFv) and an anti-B7H3 moiety in multi-chain format. For example, the tri-specific antibody may comprise a first chain comprising the V_{H} fragment of anti-B7H3 moiety and anti-CD3 V_{H} fragment and an Fc fragment (e.g., a whole Fc fragment or a portion thereof such as CH2-CH3); a second chain comprising the V_{H} fragment of anti-B7H3 moiety and anti-CD3 V_{L} fragment and an Fc fragment (e.g., a whole Fc fragment or a portion thereof such as CH2-CH3) and a third chain comprising the light chain of anti-B7H3 antibody and anti-GITR scFv fragment. Two Fc fragments with engineered CH3 domain to create a “knob” in one and a “hole” in the other to promote heterodimerization.

In some examples, the anti-B7H3/CD3/GITR tri-specific antibodies may comprise an anti-CD3 moiety in fused V_{H} and V_{L} fragment format and an anti-B7H3 moiety in multi-chain format. For example, the tri-specific antibody may comprise a first chain that comprises the heavy chain of anti-B7H3 antibody; a second chain comprising the V_{H} fragment of anti-GITR moiety and anti-CD3 V_{H} and CH1 of anti-CD3 LC and an Fc fragment (e.g., a whole Fc fragment or a portion thereof such as CH2-CH3); a third chain comprising the light chain of anti-B7H3 and anti-CD3 V_{L} and CH1 of anti-CD3 HC; and a fourth chain that is the light chain of anti-B7H3 antibody. Two Fc fragments with engineered CH3 domain to create a “knob” in one and a “hole” in the other to promote heterodimerization.

In some examples, any of the anti-B7H3/CD3/GITR tri-specific antibodies may comprise mutations for enhancing heterodimerization and/or reducing protein A binding such as those disclosed herein. Alternatively or in addition, any of the antigen binding moieties in the tri-specific antibody may be in CrossMab format.

Exemplary anti-B7H3/CD3/GITR bi-specific antibodies are provided in Example 12 and Table 12 below, which are within the scope of the present disclosure.

### (ix) Anti-B7H3/CD137/GITR Tri-Specific Antibodies

In some embodiments, the tri-specific antibodies disclosed herein binds B7H3, CD137 and GITR, for example, human B7H3, human CD137 and human GITR. Any antibody capable of binding to B7H3, CD137 and GITR can be used in constructing the tri-specific antibodies disclosed herein. In some examples, the anti-B7H3, anti-CD137 and anti-GITR portion of the tri-specific antibody described herein may be derived from any of the anti-CD137 and anti-GITR parent antibodies provided in Table 1 above. One or more of the anti-B7H3, anti-CD137 and anti-GITR antigen binding moieties may comprise the same heavy chain and/or light chain CDRs as a parent antibody. Alternatively, one or more of the antigen binding moieties may comprise substantially similar heavy chain and/or light chain CDRs as those of the parent antibody (e.g., comprising no more than 5, 4, 3, 2, or 1 amino acid residue variations as compared with the parent antibody). In some instances, one or more of the anti-B7H3, anti-CD137 and anti-GITRantigen binding moiety in the tri-specific antibody may have the same heavy chain variable region and/or the same light chain variable region as the parent antibody. For example, one or more of the antigen binding moiety in the tri-specific antibody may have the same heavy chain and/or the same light chain as the parent antibody.

In some examples, the anti-B7H3/CD137/GITR tri-specific antibodies may comprise an anti-B7H3 binding moiety in multi-chain format (IgG like), an anti-CD137 binding moiety in scFv format, and an anti-GITR binding moiety in scFv format. The anti-CD137 scFv may be fused to the light chains of the anti-B7H3 binding moiety and the anti-GITR scFv may be fused to the heavy chains of the anti-B7H3 binding moiety. Alternatively, the anti-CD137 scFv may be fused to the heavy chains of the anti-B7H3 binding moiety and the anti-GITR scFv may be fused to the light chains of the anti-B7H3 binding moiety. In another example, the anti-CD137 scFv may be fused to one heavy chain of the anti-B7H3 binding moiety and the anti-GITR scFv may be fused to the other heavy chain of the anti-B7H3 binding moiety.

In some examples, any of the anti-B7H3/CD137/GITR tri-specific antibodies may comprise mutations for enhancing heterodimerization and/or reducing protein A binding such as those disclosed herein. Alternatively or in addition, any of the antigen binding moieties in the tri-specific antibody may be in CrossMab format.

Exemplary anti-B7H3/CD137/GITR tri-specific antibodies are provided in Example 13 and Table 13 below, which are within the scope of the present disclosure.

### (xi) Anti-B7H3/CD137/OX40 Tri-Specific Antibodies

In some embodiments, the tri-specific antibodies disclosed herein binds B7H3, CD137 and OX40, for example, human B7H3, human CD137 and human OX40. Any antibody capable of binding to B7H3, CD137 and OX40 can be used in constructing the tri-specific antibodies disclosed herein. In some examples, the anti-B7H3, anti-CD137 and anti-OX40 portion of the tri-specific antibody described herein may be derived from any of the anti-CD137 and anti-OX40 parent antibodies provided in Table 1 above. One or more of the anti-B7H3, anti-CD137 and anti-OX40 antigen binding moieties may comprise the same heavy chain and/or light chain CDRs as a parent antibody. Alternatively, one or more of the antigen binding moieties may comprise substantially similar heavy chain and/or light chain CDRs as those of the parent antibody (e.g., comprising no more than 5, 4, 3, 2, or 1 amino acid residue variations as compared with the parent antibody). In some instances, one or more of the anti-B7H3, anti-CD137 and anti-OX40 antigen binding moiety in the tri-specific antibody may have the same heavy chain variable region and/or the same light chain variable region as the parent antibody. For example, one or more of the antigen binding moiety in the tri-specific antibody may have the same heavy chain and/or the same light chain as the parent antibody.

In some examples, the anti-B7H3/CD137/OX40 tri-specific antibodies may comprise an anti-B7H3 binding moiety in multi-chain format (IgG like), an anti-CD137 binding moiety in scFv format, and an anti-OX40 binding moiety in scFv format. The anti-CD137 scFv may be fused to the light chains of the anti-B7H3 binding moiety and the anti-OX40 scFv may be fused to the heavy chains of the anti-B7H3 binding moiety. Alternatively, the anti-CD137 scFv may be fused to the heavy chains of the anti-B7H3 binding moiety and the anti-OX40 scFv may be fused to the light chains of the anti-B7H3 binding moiety. In another example, the anti-CD137 scFv may be fused to one heavy chain of the anti-B7H3 binding moiety and the anti-OX40 scFv may be fused to the other heavy chain of the anti-B7H3 binding moiety.

In some examples, any of the anti-B7H3/CD137/OX40 tri-specific antibodies may comprise mutations for enhancing heterodimerization and/or reducing protein A binding such as those disclosed herein. Alternatively or in addition, any of the antigen binding moieties in the tri-specific antibody may be in CrossMab format.

Exemplary anti-B7H3/CD137/OX40 tri-specific antibodies are provided in Example 14 and Table 14 below, which are within the scope of the present disclosure.

### III. Methods for Antibody Preparation

Any of the antibodies, including bi-specific antibodies, as described herein can be made by any method known in the art. See, for example, Harlow and Lane, (1998) Antibodies: A Laboratory Manual, Cold Spring Harbor Laboratory, New York. Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target antigen as disclosed herein can be identified from the library following routine procedures.

In some examples, any of the antibodies, including bi-specific antibodies as disclosed herein can be prepared by recombinant technology as exemplified below.

Nucleic acids encoding the heavy and light chain of the antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a resmultiction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular resmultiction site in the vector.

The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987); Gossen and Bujard (1992); M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antibody (including bi-specific antibody) as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding a first chain (e.g., a heavy chain) of the antibody and the other encoding a second chain (e.g., a light chain) of the antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled mamultix.

Any of the nucleic acids encoding the first chain (e.g., the heavy chain), the second chain (e.g., the light chain), or both of an antibody as described herein, vectors (e.g., expression vectors) containing such; and host cells comprising the vectors are within the scope of the present disclosure.

### IV. Pharmaceutical Compositions

Any of the antibodies, including bi-specific antibodies disclosed herein, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable mamultices of solid hydrophobic polymers containing the antibody, which mamultices are in the form of shaped articles, e.g. films, or microcapsules. Examples of sustained-release mamultices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 m, particularly 0.1 and 0.5 m, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### V. Therapeutic Applications

Any of the anti-B7H3/CD40 bi-specific antibodies, anti-B7H3/CD137 bi-specific antibodies, anti-B7H3/GITR bi-specific antibodies, anti-B7H3/CD40 bi-specific antibodies, anti-B7H3/OX40 bi-specific antibodies, as well as any of the anti-GITR antibodies disclosed herein, may be used in clinical settings (e.g., therapeutic or diagnostic) or in non-clinical settings (e.g., for research purposes).

In some aspects, provided herein are methods of using any of the antibodies disclosed herein for modulating immune responses or for treating a targeting disease in a subject in need of the treatment. To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as a cancer or an immune disorder such as an autoimmune disease.

Examples of cancers include, but are not limited to, breast cancer; biliary tract cancer; bladder cancer; brain cancer including glioblastomas and medulloblastomas; cervical cancer; choriocarcinoma; colon cancer; endomemultial cancer; esophageal cancer; gasmultic cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

A subject having a target cancer can be identified by routine medical examination, e.g., laboratory tests, organ functional tests, CT scans, ultrasounds, and/or genetic testing. In some embodiments, the subject to be treated by the method described herein may be a human cancer patient who has undergone or is subjecting to an anti-cancer therapy, for example, chemotherapy, radiotherapy, immunotherapy, or surgery.

Immune disorders refer to a dysfunction of the immune system. Examples include autoimmune diseases, immunodeficiencies, or allergies. In some embodiments, the target disease for treatment is an autoimmune disease. Examples include, but are not limited to, rheumatoid arthritis (RA), systemic lupus erythematosus (SLE), Myasthenia Gravis (MG), Graves' Disease, Idiopathic Thrombocytopenia Purpura (ITP), Guillain-Barre Syndrome, autoimmune myocarditis, Membrane Glomerulonephritis, Hyper IgM syndrome, diabetes mellitus, Type I or Type II diabetes, multiple sclerosis, Reynaud's syndrome, autoimmune thyroiditis, gasmultitis, Celiac Disease, Vitiligo, Hepatitis, primary biliary cirrhosis, inflammatory bowel disease, spondyloarthropathies, experimental autoimmune encephalomyelitis, immune neutropenia, juvenile onset diabetes, and immune responses associated with delayed hypersensitivity mediated by cytokines, T-lymphocytes typically found in tuberculosis, sarcoidosis, and polymyositis, polyarteritis, cutaneous vasculitis, pemphigus, pemphigold, ture's syndrome, Kawasaki's disease, systemic sclerosis, anti-phospholipid syndrome, Sjogren's syndrome, graft-versus-host (GVH) disease, and immune thrombocytopenia.

A subject having a target autoimmune disease can be identified by routine medical examination, e.g., presence of antinuclear antibodies, anti-mitochondrial autoantibodies, anti-neutrophil cytoplasmic antibody, anti-phospholipid antibodies, anti-citrullinated peptide (anti-CCP), anti-rheumatoid factor, immunoglobulin A, C-reactive protein test, complement test, erythrocyte sedimentation rate (ESR) test, blood clotting profile, and protein electrophoresis/immunofixation electrophoresis, and/or genetic testings. In some embodiments, the subject to be treated by the method described herein may be a human subject with an autoimmune disease who has undergone or is subjecting to an autoimmune disease treatment, for example, immunosuppressive mediation, hormone replacement therapy, blood transfusions, anti-inflammatory medication, and/or pain medication.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will conmultibute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.003 to 5.00 mg/kg may be administered. In some examples, the dosage of the antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

When any of the antibodies described herein is used for treating a cancer, it can be combined with an anti-cancer therapy, for example, those known in the art. Additional anti-cancer therapy includes chemotherapy, surgery, radiation, immunotherapy, gene therapy, and so forth.

Alternatively, the treatment of the present disclosure can be combined with a chemotherapeutic agent, for example, pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, multiethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nimultic oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

When any of the antibodies described herein is for use in treating an immune disorder, it can be co-used with other immunomodulatory treatments such as, e.g., therapeutic vaccines (including but not limited to GVAX, DC-based vaccines, etc.), or checkpoint inhibitors (including but not limited to agents that block CTLA4, PD1, LAG3, TIM3, etc.). In some instances, the antibody can be combined with another therapy for autoimmune diseases.

Examples include, but are not limited to, intravenous Ig therapy; nonsteroidal anti-inflammatory drugs (NSAID); corticosteroids; cyclosporins, rapamycins, ascomycins; cyclophosphamide; azathioprene; methotrexate; brequinar; FTY 720; leflunomide; mizoribine; mycophenolic acid; mycophenolate mofetil; 15-deoxyspergualine; an immunosuppressive agent, or an adhesion molecule inhibitor.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

When a second therapeutic agent is used, such an agent can be administered simultaneously or sequentially (in any order) with the therapeutic agent described herein. When co-administered with an additional therapeutic agent, suitable therapeutically effective dosages for each agent may be lowered due to the additive action or synergy.

### VI. Kits Comprising Antibodies Disclosed Herein

The present disclosure also provides kits for use in treating or alleviating a target disease, such as cancer or immune disorders as described herein. Such kits can include one or more containers comprising an anti-GITR antibody, anti-B7H3/CD40 bi-specific antibody, anti-B7H3/CD137 bi-specific antibody, anti-B7H3/GITR bi-specific antibody, anti-B7H3/CD40 bi-specific antibody, and/or anti-B7H3/OX40 bi-specific antibody, e.g., any of those described herein, and optionally a second therapeutic agent to be co-used with the antibody, which is also described herein.

In some embodiments, the kit can comprise instructions for use in accordance with any of the methods described herein. The included instructions can comprise a description of administration of the antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Construction of Anti-B7H3 Antibodies

Anti-human B7H3 antibodies were generated using standard murine hybridoma technology. Exemplary anti-B7H3 antibody, Ly383 and Ly387, were developed. The amino acid sequences of the V_{H} and V_{L} chains of antibody Ly383 and antibody Ly387 were analyzed and the CDRs were identified following the Kabat CDR definitions. The V_{H} and V_{L} sequences of Ly383 and Ly387 are provided in Table 2 below (with the CDR regions identified in boldface):

Humanized Anti-B7H3 Antibodies Derived from Ly383

Sequence alignments were performed to compare the Ly383 V_{H} and V_{L} to human germline V_{H} and V_{L} sequences, respectively, following methods known in the art. See, e.g., Glanville J. et al. PNAS 2009; 106 (48) 20216-21. Based on overall sequence identity, matching interface positions and similarly classed CDR canonical positions, a germline family was identified for each of the light and heavy chains as the desired acceptor frameworks, i.e., IGKV3-11*01 for the light chain and IGHV1-46*01 for the heavy chain. Human acceptors were identified, the amino acid sequences of which are shown in Table 2.

The CDRs of the parent Ly383 antibody were grafted into the corresponding CDR regions of the above-noted human V_{H} and V_{L} acceptor sequences to generate humanized Ly383_VH-1 and Ly383_VL-1 chains (grafted humanized antibody; clone Ly1426), the amino acid sequence of each of which is provided in Table 2 below (CDRs in boldface):

Homology modeling of Ly383 antibody Fv fragments was carried out as follows. Briefly, the Ly383 V_{H} and V_{L} sequences were BLAST searched against the PDB antibody database to identify a suitable template for Fv fragments and especially for building the domain interface. Structural template 2GKI (Structural and Functional Coupling of Hsp90- and Sgt1-Centred Multi-Protein Complexes) was selected, identity=72%.

Homology models were built using customized Build Homology Models protocol. Disulfide bridges were specified and linked. Loops were optimized using DOPE method. Based on the homology model of 2GKI, the V_{H} and V_{L} sequences of the Ly383 antibody were analyzed. Framework region (FR) residues that are expected to be important for the binding activity, including canonical FR residues and V_{H}-V_{L} interface residues of the antibody were identified. The framework residues in the inner core were further analyzed and 6 residues of Ly383_VH-1 (grafted Ly383_V_{H}) were identified for back mutations, including A40K, M48I, V67A, R71S, T73K and R94S. As for Ly383_VL-1 (grafted LY383_V_{L}), 4 residues of were identified for back mutations, including L46P, L47W, G66R and F71Y. The amino acid sequences of these humanized V_{H} and V_{L} chains are provided in Table 2 below (corresponding to clone Ly1562)

Recombinant full-length human IgG/kappa of humanized Ly383 antibodies were constructed. The humanized Ly383 antibodies include:
Ly1426 (including a heavy chain of VH-1/IgG1 mut and a light chain of VL-1/kappa)
Ly1562 (including a heavy chain of VH-6/IgG1 mut and a light chain of VL-6/kappa)

The whole heavy chain and light chain amino acid sequences of clones Ly1426 and Ly1562 are also provided in Table 2 below.

PTM hot spots were identified and removed based on the humanized and backmutated antibody Ly1562, amino acid sequences with PTM removal design are provided in Table 2 below (CDRs in boldface, PTM removal underlined) (clones Ly1612, Ly1614, Ly1616, and Ly1618).

Humanized Anti-B7H3 Antibodies Derived from Ly387

Sequence alignments were performed to compare the Ly387 V_{H} and V_{L} to human germline V_{H} and V_{L} sequences, respectively, following methods known in the art. See, e.g., Glanville J. et al. PNAS 2009; 106 (48) 20216-21. Based on overall sequence identity, matching interface positions and similarly classed CDR canonical positions, a germline family was identified for each of the light and heavy chains as the desired acceptor frameworks, i.e., IGKV3-11*01 for the light chain and IGHV1-2*02 for the heavy chain. Human acceptors were identified, the amino acid sequences of which are shown in Table 2 below.

The CDRs of the parent Ly387 antibody were grafted into the corresponding CDR regions of the above-noted human V_{H} and V_{L} acceptor sequences to generate humanized Ly387_VH-1 and Ly387_VL-1 chains (grafted humanized antibody), the amino acid sequence of each of which is provided in Table 2 below (CDRs in boldface).

Recombinant full human IgG/kappa of humanized Ly387 antibodies were constructed. The humanized Ly387 antibodies include:
Ly1442 (including a heavy chain of Ly387-VH-1/IgG1mut and a light chain of Ly387-VL-1/kappa),

The amino acid sequences of the whole heavy chain and light chains of Ly1442 are also provided in Table 2 below.

TABLE 2
Anti-B7H3 Antibodies

| | | | SEQ ID |
|---|---|---|---|
| Antibody Clone | | Amino acid Sequence | NO: |
| Ly383 | VH | EVQLQQSGPELVKPGASVKMSCKASGYTFTSYVMHWVKQKPGQGLEWIGYIN | 27 |
| | | PYNDGTECTDKFKGKATLTSDKSSSTAYMELSSLTSEDSAVYYCASIYYGYD | |
| | | GTYFGVWGAGTSVTVSS | |
| | H chain | EVQLQQSGPELVKPGASVKMSCKASGYTFTSYVMHWVKQKPGQGLEWIGYIN | 28 |
| | | PYNDGTECTDKFKGKATLTSDKSSSTAYMELSSLTSEDSAVYYCASIYYGYD | |
| | | GTYFGVWGAGTSVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | QIVLSQSPAILSTSPGEKVTMTCRASSSVSYMHWYQQKPGSSPKPWIYATSN | 29 |
| | | LASGVPARFSGSRSGTSYSLTISRVEAEDAATYYCQQWSSNTLTFGGGTKLE | |
| | | LK | |
| | L chain | QIVLSQSPAILSTSPGEKVTMTCRASSSVSYMHWYQQKPGSSPKPWIYATSN | 30 |
| | | LASGVPARFSGSRSGTSYSLTISRVEAEDAATYYCQQWSSNTLTFGGGTKLE | |
| | | LKRTVAAPSVFIFPPSDEQLKSGTASVVCLLNNFYPREAKVQWKVDNALQSG | |
| | | NSQESVTEQDSKDSTYSLSSTLTLSKADYEKHKVYACEVTHQGLSSPVTKSF | |
| | | NRGEC | |
| Ly387 | VH | QVQLQQPGAELVKPGASVKLSCKASGYTFTSYWMHWVKQRPGQGLEWIGMIH | 31 |
| | | PNSGGTNYNEKFKGKGTLTVDKSSSTAYMQLSSLTSDDSAVYYCVTSQATWE | |
| | | AYWGQGTLVTVSA | |
| | H chain | QVQLQQPGAELVKPGASVKLSCKASGYTFTSYWMHWVKQRPGQGLEWIGMIH | 32 |
| | | PNSGGTNYNEKFKGKGTLTVDKSSSTAYMQLSSLTSDDSAVYYCVTSQATWF | |
| | | AYWGQGTLVTVSAASTKGPSVFPLAPSSKSTSGGTAALGCLVKDYFPEPVTV | |
| | | SWNSGALTSGVHTFPAVLQSSGLYSLSSVVTVPSSSLGTQTYICNVNHKPSN | |
| | | TKVDKKVEPKSCDKTHTCPPCPAPELLGGPSVFLFPPKPKDTLMISRTPEVT | |
| | | CVVVDVSHEDPEVKFNWYVDGVEVHNAKTKPREEQYNSTYRVVSVLTVLHQD | |
| | | WLNGKEYKCKVSNKALPAPIEKTISKAKGQPREPQVYTLPPSREEMTKNQVS | |
| | | LTCLVKGFYPSDIAVEWESNGQPENNYKTTPPVLDSDGSFFLYSKLTVDKSR | |
| | | WQQGNVFSCSVMHEALHNHYTQKSLSLSPGK | |
| | VL | ENVLTQSPAIMSVSPGEKVTMTCRASSSVSSSYLHWYQQKSGASPKFWIYST | 33 |
| | | SNLASGVPARFSGSGSGTSYSLTISSVEAEDAATYYCQHYSGYPLTFGAGTK | |
| | | LELR | |
| | L chain | ENVLTQSPAIMSVSPGEKVTMTCRASSSVSSSYLHWYQQKSGASPKFWIYST | 34 |
| | | SNLASGVPARFSGSGSGTSYSLTISSVEAEDAATYYCQHYSGYPLTFGAGTK | |
| | | LELRRTVAAPSVFIFPPSDEQLKSGTASVVCLLNNFYPREAKVQWKVDNALQ | |
| | | SGNSQESVTEQDSKDSTYSLSSTLTLSKADYEKHKVYACEVTHQGLSSPVTK | |
| | | SFNRGEC | |
| Ly1426 | VH | QVQLVQSGAEVKKPGASVKVSCKASGYTFTSYVMHWVRQAPGQGLEWMGYIN | 35 |
| | (Ly383- | PYNDGTECTDKFKGRVTMTRDTSTSTVYMELSSLRSEDTAVYYCARIYYGYD | |
| | VH-1) | GTYFGVWGQGTLVTVSS | |
| | H chain | QVQLVQSGAEVKKPGASVKVSCKASGYTFTSYVMHWVRQAPGQGLEWMGYIN | 36 |
| | | PYNDGTECTDKFKGRVTMTRDTSTSTVYMELSSLRSEDTAVYYCARIYYGYD | |
| | | GTYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | EIVLTQSPATLSLSPGERATLSCRASSSVSYMHWYQQKPGQAPRLLIYATSN | 37 |
| | (Ly383- | LASGIPARFSGSGSGTDFTLTISSLEPEDFAVYYCQQWSSNTLTFGGGTKVE | |
| | VL-1) | IK | |
| | L chain | EIVLTQSPATLSLSPGERATLSCRASSSVSYMHWYQQKPGQAPRLLIYATSN | 38 |
| | | LASGIPARFSGSGSGTDFTLTISSLEPEDFAVYYCQQWSSNTLTFGGGTKVE | |
| | | IKRTVAAPSVFIFPPSDEQLKSGTASVVCLLNNFYPREAKVQWKVDNALQSG | |
| | | NSQESVTEQDSKDSTYSLSSTLTLSKADYEKHKVYACEVTHQGLSSPVTKSF | |
| | | NRGEC | |
| Ly1562 | VH | QVQLVQSGAEVKKPGASVKVSCKASGYTFTSYVMHWVRQKPGQGLEWIGYIN | 39 |
| | (Ly383- | PYNDGTECTDKFKGRATMTSDKSTSTVYMELSSLRSEDTAVYYCASIYYGYD | |
| | VH-6) | GTYFGVWGQGTLVTVSS | |
| | H chain | QVQLVQSGAEVKKPGASVKVSCKASGYTFTSYVMHWVRQKPGQGLEWIGYIN | 40 |
| | | PYNDGTECTDKFKGRATMTSDKSTSTVYMELSSLRSEDTAVYYCASIYYGYD | |
| | | GTYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | EIVLTQSPATLSLSPGERATLSCRASSSVSYMHWYQQKPGQAPRPWIYATSN | 41 |
| | (Ly383- | LASGIPARFSGSRSGTDYTLTISSLEPEDFAVYYCQQWSSNTLTFGGGTKVE | |
| | VL-6) | IK | |
| | L chain | EIVLTQSPATLSLSPGERATLSCRASSSVSYMHWYQQKPGQAPRPWIYATSN | 42 |
| | | LASGIPARFSGSRSGTDYTLTISSLEPEDFAVYYCQQWSSNTLTFGGGTKVE | |
| | | IKRTVAAPSVFIFPPSDEQLKSGTASVVCLLNNFYPREAKVQWKVDNALQSG | |
| | | NSQESVTEQDSKDSTYSLSSTLTLSKADYEKHKVYACEVTHQGLSSPVTKSF | |
| | | NRGEC | |
| Ly1442 | VH | QVQLVQSGAEVKKPGASVKVSCKASGYTFTSYWMHWVRQAPGQGLEWMGMIH | 43 |
| | (Ly387- | PNSGGTNYNEKFKGRVTMTRDTSISTAYMELSRLRSDDTAVYYCARSQATWF | |
| | VH-1) | AYWGQGTLVTVSS | |
| | H chain | QVQLVQSGAEVKKPGASVKVSCKASGYTFTSYWMHWVRQAPGQGLEWMGMIH | 44 |
| | | PNSGGTNYNEKFKGRVTMTRDTSISTAYMELSRLRSDDTAVYYCARSQATWF | |
| | | AYWGQGTLVTVSSASTKGPSVFPLAPSSKSTSGGTAALGCLVKDYFPEPVTV | |
| | | SWNSGALTSGVHTFPAVLQSSGLYSLSSVVTVPSSSLGTQTYICNVNHKPSN | |
| | | TKVDKKVEPKSCDKTHTCPPCPAPELLGGPSVFLFPPKPKDTLMISRTPEVT | |
| | | CVVVDVSHEDPEVKFNWYVDGVEVHNAKTKPREEQYNSTYRVVSVLTVLHQD | |
| | | WLNGKEYKCKVSNKALPAPIEKTISKAKGQPREPQVYTLPPSREEMTKNQVS | |
| | | LTCLVKGFYPSDIAVEWESNGQPENNYKTTPPVLDSDGSFFLYSKLTVDKSR | |
| | | WQQGNVFSCSVMHEALHNHYTQKSLSLSPGK | |
| | VL | EIVLTQSPATLSLSPGERATLSCRASSSVSSSYLHWYQQKPGQAPRLLIYST | 45 |
| | (Ly387- | SNLASGIPARFSGSGSGTDFTLTISSLEPEDFAVYYCQHYSGYPLTFGGGTK | |
| | VL-1) | VEIK | |
| | L chain | EIVLTQSPATLSLSPGERATLSCRASSSVSSSYLHWYQQKPGQAPRLLIYST | 46 |
| | | SNLASGIPARFSGSGSGTDFTLTISSLEPEDFAVYYCQHYSGYPLTFGGGTK | |
| | | VEIKRTVAAPSVFIFPPSDEQLKSGTASVVCLLNNFYPREAKVQWKVDNALQ | |
| | | SGNSQESVTEQDSKDSTYSLSSTLTLSKADYEKHKVYACEVTHQGLSSPVTK | |
| | | SFNRGEC | |
| Ly1612 | VH | QVQLVQSGAEVKKPGASVKVSCKASGYTFTSYVMHWVRQKPGQGLEWIGYIN | 47 |
| | (Ly383-VH- | PYNEGTESTDKFKGRATMTSDKSTSTVYMELSSLRSEDTAVYYCASIYYGYE | |
| | 6_fix-6) | GTYFGVWGQGTLVTVSS | |
| | H chain | QVQLVQSGAEVKKPGASVKVSCKASGYTFTSYVMHWVRQKPGQGLEWIGYIN | 48 |
| | | PYNEGTESTDKFKGRATMTSDKSTSTVYMELSSLRSEDTAVYYCASIYYGYE | |
| | | GTYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | Same Ly383-VL-6 | 41 |
| | L chain | Same as light chain of Ly1562 | 42 |
| Ly1614 | VH | QVQLVQSGAEVKKPGASVKVSCKASGYTFTSYVMHWVRQKPGQGLEWIGYIN | 49 |
| | (Ly383- | PYNEGTESTDKFKGRATMTSDKSTSTVYMELSSLRSEDTAVYYCASIYYGYD | |
| | VH- | ATYFGVWGQGTLVTVSS | |
| | 6_fix-7) | | |
| | H chain | QVQLVQSGAEVKKPGASVKVSCKASGYTFTSYVMHWVRQKPGQGLEWIGYIN | 50 |
| | | PYNEGTESTDKFKGRATMTSDKSTSTVYMELSSLRSEDTAVYYCASIYYGYD | |
| | | ATYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | Same as Ly383-VL-6 | 41 |
| | L chain | Same as light chain of Ly1562 | 42 |
| Ly1616 | VH | QVQLVQSGAEVKKPGASVKVSCKASGYTFTSYVMHWVRQKPGQGLEWIGYIN | 51 |
| | (Ly383- | PYNDATESTDKFKGRATMTSDKSTSTVYMELSSLRSEDTAVYYCASIYYGYE | |
| | VH- | GTYFGVWGQGTLVTVSS | |
| | 6_fix-8) | | |
| | H chain | QVQLVQSGAEVKKPGASVKVSCKASGYTFTSYVMHWVRQKPGQGLEWIGYIN | 52 |
| | | PYNDATESTDKFKGRATMTSDKSTSTVYMELSSLRSEDTAVYYCASIYYGYE | |
| | | GTYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | VL | Same as Ly383-VL-6 | 41 |
| | L chain | Same as light chain of Ly1562 | 42 |
| Ly1618 | VH | QVQLVQSGAEVKKPGASVKVSCKASGYTFTSYVMHWVRQKPGQGLEWIGYIN | 53 |
| | (Ly383- | PYNDATESTDKFKGRATMTSDKSTSTVYMELSSLRSEDTAVYYCASIYYGYD | |
| | VH- | ATYFGVWGQGTLVTVSS | |
| | 6_fix-9) | | |
| | H chain | QVQLVQSGAEVKKPGASVKVSCKASGYTFTSYVMHWVRQKPGQGLEWIGYIN | 54 |
| | | PYNDATESTDKFKGRATMTSDKSTSTVYMELSSLRSEDTAVYYCASIYYGYD | |
| | | ATYFGVWGQGTLVTVSSASTKGPSVFPLAPSSKSTSGGTAALGCLVKDYFPE | |
| | | PVTVSWNSGALTSGVHTFPAVLQSSGLYSLSSVVTVPSSSLGTQTYICNVNH | |
| | | KPSNTKVDKKVEPKSCDKTHTCPPCPAPELLGGPSVFLFPPKPKDTLMISRT | |
| | | PEVTCVVVDVSHEDPEVKFNWYVDGVEVHNAKTKPREEQYNSTYRVVSVLTV | |
| | | LHQDWLNGKEYKCKVSNKALPAPIEKTISKAKGQPREPQVYTLPPSREEMTK | |
| | | NQVSLTCLVKGFYPSDIAVEWESNGQPENNYKTTPPVLDSDGSFFLYSKLTV | |
| | | DKSRWQQGNVF SCSVMHEALHNHYTQKSLSLSPGK | |
| | VL | Same as Ly383-VL-6 | 41 |
| | L chain | Same as light chain of Ly1562 | 42 |
| Human | VH | QVQLVQSGAEVKKPGASVKVSCKASGYTFTSYYMHWVRQAPGQGLEWMGIIN | 55 |
| Acceptor | | PSGGSTSYAQKFQGRVTMTRDTSTSTVYMELSSLRSEDTAVYYCARIYYGYD | |
| for | | GTYFGVWGQGTLVTVSS | |
| Ly383 | VL | EIVLTQSPATLSLSPGERATLSCRASQSVSSYLAWYQQKPGQAPRLLIYDAS | 56 |
| | | NRATGIPARFSGSGSGTDFTLTISSLEPEDFAVYYCQQWSSNTLTFGGGTKV | |
| | | EIK | |
| Human | VH | QVQLVQSGAEVKKPGASVKVSCKASGYTFTGYYMHWVRQAPGQGLEWMGWIN | 57 |
| Acceptor | | PNSGGTNYAQKFQGRVTMTRDTSISTAYMELSRLRSDDTAVYYCARSQATWE | |
| for | | AYWGQGTLVTVSS | |
| Ly387 | VL | EIVLTQSPATLSLSPGERATLSCRASQSVSSYLAWYQQKPGQAPRLLIYDAS | 58 |
| | | NRATGIPARFSGSGSGTDFTLTISSLEPEDFAVYYCQHYSGYPLTFGGGTKV | |
| | | EIK | |

### Example 2: Characterization of Anti-B7H3 Antibodies

### (a) Binding Activity to Cell Surface B7H3

FACS analysis was performed to evaluate the binding properties of exemplary anti-B7H3 humanized antibodies. Briefly, CHO cells over-expressing human B7H3 were harvested using trypsin-EDTA partial digestion followed by centrifugation at 1000 g for 3 minutes. The cells were re-suspended in cold PBS-BSA (2%) at 2×10⁶/mL and aliquoted to 100 μL/tube. The anti-B7H3 humanized antibodies were serially diluted in PBS-BSA and 50 μL of each was added to the CHO-B7H3 cells. The cell were mixed and incubated at 4° C. in the dark for 2 hours, then washed with PBS-BSA twice. Secondary antibody conjugates (goat F(ab′)₂ anti-human IgG-Fc (PE), pre-adsorbed, Abcam #ab98596) at 1/500 dilution, 100 μL/well, was used to resuspend the cells. The cells were incubated 4° C. in dark for another 1 hour and washed twice with PBS-BSA, followed by fixation in 2% PFA/PBS, and then subjected to FACS analysis.

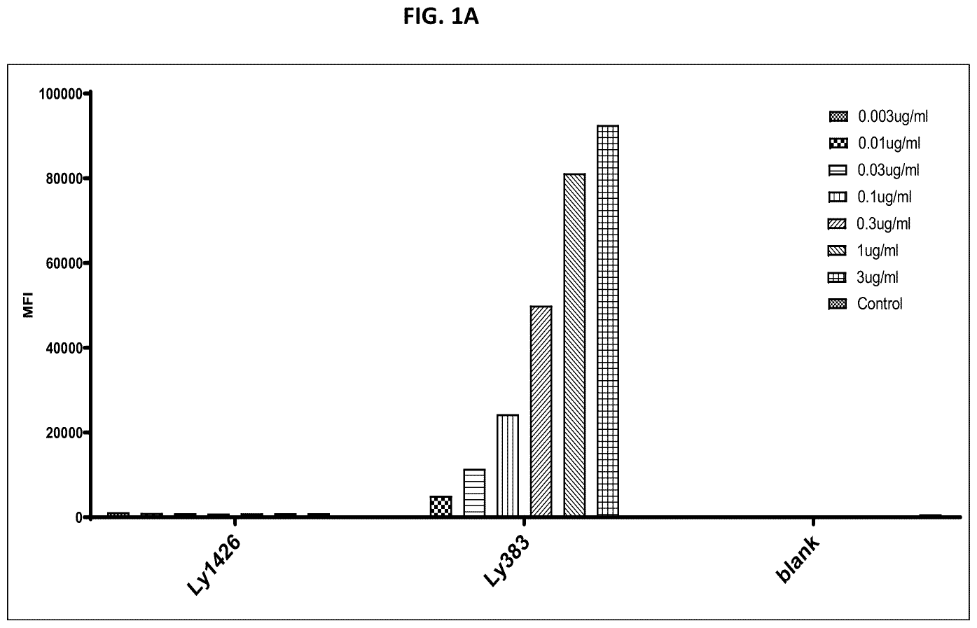

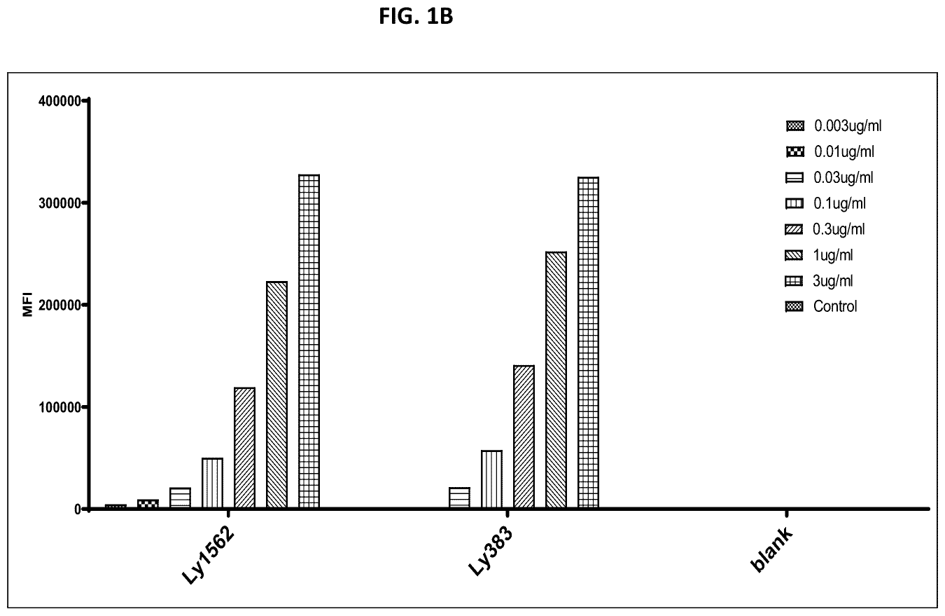

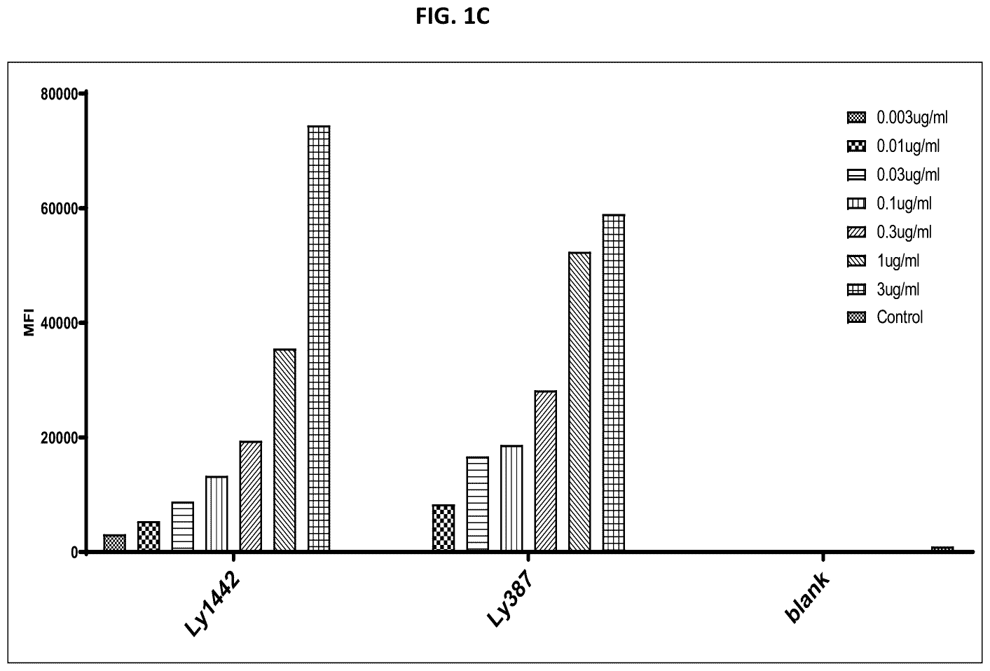

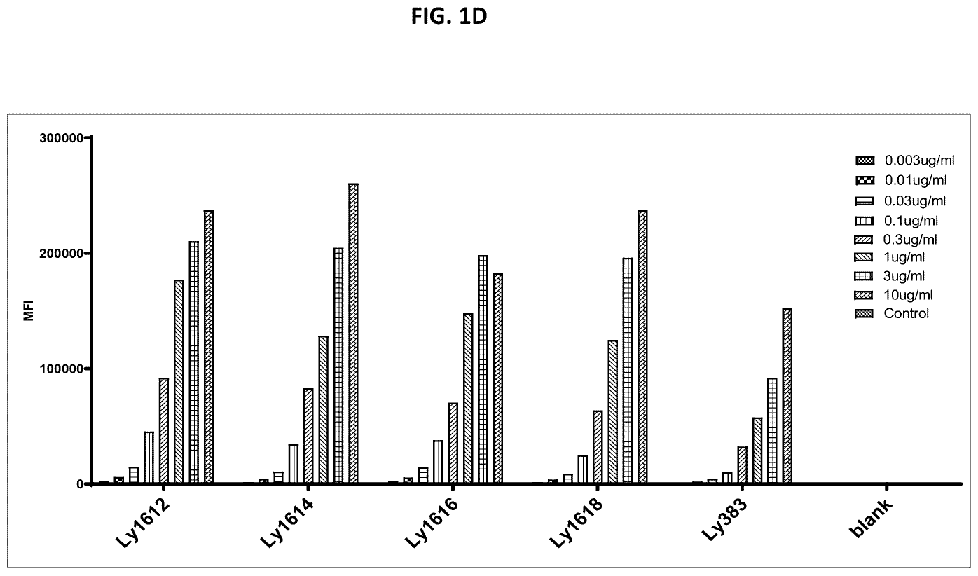

Binding of the antibodies to human B7H3 expressing CHO cells were evaluated and the mean fluorescence intensity is plotted in histograms as shown in FIGS. 1A-1C. Both Ly1562 (with back mutation) and Ly1442 (CDR grafted), humanized antibodies of the anti-B7H3 antibody Ly383 and Ly387 showed similar binding activity to the cellular B7H3 as parental chimeric antibody Ly383 and Ly387. However, Ly1426 (CDR grafted, without back mutation) didn't bind to cellular B7H3. As shown in FIG. 1D, antibodies with PTM removal design derived from Ly1562 showed similar binding activity as parent chimeric (Ly383) and humanized antibody (Ly1562).

### (b) Other Activities for B7H3

These humanized anti-B7H3 antibodies are evaluated for their in vitro and in vivo activity.

### Example 3: Anti-B7H3/CD40 Bi-Specific Antibodies

Anti-B7H3/CD40 bi-specific antibodies were produced using the anti-CD40 agonist antibody CD40 Ab1 (Ly253-G2) and anti-B7H3 antibodies B7H3 Ab1 (Ly1612) and B7H3 Ab2 (Ly1442). The amino acid sequences of the V_{H} and the V_{L} of the parent clones are provided in Table 1 above. The heavy chain and light chain complementary determining regions determined by the Kabat scheme are in boldface.

cDNAs encoding the V_{H} and V_{L} chains of those anti-CD40 and anti-7H3 antibodies (sequences provided above) were used as the starting materials for constructing anti-B7H3/CD40 bispecific antibodies. CHO-cell transient expression was carried out with am plasmids configured for expressing polypeptide chains of the bi-specific antibodies. These antibodies were purified by protein A affinity chromatography.

The amino acid sequences of the polypeptides of the bi-specific antibodies are provided I Table 3 below.

TABLE 3
Exemplary Bispecific Antibodies to B7H3 and CD40

| | | | SED ID |
|---|---|---|---|
| Antibody Clones | | Amino acid sequence | NO: |
| Ly1579 | 1^{st} | QVQLVQSGAEVKKPGASVKVSCKASGYTFTSYWMHWVRQAPGQGLEWMG | 59 |
| | chain | MIHPNSGGTNYNEKFKGRVTMTRDTSISTAYMELSRLRSDDTAVYYCAR | |
| | | SQATWFAYWGQGTLVTVSSASTKGPSVFPLAPSSKSTSGGTAALGCLVK | |
| | | DYFPEPVTVSWNSGALTSGVHTFPAVLQSSGLYSLSSVVTVPSSSLGTQ | |
| | | TYICNVNHKPSNTKVDKKVEPKSCDKTHTCPPCPAPELLGPSVFLFPPK | |
| | | PKDTLMISRTPEVTCVVVDVSHEDPEVKFNWYVDGVEVHNAKTKPREEQ | |
| | | YNSTYRVVSVLTVLHQDWLNGKEYKCKVSNKALPAPIEKTISKAKGQPR | |
| | | EPQVYTLPPSREEMTKNQVSLTCLVKGFYPSDIAVEWESNGQPENNYKT | |
| | | TPPVLDSDGSFFLYSKLTVDKSRWQQGNVFSCSVMHEALHNHYTQKSLS | |
| | | LSPGGGGGSGGGGSGGGGSGGGGSDIQMTQSPSSVSASVGDRVTITCRA | |
| | | SQGIYSWLAWYQQKPGKAPNLLIYTASTLQSGVPSRFSGSGSGTDFTLT | |
| | | ISSLQPEDFATYYCQQANIFPLTFGGGTKVEIKGGGGSGGGGSGGGGSG | |
| | | GGGSQVQLVQSGAEVKKPGASVKVSCKASGYTFTGYYMHWVRQAPGQGL | |
| | | EWMGWINPDSGGTNYAQKFQGRVTMTRDTSISTAYMELNRLRSDDTAVY | |
| | | YCARDQPLGYCTNGVCSYFDYWGQGTLVTVSS | |
| | 2^{nd} | EIVLTQSPATLSLSPGERATLSCRASSSVSSSYLHWYQQKPGQAPRLLI | 60 |
| | chain | YSTSNLASGIPARFSGSGSGTDFTLTISSLEPEDFAVYYCQHYSGYPLT | |
| | | FGGGTKVEIKRTVAAPSVFIFPPSDEQLKSGTASVVCLLNNFYPREAKV | |
| | | QWKVDNALQSGNSQESVTEQDSKDSTYSLSSTLTLSKADYEKHKVYACE | |
| | | VTHQGLSSPVTKSFNRGEC | |
| Ly1581 | 1^{st} | QVQLVQSGAEVKKPGASVKVSCKASGYTFTSYVMHWVRQKPGQGLEWIG | 61 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSVSASVGDRVTI | |
| | | TCRASQGIYSWLAWYQQKPGKAPNLLIYTASTLQSGVPSRFSGSGSGTD | |
| | | FTLTISSLQPEDFATYYCQQANIFPLTFGGGTKVEIKGGGGSGGGGSGG | |
| | | GGSGGGGSQVQLVQSGAEVKKPGASVKVSCKASGYTFTGYYMHWVRQAP | |
| | | GQGLEWMGWINPDSGGTNYAQKFQGRVTMTRDTSISTAYMELNRLRSDD | |
| | | TAVYYCARDQPLGYCTNGVCSYFDYWGQGTLVTVSS | |
| | 2^{nd} | EIVLTQSPATLSLSPGERATLSCRASSSVSYMHWYQQKPGQAPRPWIYA | 62 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1663 | 1^{st} | QVQLVQSGAEVKKPGASVKVSCKASGYTFTSYVMHWVRQKPGQGLEWIG | 63 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 64 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSVS | |
| | | ASVGDRVTITCRASQGIYSWLAWYQQKPGKAPNLLIYTASTLQSGVPSR | |
| | | FSGSGSGTDFTLTISSLQPEDFATYYCQQANIFPLTFGGGTKVEIKGGG | |
| | | GSGGGGSGGGGSGGGGSQVQLVQSGAEVKKPGASVKVSCKASGYTFTGY | |
| | | YMHWVRQAPGQGLEWMGWINPDSGGTNYAQKFQGRVTMTRDTSISTAYM | |
| | | ELNRLRSDDTAVYYCARDQPLGYCTNGVCSYFDYWGQGTLVTVSS | |
| Ly1662 | 1^{st} | DIQMTQSPSSVSASVGDRVTITCRASQGIYSWLAWYQQKPGKAPNLLIY | 65 |
| | chain | TASTLQSGVPSRFSGSGSGTDFTLTISSLQPEDFATYYCQQANIFPLTF | |
| | | GGGTKVEIKGGGGSGGGGSGGGGSGGGGSQVQLVQSGAEVKKPGASVKV | |
| | | SCKASGYTFTGYYMHWVRQAPGQGLEWMGWINPDSGGTNYAQKFQGRVT | |
| | | MTRDTSISTAYMELNRLRSDDTAVYYCARDQPLGYCTNGVCSYFDYWGQ | |
| | | GTLVTVSSGGGGSGGGGSGGGGSGGGGSQVQLVQSGAEVKKPGASVKVS | |
| | | CKASGYTFTSYVMHWVRQKPGQGLEWIGYINPYNEGTESTDKFKGRATM | |
| | | TSDKSTSTVYMELSSLRSEDTAVYYCASIYYGYEGTYFGVWGQGTLVTV | |
| | | SSASTKGPSVFPLAPSSKSTSGGTAALGCLVKDYFPEPVTVSWNSGALT | |
| | | SGVHTFPAVLQSSGLYSLSSVVTVPSSSLGTQTYICNVNHKPSNTKVDK | |
| | | KVEPKSCDKTHTCPPCPAPELLGPSVFLFPPKPKDTLMISRTPEVTCVV | |
| | | VDVSHEDPEVKFNWYVDGVEVHNAKTKPREEQYNSTYRVVSVLTVLHQD | |
| | | WLNGKEYKCKVSNKALPAPIEKTISKAKGQPREPQVYTLPPSREEMTKN | |
| | | QVSLTCLVKGFYPSDIAVEWESNGQPENNYKTTPPVLDSDGSFFLYSKL | |
| | | TVDKSRWQQGNVFSCSVMHEALHNHYTQKSLSLSPGK | |
| | 2^{nd} | EIVLTQSPATLSLSPGERATLSCRASSSVSYMHWYQQKPGQAPRPWIYA | 66 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1660 | 1^{st} | QVQLVQSGAEVKKPGASVKVSCKASGYTFTSYVMHWVRQKPGQGLEWIG | 67 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTGYYMHWVRQAPGQGLEWMG | 68 |
| | chain | WINPDSGGTNYAQKFQGRVTMTRDTSISTAYMELNRLRSDDTAVYYCAR | |
| | | DQPLGYCTNGVCSYFDYWGQGTLVTVSSASVAAPSVFIFPPSDEQLKSG | |
| | | TASVVCLLNNFYPREAKVQWKVDNALQSGNSQESVTEQDSKDSTYSLSS | |
| | | TLTLSKADYEKHKVYACEVTHQGLSSPVTKSFNRGECDKTHTCPPCPAP | |
| | | ELLGPSVFLFPPKPKDTLMISRTPEVTCVVVDVSHEDPEVKFNWYVDGV | |
| | | EVHNAKTKPREEQYNSTYRVVSVLTVLHQDWLNGKEYKCKVSNKALPAP | |
| | | IEKTISKAKGQPREPQVCTLPPSREEMTKNQVSLSCAVKGFYPSDIAVE | |
| | | WESNGQPENNYKTTPPVLDSDGSFFLVSKLTVDKSRWQQGNVFSCSVMH | |
| | | EALHNRFTQKSLSLSPGK | |
| | 3^{rd} | EIVLTQSPATLSLSPGERATLSCRASSSVSYMHWYQQKPGQAPRPWIYA | 69 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSVSASVGDRVTITCRASQGIYSWLAWYQQKPGKAPNLLIY | 70 |
| | chain | TASTLQSGVPSRFSGSGSGTDFTLTISSLQPEDFATYYCQQANIFPLTF | |
| | | GGGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1661 | 1^{st} | QVQLVQSGAEVKKPGASVKVSCKASGYTFTSYVMHWVRQKPGQGLEWIG | 71 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTGYYMHWVRQAPGQGLEWMG | 72 |
| | chain | WINPDSGGTNYAQKFQGRVTMTRDTSISTAYMELNRLRSDDTAVYYCAR | |
| | | DQPLGYCTNGVCSYFDYWGQGTLVTVSSASVAAPSVFIFPPSDEQLKSG | |
| | | TASVVCLLNNFYPREAKVQWKVDNALQSGNSQESVTEQDSKDSTYSLSS | |
| | | TLTLSKADYEKHKVYACEVTHQGLSSPVTKSFNRGECDKTHTCPPCPAP | |
| | | ELLGPSVFLFPPKPKDTLMISRTPEVTCVVVDVSHEDPEVKFNWYVDGV | |
| | | EVHNAKTKPREEQYNSTYRVVSVLTVLHQDWLNGKEYKCKVSNKALPAP | |
| | | IEKTISKAKGQPREPQVYTKPPSREEMTKNQVSLTCKVKGFYPSDIAVE | |
| | | WESNGQPENNYKTTPPVLDSDGSFFLYSKLTVDKSRWQQGNVFSCSVMH | |
| | | EALHNRFTQKSLSLSPGK | |
| | 3^{rd} | EIVLTQSPATLSLSPGERATLSCRASSSVSYMHWYQQKPGQAPRPWIYA | 73 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSVSASVGDRVTITCRASQGIYSWLAWYQQKPGKAPNLLIY | 74 |
| | chain | TASTLQSGVPSRFSGSGSGTDFTLTISSLQPEDFATYYCQQANIFPLTF | |
| | | GGGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1679 | 1^{st} | QVQLVQSGAEVKKPGASVKVSCKASGYTFTSYVMHWVRQKPGQGLEWIG | 75 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | QVQLVQSGAEVKKPGASVKVSCKASGYTFTGYYMHWVRQAPGQGLEWMG | 76 |
| | chain | WINPDSGGTNYAQKFQGRVTMTRDTSISTAYMELNRLRSDDTAVYYCAR | |
| | | DQPLGYCTNGVCSYFDYWGQGTLVTVSSASVAAPSVFIFPPSDEQLKSG | |
| | | TASVVCLLNNFYPREAKVQWKVDNALQSGNSQESVTEQDSKDSTYSLSS | |
| | | TLTLSKADYEKHKVYACEVTHQGLSSPVTKSFNRGECDKTHTCPPCPAP | |
| | | ELLGPSVFLFPPKPKDTLMISRTPEVTCVVVDVSHEDPEVKFNWYVDGV | |
| | | EVHNAKTKPREEQYNSTYRVVSVLTVLHQDWLNGKEYKCKVSNKALPAP | |
| | | IEKTISKAKGQPREPQVYVLPPSREEMTKNQVSLLCLVKGFYPSDIAVE | |
| | | WESNGQPENNYLTWPPVLDSDGSFFLYSKLTVDKSRWQQGNVFSCSVMH | |
| | | EALHNRFTQKSLSLSPGK | |
| | 3^{rd} | EIVLTQSPATLSLSPGERATLSCRASSSVSYMHWYQQKPGQAPRPWIYA | 77 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSVSASVGDRVTITCRASQGIYSWLAWYQQKPGKAPNLLIY | 78 |
| | chain | TASTLQSGVPSRFSGSGSGTDFTLTISSLQPEDFATYYCQQANIFPLTF | |
| | | GGGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1935 | 1^{st} | QVQLVQSGAEVKKPGASVKVSCKASGYTFTSYVMHWVRQKPGQGLEWIG | 79 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTGYYMHWVRQAPGQGLEWMG | 80 |
| | chain | WINPDSGGTNYAQKFQGRVTMTRDTSISTAYMELNRLRSDDTAVYYCAR | |
| | | DQPLGYCTNGVCSYFDYWGQGTLVTVSSDLKNVFPPEVAVFEPSEAEIS | |
| | | HTQKATLVCLATGFYPDHVELSWWVNGKEVHSGVCTDPQPLKEQPALQD | |
| | | SRYALSSRLRVSATFWQNPRNHFRCQVQFYGLSENDEWTQDRAKPVTQI | |
| | | VSAEAWGRASDKTHTCPPCPAPELLGPSVFLFPPKPKDTLMISRTPEVT | |
| | | CVVVDVSHEDPEVKFNWYVDGVEVHNAKTKPREEQYNSTYRVVSVLTVL | |
| | | HQDWLNGKEYKCKVSNKALPAPIEKTISKAKGQPREPQVCTLPPSREEM | |
| | | TKNQVSLSCAVKGFYPSDIAVEWESNGQPENNYKTTPPVLDSDGSFFLV | |
| | | SKLTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 81 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSVSASVGDRVTITCRASQGIYSWLAWYQQKPGKAPNLLIY | 82 |
| | chain | TASTLQSGVPSRFSGSGSGTDFTLTISSLQPEDFATYYCQQANIFPLTF | |
| | | GGGTKVEIKPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |
| Ly1936 | 1^{st} | QVQLVQSGAEVKKPGASVKVSCKASGYTFTSYVMHWVRQKPGQGLEWIG | 83 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 84 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | VQLVQSGAEVKKPGASVKVSCKASGYTFTGYYMHWVRQAPGQGLEWMGW | |
| | | INPDSGGTNYAQKFQGRVTMTRDTSISTAYMELNRLRSDDTAVYYCARD | |
| | | QPLGYCTNGVCSYFDYWGQGTLVTVSSDLKNVFPPEVAVFEPSEAEISH | |
| | | TQKATLVCLATGFYPDHVELSWWVNGKEVHSGVCTDPQPLKEQPALQDS | |
| | | RYALSSRLRVSATFWQNPRNHERCQVQFYGLSENDEWTQDRAKPVTQIV | |
| | | SAEAWGRASDKTHTCPPCPAPELLGPSVFLFPPKPKDTLMISRTPEVTC | |
| | | VVVDVSHEDPEVKFNWYVDGVEVHNAKTKPREEQYNSTYRVVSVLTVLH | |
| | | QDWLNGKEYKCKVSNKALPAPIEKTISKAKGQPREPQVYTLPPSREEMT | |
| | | KNQVSLSCAVKGFYPSDIAVEWESNGQPENNYKTTPPVLDSDGSFFLVS | |
| | | KLTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 85 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSVSASVGDRVTITCRASQGIYSWLAWYQQKPGKAPNLLIY | 86 |
| | chain | TASTLQSGVPSRFSGSGSGTDFTLTISSLQPEDFATYYCQQANIFPLTF | |
| | | GGGTKVEIKPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |

### Characterization of Anti-B7H3/CD40 Bi-Specific Antibodies

### (i) Binding Activity

Anti-B7H3/CD40 bi-specific antibodies were analyzed by FACS for their binding properties to human CD40 and/or human B7H3 expressed on CHO cells as described above.

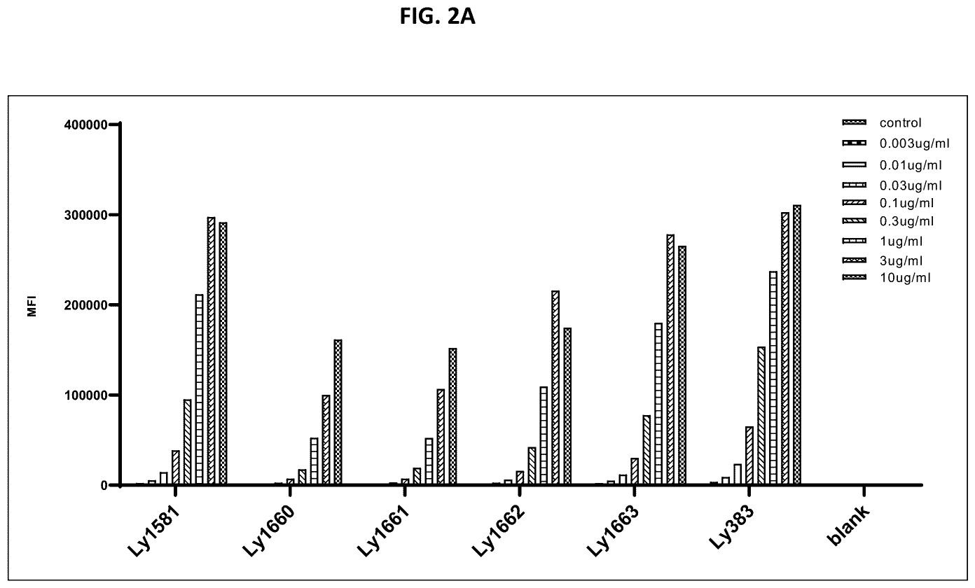

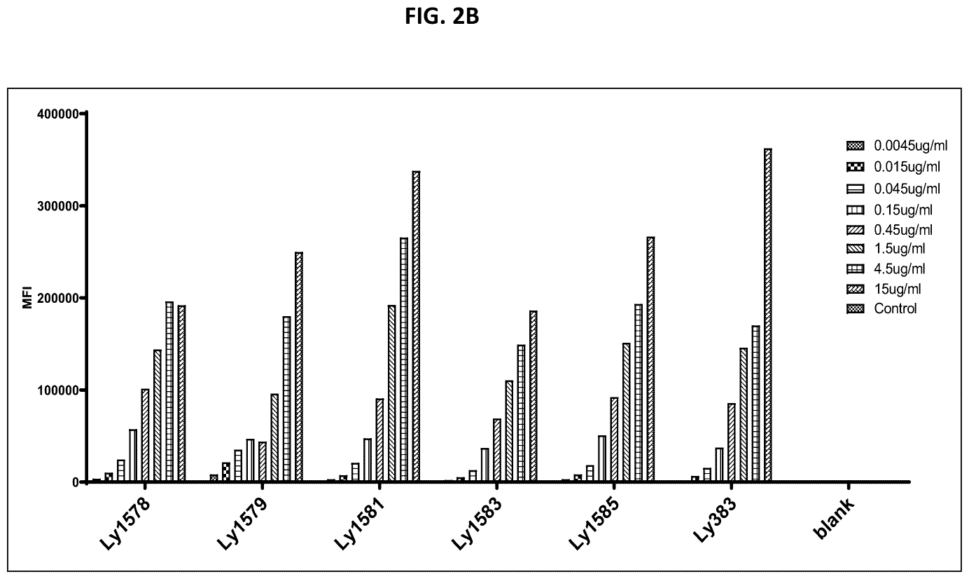

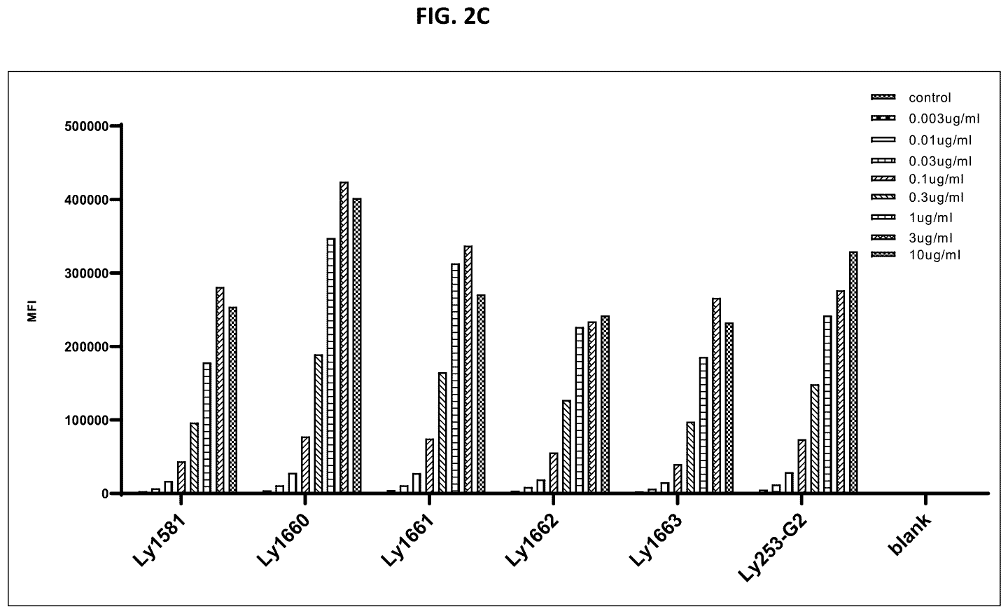

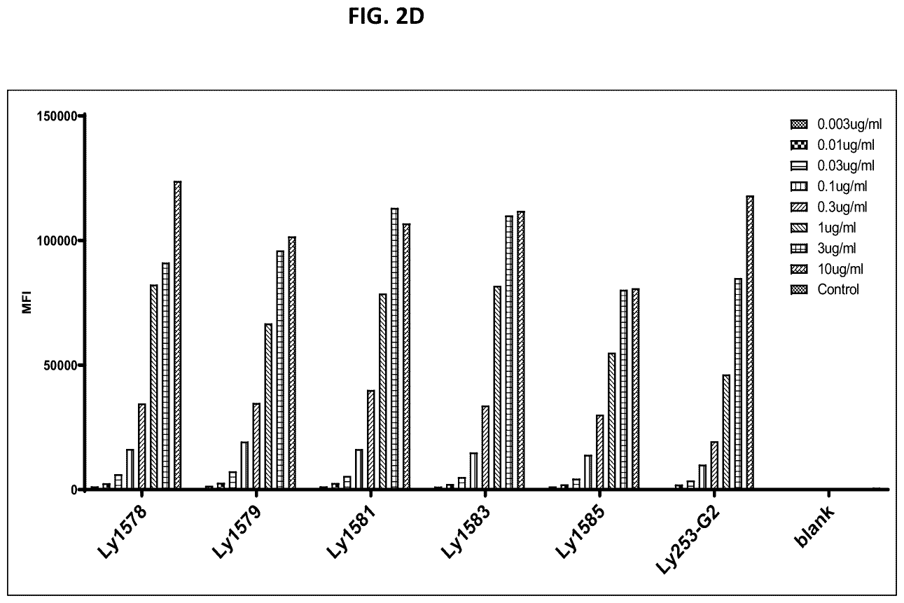

As shown in FIGS. 2A and 2B, the exemplary anti-CD40/B7H3 bi-specific antibodies exhibited a various range of binding affinity to human B7H3 expressed on the CHO cells. As shown in FIGS. 2C and 2D, the bi-specific antibodies exhibited different levels of binding affinity to humanCD40 expressed on CHO cells.

### (ii) Agonistic Activity for CD40

To determine the agonist activity of these anti-B7H3/CD40 bi-specific antibodies, a CD40 reporter assay was developed, which involves reporter cells over-expressing human CD40. The CD40 reporter assay was performed in co-culture with or without B7H3-expressing CHO cells following the procedures as described below. This GS-H2-huCD40 reporter cells were re-suspended and diluted to 1×104 cells/mL with assay buffer (MEM containing 1% FBS). The cells were added at 100 μL/well, such that the final cell number was 1000 cells/well in the assay plate (Nunc, Cat #167425). Samples were added at 100 uL/well test sample at 2× final concentrations to the assay plate. The assay plate was incubated in 37° C., 5% CO2 incubator for 18-20 hours. After the 18-20 hour incubation, 8 μL of the supernatant from each well of the assay plate was collected and added to HTRF detection assay plate (Nunc). A Human Interleukin 8 (reporter of CD40 activation) detection assay was performed using a Human IL-8 Assay Kit (Cisbio, Cat #62IL8PEB). In particular, 16 μL assay volume was used. The results were read using Time Resolved Fluorescence by Tecan F200pro and the relative light unit data was recorded.

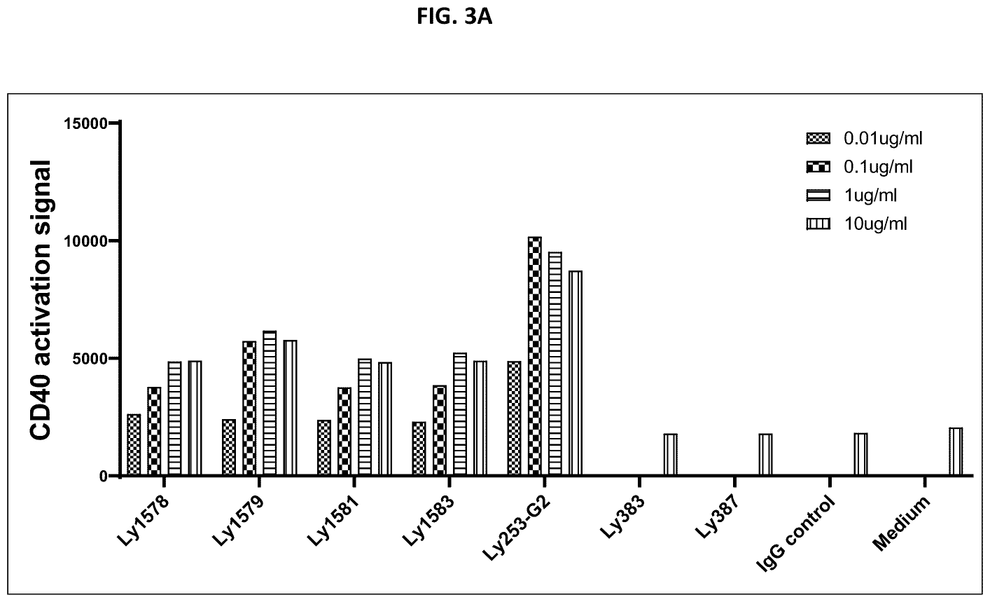

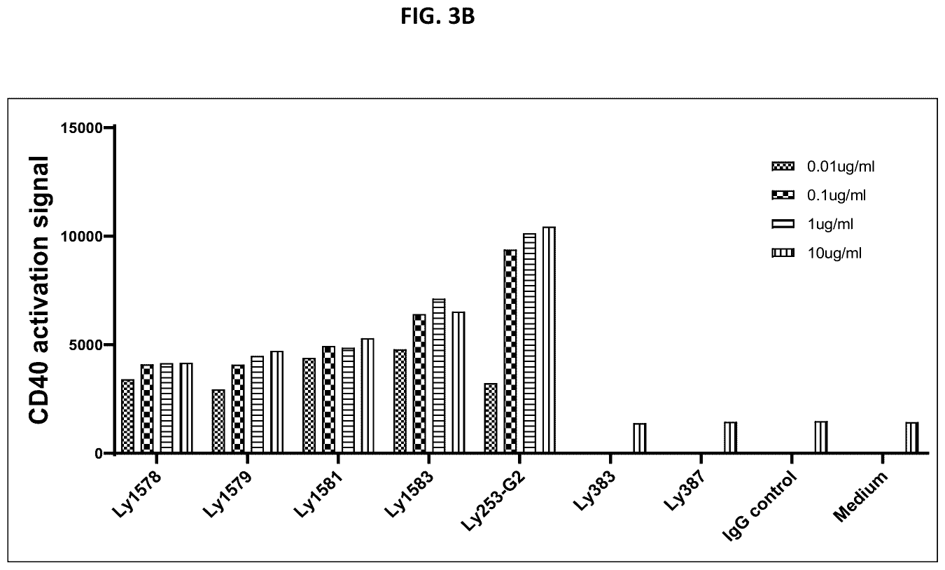

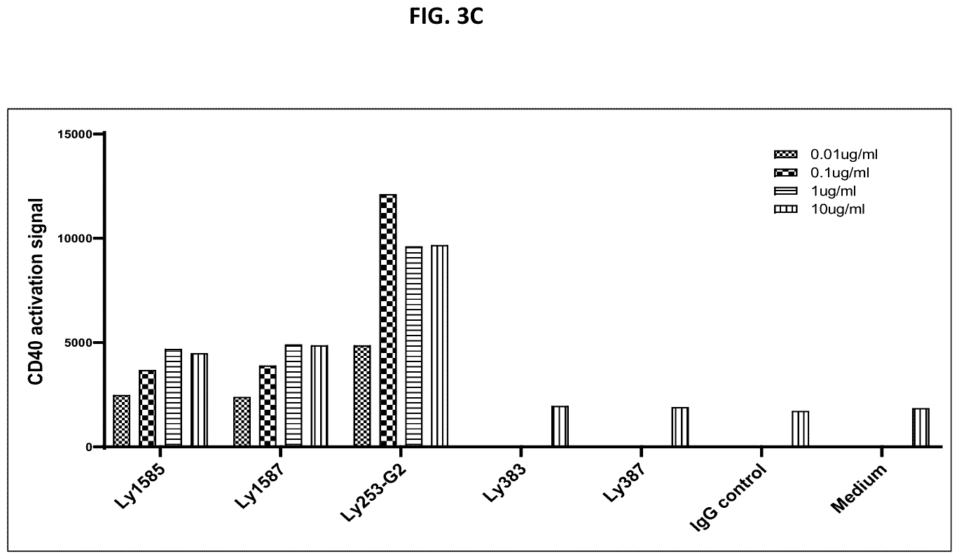

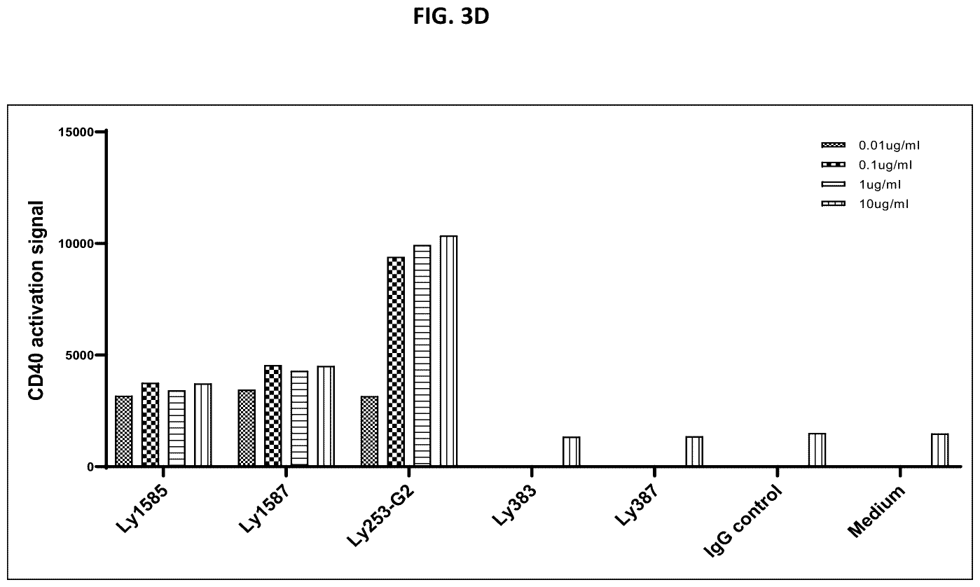

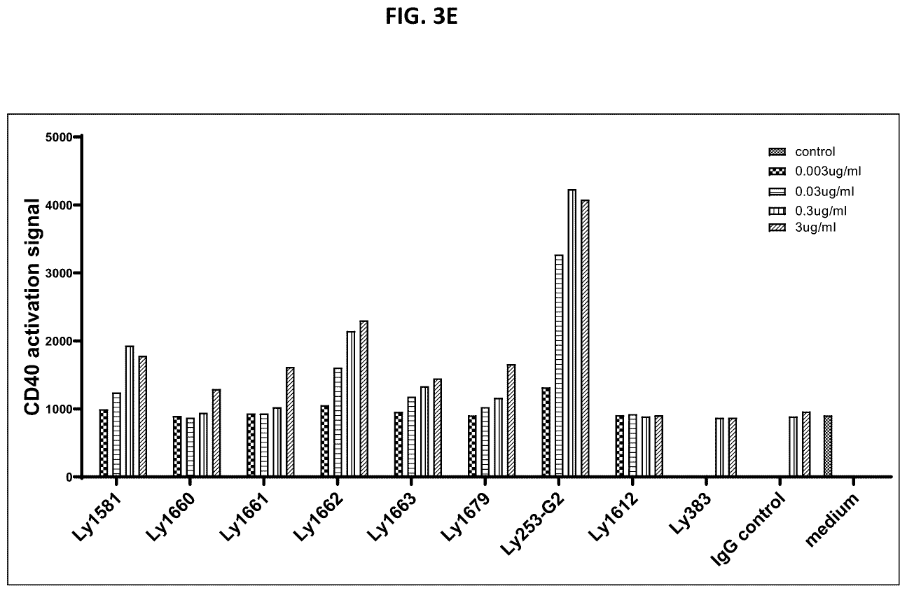

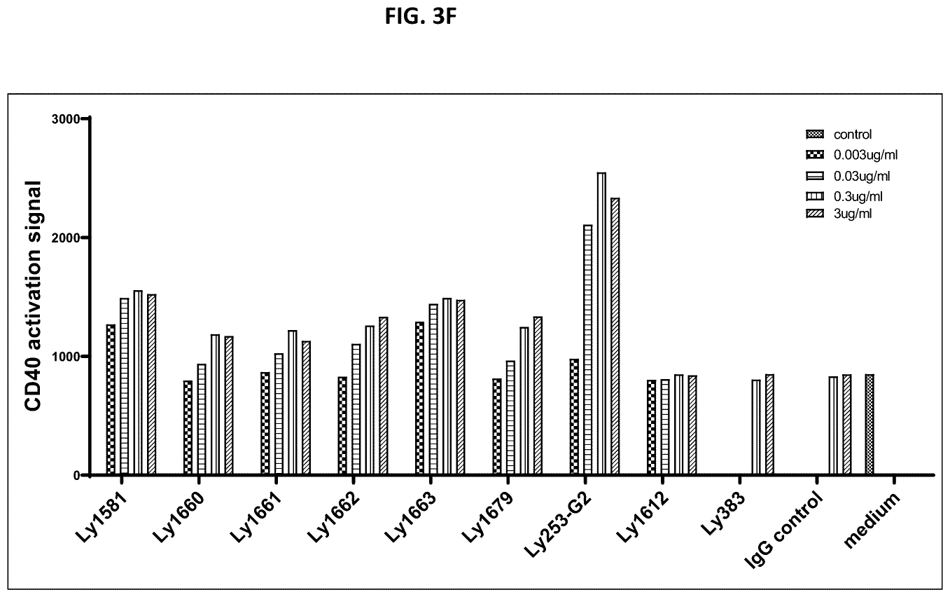

As shown in FIGS. 3A-3F, the tested examplary bi-specific antibodies showed enhanced or varied CD40 agonist activity in presence of human B7H3 expressing cells, as compared to the condition at the absence of human B7H3 expressing cells. Binding to both CD40 and B7H3 by the tested bi-specific antibodies simultaneously in a microenvironment would affect individual binding and therefore CD40 agonist activities.

(iii) Anti-Tumor Activity

Exemplary anti-B7H3/CD40 antibodies were tested in mouse syngeneic tumor models in vivo to determine the anti-tumor efficacy of these antibodies. C57BL6 mice with human CD40 knock-in were used to develop syngeneic mouse tumor models. Human B7H3 overexpressing murine colon cancer MC38 tumor cells were subcutaneously implanted into the mice. Mice were grouped when the tumor size was approximately 150±50 mm3 (n=6). Anti-B7H3/CD40 antibodies were administered by intraperitoneal injections and tumor sizes were measure during 4-6 weeks of antibody treatment. Tumor sizes were measured by caliber 2 times a week and calculated as tumor volume using formula of 0.5×length×width2.

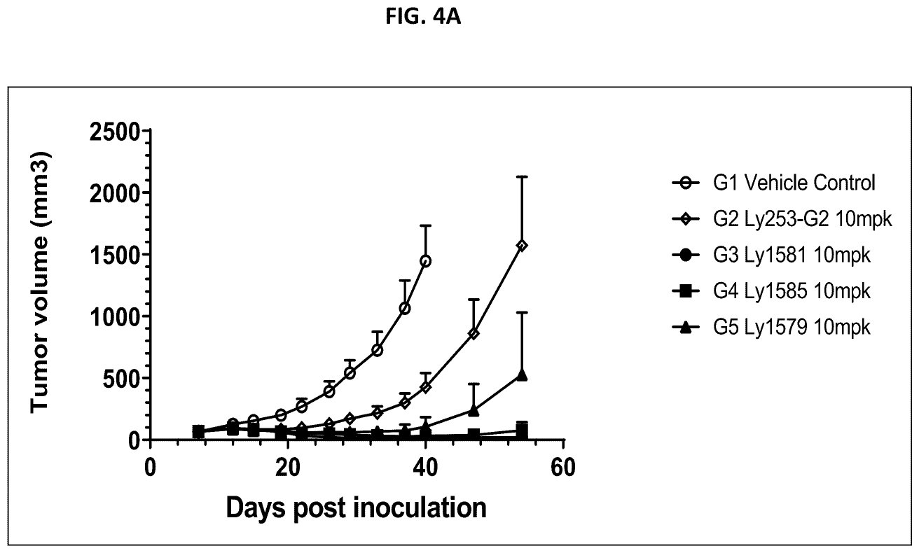

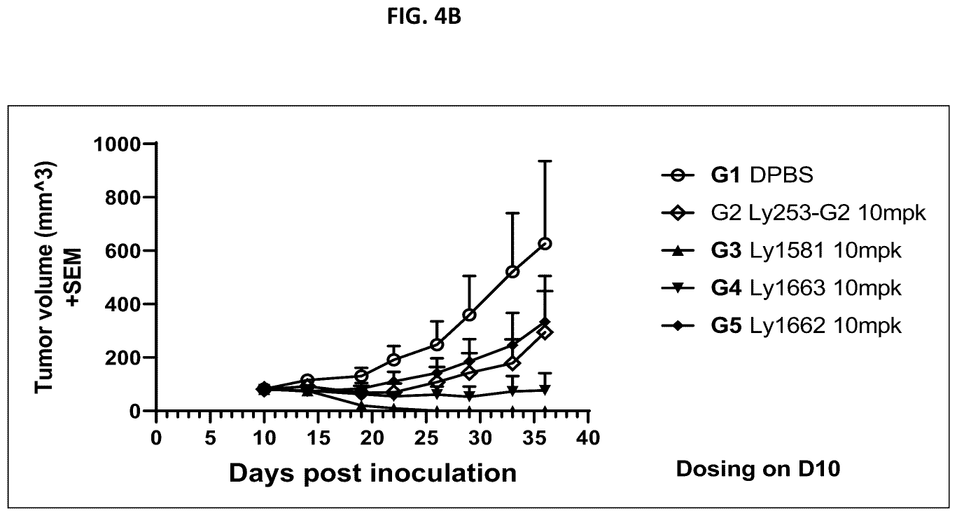

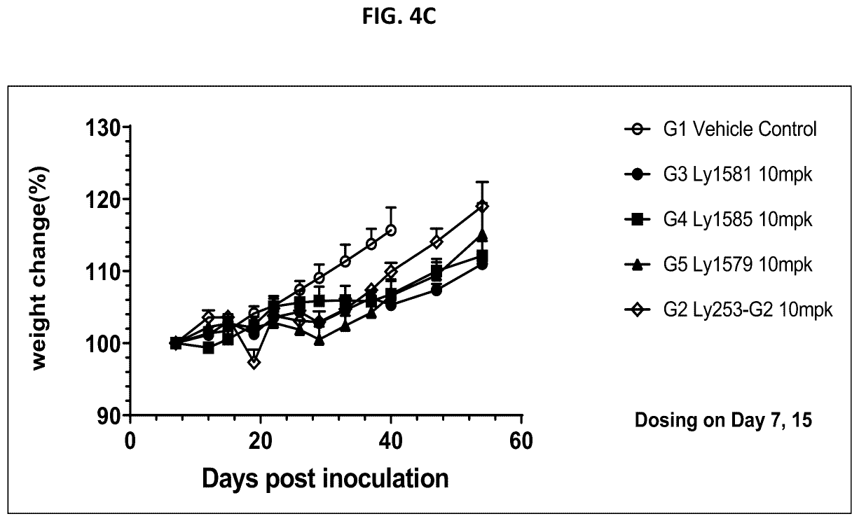

Anti-tumor efficacy was evaluated between tumor sizes of the control group and antibody treatment group as shown in FIGS. 4A-4C. The parent anti-CD40 antibody was used as reference. Several of the bispecific antibodies including Ly1581, Ly1579, Ly1663 and Ly1585 showed much stronger efficacy compared to the parent anti-CD40 antibody (FIGS. 4A-4B), and better safety (FIG. 4C).

Anti-B7H3/CD40 antibodies are evaluated for their in vitro and in vivo activity.

### Example 4: Anti-B7H3/CD137 Bi-Specific Antibodies

Anti-B7H3/CD137 bi-specific antibodies were produced and characterized using parent anti-B7H3 antibody clone Ly1612, parent anti-CD137 antibody clone CD137 Ab1. The amino acid sequences of the V_{H} and the V_{L} of the parent clones are provided in Table 1 above. The heavy chain and light chain complementary determining regions determined by the Kabat scheme are in boldface.

cDNAs encoding the heavy chain variable region (V_{H}) and the light chain variable region (V_{L}) of the parent clones were used as the starting materials for making these bi-specific antibodies. CHO-cell transient expression was carried out with plasmids configured for expressing the polypeptide chains of the bi-specific antibodies. These resultant bispecific antibodies were purified by protein A affinity chromatography.

The amino acid sequences of the polypeptides of exemplary bi-specific antibodies are provided in Table 4 below.

TABLE 4
Exemplary Anti-B7H3/CD137 Bi-Specific Antibodies

| | | | SED ID |
|---|---|---|---|
| Antibody Clones | | Amino acid sequence | NO: |
| Ly1937 | 1^{st} | QVQLVQSGAEVKKPGASVKVSCKASGYTFTSYVMHWVRQKPGQGLEWIG | 87 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | EIVLTQSPATLSLSPGERATLSCRASSSVSYMHWYQQKPGQAPRPWIYA | 88 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1938 | 1^{st} | QVQLVQSGAEVKKPGASVKVSCKASGYTFTSYVMHWVRQKPGQGLEWIG | 89 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 90 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLVQSGAEVK | |
| | | KPGASVKVSCKASGYTFAGFEMHWVRQAPGQGLEWMGAIDPKTGGTDYN | |
| | | QKFKDRVTMTRDTSISTAYMELSRLRSDDTAVYYCARDLGYFDVWGQGT | |
| | | LVTVSSGGGGSGGGGSGGGGSGGGGSDIQMTQSPSSLSASVGDRVTITC | |
| | | RASQDIRSNLNWYQQKPGGAVKLLIYYTSRLHSGVPSRFSGSGSGTDYT | |
| | | LTISSLQPEDFATYFCQQSEKLPRTFGGGTKVEIR | |
| Ly1939 | 1^{st} | QVQLVQSGAEVKKPGASVKVSCKASGYTFAGFEMHWVRQAPGQGLEWMG | 91 |
| | chain | AIDPKTGGTDYNQKFKDRVTMTRDTSISTAYMELSRLRSDDTAVYYCAR | |
| | | DLGYFDVWGQGTLVTVSSGGGGSGGGGSGGGGSGGGGSDIQMTQSPSSL | |
| | | SASVGDRVTITCRASQDIRSNLNWYQQKPGGAVKLLIYYTSRLHSGVPS | |
| | | RFSGSGSGTDYTLTISSLQPEDFATYFCQQSEKLPRTFGGGTKVEIRGG | |
| | | GGSGGGGSGGGGSGGGGSQVQLVQSGAEVKKPGASVKVSCKASGYTFTS | |
| | | YVMHWVRQKPGQGLEWIGYINPYNEGTESTDKFKGRATMTSDKSTSTVY | |
| | | MELSSLRSEDTAVYYCASIYYGYEGTYFGVWGQGTLVTVSSASTKGPSV | |
| | | FPLAPSSKSTSGGTAALGCLVKDYFPEPVTVSWNSGALTSGVHTFPAVL | |
| | | QSSGLYSLSSVVTVPSSSLGTQTYICNVNHKPSNTKVDKKVEPKSCDKT | |
| | | HTCPPCPAPELLGPSVFLFPPKPKDTLMISRTPEVTCVVVDVSHEDPEV | |
| | | KFNWYVDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVKG | |
| | | FYPSDIAVEWESNGQPENNYKTTPPVLDSDGSFFLYSKLTVDKSRWQQG | |
| | | NVFSCSVMHEALHNHYTQKSLSLSPGK | |
| | 2^{nd} | EIVLTQSPATLSLSPGERATLSCRASSSVSYMHWYQQKPGQAPRPWIYA | 92 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1940 | 1^{st} | QVQLVQSGAEVKKPGASVKVSCKASGYTFTSYVMHWVRQKPGQGLEWIG | 93 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFAGFEMHWVRQAPGQGLEWMG | 94 |
| | chain | AIDPKTGGTDYNQKFKDRVTMTRDTSISTAYMELSRLRSDDTAVYYCAR | |
| | | DLGYFDVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECDKTHTCPPCPAPELLGPSVFLF | |
| | | PPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKG | |
| | | QPREPQVCTLPPSREEMTKNQVSLSCAVKGFYPSDIAVEWESNGQPENN | |
| | | YKTTPPVLDSDGSFFLVSKLTVDKSRWQQGNVFSCSVMHEALHNRFTQK | |
| | | SLSLSPGK | |
| | 3^{rd} | EIVLTQSPATLSLSPGERATLSCRASSSVSYMHWYQQKPGQAPRPWIYA | 95 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 96 |
| | chain | YTSRLHSGVPSRFSGSGSGTDYTLTISSLQPEDFATYFCQQSEKLPRTF | |
| | | GGGTKVEIRSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1941 | 1^{st} | QVQLVQSGAEVKKPGASVKVSCKASGYTFTSYVMHWVRQKPGQGLEWIG | 97 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFAGFEMHWVRQAPGQGLEWMG | 98 |
| | chain | AIDPKTGGTDYNQKFKDRVTMTRDTSISTAYMELSRLRSDDTAVYYCAR | |
| | | DLGYFDVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECDKTHTCPPCPAPELLGPSVFLF | |
| | | PPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKG | |
| | | QPREPQVYTKPPSREEMTKNQVSLTCKVKGFYPSDIAVEWESNGQPENN | |
| | | YKTTPPVLDSDGSFFLYSKLTVDKSRWQQGNVFSCSVMHEALHNRFTQK | |
| | | SLSLSPGK | |
| | 3^{rd} | EIVLTQSPATLSLSPGERATLSCRASSSVSYMHWYQQKPGQAPRPWIYA | 99 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 100 |
| | chain | YTSRLHSGVPSRFSGSGSGTDYTLTISSLQPEDFATYFCQQSEKLPRTF | |
| | | GGGTKVEIRSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1942 | 1^{st} | QVQLVQSGAEVKKPGASVKVSCKASGYTFTSYVMHWVRQKPGQGLEWIG | 101 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | QVQLVQSGAEVKKPGASVKVSCKASGYTFAGFEMHWVRQAPGQGLEWMG | 102 |
| | chain | AIDPKTGGTDYNQKFKDRVTMTRDTSISTAYMELSRLRSDDTAVYYCAR | |
| | | DLGYFDVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECDKTHTCPPCPAPELLGPSVFLF | |
| | | PPKPKDTLMISRTPEVTCVVVDVSHEDPEVKFNWYVDGVEVHNAKTKPR | |
| | | EEQYNSTYRVVSVLTVLHQDWLNGKEYKCKVSNKALPAPIEKTISKAKG | |
| | | QPREPQVYVLPPSREEMTKNQVSLLCLVKGFYPSDIAVEWESNGQPENN | |
| | | YLTWPPVLDSDGSFFLYSKLTVDKSRWQQGNVFSCSVMHEALHNRFTQK | |
| | | SLSLSPGK | |
| | 3^{rd} | EIVLTQSPATLSLSPGERATLSCRASSSVSYMHWYQQKPGQAPRPWIYA | 103 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 104 |
| | chain | YTSRLHSGVPSRFSGSGSGTDYTLTISSLQPEDFATYFCQQSEKLPRTF | |
| | | GGGTKVEIRSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1943 | 1^{st} | QVQLVQSGAEVKKPGASVKVSCKASGYTFTSYVMHWVRQKPGQGLEWIG | 105 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFAGFEMHWVRQAPGQGLEWMG | 106 |
| | chain | AIDPKTGGTDYNQKFKDRVTMTRDTSISTAYMELSRLRSDDTAVYYCAR | |
| | | DLGYFDVWGQGTLVTVSSDLKNVFPPEVAVFEPSEAEISHTQKATLVCL | |
| | | ATGFYPDHVELSWWVNGKEVHSGVCTDPQPLKEQPALQDSRYALSSRLR | |
| | | VSATFWQNPRNHFRCQVQFYGLSENDEWTQDRAKPVTQIVSAEAWGRAS | |
| | | DKTHTCPPCPAPELLGPSVFLFPPKPKDTLMISRTPEVTCVVVDVSHED | |
| | | PEVKFNWYVDGVEVHNAKTKPREEQYNSTYRVVSVLTVLHQDWLNGKEY | |
| | | KCKVSNKALPAPIEKTISKAKGQPREPQVCTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 107 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 108 |
| | chain | YTSRLHSGVPSRFSGSGSGTDYTLTISSLQPEDFATYFCQQSEKLPRTF | |
| | | GGGTKVEIRPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |
| Ly1944 | 1^{st} | QVQLVQSGAEVKKPGASVKVSCKASGYTFTSYVMHWVRQKPGQGLEWIG | 109 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 110 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | VQLVQSGAEVKKPGASVKVSCKASGYTFAGFEMHWVRQAPGQGLEWMGA | |
| | | IDPKTGGTDYNQKFKDRVTMTRDTSISTAYMELSRLRSDDTAVYYCARD | |
| | | LGYFDVWGQGTLVTVSSDLKNVFPPEVAVFEPSEAEISHTQKATLVCLA | |
| | | TGFYPDHVELSWWVNGKEVHSGVCTDPQPLKEQPALQDSRYALSSRLRV | |
| | | SATFWQNPRNHFRCQVQFYGLSENDEWTQDRAKPVTQIVSAEAWGRASD | |
| | | KTHTCPPCPAPELLGPSVFLFPPKPKDTLMISRTPEVTCVVVDVSHEDP | |
| | | EVKFNWYVDGVEVHNAKTKPREEQYNSTYRVVSVLTVLHQDWLNGKEYK | |
| | | CKVSNKALPAPIEKTISKAKGQPREPQVYTLPPSREEMTKNQVSLSCAV | |
| | | KGFYPSDIAVEWESNGQPENNYKTTPPVLDSDGSFFLVSKLTVDKSRWQ | |
| | | QGNVFSCSVMHEALHNRFTQKSLSLSPGK | |
| | 3^{rd} | EIVLTQSPATLSLSPGERATLSCRASSSVSYMHWYQQKPGQAPRPWIYA | 111 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 112 |
| | chain | YTSRLHSGVPSRFSGSGSGTDYTLTISSLQPEDFATYFCQQSEKLPRTF | |
| | | GGGTKVEIRPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |

### Characterization of Anti-B7H3/CD137 Bi-Specific Antibodies

These bispecific antibodies are evaluated for their in vitro and in vivo activity, including binding to target antigen (B7H3 and CD137), agonistic activity in CD137 reporter assay system, activation of immune cells, anti-tumor activity in mouse models.

### Example 5: Anti-B7H3/GITR Bi-Specific Antibodies

Anti-B7H3/GITR bi-specific antibodies were produced and characterized using parent anti-B7H-3 antibody clone Ly1612, parent anti-GITR antibody clones including TM677 (GITR Ab1). The amino acid sequences of the V_{H} and the V_{L} of the parent clones are provided in Table 1 above. The heavy chain and light chain complementary determining regions determined by the Kabat scheme are in boldface.

cDNAs encoding the heavy chain variable region (V_{H}) and the light chain variable region (V_{L}) of the parent clones were used as the starting materials for making these bi-specific antibodies. CHO-cell transient expression was carried out with plasmids configured for expressing the polypeptide chains of the bi-specific antibodies. These resultant bispecific antibodies were purified by protein A affinity chromatography.

The amino acid sequences of the polypeptides of exemplary bi-specific antibodies are provided in Table 5 below:

TABLE 5
Exemplary Anti-B7H3/GITR Bi-Specific Antibodies

| | | | SED ID |
|---|---|---|---|
| Antibody Clones | | Amino acid sequence | NO: |
| Ly1945 | 1^{st} | QVQLVQSGAEVKKPGASVKVSCKASGYTFTSYVMHWVRQKPGQGLEWIG | 113 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPATLSLSPGERATL | |
| | | SCRASKSVRTGMHWYQQKPGQAPRLLIYGASNLESGIPARFSGSGSGTD | |
| | | FTLTISSLEPEDFAVYYCQQSWNHFTFGQGTKLEIKGGGGSGGGGSGGG | |
| | | GSGGGGSQVQLQESGPGLVKPSETLSLTCTVSGFSLTSYNVHWIRQPPG | |
| | | KGLEWIGVIWSGVRTDYNSVLKPRVTISVDTSKNQFSLKLSSVTAADTA | |
| | | VYYCARGTYDDNYHDVMDAWGQGTLVTVSS | |
| | 2^{nd} | EIVLTQSPATLSLSPGERATLSCRASSSVSYMHWYQQKPGQAPRPWIYA | 114 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1946 | 1^{st} | QVQLVQSGAEVKKPGASVKVSCKASGYTFTSYVMHWVRQKPGQGLEWIG | 115 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 116 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSEIVLTQSPATLS | |
| | | LSPGERATLSCRASKSVRTGMHWYQQKPGQAPRLLIYGASNLESGIPAR | |
| | | FSGSGSGTDFTLTISSLEPEDFAVYYCQQSWNHFTFGQGTKLEIKGGGG | |
| | | SGGGGSGGGGSGGGGSQVQLQESGPGLVKPSETLSLTCTVSGFSLTSYN | |
| | | VHWIRQPPGKGLEWIGVIWSGVRTDYNSVLKPRVTISVDTSKNQFSLKL | |
| | | SSVTAADTAVYYCARGTYDDNYHDVMDAWGQGTLVTVSS | |
| Ly1947 | 1^{st} | EIVLTQSPATLSLSPGERATLSCRASKSVRTGMHWYQQKPGQAPRLLIY | 117 |
| | chain | GASNLESGIPARFSGSGSGTDFTLTISSLEPEDFAVYYCQQSWNHFTFG | |
| | | QGTKLEIKGGGGSGGGGSGGGGSGGGGSQVQLQESGPGLVKPSETLSLT | |
| | | CTVSGFSLTSYNVHWIRQPPGKGLEWIGVIWSGVRTDYNSVLKPRVTIS | |
| | | VDTSKNQFSLKLSSVTAADTAVYYCARGTYDDNYHDVMDAWGQGTLVTV | |
| | | SSGGGGSGGGGSGGGGSGGGGSQVQLVQSGAEVKKPGASVKVSCKASGY | |
| | | TFTSYVMHWVRQKPGQGLEWIGYINPYNEGTESTDKFKGRATMTSDKST | |
| | | STVYMELSSLRSEDTAVYYCASIYYGYEGTYFGVWGQGTLVTVSSASTK | |
| | | GPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKS | |
| | | CDKTHTCPPCPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLTC | |
| | | LVKGFYPSDIAVEWESNGQPENNYKTTPPVLDSDGSFFLYSKLTVDKSR | |
| | | WQQGNVFSCSVMHEALHNHYTQKSLSLSPGK | |
| | 2^{nd} | EIVLTQSPATLSLSPGERATLSCRASSSVSYMHWYQQKPGQAPRPWIYA | 118 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1948 | 1^{st} | QVQLVQSGAEVKKPGASVKVSCKASGYTFTSYVMHWVRQKPGQGLEWIG | 119 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLQESGPGLVKPSETLSLTCTVSGFSLTSYNVHWIRQPPGKGLEWIG | 120 |
| | chain | VIWSGVRTDYNSVLKPRVTISVDTSKNQFSLKLSSVTAADTAVYYCARG | |
| | | TYDDNYHDVMDAWGQGTLVTVSSASVAAPSVFIFPPSDEQLKSGTASVV | |
| | | CLLNNFYPREAKVQWKVDNALQSGNSQESVTEQDSKDSTYSLSSTLTLS | |
| | | KADYEKHKVYACEVTHQGLSSPVTKSFNRGECDKTHTCPPCPAPELLGP | |
| | | SVFLFPPKPKDTLMISRTPEVTCVVVDVSHEDPEVKFNWYVDGVEVHNA | |
| | | KTKPREEQYNSTYRVVSVLTVLHQDWLNGKEYKCKVSNKALPAPIEKTI | |
| | | SKAKGQPREPQVCTLPPSREEMTKNQVSLSCAVKGFYPSDIAVEWESNG | |
| | | QPENNYKTTPPVLDSDGSFFLVSKLTVDKSRWQQGNVFSCSVMHEALHN | |
| | | RFTQKSLSLSPGK | |
| | 3^{rd} | EIVLTQSPATLSLSPGERATLSCRASSSVSYMHWYQQKPGQAPRPWIYA | 121 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPATLSLSPGERATLSCRASKSVRTGMHWYQQKPGQAPRLLIY | 122 |
| | chain | GASNLESGIPARFSGSGSGTDFTLTISSLEPEDFAVYYCQQSWNHFTFG | |
| | | QGTKLEIKSSASTKGPSVFPLAPSSKSTSGGTAALGCLVKDYFPEPVTV | |
| | | SWNSGALTSGVHTFPAVLQSSGLYSLSSVVTVPSSSLGTQTYICNVNHK | |
| | | PSNTKVDKKVEPKSC | |
| Ly1949 | 1^{st} | QVQLVQSGAEVKKPGASVKVSCKASGYTFTSYVMHWVRQKPGQGLEWIG | 123 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLQESGPGLVKPSETLSLTCTVSGFSLTSYNVHWIRQPPGKGLEWIG | 124 |
| | chain | VIWSGVRTDYNSVLKPRVTISVDTSKNQFSLKLSSVTAADTAVYYCARG | |
| | | TYDDNYHDVMDAWGQGTLVTVSSASVAAPSVFIFPPSDEQLKSGTASVV | |
| | | CLLNNFYPREAKVQWKVDNALQSGNSQESVTEQDSKDSTYSLSSTLTLS | |
| | | KADYEKHKVYACEVTHQGLSSPVTKSFNRGECDKTHTCPPCPAPELLGP | |
| | | SVFLFPPKPKDTLMISRTPEVTCVVVDVSHEDPEVKFNWYVDGVEVHNA | |
| | | KTKPREEQYNSTYRVVSVLTVLHQDWLNGKEYKCKVSNKALPAPIEKTI | |
| | | SKAKGQPREPQVYTKPPSREEMTKNQVSLTCKVKGFYPSDIAVEWESNG | |
| | | QPENNYKTTPPVLDSDGSFFLYSKLTVDKSRWQQGNVFSCSVMHEALHN | |
| | | RFTQKSLSLSPGK | |
| | 3^{rd} | EIVLTQSPATLSLSPGERATLSCRASSSVSYMHWYQQKPGQAPRPWIYA | 125 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPATLSLSPGERATLSCRASKSVRTGMHWYQQKPGQAPRLLIY | 126 |
| | chain | GASNLESGIPARFSGSGSGTDFTLTISSLEPEDFAVYYCQQSWNHFTFG | |
| | | QGTKLEIKSSASTKGPSVFPLAPSSKSTSGGTAALGCLVKDYFPEPVTV | |
| | | SWNSGALTSGVHTFPAVLQSSGLYSLSSVVTVPSSSLGTQTYICNVNHK | |
| | | PSNTKVDKKVEPKSC | |
| Ly1950 | 1^{st} | QVQLVQSGAEVKKPGASVKVSCKASGYTFTSYVMHWVRQKPGQGLEWIG | 127 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | QVQLQESGPGLVKPSETLSLTCTVSGFSLTSYNVHWIRQPPGKGLEWIG | 128 |
| | chain | VIWSGVRTDYNSVLKPRVTISVDTSKNQFSLKLSSVTAADTAVYYCARG | |
| | | TYDDNYHDVMDAWGQGTLVTVSSASVAAPSVFIFPPSDEQLKSGTASVV | |
| | | CLLNNFYPREAKVQWKVDNALQSGNSQESVTEQDSKDSTYSLSSTLTLS | |
| | | KADYEKHKVYACEVTHQGLSSPVTKSFNRGECDKTHTCPPCPAPELLGP | |
| | | SVFLFPPKPKDTLMISRTPEVTCVVVDVSHEDPEVKFNWYVDGVEVHNA | |
| | | KTKPREEQYNSTYRVVSVLTVLHQDWLNGKEYKCKVSNKALPAPIEKTI | |
| | | SKAKGQPREPQVYVLPPSREEMTKNQVSLLCLVKGFYPSDIAVEWESNG | |
| | | QPENNYLTWPPVLDSDGSFFLYSKLTVDKSRWQQGNVFSCSVMHEALHN | |
| | | RFTQKSLSLSPGK | |
| | 3^{rd} | EIVLTQSPATLSLSPGERATLSCRASSSVSYMHWYQQKPGQAPRPWIYA | 129 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPATLSLSPGERATLSCRASKSVRTGMHWYQQKPGQAPRLLIY | 130 |
| | chain | GASNLESGIPARFSGSGSGTDFTLTISSLEPEDFAVYYCQQSWNHFTFG | |
| | | QGTKLEIKSSASTKGPSVFPLAPSSKSTSGGTAALGCLVKDYFPEPVTV | |
| | | SWNSGALTSGVHTFPAVLQSSGLYSLSSVVTVPSSSLGTQTYICNVNHK | |
| | | PSNTKVDKKVEPKSC | |
| Ly1951 | 1^{st} | QVQLVQSGAEVKKPGASVKVSCKASGYTFTSYVMHWVRQKPGQGLEWIG | 131 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLQESGPGLVKPSETLSLTCTVSGFSLTSYNVHWIRQPPGKGLEWIG | 132 |
| | chain | VIWSGVRTDYNSVLKPRVTISVDTSKNQFSLKLSSVTAADTAVYYCARG | |
| | | TYDDNYHDVMDAWGQGTLVTVSSDLKNVFPPEVAVFEPSEAEISHTQKA | |
| | | TLVCLATGFYPDHVELSWWVNGKEVHSGVCTDPQPLKEQPALQDSRYAL | |
| | | SSRLRVSATFWQNPRNHFRCQVQFYGLSENDEWTQDRAKPVTQIVSAEA | |
| | | WGRASDKTHTCPPCPAPELLGPSVFLFPPKPKDTLMISRTPEVTCVVVD | |
| | | VSHEDPEVKFNWYVDGVEVHNAKTKPREEQYNSTYRVVSVLTVLHQDWL | |
| | | NGKEYKCKVSNKALPAPIEKTISKAKGQPREPQVCTLPPSREEMTKNQV | |
| | | SLSCAVKGFYPSDIAVEWESNGQPENNYKTTPPVLDSDGSFFLVSKLTV | |
| | | DKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 133 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPATLSLSPGERATLSCRASKSVRTGMHWYQQKPGQAPRLLIY | 134 |
| | chain | GASNLESGIPARFSGSGSGTDFTLTISSLEPEDFAVYYCQQSWNHFTFG | |
| | | QGTKLEIKPDIQNPDPAVYQLRDSKSSDKSVCLFTDFDSQTQVSQSKDS | |
| | | DVYITDKCVLDMRSMDFKSNSAVAWSQKSDFACANAFQNSIIPEDTFFP | |
| | | SPESS | |
| Ly1952 | 1^{st} | QVQLVQSGAEVKKPGASVKVSCKASGYTFTSYVMHWVRQKPGQGLEWIG | 135 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 136 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | VQLQESGPGLVKPSETLSLTCTVSGFSLTSYNVHWIRQPPGKGLEWIGV | |
| | | IWSGVRTDYNSVLKPRVTISVDTSKNQFSLKLSSVTAADTAVYYCARGT | |
| | | YDDNYHDVMDAWGQGTLVTVSSDLKNVFPPEVAVFEPSEAEISHTQKAT | |
| | | LVCLATGFYPDHVELSWWVNGKEVHSGVCTDPQPLKEQPALQDSRYALS | |
| | | SRLRVSATFWQNPRNHFRCQVQFYGLSENDEWTQDRAKPVTQIVSAEAW | |
| | | GRASDKTHTCPPCPAPELLGPSVFLFPPKPKDTLMISRTPEVTCVVVDV | |
| | | SHEDPEVKFNWYVDGVEVHNAKTKPREEQYNSTYRVVSVLTVLHQDWLN | |
| | | GKEYKCKVSNKALPAPIEKTISKAKGQPREPQVYTLPPSREEMTKNQVS | |
| | | LSCAVKGFYPSDIAVEWESNGQPENNYKTTPPVLDSDGSFFLVSKLTVD | |
| | | KSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 137 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPATLSLSPGERATLSCRASKSVRTGMHWYQQKPGQAPRLLIY | 138 |
| | chain | GASNLESGIPARFSGSGSGTDFTLTISSLEPEDFAVYYCQQSWNHFTFG | |
| | | QGTKLEIKPDIQNPDPAVYQLRDSKSSDKSVCLFTDFDSQTQVSQSKDS | |
| | | DVYITDKCVLDMRSMDFKSNSAVAWSQKSDFACANAFQNSIIPEDTFFP | |
| | | SPESS | |

### Characterization of Anti-B7H3/GITR Bi-Specific Antibodies

These bispecific antibodies are evaluated for their in vitro and in vivo activity, including binding to target antigen (B7H3 and GITR), agonistic activity in GITR reporter assay system, activation of immune cells, anti-tumor activity in mouse models.

### Example 6: Anti-B7H3/OX40 Bi-Specific Antibodies

Anti-B7H3/OX40 bi-specific antibodies were produced and characterized using parent anti-B7H3 antibody clone Ly1612 and parent anti-OX40 antibody clone Ly598 (OX40 Ab1). The amino acid sequences of the V_{H} and the V_{L} of the parent clones are provided in Table 1 above. The heavy chain and light chain complementary determining regions determined by the Kabat scheme are in boldface.

cDNAs encoding the heavy chain variable region (V_{H}) and the light chain variable region (V_{L}) of the parent clones were used as the starting materials for making these bi-specific antibodies. CHO-cell transient expression was carried out with plasmids configured for expressing the polypeptide chains of the bi-specific antibodies. These resultant bispecific antibodies were purified by protein A affinity chromatography.

The amino acid sequences of the polypeptides of exemplary bi-specific antibodies are provided in Table 6 below:

TABLE 6
Exemplary Anti-B7H3/OX40 Bi-Specific Antibodies

| | | | SED ID |
|---|---|---|---|
| Antibody Clones | | Amino acid sequence | NO: |
| Ly1953 | 1^{st} | QVQLVQSGAEVKKPGASVKVSCKASGYTFTSYVMHWVRQKPGQGLEWIG | 139 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | EIVLTQSPATLSLSPGERATLSCRASSSVSYMHWYQQKPGQAPRPWIYA | 140 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1954 | 1^{st} | QVQLVQSGAEVKKPGASVKVSCKASGYTFTSYVMHWVRQKPGQGLEWIG | 141 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 142 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly1955 | 1^{st} | DIQMTQSPSSLSASVGDRVTITCRASQDISNYLNWYQQKPGKAPKLLIY | 143 |
| | chain | YTSRLRSGVPSRFSGSGSGTDFTLTISSLQPEDFATYYCQQGHTLPPTF | |
| | | GQGTKVEIKGGGGSGGGGSGGGGSGGGGSEVQLVQSGAEVKKPGASVKV | |
| | | SCKASGYTFTDSYMSWVRQAPGQGLEWIGDMYPDNGDSSYNQKFRERVT | |
| | | ITRDTSTSTAYLELSSLRSEDTAVYYCVLAPRWYFSVWGQGTLVTVSSG | |
| | | GGGSGGGGSGGGGSGGGGSQVQLVQSGAEVKKPGASVKVSCKASGYTFT | |
| | | SYVMHWVRQKPGQGLEWIGYINPYNEGTESTDKFKGRATMTSDKSTSTV | |
| | | YMELSSLRSEDTAVYYCASIYYGYEGTYFGVWGQGTLVTVSSASTKGPS | |
| | | VFPLAPSSKSTSGGTAALGCLVKDYFPEPVTVSWNSGALTSGVHTFPAV | |
| | | LQSSGLYSLSSVVTVPSSSLGTQTYICNVNHKPSNTKVDKKVEPKSCDK | |
| | | THTCPPCPAPELLGPSVFLFPPKPKDTLMISRTPEVTCVVVDVSHEDPE | |
| | | VKFNWYVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRWQQ | |
| | | GNVFSCSVMHEALHNHYTQKSLSLSPGK | |
| | 2^{nd} | EIVLTQSPATLSLSPGERATLSCRASSSVSYMHWYQQKPGQAPRPWIYA | 144 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1956 | 1^{st} | QVQLVQSGAEVKKPGASVKVSCKASGYTFTSYVMHWVRQKPGQGLEWIG | 145 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EVQLVQSGAEVKKPGASVKVSCKASGYTFTDSYMSWVRQAPGQGLEWIG | 146 |
| | chain | DMYPDNGDSSYNQKFRERVTITRDTSTSTAYLELSSLRSEDTAVYYCVL | |
| | | APRWYFSVWGQGTLVTVSSASVAAPSVFIFPPSDEQLKSGTASVVCLLN | |
| | | NFYPREAKVQWKVDNALQSGNSQESVTEQDSKDSTYSLSSTLILSKADY | |
| | | EKHKVYACEVTHQGLSSPVTKSFNRGECDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVCTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGK | |
| | 3^{rd} | EIVLTQSPATLSLSPGERATLSCRASSSVSYMHWYQQKPGQAPRPWIYA | 147 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 148 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1957 | 1^{st} | QVQLVQSGAEVKKPGASVKVSCKASGYTFTSYVMHWVRQKPGQGLEWIG | 149 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EVQLVQSGAEVKKPGASVKVSCKASGYTFTDSYMSWVRQAPGQGLEWIG | 150 |
| | chain | DMYPDNGDSSYNQKFRERVTITRDTSTSTAYLELSSLRSEDTAVYYCVL | |
| | | APRWYFSVWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECDKTHTCPPCPAPELLGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTKPPSREEMTKNQVSLTCKVKGFYPSDIAVEWESNGQPEN | |
| | | NYKTTPPVLDSDGSFFLYSKLTVDKSRWQQGNVFSCSVMHEALHNRFTQ | |
| | | KSLSLSPGK | |
| | 3^{rd} | EIVLTQSPATLSLSPGERATLSCRASSSVSYMHWYQQKPGQAPRPWIYA | 151 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 152 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1958 | 1^{st} | QVQLVQSGAEVKKPGASVKVSCKASGYTFTSYVMHWVRQKPGQGLEWIG | 153 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | EVQLVQSGAEVKKPGASVKVSCKASGYTFTDSYMSWVRQAPGQGLEWIG | 154 |
| | chain | DMYPDNGDSSYNQKFRERVTITRDTSTSTAYLELSSLRSEDTAVYYCVL | |
| | | APRWYFSVWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVLPPSREEMTKNQVSLLCLVKGFYPSDIAVEWESNGQPEN | |
| | | NYLTWPPVLDSDGSFFLYSKLTVDKSRWQQGNVFSCSVMHEALHNRFTQ | |
| | | KSLSLSPGK | |
| | 3^{rd} | EIVLTQSPATLSLSPGERATLSCRASSSVSYMHWYQQKPGQAPRPWIYA | 155 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 156 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1959 | 1^{st} | QVQLVQSGAEVKKPGASVKVSCKASGYTFTSYVMHWVRQKPGQGLEWIG | 157 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EVQLVQSGAEVKKPGASVKVSCKASGYTFTDSYMSWVRQAPGQGLEWIG | 158 |
| | chain | DMYPDNGDSSYNQKFRERVTITRDTSTSTAYLELSSLRSEDTAVYYCVL | |
| | | APRWYFSVWGQGTLVTVSSDLKNVFPPEVAVFEPSEAEISHTQKATLVC | |
| | | LATGFYPDHVELSWWVNGKEVHSGVCTDPQPLKEQPALQDSRYALSSRL | |
| | | RVSATFWQNPRNHFRCQVQFYGLSENDEWTQDRAKPVTQIVSAEAWGRA | |
| | | SDKTHTCPPCPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVCTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 159 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 160 |
| | chain | YTSRLRSGVPSRFSGSGSGTDFTLTISSLQPEDFATYYCQQGHTLPPTF | |
| | | GQGTKVEIKPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |
| Ly1960 | 1^{st} | QVQLVQSGAEVKKPGASVKVSCKASGYTFTSYVMHWVRQKPGQGLEWIG | 161 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 162 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVQSGAEVKKPGASVKVSCKASGYTFTDSYMSWVRQAPGQGLEWIGD | |
| | | MYPDNGDSSYNQKFRERVTITRDTSTSTAYLELSSLRSEDTAVYYCVLA | |
| | | PRWYFSVWGQGTLVTVSSDLKNVFPPEVAVFEPSEAEISHTQKATLVCL | |
| | | ATGFYPDHVELSWWVNGKEVHSGVCTDPQPLKEQPALQDSRYALSSRLR | |
| | | VSATFWQNPRNHERCQVQFYGLSENDEWTQDRAKPVTQIVSAEAWGRAS | |
| | | DKTHTCPPCPAPELLGPSVFLFPPKPKDTLMISRTPEVTCVVVDVSHED | |
| | | PEVKFNWYVDGVEVHNAKTKPREEQYNSTYRVVSVLTVLHQDWLNGKEY | |
| | | KCKVSNKALPAPIEKTISKAKGQPREPQVYTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 163 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 164 |
| | chain | YTSRLRSGVPSRFSGSGSGTDFTLTISSLQPEDFATYYCQQGHTLPPTF | |
| | | GQGTKVEIKPDIQNPDPAVYQLRDSKSSDKSVCLFTDFDSQTQVSQSKD | |
| | | SDVYITDKCVLDMRSMDFKSNSAVAWSQKSDFACANAFQNSIIPEDTFF | |
| | | PSPESS | |

### Characterization of Anti-B7H3/OX40 Bi-Specific Antibodies

These bispecific antibodies are evaluated for their in vitro and in vivo activity, including binding to target antigen (B7H3 and OX40), agonistic activity in OX40 reporter assay system, activation of immune cells, anti-tumor activity in mouse models.

### Example 7: Anti-B7H3/CD47 Bi-Specific Antibodies

Anti-B7H3/CD47 bi-specific antibodies were produced using the parent anti-B7H3 antibodies Ly1612 (B7H3 Ab1) and anti-CD47 antibodies including Ly1667 (CD47 Ab1) and Ly1668 (CD47 Ab2). The V_{H} and V_{L} sequences of the parent antibodies are provided in Table 1 above (CDRs determined pursuant to the Kabat scheme are in boldface)

cDNAs encoding the V_{H} and V_{L} chains of both of the parent antibodies were used as the starting materials for constructing anti-B7H3/CD47 bispecific antibodies. CHO-cell transient expression was carried out with plasmids configured for expressing polypeptide chains of the bi-specific antibodies. These antibodies were purified by protein A affinity chromatography.

The amino acid sequences of the exemplary anti-B7H3/CD47 bispecific antibodies are provided in Table 7 below:

TABLE 7
Exemplary Anti-B7H3/CD47 Bi-Specific Antibodies

| | | | SED ID |
|---|---|---|---|
| Antibody Clones | | Amino acid sequence | NO: |
| Ly2043 | 1^{st} | QVQLVQSGAEVKKPGASVKVSCKASGYTFTSYVMHWVRQKPGQGLEWIG | 165 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIVMTQSPLSLPVTPGEPASI | |
| | | SCRSSQSIVYSNGNTYLGWYLQKPGQSPQLLIYKVSNRFSGVPDRESGS | |
| | | GSGTDFTLKISRVEAEDVGVYYCFQGSHVPYTFGQGTKLEIKGGGGSGG | |
| | | GGSGGGGSGGGGSQVQLVQSGAEVKKPGASVKVSCKASGYTFTNYNMHW | |
| | | VRQAPGQRLEWMGTIYPGNDDTSYNQKFKDRVTITADTSASTAYMELSS | |
| | | LRSEDTAVYYCARGGYRAMDYWGQGTLVTVSS | |
| | 2^{nd} | EIVLTQSPATLSLSPGERATLSCRASSSVSYMHWYQQKPGQAPRPWIYA | 166 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2044 | 1^{st} | QVQLVQSGAEVKKPGASVKVSCKASGYTFTSYVMHWVRQKPGQGLEWIG | 167 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTNYNMHWVRQAPGQRLEWMGTIYPGNDDTSYNQKFKDRVTI | |
| | | TADTSASTAYMELSSLRSEDTAVYYCARGGYRAMDYWGQGTLVTVSSGG | |
| | | GGSGGGGSGGGGSGGGGSDIVMTQSPLSLPVTPGEPASISCRSSQSIVY | |
| | | SNGNTYLGWYLQKPGQSPQLLIYKVSNRFSGVPDRFSGSGSGTDFTLKI | |
| | | SRVEAEDVGVYYCFQGSHVPYTFGQGTKLEIK | |
| | 2^{nd} | EIVLTQSPATLSLSPGERATLSCRASSSVSYMHWYQQKPGQAPRPWIYA | 168 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2045 | 1^{st} | QVQLVQSGAEVKKPGASVKVSCKASGYTFTSYVMHWVRQKPGQGLEWIG | 169 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 170 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIVMTQSPLSLP | |
| | | VTPGEPASISCRSSQSIVYSNGNTYLGWYLQKPGQSPQLLIYKVSNRFS | |
| | | GVPDRFSGSGSGTDFTLKISRVEAEDVGVYYCFQGSHVPYTFGQGTKLE | |
| | | IKGGGGSGGGGSGGGGSGGGGSQVQLVQSGAEVKKPGASVKVSCKASGY | |
| | | TFTNYNMHWVRQAPGQRLEWMGTIYPGNDDTSYNQKFKDRVTITADTSA | |
| | | STAYMELSSLRSEDTAVYYCARGGYRAMDYWGQGTLVTVSS | |
| Ly2046 | 1^{st} | QVQLVQSGAEVKKPGASVKVSCKASGYTFTSYVMHWVRQKPGQGLEWIG | 171 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 172 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLVQSGAEVK | |
| | | KPGASVKVSCKASGYTFTNYNMHWVRQAPGQRLEWMGTIYPGNDDTSYN | |
| | | QKFKDRVTITADTSASTAYMELSSLRSEDTAVYYCARGGYRAMDYWGQG | |
| | | TLVTVSSGGGGSGGGGSGGGGSGGGGSDIVMTQSPLSLPVTPGEPASIS | |
| | | CRSSQSIVYSNGNTYLGWYLQKPGQSPQLLIYKVSNRFSGVPDRESGSG | |
| | | SGTDFTLKISRVEAEDVGVYYCFQGSHVPYTFGQGTKLEIK | |
| Ly2047 | 1^{st} | DIVMTQSPLSLPVTPGEPASISCRSSQSIVYSNGNTYLGWYLQKPGQSP | 173 |
| | chain | QLLIYKVSNRFSGVPDRFSGSGSGTDFTLKISRVEAEDVGVYYCFQGSH | |
| | | VPYTFGQGTKLEIKGGGGSGGGGSGGGGSGGGGSQVQLVQSGAEVKKPG | |
| | | ASVKVSCKASGYTFTNYNMHWVRQAPGQRLEWMGTIYPGNDDTSYNQKF | |
| | | KDRVTITADTSASTAYMELSSLRSEDTAVYYCARGGYRAMDYWGQGTLV | |
| | | TVSSGGGGSGGGGSGGGGSGGGGSQVQLVQSGAEVKKPGASVKVSCKAS | |
| | | GYTFTSYVMHWVRQKPGQGLEWIGYINPYNEGTESTDKFKGRATMTSDK | |
| | | STSTVYMELSSLRSEDTAVYYCASIYYGYEGTYFGVWGQGTLVTVSSAS | |
| | | TKGPSVFPLAPSSKSTSGGTAALGCLVKDYFPEPVTVSWNSGALTSGVH | |
| | | TFPAVLQSSGLYSLSSVVTVPSSSLGTQTYICNVNHKPSNTKVDKKVEP | |
| | | KSCDKTHTCPPCPAPELLGPSVFLFPPKPKDTLMISRTPEVTCVVVDVS | |
| | | HEDPEVKFNWYVDGVEVHNAKTKPREEQYNSTYRVVSVLIVLHQDWLNG | |
| | | KEYKCKVSNKALPAPIEKTISKAKGQPREPQVYTLPPSREEMTKNQVSL | |
| | | TCLVKGFYPSDIAVEWESNGQPENNYKTTPPVLDSDGSFFLYSKLTVDK | |
| | | SRWQQGNVFSCSVMHEALHNHYTQKSLSLSPGK | |
| | 2^{nd} | EIVLTQSPATLSLSPGERATLSCRASSSVSYMHWYQQKPGQAPRPWIYA | 174 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2048 | 1^{st} | QVQLVQSGAEVKKPGASVKVSCKASGYTFTNYNMHWVRQAPGQRLEWMG | 175 |
| | chain | TIYPGNDDTSYNQKFKDRVTITADTSASTAYMELSSLRSEDTAVYYCAR | |
| | | GGYRAMDYWGQGTLVTVSSGGGGSGGGGSGGGGSGGGGSDIVMTQSPLS | |
| | | LPVTPGEPASISCRSSQSIVYSNGNTYLGWYLQKPGQSPQLLIYKVSNR | |
| | | FSGVPDRFSGSGSGTDFTLKISRVEAEDVGVYYCFQGSHVPYTFGQGTK | |
| | | LEIKGGGGSGGGGSGGGGSGGGGSQVQLVQSGAEVKKPGASVKVSCKAS | |
| | | GYTFTSYVMHWVRQKPGQGLEWIGYINPYNEGTESTDKFKGRATMTSDK | |
| | | STSTVYMELSSLRSEDTAVYYCASIYYGYEGTYFGVWGQGTLVTVSSAS | |
| | | TKGPSVFPLAPSSKSTSGGTAALGCLVKDYFPEPVTVSWNSGALTSGVH | |
| | | TFPAVLQSSGLYSLSSVVTVPSSSLGTQTYICNVNHKPSNTKVDKKVEP | |
| | | KSCDKTHTCPPCPAPELLGPSVFLFPPKPKDTLMISRTPEVTCVVVDVS | |
| | | HEDPEVKFNWYVDGVEVHNAKTKPREEQYNSTYRVVSVLTVLHQDWLNG | |
| | | KEYKCKVSNKALPAPIEKTISKAKGQPREPQVYTLPPSREEMTKNQVSL | |
| | | TCLVKGFYPSDIAVEWESNGQPENNYKTTPPVLDSDGSFFLYSKLTVDK | |
| | | SRWQQGNVFSCSVMHEALHNHYTQKSLSLSPGK | |
| | 2^{nd} | EIVLTQSPATLSLSPGERATLSCRASSSVSYMHWYQQKPGQAPRPWIYA | 176 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2049 | 1^{st} | QVQLVQSGAEVKKPGASVKVSCKASGYTFTSYVMHWVRQKPGQGLEWIG | 177 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTNYNMHWVRQAPGQRLEWMG | 178 |
| | chain | TIYPGNDDTSYNQKFKDRVTITADTSASTAYMELSSLRSEDTAVYYCAR | |
| | | GGYRAMDYWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVCTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGK | |
| | 3^{rd} | EIVLTQSPATLSLSPGERATLSCRASSSVSYMHWYQQKPGQAPRPWIYA | 179 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPLSLPVTPGEPASISCRSSQSIVYSNGNTYLGWYLQKPGQSP | 180 |
| | chain | QLLIYKVSNRFSGVPDRFSGSGSGTDFTLKISRVEAEDVGVYYCFQGSH | |
| | | VPYTFGQGTKLEIKSSASTKGPSVFPLAPSSKSTSGGTAALGCLVKDYF | |
| | | PEPVTVSWNSGALTSGVHTFPAVLQSSGLYSLSSVVTVPSSSLGTQTYI | |
| | | CNVNHKPSNTKVDKKVEPKSC | |
| Ly2050 | 1^{st} | QVQLVQSGAEVKKPGASVKVSCKASGYTFTSYVMHWVRQKPGQGLEWIG | 181 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTNYNMHWVRQAPGQRLEWMG | 182 |
| | chain | TIYPGNDDTSYNQKFKDRVTITADTSASTAYMELSSLRSEDTAVYYCAR | |
| | | GGYRAMDYWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTKPPSREEMTKNQVSLTCKVKGFYPSDIAVEWESNGQPEN | |
| | | NYKTTPPVLDSDGSFFLYSKLTVDKSRWQQGNVFSCSVMHEALHNRETQ | |
| | | KSLSLSPGK | |
| | 3^{rd} | EIVLTQSPATLSLSPGERATLSCRASSSVSYMHWYQQKPGQAPRPWIYA | 183 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPLSLPVTPGEPASISCRSSQSIVYSNGNTYLGWYLQKPGQSP | 184 |
| | chain | QLLIYKVSNRFSGVPDRFSGSGSGTDFTLKISRVEAEDVGVYYCFQGSH | |
| | | VPYTFGQGTKLEIKSSASTKGPSVFPLAPSSKSTSGGTAALGCLVKDYF | |
| | | PEPVTVSWNSGALTSGVHTFPAVLQSSGLYSLSSVVTVPSSSLGTQTYI | |
| | | CNVNHKPSNTKVDKKVEPKSC | |
| Ly2051 | 1^{st} | QVQLVQSGAEVKKPGASVKVSCKASGYTFTSYVMHWVRQKPGQGLEWIG | 185 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | QVQLVQSGAEVKKPGASVKVSCKASGYTFTNYNMHWVRQAPGQRLEWMG | 186 |
| | chain | TIYPGNDDTSYNQKFKDRVTITADTSASTAYMELSSLRSEDTAVYYCAR | |
| | | GGYRAMDYWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVLPPSREEMTKNQVSLLCLVKGFYPSDIAVEWESNGQPEN | |
| | | NYLTWPPVLDSDGSFFLYSKLTVDKSRWQQGNVFSCSVMHEALHNRFTQ | |
| | | KSLSLSPGK | |
| | 3^{rd} | EIVLTQSPATLSLSPGERATLSCRASSSVSYMHWYQQKPGQAPRPWIYA | 187 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPLSLPVTPGEPASISCRSSQSIVYSNGNTYLGWYLQKPGQSP | 188 |
| | chain | QLLIYKVSNRFSGVPDRFSGSGSGTDFTLKISRVEAEDVGVYYCFQGSH | |
| | | VPYTFGQGTKLEIKSSASTKGPSVFPLAPSSKSTSGGTAALGCLVKDYF | |
| | | PEPVTVSWNSGALTSGVHTFPAVLQSSGLYSLSSVVTVPSSSLGTQTYI | |
| | | CNVNHKPSNTKVDKKVEPKSC | |
| Ly2052 | 1^{st} | QVQLVQSGAEVKKPGASVKVSCKASGYTFTSYVMHWVRQKPGQGLEWIG | 189 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTNYNMHWVRQAPGQRLEWMG | 190 |
| | chain | TIYPGNDDTSYNQKFKDRVTITADTSASTAYMELSSLRSEDTAVYYCAR | |
| | | GGYRAMDYWGQGTLVTVSSDLKNVFPPEVAVFEPSEAEISHTQKATLVC | |
| | | LATGFYPDHVELSWWVNGKEVHSGVCTDPQPLKEQPALQDSRYALSSRL | |
| | | RVSATFWQNPRNHFRCQVQFYGLSENDEWTQDRAKPVTQIVSAEAWGRA | |
| | | SDKTHTCPPCPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVCTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 191 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPLSLPVTPGEPASISCRSSQSIVYSNGNTYLGWYLQKPGQSP | 192 |
| | chain | QLLIYKVSNRFSGVPDRFSGSGSGTDFTLKISRVEAEDVGVYYCFQGSH | |
| | | VPYTFGQGTKLEIKPDIQNPDPAVYQLRDSKSSDKSVCLFTDFDSQTQV | |
| | | SQSKDSDVYITDKCVLDMRSMDFKSNSAVAWSQKSDFACANAFQNSIIP | |
| | | EDTFFPSPESS | |
| Ly2053 | 1^{st} | QVQLVQSGAEVKKPGASVKVSCKASGYTFTSYVMHWVRQKPGQGLEWIG | 193 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 194 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | VQLVQSGAEVKKPGASVKVSCKASGYTFTNYNMHWVRQAPGQRLEWMGT | |
| | | IYPGNDDTSYNQKFKDRVTITADTSASTAYMELSSLRSEDTAVYYCARG | |
| | | GYRAMDYWGQGTLVTVSSDLKNVFPPEVAVFEPSEAEISHTQKATLVCL | |
| | | ATGFYPDHVELSWWVNGKEVHSGVCTDPQPLKEQPALQDSRYALSSRLR | |
| | | VSATFWQNPRNHFRCQVQFYGLSENDEWTQDRAKPVTQIVSAEAWGRAS | |
| | | DKTHTCPPCPAPELLGPSVFLFPPKPKDTLMISRTPEVTCVVVDVSHED | |
| | | PEVKFNWYVDGVEVHNAKTKPREEQYNSTYRVVSVLTVLHQDWLNGKEY | |
| | | KCKVSNKALPAPIEKTISKAKGQPREPQVYTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 195 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPLSLPVTPGEPASISCRSSQSIVYSNGNTYLGWYLQKPGQSP | 196 |
| | chain | QLLIYKVSNRFSGVPDRFSGSGSGTDFTLKISRVEAEDVGVYYCFQGSH | |
| | | VPYTFGQGTKLEIKPDIQNPDPAVYQLRDSKSSDKSVCLFTDFDSQTQV | |
| | | SQSKDSDVYITDKCVLDMRSMDFKSNSAVAWSQKSDFACANAFQNSIIP | |
| | | EDTFFPSPESS | |
| Ly2054 | 1^{st} | QVQLVQSGAEVKKPGASVKVSCKASGYTFTSYVMHWVRQKPGQGLEWIG | 197 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIVMTQSPDSLAVSLGERATI | |
| | | NCKSSQSVLYSSNNRNYLAWYQQKPGQPPKLLINQASTRASGVPDRESG | |
| | | SGSGTEFTLIISSLHAEDVAIYYCQQYYTPPLAFGGGTKLEIKGGGGSG | |
| | | GGGSGGGGSGGGGSKVQLVESGGGLVKPGGSLRLSCAASGFTFSNAWMN | |
| | | WVRQAPGKGLEWVGRIKRKTDGETTDYAAPVKGRFSISRDDSKNTLYLQ | |
| | | MNSLKTEDTAVYYCAGSNRAFDIWGQGTMVTVSA | |
| | 2^{nd} | EIVLTQSPATLSLSPGERATLSCRASSSVSYMHWYQQKPGQAPRPWIYA | 198 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2055 | 1^{st} | QVQLVQSGAEVKKPGASVKVSCKASGYTFTSYVMHWVRQKPGQGLEWIG | 199 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSKVQLVESGGGLVKPGGSLRLS | |
| | | CAASGFTFSNAWMNWVRQAPGKGLEWVGRIKRKTDGETTDYAAPVKGRF | |
| | | SISRDDSKNTLYLQMNSLKTEDTAVYYCAGSNRAFDIWGQGTMVTVSAG | |
| | | GGGSGGGGSGGGGSGGGGSDIVMTQSPDSLAVSLGERATINCKSSQSVL | |
| | | YSSNNRNYLAWYQQKPGQPPKLLINQASTRASGVPDRFSGSGSGTEFTL | |
| | | IISSLHAEDVAIYYCQQYYTPPLAFGGGTKLEIK | |
| | 2^{nd} | EIVLTQSPATLSLSPGERATLSCRASSSVSYMHWYQQKPGQAPRPWIYA | 200 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2056 | 1^{st} | QVQLVQSGAEVKKPGASVKVSCKASGYTFTSYVMHWVRQKPGQGLEWIG | 201 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 202 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIVMTQSPDSLA | |
| | | VSLGERATINCKSSQSVLYSSNNRNYLAWYQQKPGQPPKLLINQASTRA | |
| | | SGVPDRFSGSGSGTEFTLIISSLHAEDVAIYYCQQYYTPPLAFGGGTKL | |
| | | EIKGGGGSGGGGSGGGGSGGGGSKVQLVESGGGLVKPGGSLRLSCAASG | |
| | | FTFSNAWMNWVRQAPGKGLEWVGRIKRKTDGETTDYAAPVKGRFSISRD | |
| | | DSKNTLYLQMNSLKTEDTAVYYCAGSNRAFDIWGQGTMVTVSA | |
| Ly2057 | 1^{st} | QVQLVQSGAEVKKPGASVKVSCKASGYTFTSYVMHWVRQKPGQGLEWIG | 203 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | EIVLTQSPATLSLSPGERATLSCRASSSVSYMHWYQQKPGQAPRPWIYA | 204 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSKVQLVESGGGLV | |
| | | KPGGSLRLSCAASGFTFSNAWMNWVRQAPGKGLEWVGRIKRKTDGETTD | |
| | | YAAPVKGRFSISRDDSKNTLYLQMNSLKTEDTAVYYCAGSNRAFDIWGQ | |
| | | GTMVTVSAGGGGSGGGGSGGGGSGGGGSDIVMTQSPDSLAVSLGERATI | |
| | | NCKSSQSVLYSSNNRNYLAWYQQKPGQPPKLLINQASTRASGVPDRFSG | |
| | | SGSGTEFTLIISSLHAEDVAIYYCQQYYTPPLAFGGGTKLEIK | |
| Ly2058 | 1^{st} | DIVMTQSPDSLAVSLGERATINCKSSQSVLYSSNNRNYLAWYQQKPGQP | 205 |
| | chain | PKLLINQASTRASGVPDRFSGSGSGTEFTLIISSLHAEDVAIYYCQQYY | |
| | | TPPLAFGGGTKLEIKGGGGSGGGGSGGGGSGGGGSKVQLVESGGGLVKP | |
| | | GGSLRLSCAASGFTFSNAWMNWVRQAPGKGLEWVGRIKRKTDGETTDYA | |
| | | APVKGRFSISRDDSKNTLYLQMNSLKTEDTAVYYCAGSNRAFDIWGQGT | |
| | | MVTVSAGGGGSGGGGSGGGGSGGGGSQVQLVQSGAEVKKPGASVKVSCK | |
| | | ASGYTFTSYVMHWVRQKPGQGLEWIGYINPYNEGTESTDKFKGRATMTS | |
| | | DKSTSTVYMELSSLRSEDTAVYYCASIYYGYEGTYFGVWGQGTLVTVSS | |
| | | ASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKV | |
| | | EPKSCDKTHTCPPCPAPELLGPSVFLFPPKPKDTLMISRTPEVTCVVVD | |
| | | VSHEDPEVKFNWYVDGVEVHNAKTKPREEQYNSTYRVVSVLTVLHQDWL | |
| | | NGKEYKCKVSNKALPAPIEKTISKAKGQPREPQVYTLPPSREEMTKNQV | |
| | | SLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | 2^{nd} | EIVLTQSPATLSLSPGERATLSCRASSSVSYMHWYQQKPGQAPRPWIYA | 206 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2059 | 1^{st} | KVQLVESGGGLVKPGGSLRLSCAASGFTFSNAWMNWVRQAPGKGLEWVG | 207 |
| | chain | RIKRKTDGETTDYAAPVKGRFSISRDDSKNTLYLQMNSLKTEDTAVYYC | |
| | | AGSNRAFDIWGQGTMVTVSAGGGGSGGGGSGGGGSGGGGSDIVMTQSPD | |
| | | SLAVSLGERATINCKSSQSVLYSSNNRNYLAWYQQKPGQPPKLLINQAS | |
| | | TRASGVPDRESGSGSGTEFTLIISSLHAEDVAIYYCQQYYTPPLAFGGG | |
| | | TKLEIKGGGGSGGGGSGGGGSGGGGSQVQLVQSGAEVKKPGASVKVSCK | |
| | | ASGYTFTSYVMHWVRQKPGQGLEWIGYINPYNEGTESTDKFKGRATMTS | |
| | | DKSTSTVYMELSSLRSEDTAVYYCASIYYGYEGTYFGVWGQGTLVTVSS | |
| | | ASTKGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVDKKV | |
| | | EPKSCDKTHTCPPCPAPELLGPSVFLFPPKPKDTLMISRTPEVTCVVVD | |
| | | VSHEDPEVKFNWYVDGVEVHNAKTKPREEQYNSTYRVVSVLTVLHQDWL | |
| | | NGKEYKCKVSNKALPAPIEKTISKAKGQPREPQVYTLPPSREEMTKNQV | |
| | | SLTCLVKGFYPSDIAVEWESNGQPENNYKTTPPVLDSDGSFFLYSKLTV | |
| | | DKSRWQQGNVFSCSVMHEALHNHYTQKSLSLSPGK | |
| | 2^{nd} | EIVLTQSPATLSLSPGERATLSCRASSSVSYMHWYQQKPGQAPRPWIYA | 208 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2060 | 1^{st} | QVQLVQSGAEVKKPGASVKVSCKASGYTFTSYVMHWVRQKPGQGLEWIG | 209 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | KVQLVESGGGLVKPGGSLRLSCAASGFTFSNAWMNWVRQAPGKGLEWVG | 210 |
| | chain | RIKRKTDGETTDYAAPVKGRFSISRDDSKNTLYLQMNSLKTEDTAVYYC | |
| | | AGSNRAFDIWGQGTMVTVSAASVAAPSVFIFPPSDEQLKSGTASVVCLL | |
| | | NNFYPREAKVQWKVDNALQSGNSQESVTEQDSKDSTYSLSSTLTLSKAD | |
| | | YEKHKVYACEVTHQGLSSPVTKSFNRGECDKTHTCPPCPAPELLGPSVF | |
| | | LFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKA | |
| | | KGQPREPQVCTLPPSREEMTKNQVSLSCAVKGFYPSDIAVEWESNGQPE | |
| | | NNYKTTPPVLDSDGSFFLVSKLTVDKSRWQQGNVFSCSVMHEALHNRFT | |
| | | QKSLSLSPGK | |
| | 3^{rd} | EIVLTQSPATLSLSPGERATLSCRASSSVSYMHWYQQKPGQAPRPWIYA | 211 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPDSLAVSLGERATINCKSSQSVLYSSNNRNYLAWYQQKPGQP | 212 |
| | chain | PKLLINQASTRASGVPDRFSGSGSGTEFTLIISSLHAEDVAIYYCQQYY | |
| | | TPPLAFGGGTKLEIKSSASTKGPSVFPLAPSSKSTSGGTAALGCLVKDY | |
| | | FPEPVTVSWNSGALTSGVHTFPAVLQSSGLYSLSSVVTVPSSSLGTQTY | |
| | | ICNVNHKPSNTKVDKKVEPKSC | |
| Ly2061 | 1^{st} | QVQLVQSGAEVKKPGASVKVSCKASGYTFTSYVMHWVRQKPGQGLEWIG | 213 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTDPPSREEMTKNQVSLTCEVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | KVQLVESGGGLVKPGGSLRLSCAASGFTFSNAWMNWVRQAPGKGLEWVG | 214 |
| | chain | RIKRKTDGETTDYAAPVKGRFSISRDDSKNTLYLQMNSLKTEDTAVYYC | |
| | | AGSNRAFDIWGQGTMVTVSAASVAAPSVFIFPPSDEQLKSGTASVVCLL | |
| | | NNFYPREAKVQWKVDNALQSGNSQESVTEQDSKDSTYSLSSTLTLSKAD | |
| | | YEKHKVYACEVTHQGLSSPVTKSFNRGECDKTHTCPPCPAPELLGPSVF | |
| | | LFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKA | |
| | | KGQPREPQVYTKPPSREEMTKNQVSLTCKVKGFYPSDIAVEWESNGQPE | |
| | | NNYKTTPPVLDSDGSFFLYSKLTVDKSRWQQGNVFSCSVMHEALHNRFT | |
| | | QKSLSLSPGK | |
| | 3^{rd} | EIVLTQSPATLSLSPGERATLSCRASSSVSYMHWYQQKPGQAPRPWIYA | 215 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPDSLAVSLGERATINCKSSQSVLYSSNNRNYLAWYQQKPGQP | 216 |
| | chain | PKLLINQASTRASGVPDRFSGSGSGTEFTLIISSLHAEDVAIYYCQQYY | |
| | | TPPLAFGGGTKLEIKSSASTKGPSVFPLAPSSKSTSGGTAALGCLVKDY | |
| | | FPEPVTVSWNSGALTSGVHTFPAVLQSSGLYSLSSVVTVPSSSLGTQTY | |
| | | ICNVNHKPSNTKVDKKVEPKSC | |
| Ly2062 | 1^{st} | QVQLVQSGAEVKKPGASVKVSCKASGYTFTSYVMHWVRQKPGQGLEWIG | 217 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYVYPPSREEMTKNQVSLTCLVKGFYPSDIAVEWESNGQPEN | |
| | | NYKTTPPVLDSDGSFALVSKLTVDKSRWQQGNVFSCSVMHEALHNHYTQ | |
| | | KSLSLSPGK | |
| | 2^{nd} | KVQLVESGGGLVKPGGSLRLSCAASGFTFSNAWMNWVRQAPGKGLEWVG | 218 |
| | chain | RIKRKTDGETTDYAAPVKGRFSISRDDSKNTLYLQMNSLKTEDTAVYYC | |
| | | AGSNRAFDIWGQGTMVTVSAASVAAPSVFIFPPSDEQLKSGTASVVCLL | |
| | | NNFYPREAKVQWKVDNALQSGNSQESVTEQDSKDSTYSLSSTLTLSKAD | |
| | | YEKHKVYACEVTHQGLSSPVTKSFNRGECDKTHTCPPCPAPELLGPSVF | |
| | | LFPPKPKDTLMISRTPEVTCVVVDVSHEDPEVKFNWYVDGVEVHNAKTK | |
| | | PREEQYNSTYRVVSVLTVLHQDWLNGKEYKCKVSNKALPAPIEKTISKA | |
| | | KGQPREPQVYVLPPSREEMTKNQVSLLCLVKGFYPSDIAVEWESNGQPE | |
| | | NNYLTWPPVLDSDGSFFLYSKLTVDKSRWQQGNVFSCSVMHEALHNRFT | |
| | | QKSLSLSPGK | |
| | 3^{rd} | EIVLTQSPATLSLSPGERATLSCRASSSVSYMHWYQQKPGQAPRPWIYA | 219 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPDSLAVSLGERATINCKSSQSVLYSSNNRNYLAWYQQKPGQP | 220 |
| | chain | PKLLINQASTRASGVPDRFSGSGSGTEFTLIISSLHAEDVAIYYCQQYY | |
| | | TPPLAFGGGTKLEIKSSASTKGPSVFPLAPSSKSTSGGTAALGCLVKDY | |
| | | FPEPVTVSWNSGALTSGVHTFPAVLQSSGLYSLSSVVTVPSSSLGTQTY | |
| | | ICNVNHKPSNTKVDKKVEPKSC | |
| Ly2063 | 1^{st} | QVQLVQSGAEVKKPGASVKVSCKASGYTFTSYVMHWVRQKPGQGLEWIG | 221 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | KVQLVESGGGLVKPGGSLRLSCAASGFTFSNAWMNWVRQAPGKGLEWVG | 222 |
| | chain | RIKRKTDGETTDYAAPVKGRFSISRDDSKNTLYLQMNSLKTEDTAVYYC | |
| | | AGSNRAFDIWGQGTMVTVSADLKNVFPPEVAVFEPSEAEISHTQKATLV | |
| | | CLATGFYPDHVELSWWVNGKEVHSGVCTDPQPLKEQPALQDSRYALSSR | |
| | | LRVSATFWQNPRNHFRCQVQFYGLSENDEWTQDRAKPVTQIVSAEAWGR | |
| | | ASDKTHTCPPCPAPELLGPSVFLFPPKPKDTLMISRTPEVTCVVVDVSH | |
| | | EDPEVKFNWYVDGVEVHNAKTKPREEQYNSTYRVVSVLTVLHQDWLNGK | |
| | | EYKCKVSNKALPAPIEKTISKAKGQPREPQVCTLPPSREEMTKNQVSLS | |
| | | CAVKGFYPSDIAVEWESNGQPENNYKTTPPVLDSDGSFFLVSKLTVDKS | |
| | | RWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 223 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPDSLAVSLGERATINCKSSQSVLYSSNNRNYLAWYQQKPGQP | 224 |
| | chain | PKLLINQASTRASGVPDRFSGSGSGTEFTLIISSLHAEDVAIYYCQQYY | |
| | | TPPLAFGGGTKLEIKPDIQNPDPAVYQLRDSKSSDKSVCLFTDFDSQTQ | |
| | | VSQSKDSDVYITDKCVLDMRSMDFKSNSAVAWSQKSDFACANAFQNSII | |
| | | PEDTFFPSPESS | |
| Ly2064 | 1^{st} | QVQLVQSGAEVKKPGASVKVSCKASGYTFTSYVMHWVRQKPGQGLEWIG | 225 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 226 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSK | |
| | | VQLVESGGGLVKPGGSLRLSCAASGFTFSNAWMNWVRQAPGKGLEWVGR | |
| | | IKRKTDGETTDYAAPVKGRFSISRDDSKNTLYLQMNSLKTEDTAVYYCA | |
| | | GSNRAFDIWGQGTMVTVSADLKNVFPPEVAVFEPSEAEISHTQKATLVC | |
| | | LATGFYPDHVELSWWVNGKEVHSGVCTDPQPLKEQPALQDSRYALSSRL | |
| | | RVSATFWQNPRNHFRCQVQFYGLSENDEWTQDRAKPVTQIVSAEAWGRA | |
| | | SDKTHTCPPCPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 227 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIVMTQSPDSLAVSLGERATINCKSSQSVLYSSNNRNYLAWYQQKPGQP | 228 |
| | chain | PKLLINQASTRASGVPDRFSGSGSGTEFTLIISSLHAEDVAIYYCQQYY | |
| | | TPPLAFGGGTKLEIKPDIQNPDPAVYQLRDSKSSDKSVCLFTDFDSQTQ | |
| | | VSQSKDSDVYITDKCVLDMRSMDFKSNSAVAWSQKSDFACANAFQNSII | |
| | | PEDTFFPSPESS | |

### Characterization of Anti-B7H3/CD47 Bi-Specific Antibodies

These bispecific antibodies are to be evaluated for their in vitro and in vivo activity, including binding to target antigen (B7H3 and CD47), antagonistic activity in B7H3 and CD47 reporter assay system, activation of anti-tumor activity in mouse models.

### Example 8: Anti-B7H3/CD3 Bi-Specific Antibodies

Anti-B7H3/CD3 bi-specific antibodies were produced using the anti-B7H3 antibodies Ly1612 (B7H3 Ab1), anta-CD3 antibody Ly305 (CD3 Ab) and CD3 Ab2. The amino acid sequences of the parent antibodies are provided in Table 1 above.

cDNAs encoding the V_{H} and V_{L} chains of these parent antibodies were used as the starting materials for making the bi-specific antibodies. CHO-cell transient expression was carried out with plasmids configured for expressing polypeptide chains of the bi-specific antibodies. These antibodies were purified by protein A affinity chromatography.

The amino acid sequences of the polypeptide chains of the bi-specific antibodies are provided in Table 8 below:

TABLE 8
Exemplary Anti-B7H3/CD3 Bi-Specific Antibodies

| | | | SED ID |
|---|---|---|---|
| Antibody Clones | | Amino acid sequence | NO: |
| Ly1900 | 1^{st} | QVQLVQSGAEVKKPGASVKVSCKASGYTFTSYVMHWVRQKPGQGLEWIG | 229 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 230 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 231 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 232 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1902 | 1^{st} | QVQLVQSGAEVKKPGASVKVSCKASGYTFTSYVMHWVRQKPGQGLEWIG | 233 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 234 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 235 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1904 | 1^{st} | QVQLVQSGAEVKKPGASVKVSCKASGYTFTSYVMHWVRQKPGQGLEWIG | 236 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 237 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 238 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1901 | 1^{st} | QVQLVQSGAEVKKPGASVKVSCKASGYTFTSYVMHWVRQKPGQGLEWIG | 239 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSGQPREPQVYTLPPSREEMTKNQV | |
| | | SLWCLVKGFYPSDIAVEWESNGQPENNYKTTPPVLDSDGSFFLYSKLTV | |
| | | DKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 240 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGQPREPQVYTLPPSREEMTKNQVSLSCAVKGFYPSDIAV | |
| | | EWESNGQPENNYKTTPPVLDSDGSFFLVSKLTVDKSRWQQGNVFSCSVM | |
| | | HEALHNHYTQKSLSLSPGK | |
| | 3^{rd} | EIVLTQSPATLSLSPGERATLSCRASSSVSYMHWYQQKPGQAPRPWIYA | 241 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1903 | 1^{st} | QVQLVQSGAEVKKPGASVKVSCKASGYTFTSYVMHWVRQKPGQGLEWIG | 242 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLIVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 243 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 244 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |

### Characterization of Anti-B7H3/CD3 Bi-Specific Antibodies

These bispecific antibodies are evaluated for their in vitro and in vivo activity, including binding to target antigen (B7H-3 and CD3), agonistic activity in CD3 reporter assay system, activation of immune cells, anti-tumor activity in mouse models.

### Example 9: Anti-B7H3/CD3/CD137 Tri-Specific Antibodies

Anti-B7H3/CD3/CD137 tri-specific were produced using the anti-B7H3 antibodies Ly1612 (B7H3 Ab1), anti-CD3 antibody Ly305 (CD3 Ab1) and CD3 Ab2, and anti-CD137 antibody Ly1630 (CD137 Ab1). The sequences of the parent antibodies are provided in Table 1 above.

cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 9 below:

TABLE 9
Exemplary Anti-B7H3/CD3/CD137 Tri-Specific Antibodies

| | | | SED ID |
|---|---|---|---|
| Antibody Clones | | Amino acid sequence | NO: |
| Ly1785 | 1^{st} | QVQLVQSGAEVKKPGASVKVSCKASGYTFTSYVMHWVRQKPGQGLEWIG | 245 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFAGFEMHWVRQAPGQGLEWMG | 246 |
| | chain | AIDPKTGGTDYNQKFKDRVTMTRDTSISTAYMELSRLRSDDTAVYYCAR | |
| | | DLGYFDVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECGGGGSGGGGSGGGGSGGGGSQA | |
| | | VVTQEPSLTVSPGGTVTLTCRSSTGAVTTSNYANWVQQKPGQAPRGLIG | |
| | | GTNKRAPWTPARFSGSLLGGKAALTITGAQAEDEADYYCALWYSNLWVF | |
| | | GGGTKLTVLPAPELLGPSVFLFPPKPKDTLMISRTPEVTCVVVDVSHED | |
| | | PEVKFNWYVDGVEVHNAKTKPREEQYNSTYRVVSVLIVLHQDWLNGKEY | |
| | | KCKVSNKALPAPIEKTISKAKGQPREPQVYTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 247 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 248 |
| | chain | YTSRLHSGVPSRFSGSGSGTDYTLTISSLQPEDFATYFCQQSEKLPRTF | |
| | | GGGTKVEIRSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1666 | 1^{st} | QVQLVQSGAEVKKPGASVKVSCKASGYTFTSYVMHWVRQKPGQGLEWIG | 249 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPCREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFAGFEMHWVRQAPGQGLEWMG | 250 |
| | chain | AIDPKTGGTDYNQKFKDRVTMTRDTSISTAYMELSRLRSDDTAVYYCAR | |
| | | DLGYFDVWGQGTLVTVSSASTKGPSVFPLAPSSKSTSGGTAALGCLVKD | |
| | | YFPEPVTVSWNSGALTSGVHTFPAVLQSSGLYSLSSVVTVPSSSLGTQT | |
| | | YICNVNHKPSNTKVDKKVEPKSCGGGGSGGGGSGGGGSGGGGSEVQLVE | |
| | | SGGGLVQPGGSLRLSCAASGFTFSTYAMNWVRQAPGKGLEWVGRIRSKY | |
| | | NNYATYYADSVKGRFTISRDDSKNSLYLQMNSLKTEDTAVYYCVRHGNF | |
| | | GNSYVSWFAYWGQGTLVTVSSASVAAPSVFIFPPSDEQLKSGTASVVCL | |
| | | LNNFYPREAKVQWKVDNALQSGNSQESVTEQDSKDSTYSLSSTLTLSKA | |
| | | DYEKHKVYACEVTHQGLSSPVTKSFNRGECDKTHTCPPCPAPELLGPSV | |
| | | FLFPPKPKDTLMISRTPEVTCVVVDVSHEDPEVKFNWYVDGVEVHNAKT | |
| | | KPREEQYNSTYRVVSVLTVLHQDWLNGKEYKCKVSNKALPAPIEKTISK | |
| | | AKGQPREPQVCTLPPSREEMTKNQVSLSCAVKGFYPSDIAVEWESNGQP | |
| | | ENNYKTTPPVLDSDGSFFLVSKLTVDKSRWQQGNVFSCSVMHEALHNRF | |
| | | TQKSLSLSPGK | |
| | 3^{rd} | EIVLTQSPATLSLSPGERATLSCRASSSVSYMHWYQQKPGQAPRPWIYA | 251 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDIRSNLNWYQQKPGGAVKLLIY | 252 |
| | chain | YTSRLHSGVPSRFSGSGSGTDYTLTISSLQPEDFATYFCQQSEKLPRTF | |
| | | GGGTKVEIRRTVAAPSVFIFPPSDEQLKSGTASVVCLLNNFYPREAKVQ | |
| | | WKVDNALQSGNSQESVTEQDSKDSTYSLSSTLTLSKADYEKHKVYACEV | |
| | | THQGLSSPVTKSFNRGECGGGGSGGGGSGGGGSGGGGSQAVVTQEPSLT | |
| | | VSPGGTVTLTCRSSTGAVTTSNYANWVQQKPGQAPRGLIGGTNKRAPWT | |
| | | PARFSGSLLGGKAALTITGAQAEDEADYYCALWYSNLWVFGGGTKLTVL | |
| | | SSASTKGPSVFPLAPSSKSTSGGTAALGCLVKDYFPEPVTVSWNSGALT | |
| | | SGVHTFPAVLQSSGLYSLSSVVTVPSSSLGTQTYICNVNHKPSNTKVDK | |
| | | KVEPKSC | |
| Ly1793 | 1^{st} | QVQLVQSGAEVKKPGASVKVSCKASGYTFTSYVMHWVRQKPGQGLEWIG | 253 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | QVQLVQSGAEVKKPGASVKVSCKASGYTFTSYVMHWVRQKPGQGLEWIG | 254 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLIVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVKKP | |
| | | GASVKVSCKASGYTFAGFEMHWVRQAPGQGLEWMGAIDPKTGGTDYNQK | |
| | | FKDRVTMTRDTSISTAYMELSRLRSDDTAVYYCARDLGYFDVWGQGTLV | |
| | | TVSSGGGGSGGGGSGGGGSGGGGSDIQMTQSPSSLSASVGDRVTITCRA | |
| | | SQDIRSNLNWYQQKPGGAVKLLIYYTSRLHSGVPSRFSGSGSGTDYTLT | |
| | | ISSLQPEDFATYFCQQSEKLPRTFGGGTKVEIR | |
| | 3^{rd} | EIVLTQSPATLSLSPGERATLSCRASSSVSYMHWYQQKPGQAPRPWIYA | 255 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 256 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1794 | 1^{st} | QVQLVQSGAEVKKPGASVKVSCKASGYTFTSYVMHWVRQKPGQGLEWIG | 257 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 258 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVKKP | |
| | | GASVKVSCKASGYTFAGFEMHWVRQAPGQGLEWMGAIDPKTGGTDYNQK | |
| | | FKDRVTMTRDTSISTAYMELSRLRSDDTAVYYCARDLGYFDVWGQGTLV | |
| | | TVSSGGGGSGGGGSGGGGSGGGGSDIQMTQSPSSLSASVGDRVTITCRA | |
| | | SQDIRSNLNWYQQKPGGAVKLLIYYTSRLHSGVPSRFSGSGSGTDYTLT | |
| | | ISSLQPEDFATYFCQQSEKLPRTFGGGTKVEIR | |
| | 3^{rd} | EIVLTQSPATLSLSPGERATLSCRASSSVSYMHWYQQKPGQAPRPWIYA | 259 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 260 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1795 | 1^{st} | QVQLVQSGAEVKKPGASVKVSCKASGYTFTSYVMHWVRQKPGQGLEWIG | 261 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | QVQLVQSGAEVKKPGASVKVSCKASGYTFTSYVMHWVRQKPGQGLEWIG | 262 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 263 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 264 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Lv1796 | 1^{st} | QVQLVQSGAEVKKPGASVKVSCKASGYTFTSYVMHWVRQKPGQGLEWIG | 265 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSS | |
| | 2^{nd} | QVQLVQSGAEVKKPGASVKVSCKASGYTFTSYVMHWVRQKPGQGLEWIG | 266 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDIRSNLNWYQQKPGGAVKLLIYYTSRLHSGVPSRFS | |
| | | GSGSGTDYTLTISSLQPEDFATYFCQQSEKLPRTFGGGTKVEIR | |
| | 3^{rd} | EIVLTQSPATLSLSPGERATLSCRASSSVSYMHWYQQKPGQAPRPWIYA | 267 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 268 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1797 | 1^{st} | QVQLVQSGAEVKKPGASVKVSCKASGYTFTSYVMHWVRQKPGQGLEWIG | 269 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | QVQLVQSGAEVKKPGASVKVSCKASGYTFTSYVMHWVRQKPGQGLEWIG | 270 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 271 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1798 | ^{1st} | QVQLVQSGAEVKKPGASVKVSCKASGYTFTSYVMHWVRQKPGQGLEWIG | 272 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | QVQLVQSGAEVKKPGASVKVSCKASGYTFTSYVMHWVRQKPGQGLEWIG | 273 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSSGGGGSGGGGSGGGGSGGGGSDIQ | |
| | | MTQSPSSLSASVGDRVTITCRASQDIRSNLNWYQQKPGGAVKLLIYYTS | |
| | | RLHSGVPSRFSGSGSGTDYTLTISSLQPEDFATYFCQQSEKLPRTFGGG | |
| | | TKVEIR | |
| | 3^{rd} | EIVLTQSPATLSLSPGERATLSCRASSSVSYMHWYQQKPGQAPRPWIYA | 274 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Lv1799 | 1^{st} | QVQLVQSGAEVKKPGASVKVSCKASGYTFTSYVMHWVRQKPGQGLEWIG | 275 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 276 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSSGGGGSGGGGSGGGGSGGGGSDIQ | |
| | | MTQSPSSLSASVGDRVTITCRASQDIRSNLNWYQQKPGGAVKLLIYYTS | |
| | | RLHSGVPSRFSGSGSGTDYTLTISSLQPEDFATYFCQQSEKLPRTFGGG | |
| | | TKVEIR | |
| | 3^{rd} | EIVLTQSPATLSLSPGERATLSCRASSSVSYMHWYQQKPGQAPRPWIYA | 277 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1800 | 1^{st} | QVQLVQSGAEVKKPGASVKVSCKASGYTFTSYVMHWVRQKPGQGLEWIG | 278 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFAGF | |
| | | EMHWVRQAPGQGLEWMGAIDPKTGGTDYNQKFKDRVTMTRDTSISTAYM | |
| | | ELSRLRSDDTAVYYCARDLGYFDVWGQGTLVTVSSGGGGSGGGGSGGGG | |
| | | SGGGGSDIQMTQSPSSLSASVGDRVTITCRASQDIRSNLNWYQQKPGGA | |
| | | VKLLIYYTSRLHSGVPSRFSGSGSGTDYTLTISSLQPEDFATYFCQQSE | |
| | | KLPRTFGGGTKVEIR | |
| | 2^{nd} | QVQLVQSGAEVKKPGASVKVSCKASGYTFTSYVMHWVRQKPGQGLEWIG | 279 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVKKPGASVKVSCKASGYTFAGFEMHWVRQAPGQGLEWM | |
| | | GAIDPKTGGTDYNQKFKDRVTMTRDTSISTAYMELSRLRSDDTAVYYCA | |
| | | RDLGYFDVWGQGTLVTVSSGGGGSGGGGSGGGGSGGGGSDIQMTQSPSS | |
| | | LSASVGDRVTITCRASQDIRSNLNWYQQKPGGAVKLLIYYTSRLHSGVP | |
| | | SRFSGSGSGTDYTLTISSLQPEDFATYFCQQSEKLPRTFGGGTKVEIR | |
| | 3^{rd} | EIVLTQSPATLSLSPGERATLSCRASSSVSYMHWYQQKPGQAPRPWIYA | 280 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1801 | 1^{st} | QVQLVQSGAEVKKPGASVKVSCKASGYTFTSYVMHWVRQKPGQGLEWIG | 281 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFAGF | |
| | | EMHWVRQAPGQGLEWMGAIDPKTGGTDYNQKFKDRVTMTRDTSISTAYM | |
| | | ELSRLRSDDTAVYYCARDLGYFDVWGQGTLVTVSSGGGGSGGGGSGGGG | |
| | | SGGGGSDIQMTQSPSSLSASVGDRVTITCRASQDIRSNLNWYQQKPGGA | |
| | | VKLLIYYTSRLHSGVPSRFSGSGSGTDYTLTISSLQPEDFATYFCQQSE | |
| | | KLPRTFGGGTKVEIR | |
| | 2^{nd} | QVQLVQSGAEVKKPGASVKVSCKASGYTFTSYVMHWVRQKPGQGLEWIG | 282 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 283 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1802 | 1^{st} | QVQLVQSGAEVKKPGASVKVSCKASGYTFTSYVMHWVRQKPGQGLEWIG | 284 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 285 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVKKPGASVKVSCKASGYTFAGFEMHWVRQAPGQGLEWM | |
| | | GAIDPKTGGTDYNQKFKDRVTMTRDTSISTAYMELSRLRSDDTAVYYCA | |
| | | RDLGYFDVWGQGTLVTVSSGGGGSGGGGSGGGGSGGGGSDIQMTQSPSS | |
| | | LSASVGDRVTITCRASQDIRSNLNWYQQKPGGAVKLLIYYTSRLHSGVP | |
| | | SRFSGSGSGTDYTLTISSLQPEDFATYFCQQSEKLPRTFGGGTKVEIR | |
| | 3^{rd} | EIVLTQSPATLSLSPGERATLSCRASSSVSYMHWYQQKPGQAPRPWIYA | 286 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1803 | 1^{st} | QVQLVQSGAEVKKPGASVKVSCKASGYTFTSYVMHWVRQKPGQGLEWIG | 287 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFAGF | |
| | | EMHWVRQAPGQGLEWMGAIDPKTGGTDYNQKFKDRVTMTRDTSISTAYM | |
| | | ELSRLRSDDTAVYYCARDLGYFDVWGQGTLVTVSS | |
| | 2^{nd} | QVQLVQSGAEVKKPGASVKVSCKASGYTFTSYVMHWVRQKPGQGLEWIG | 288 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDIRSNLNWYQQKPGGAVKLLI | |
| | | YYTSRLHSGVPSRFSGSGSGTDYTLTISSLQPEDFATYFCQQSEKLPRT | |
| | | FGGGTKVEIR | |
| | 3^{rd} | EIVLTQSPATLSLSPGERATLSCRASSSVSYMHWYQQKPGQAPRPWIYA | 289 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1804 | 1^{st} | QVQLVQSGAEVKKPGASVKVSCKASGYTFTSYVMHWVRQKPGQGLEWIG | 290 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 291 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 292 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLVQSGAEVK | |
| | | KPGASVKVSCKASGYTFAGFEMHWVRQAPGQGLEWMGAIDPKTGGTDYN | |
| | | QKFKDRVTMTRDTSISTAYMELSRLRSDDTAVYYCARDLGYFDVWGQGT | |
| | | LVTVSSGGGGSGGGGSGGGGSGGGGSDIQMTQSPSSLSASVGDRVTITC | |
| | | RASQDIRSNLNWYQQKPGGAVKLLIYYTSRLHSGVPSRFSGSGSGTDYT | |
| | | LTISSLQPEDFATYFCQQSEKLPRTFGGGTKVEIR | |
| Ly1805 | 1^{st} | QVQLVQSGAEVKKPGASVKVSCKASGYTFTSYVMHWVRQKPGQGLEWIG | 293 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFAGFEMHWVRQAPGQGLEWMGAIDPKTGGTDYNQKFKDRVTM | |
| | | TRDTSISTAYMELSRLRSDDTAVYYCARDLGYFDVWGQGTLVTVSSGGG | |
| | | GSGGGGSGGGGSGGGGSDIQMTQSPSSLSASVGDRVTITCRASQDIRSN | |
| | | LNWYQQKPGGAVKLLIYYTSRLHSGVPSRFSGSGSGTDYTLTISSLQPE | |
| | | DFATYFCQQSEKLPRTFGGGTKVEIR | |
| | 2^{nd} | QVQLVQSGAEVKKPGASVKVSCKASGYTFTSYVMHWVRQKPGQGLEWIG | 294 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 295 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1849 | 1^{st} | QVQLVQSGAEVKKPGASVKVSCKASGYTFTSYVMHWVRQKPGQGLEWIG | 296 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 297 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | VQLVQSGAEVKKPGASVKVSCKASGYTFAGFEMHWVRQAPGQGLEWMGA | |
| | | IDPKTGGTDYNQKFKDRVTMTRDTSISTAYMELSRLRSDDTAVYYCARD | |
| | | LGYFDVWGQGTLVTVSSASVAAPSVFIFPPSDEQLKSGTASVVCLLNNF | |
| | | YPREAKVQWKVDNALQSGNSQESVTEQDSKDSTYSLSSTLTLSKADYEK | |
| | | HKVYACEVTHQGLSSPVTKSFNRGECGGGGSGGGGSGGGGSGGGGSQAV | |
| | | VTQEPSLTVSPGGTVTLTCRSSTGAVTTSNYANWVQQKPGQAPRGLIGG | |
| | | TNKRAPWTPARFSGSLLGGKAALTITGAQAEDEADYYCALWYSNLWVFG | |
| | | GGTKLTVLPAPEAAGPSVFLFPPKPKDTLMISRTPEVTCVVVDVSHEDP | |
| | | EVKFNWYVDGVEVHNAKTKPREEQYNSTYRVVSVLTVLHQDWLNGKEYK | |
| | | CKVSNKALPAPIEKTISKAKGQPREPQVYTLPPSREEMTKNQVSLSCAV | |
| | | KGFYPSDIAVEWESNGQPENNYKTTPPVLDSDGSFFLVSKLTVDKSRWQ | |
| | | QGNVFSCSVMHEALHNRFTQKSLSLSPGK | |
| | 3^{rd} | EIVLTQSPATLSLSPGERATLSCRASSSVSYMHWYQQKPGQAPRPWIYA | 298 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 299 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |

### Characterization of Anti-B7H3/CD3/CD137 Tri-Specific Antibodies

These tri-specific antibodies are evaluated for their in vitro and in vivo activity, including binding to target antigen (B7H3, CD3 and CD137), agonistic activity in CD3 and CD137 reporter assay system, activation of immune cells, anti-tumor activity in mouse models.

### Example 10: Anti-B7H3/CD3/CD28 tri-specific antibodies

Anti-B7H3/CD3/CD28 tri-specific were produced using the same anti-B7H3 and anti-CD3 parent clones disclosed in Example 9 above and the anti-CD28 antibody parent clones CD28 Ab1 or CD28 Ab2. The sequences of the parent antibody clones are provided in Table 1 above.

cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 10 below:

TABLE 10
Exemplary Anti-B7H3/CD3/CD28 Tri-Specific Antibodies

| | | | SED ID |
|---|---|---|---|
| Antibody Clones | | Amino acid sequence | NO: |
| Ly1968 | 1^{st} | QVQLVQSGAEVKKPGASVKVSCKASGYTFTSYVMHWVRQKPGQGLEWIG | 300 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVVKPGASVKVSCKASGYTFTSYYIHWVRQAPGQGLEWIG | 301 |
| | chain | SIYPGNVNTNYAQKFQGRATLTVDTSISTAYMELSRLRSDDTAVYYCTR | |
| | | SHYGLDWNFDVWGKGTTVTVSSASVAAPSVFIFPPSDEQLKSGTASVVC | |
| | | LLNNFYPREAKVQWKVDNALQSGNSQESVTEQDSKDSTYSLSSTLTLSK | |
| | | ADYEKHKVYACEVTHQGLSSPVTKSFNRGECGGGGSGGGGSGGGGSGGG | |
| | | GSQAVVTQEPSLTVSPGGTVTLTCRSSTGAVTTSNYANWVQQKPGQAPR | |
| | | GLIGGTNKRAPWTPARFSGSLLGGKAALTITGAQAEDEADYYCALWYSN | |
| | | LWVFGGGTKLTVLPAPELLGPSVFLFPPKPKDTLMISRTPEVTCVVVDV | |
| | | SHEDPEVKFNWYVDGVEVHNAKTKPREEQYNSTYRVVSVLTVLHQDWLN | |
| | | GKEYKCKVSNKALPAPIEKTISKAKGQPREPQVYTLPPSREEMTKNQVS | |
| | | LSCAVKGFYPSDIAVEWESNGQPENNYKTTPPVLDSDGSFFLVSKLTVD | |
| | | KSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 302 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCQASQNIYVWLNWYQQKPGKAPKLLIY | 303 |
| | chain | KASNLHTGVPSRFSGSGSGTDFTLTISSLQPEDIATYYCQQGQTYPYTF | |
| | | GQGTKLEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1969 | 1^{st} | QVQLVQSGAEVKKPGASVKVSCKASGYTFTSYVMHWVRQKPGQGLEWIG | 304 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIKGGGGsGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 305 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCQASQNIYVWLNWYQQKPGKAPKLLIYKASNLHTGVPSRFS | |
| | | GSGSGTDFTLTISSLQPEDIATYYCQQGQTYPYTFGQGTKLEIKGGGGS | |
| | | GGGGSGGGGSGGGGSQVQLVQSGAEVVKPGASVKVSCKASGYTFTSYYI | |
| | | HWVRQAPGQGLEWIGSIYPGNVNTNYAQKFQGRATLTVDTSISTAYMEL | |
| | | SRLRSDDTAVYYCTRSHYGLDWNFDVWGKGTTVTVSS | |
| | 3^{rd} | EIVLTQSPATLSLSPGERATLSCRASSSVSYMHWYQQKPGQAPRPWIYA | 306 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 307 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1970 | 1^{st} | QVQLVQSGAEVKKPGASVKVSCKASGYTFTSYVMHWVRQKPGQGLEWIG | 308 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | SGGGGSGGGGSGGGGSGGGGSDIQMTQSPSSLSASVGDRVTITCQASQN | |
| | | IYVWLNWYQQKPGKAPKLLIYKASNLHTGVPSRFSGSGSGTDFTLTISS | |
| | | LQPEDIATYYCQQGQTYPYTFGQGTKLEIK | |
| | 2^{nd} | QVQLVQSGAEVKKPGASVKVSCKASGYTFTSYVMHWVRQKPGQGLEWIG | 309 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVVKP | |
| | | GASVKVSCKASGYTFTSYYIHWVRQAPGQGLEWIGSIYPGNVNTNYAQK | |
| | | FQGRATLTVDTSISTAYMELSRLRSDDTAVYYCTRSHYGLDWNFDVWGK | |
| | | GTTVTVSSGGGGSGGGGsGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIK | |
| | 3^{rd} | EIVLTQSPATLSLSPGERATLSCRASSSVSYMHWYQQKPGQAPRPWIYA | 310 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 311 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1971 | 1^{st} | QVQLVQSGAEVKKPGASVKVSCKASGYTFTSYVMHWVRQKPGQGLEWIG | 312 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 313 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCQASQNIYVWLNWYQQKPGKAPKLLIYKASNLHTGVPSRFS | |
| | | GSGSGTDFTLTISSLQPEDIATYYCQQGQTYPYTFGQGTKLEIKGGGGS | |
| | | GGGGSGGGGSGGGGSQVQLVQSGAEVVKPGASVKVSCKASGYTFTSYYI | |
| | | HWVRQAPGQGLEWIGSIYPGNVNTNYAQKFQGRATLTVDTSISTAYMEL | |
| | | SRLRSDDTAVYYCTRSHYGLDWNFDVWGKGTTVTVSS | |
| | 3^{rd} | EIVLTQSPATLSLSPGERATLSCRASSSVSYMHWYQQKPGQAPRPWIYA | 314 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 315 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1972 | 1^{st} | QVQLVQSGAEVKKPGASVKVSCKASGYTFTSYVMHWVRQKPGQGLEWIG | 316 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 317 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVVKP | |
| | | GASVKVSCKASGYTFTSYYIHWVRQAPGQGLEWIGSIYPGNVNTNYAQK | |
| | | FQGRATLTVDTSISTAYMELSRLRSDDTAVYYCTRSHYGLDWNFDVWGK | |
| | | GTTVTVSSGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIK | |
| | 3^{rd} | EIVLTQSPATLSLSPGERATLSCRASSSVSYMHWYQQKPGQAPRPWIYA | 318 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 319 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1973 | 1^{st} | QVQLVQSGAEVKKPGASVKVSCKASGYTFTSYVMHWVRQKPGQGLEWIG | 320 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIKGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 321 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 322 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 323 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1974 | 1^{st} | QVQLVQSGAEVKKPGASVKVSCKASGYTFTSYVMHWVRQKPGQGLEWIG | 324 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | SGGGGSGGGGSGGGGSGGGGSDIQMTQSPSSLSASVGDRVTITCQASQN | |
| | | IYVWLNWYQQKPGKAPKLLIYKASNLHTGVPSRFSGSGSGTDFTLTISS | |
| | | LQPEDIATYYCQQGQTYPYTFGQGTKLEIK | |
| | 2^{nd} | QVQLVQSGAEVKKPGASVKVSCKASGYTFTSYVMHWVRQKPGQGLEWIG | 325 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 326 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 327 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1975 | 1^{st} | QVQLVQSGAEVKKPGASVKVSCKASGYTFTSYVMHWVRQKPGQGLEWIG | 328 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | S | |
| | 2^{nd} | QVQLVQSGAEVKKPGASVKVSCKASGYTFTSYVMHWVRQKPGQGLEWIG | 329 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCQASQNIYVWLNWYQQKPGKAPKLLIYKASNLHTGVPSRFS | |
| | | GSGSGTDFTLTISSLQPEDIATYYCQQGQTYPYTFGQGTKLEIK | |
| | 3^{rd} | EIVLTQSPATLSLSPGERATLSCRASSSVSYMHWYQQKPGQAPRPWIYA | 330 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 331 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1976 | 1^{st} | QVQLVQSGAEVKKPGASVKVSCKASGYTFTSYVMHWVRQKPGQGLEWIG | 332 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIKGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 333 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 334 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1977 | 1^{st} | QVQLVQSGAEVKKPGASVKVSCKASGYTFTSYVMHWVRQKPGQGLEWIG | 335 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | SGGGGSGGGGSGGGGSGGGGSDIQMTQSPSSLSASVGDRVTITCQASQN | |
| | | IYVWLNWYQQKPGKAPKLLIYKASNLHTGVPSRFSGSGSGTDFTLTISS | |
| | | LQPEDIATYYCQQGQTYPYTFGQGTKLEIK | |
| | 2^{nd} | QVQLVQSGAEVKKPGASVKVSCKASGYTFTSYVMHWVRQKPGQGLEWIG | 336 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 337 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1978 | 1^{st} | QVQLVQSGAEVKKPGASVKVSCKASGYTFTSYVMHWVRQKPGQGLEWIG | 338 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIKGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 339 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCQASQNIYVWLNWYQQKPG | |
| | | KAPKLLIYKASNLHTGVPSRFSGSGSGTDFTLTISSLQPEDIATYYCQQ | |
| | | GQTYPYTFGQGTKLEIKGGGGSGGGGSGGGGSGGGGSQVQLVQSGAEVV | |
| | | KPGASVKVSCKASGYTFTSYYIHWVRQAPGQGLEWIGSIYPGNVNTNYA | |
| | | QKFQGRATLTVDTSISTAYMELSRLRSDDTAVYYCTRSHYGLDWNFDVW | |
| | | GKGTTVTVSS | |
| | 3^{rd} | EIVLTQSPATLSLSPGERATLSCRASSSVSYMHWYQQKPGQAPRPWIYA | 340 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1979 | 1^{st} | QVQLVQSGAEVKKPGASVKVSCKASGYTFTSYVMHWVRQKPGQGLEWIG | 341 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | SGGGGSGGGGSGGGGSGGGGSDIQMTQSPSSLSASVGDRVTITCQASQN | |
| | | IYVWLNWYQQKPGKAPKLLIYKASNLHTGVPSRFSGSGSGTDFTLTISS | |
| | | LQPEDIATYYCQQGQTYPYTFGQGTKLEIK | |
| | 2^{nd} | QVQLVQSGAEVKKPGASVKVSCKASGYTFTSYVMHWVRQKPGQGLEWIG | 342 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSSGGGGSGGGGSGGGGSGGGG | |
| | | SDIQMTQSPSSLSASVGDRVTITCQASQNIYVWLNWYQQKPGKAPKLLI | |
| | | YKASNLHTGVPSRFSGSGSGTDFTLTISSLQPEDIATYYCQQGQTYPYT | |
| | | FGQGTKLEIK | |
| | 3^{rd} | EIVLTQSPATLSLSPGERATLSCRASSSVSYMHWYQQKPGQAPRPWIYA | 343 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1980 | 1^{st} | QVQLVQSGAEVKKPGASVKVSCKASGYTFTSYVMHWVRQKPGQGLEWIG | 344 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 345 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCQASQNIYVWLNWYQQKPG | |
| | | KAPKLLIYKASNLHTGVPSRFSGSGSGTDFTLTISSLQPEDIATYYCQQ | |
| | | GQTYPYTFGQGTKLEIKGGGGSGGGGSGGGGSGGGGSQVQLVQSGAEVV | |
| | | KPGASVKVSCKASGYTFTSYYIHWVRQAPGQGLEWIGSIYPGNVNTNYA | |
| | | QKFQGRATLTVDTSISTAYMELSRLRSDDTAVYYCTRSHYGLDWNFDVW | |
| | | GKGTTVTVSS | |
| | 3^{rd} | EIVLTQSPATLSLSPGERATLSCRASSSVSYMHWYQQKPGQAPRPWIYA | 346 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1981 | 1^{st} | QVQLVQSGAEVKKPGASVKVSCKASGYTFTSYVMHWVRQKPGQGLEWIG | 347 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 348 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSSGGGGSGGGGSGGGGSGGGG | |
| | | SDIQMTQSPSSLSASVGDRVTITCQASQNIYVWLNWYQQKPGKAPKLLI | |
| | | YKASNLHTGVPSRFSGSGSGTDFTLTISSLQPEDIATYYCQQGQTYPYT | |
| | | FGQGTKLEIK | |
| | 3^{rd} | EIVLTQSPATLSLSPGERATLSCRASSSVSYMHWYQQKPGQAPRPWIYA | 349 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1982 | 1^{st} | QVQLVQSGAEVKKPGASVKVSCKASGYTFTSYVMHWVRQKPGQGLEWIG | 350 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCQASQNIYVW | |
| | | LNWYQQKPGKAPKLLIYKASNLHTGVPSRFSGSGSGTDFTLTISSLQPE | |
| | | DIATYYCQQGQTYPYTFGQGTKLEIKGGGGSGGGGSGGGGSGGGGSQVQ | |
| | | LVQSGAEVVKPGASVKVSCKASGYTFTSYYIHWVRQAPGQGLEWIGSIY | |
| | | PGNVNTNYAQKFQGRATLTVDTSISTAYMELSRLRSDDTAVYYCTRSHY | |
| | | GLDWNFDVWGKGTTVTVSS | |
| | 2^{nd} | QVQLVQSGAEVKKPGASVKVSCKASGYTFTSYVMHWVRQKPGQGLEWIG | 351 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCQASQNIYVWLNWYQQKPGKAPKLLI | |
| | | YKASNLHTGVPSRFSGSGSGTDFTLTISSLQPEDIATYYCQQGQTYPYT | |
| | | FGQGTKLEIKGGGGSGGGGSGGGGSGGGGSQVQLVQSGAEVVKPGASVK | |
| | | VSCKASGYTFTSYYIHWVRQAPGQGLEWIGSIYPGNVNTNYAQKFQGRA | |
| | | TLTVDTSISTAYMELSRLRSDDTAVYYCTRSHYGLDWNFDVWGKGTTVT | |
| | | VSS | |
| | 3^{rd} | EIVLTQSPATLSLSPGERATLSCRASSSVSYMHWYQQKPGQAPRPWIYA | 352 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1983 | 1^{st} | QVQLVQSGAEVKKPGASVKVSCKASGYTFTSYVMHWVRQKPGQGLEWIG | 353 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVVKPGASVKVSCKASGYTFTSY | |
| | | YIHWVRQAPGQGLEWIGSIYPGNVNTNYAQKFQGRATLTVDTSISTAYM | |
| | | ELSRLRSDDTAVYYCTRSHYGLDWNFDVWGKGTTVTVSSGGGGSGGGGS | |
| | | GGGGSGGGGSDIQMTQSPSSLSASVGDRVTITCQASQNIYVWLNWYQQK | |
| | | PGKAPKLLIYKASNLHTGVPSRFSGSGSGTDFTLTISSLQPEDIATYYC | |
| | | QQGQTYPYTFGQGTKLEIK | |
| | 2^{nd} | QVQLVQSGAEVKKPGASVKVSCKASGYTFTSYVMHWVRQKPGQGLEWIG | 354 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVVKPGASVKVSCKASGYTFTSYYIHWVRQAPGQGLEWI | |
| | | GSIYPGNVNTNYAQKFQGRATLTVDTSISTAYMELSRLRSDDTAVYYCT | |
| | | RSHYGLDWNFDVWGKGTTVTVSSGGGGSGGGGSGGGGSGGGGSDIQMTQ | |
| | | SPSSLSASVGDRVTITCQASQNIYVWLNWYQQKPGKAPKLLIYKASNLH | |
| | | TGVPSRFSGSGSGTDFTLTISSLQPEDIATYYCQQGQTYPYTFGQGTKL | |
| | | EIK | |
| | 3^{rd} | EIVLTQSPATLSLSPGERATLSCRASSSVSYMHWYQQKPGQAPRPWIYA | 355 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1984 | 1^{st} | QVQLVQSGAEVKKPGASVKVSCKASGYTFTSYVMHWVRQKPGQGLEWIG | 356 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCQASQNIYVW | |
| | | LNWYQQKPGKAPKLLIYKASNLHTGVPSRFSGSGSGTDFTLTISSLQPE | |
| | | DIATYYCQQGQTYPYTFGQGTKLEIKGGGGSGGGGSGGGGSGGGGSQVQ | |
| | | LVQSGAEVVKPGASVKVSCKASGYTFTSYYIHWVRQAPGQGLEWIGSIY | |
| | | PGNVNTNYAQKFQGRATLTVDTSISTAYMELSRLRSDDTAVYYCTRSHY | |
| | | GLDWNFDVWGKGTTVTVSS | |
| | 2^{nd} | QVQLVQSGAEVKKPGASVKVSCKASGYTFTSYVMHWVRQKPGQGLEWIG | 357 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 358 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1985 | 1^{st} | QVQLVQSGAEVKKPGASVKVSCKASGYTFTSYVMHWVRQKPGQGLEWIG | 359 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVVKPGASVKVSCKASGYTFTSY | |
| | | YIHWVRQAPGQGLEWIGSIYPGNVNTNYAQKFQGRATLTVDTSISTAYM | |
| | | ELSRLRSDDTAVYYCTRSHYGLDWNFDVWGKGTTVTVSSGGGGSGGGGS | |
| | | GGGGSGGGGSDIQMTQSPSSLSASVGDRVTITCQASQNIYVWLNWYQQK | |
| | | PGKAPKLLIYKASNLHTGVPSRFSGSGSGTDFTLTISSLQPEDIATYYC | |
| | | QQGQTYPYTFGQGTKLEIK | |
| | 2^{nd} | QVQLVQSGAEVKKPGASVKVSCKASGYTFTSYVMHWVRQKPGQGLEWIG | 360 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 361 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1986 | 1^{st} | QVQLVQSGAEVKKPGASVKVSCKASGYTFTSYVMHWVRQKPGQGLEWIG | 362 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 363 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCQASQNIYVWLNWYQQKPGKAPKLLI | |
| | | YKASNLHTGVPSRFSGSGSGTDFTLTISSLQPEDIATYYCQQGQTYPYT | |
| | | FGQGTKLEIKGGGGSGGGGSGGGGSGGGGSQVQLVQSGAEVVKPGASVK | |
| | | VSCKASGYTFTSYYIHWVRQAPGQGLEWIGSIYPGNVNTNYAQKFQGRA | |
| | | TLTVDTSISTAYMELSRLRSDDTAVYYCTRSHYGLDWNFDVWGKGTTVT | |
| | | VSS | |
| | 3^{rd} | EIVLTQSPATLSLSPGERATLSCRASSSVSYMHWYQQKPGQAPRPWIYA | 364 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1987 | 1^{st} | QVQLVQSGAEVKKPGASVKVSCKASGYTFTSYVMHWVRQKPGQGLEWIG | 365 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 366 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVVKPGASVKVSCKASGYTFTSYYIHWVRQAPGQGLEWI | |
| | | GSIYPGNVNTNYAQKFQGRATLTVDTSISTAYMELSRLRSDDTAVYYCT | |
| | | RSHYGLDWNFDVWGKGTTVTVSSGGGGSGGGGSGGGGSGGGGSDIQMTQ | |
| | | SPSSLSASVGDRVTITCQASQNIYVWLNWYQQKPGKAPKLLIYKASNLH | |
| | | TGVPSRFSGSGSGTDFTLTISSLQPEDIATYYCQQGQTYPYTFGQGTKL | |
| | | EIK | |
| | 3^{rd} | EIVLTQSPATLSLSPGERATLSCRASSSVSYMHWYQQKPGQAPRPWIYA | 367 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1988 | 1^{st} | QVQLVQSGAEVKKPGASVKVSCKASGYTFTSYVMHWVRQKPGQGLEWIG | 368 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVVKPGASVKVSCKASGYTFTSY | |
| | | YIHWVRQAPGQGLEWIGSIYPGNVNTNYAQKFQGRATLTVDTSISTAYM | |
| | | ELSRLRSDDTAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 369 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCQASQNIYVWLNWYQQKPGKAPKLLI | |
| | | YKASNLHTGVPSRFSGSGSGTDFTLTISSLQPEDIATYYCQQGQTYPYT | |
| | | FGQGTKLEIK | |
| | 3^{rd} | EIVLTQSPATLSLSPGERATLSCRASSSVSYMHWYQQKPGQAPRPWIYA | 370 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1989 | 1^{st} | QVQLVQSGAEVKKPGASVKVSCKASGYTFTSYVMHWVRQKPGQGLEWIG | 371 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 372 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 373 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCQASQNIYVWLNWYQQKPGKAPKLLIYKASNLHTGVPSR | |
| | | FSGSGSGTDFTLTISSLQPEDIATYYCQQGQTYPYTFGQGTKLEIKGGG | |
| | | GSGGGGSGGGGSGGGGSQVQLVQSGAEVVKPGASVKVSCKASGYTFTSY | |
| | | YIHWVRQAPGQGLEWIGSIYPGNVNTNYAQKFQGRATLTVDTSISTAYM | |
| | | ELSRLRSDDTAVYYCTRSHYGLDWNFDVWGKGTTVTVSS | |
| Ly1990 | 1^{st} | QVQLVQSGAEVKKPGASVKVSCKASGYTFTSYVMHWVRQKPGQGLEWIG | 374 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 375 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 376 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLVQSGAEVV | |
| | | KPGASVKVSCKASGYTFTSYYIHWVRQAPGQGLEWIGSIYPGNVNTNYA | |
| | | QKFQGRATLTVDTSISTAYMELSRLRSDDTAVYYCTRSHYGLDWNFDVW | |
| | | GKGTTVTVSSGGGGSGGGGSGGGGSGGGGSDIQMTQSPSSLSASVGDRV | |
| | | TITCQASQNIYVWLNWYQQKPGKAPKLLIYKASNLHTGVPSRFSGSGSG | |
| | | TDFTLTISSLQPEDIATYYCQQGQTYPYTFGQGTKLEIK | |
| Ly1991 | 1^{st} | QVQLVQSGAEVKKPGASVKVSCKASGYTFTSYVMHWVRQKPGQGLEWIG | 377 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCQASQNIYVWLNWYQQKPGKAPKLLIYKASNLHTGVPSRFSGSGSGTD | |
| | | FTLTISSLQPEDIATYYCQQGQTYPYTFGQGTKLEIKGGGGSGGGGSGG | |
| | | GGSGGGGSQVQLVQSGAEVVKPGASVKVSCKASGYTFTSYYIHWVRQAP | |
| | | GQGLEWIGSIYPGNVNTNYAQKFQGRATLTVDTSISTAYMELSRLRSDD | |
| | | TAVYYCTRSHYGLDWNFDVWGKGTTVTVSS | |
| | 2^{nd} | QVQLVQSGAEVKKPGASVKVSCKASGYTFTSYVMHWVRQKPGQGLEWIG | 378 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 379 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1992 | 1^{st} | QVQLVQSGAEVKKPGASVKVSCKASGYTFTSYVMHWVRQKPGQGLEWIG | 380 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVVKPGASVKVS | |
| | | CKASGYTFTSYYIHWVRQAPGQGLEWIGSIYPGNVNTNYAQKFQGRATL | |
| | | TVDTSISTAYMELSRLRSDDTAVYYCTRSHYGLDWNFDVWGKGTTVTVS | |
| | | SGGGGSGGGGSGGGGSGGGGSDIQMTQSPSSLSASVGDRVTITCQASQN | |
| | | IYVWLNWYQQKPGKAPKLLIYKASNLHTGVPSRFSGSGSGTDFTLTISS | |
| | | LQPEDIATYYCQQGQTYPYTFGQGTKLEIK | |
| | 2^{nd} | QVQLVQSGAEVKKPGASVKVSCKASGYTFTSYVMHWVRQKPGQGLEWIG | 381 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 382 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1993 | 1^{st} | QVQLVQSGAEVKKPGASVKVSCKASGYTFTSYVMHWVRQKPGQGLEWIG | 383 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYYIHWVRQAPGQGLEWIG | 384 |
| | chain | CIYPGNVNTNYNEKFKDRATLTVDTSISTAYMELSRLRSDDTAVYFCTR | |
| | | SHYGLDWNFDVWGQGTTVTVSSASVAAPSVFIFPPSDEQLKSGTASVVC | |
| | | LLNNFYPREAKVQWKVDNALQSGNSQESVTEQDSKDSTYSLSSTLTLSK | |
| | | ADYEKHKVYACEVTHQGLSSPVTKSFNRGECGGGGSGGGGSGGGGSGGG | |
| | | GSQAVVTQEPSLTVSPGGTVTLTCRSSTGAVTTSNYANWVQQKPGQAPR | |
| | | GLIGGTNKRAPWTPARFSGSLLGGKAALTITGAQAEDEADYYCALWYSN | |
| | | LWVFGGGTKLTVLPAPELLGPSVFLFPPKPKDTLMISRTPEVTCVVVDV | |
| | | SHEDPEVKFNWYVDGVEVHNAKTKPREEQYNSTYRVVSVLTVLHQDWLN | |
| | | GKEYKCKVSNKALPAPIEKTISKAKGQPREPQVYTLPPSREEMTKNQVS | |
| | | LSCAVKGFYPSDIAVEWESNGQPENNYKTTPPVLDSDGSFFLVSKLTVD | |
| | | KSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 385 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCHASQNIYVWLNWYQQKPGKAPKLLIY | 386 |
| | chain | KASNLHTGVPSRFSGSGSGTDFTLTISSLQPEDFATYYCQQGQTYPYTF | |
| | | GGGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1994 | 1^{st} | QVQLVQSGAEVKKPGASVKVSCKASGYTFTSYVMHWVRQKPGQGLEWIG | 387 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIKGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 388 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCHASQNIYVWLNWYQQKPGKAPKLLIYKASNLHTGVPSRFS | |
| | | GSGSGTDFTLTISSLQPEDFATYYCQQGQTYPYTFGGGTKVEIKGGGGS | |
| | | GGGGSGGGGSGGGGSQVQLVQSGAEVKKPGASVKVSCKASGYTFTSYYI | |
| | | HWVRQAPGQGLEWIGCIYPGNVNTNYNEKFKDRATLTVDTSISTAYMEL | |
| | | SRLRSDDTAVYFCTRSHYGLDWNFDVWGQGTTVTVSS | |
| | 3^{rd} | EIVLTQSPATLSLSPGERATLSCRASSSVSYMHWYQQKPGQAPRPWIYA | 389 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 390 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1995 | 1^{st} | QVQLVQSGAEVKKPGASVKVSCKASGYTFTSYVMHWVRQKPGQGLEWIG | 391 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNFDVWGQGTTVTVS | |
| | | SGGGGSGGGGSGGGGSGGGGSDIQMTQSPSSLSASVGDRVTITCHASQN | |
| | | IYVWLNWYQQKPGKAPKLLIYKASNLHTGVPSRFSGSGSGTDFTLTISS | |
| | | LQPEDFATYYCQQGQTYPYTFGGGTKVEIK | |
| | 2^{nd} | QVQLVQSGAEVKKPGASVKVSCKASGYTFTSYVMHWVRQKPGQGLEWIG | 392 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVKKP | |
| | | GASVKVSCKASGYTFTSYYIHWVRQAPGQGLEWIGCIYPGNVNTNYNEK | |
| | | FKDRATLTVDTSISTAYMELSRLRSDDTAVYFCTRSHYGLDWNEDVWGQ | |
| | | GTTVTVSSGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIK | |
| | 3^{rd} | EIVLTQSPATLSLSPGERATLSCRASSSVSYMHWYQQKPGQAPRPWIYA | 393 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 394 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1996 | 1^{st} | QVQLVQSGAEVKKPGASVKVSCKASGYTFTSYVMHWVRQKPGQGLEWIG | 395 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 396 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCHASQNIYVWLNWYQQKPGKAPKLLIYKASNLHTGVPSRFS | |
| | | GSGSGTDFTLTISSLQPEDFATYYCQQGQTYPYTFGGGTKVEIKGGGGS | |
| | | GGGGSGGGGSGGGGSQVQLVQSGAEVKKPGASVKVSCKASGYTFTSYYI | |
| | | HWVRQAPGQGLEWIGCIYPGNVNTNYNEKFKDRATLTVDTSISTAYMEL | |
| | | SRLRSDDTAVYFCTRSHYGLDWNFDVWGQGTTVTVSS | |
| | 3^{rd} | EIVLTQSPATLSLSPGERATLSCRASSSVSYMHWYQQKPGQAPRPWIYA | 397 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 398 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1997 | 1^{st} | QVQLVQSGAEVKKPGASVKVSCKASGYTFTSYVMHWVRQKPGQGLEWIG | 399 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 400 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLVQSGAEVKKP | |
| | | GASVKVSCKASGYTFTSYYIHWVRQAPGQGLEWIGCIYPGNVNTNYNEK | |
| | | FKDRATLTVDTSISTAYMELSRLRSDDTAVYFCTRSHYGLDWNFDVWGQ | |
| | | GTTVTVSSGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIK | |
| | 3^{rd} | EIVLTQSPATLSLSPGERATLSCRASSSVSYMHWYQQKPGQAPRPWIYA | 401 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 402 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1998 | 1^{st} | QAVVTQEPSLTVSPGGTVTLTCRSSTGAVTTSNYANWVQQKPGQAPRGL | 403 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| | | QVQLVQSGAEVKKPGASVKVSCKASGYTFTSYVMHWVRQKPGQGLEWIG | |
| | | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIKGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 404 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 405 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 406 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1999 | 1^{st} | QVQLVQSGAEVKKPGASVKVSCKASGYTFTSYVMHWVRQKPGQGLEWIG | 407 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNFDVWGQGTTVTVS | |
| | | SGGGGSGGGGGGGGSGGGGSDIQMTQSPSSLSASVGDRVTITCHASQN | |
| | | IYVWLNWYQQKPGKAPKLLIYKASNLHTGVPSRFSGSGSGTDFTLTISS | |
| | | LQPEDFATYYCQQGQTYPYTFGGGTKVEIK | |
| | 2^{nd} | QVQLVQSGAEVKKPGASVKVSCKASGYTFTSYVMHWVRQKPGQGLEWIG | 408 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 409 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 410 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2000 | 1^{st} | QVQLVQSGAEVKKPGASVKVSCKASGYTFTSYVMHWVRQKPGQGLEWIG | 411 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNEDVWGQGTTVTVS | |
| | | S | |
| | 2^{nd} | QVQLVQSGAEVKKPGASVKVSCKASGYTFTSYVMHWVRQKPGQGLEWIG | 412 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCHASQNIYVWLNWYQQKPGKAPKLLIYKASNLHTGVPSRFS | |
| | | GSGSGTDFTLTISSLQPEDFATYYCQQGQTYPYTFGGGTKVEIK | |
| | 3^{rd} | EIVLTQSPATLSLSPGERATLSCRASSSVSYMHWYQQKPGQAPRPWIYA | 413 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 414 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2001 | 1^{st} | QVQLVQSGAEVKKPGASVKVSCKASGYTFTSYVMHWVRQKPGQGLEWIG | 415 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIKGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 416 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 417 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2002 | 1^{st} | QVQLVQSGAEVKKPGASVKVSCKASGYTFTSYVMHWVRQKPGQGLEWIG | 418 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNFDVWGQGTTVTVS | |
| | | SGGGGSGGGGSGGGGSGGGGSDIQMTQSPSSLSASVGDRVTITCHASQN | |
| | | IYVWLNWYQQKPGKAPKLLIYKASNLHTGVPSRFSGSGSGTDFTLTISS | |
| | | LQPEDFATYYCQQGQTYPYTFGGGTKVEIK | |
| | 2^{nd} | QVQLVQSGAEVKKPGASVKVSCKASGYTFTSYVMHWVRQKPGQGLEWIG | 419 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 420 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2003 | 1^{st} | QVQLVQSGAEVKKPGASVKVSCKASGYTFTSYVMHWVRQKPGQGLEWIG | 421 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIKGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 422 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCHASQNIYVWLNWYQQKPG | |
| | | KAPKLLIYKASNLHTGVPSRFSGSGSGTDFTLTISSLQPEDFATYYCQQ | |
| | | GQTYPYTFGGGTKVEIKGGGGSGGGGSGGGGSGGGGSQVQLVQSGAEVK | |
| | | KPGASVKVSCKASGYTFTSYYIHWVRQAPGQGLEWIGCIYPGNVNTNYN | |
| | | EKFKDRATLTVDTSISTAYMELSRLRSDDTAVYFCTRSHYGLDWNFDVW | |
| | | GQGTTVTVSS | |
| | 3^{rd} | EIVLTQSPATLSLSPGERATLSCRASSSVSYMHWYQQKPGQAPRPWIYA | 423 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2004 | 1^{st} | QVQLVQSGAEVKKPGASVKVSCKASGYTFTSYVMHWVRQKPGQGLEWIG | 424 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNFDVWGQGTTVTVS | |
| | | SGGGGSGGGGSGGGGSGGGGSDIQMTQSPSSLSASVGDRVTITCHASQN | |
| | | IYVWLNWYQQKPGKAPKLLIYKASNLHTGVPSRFSGSGSGTDFTLTISS | |
| | | LQPEDFATYYCQQGQTYPYTFGGGTKVEIK | |
| | 2^{nd} | QVQLVQSGAEVKKPGASVKVSCKASGYTFTSYVMHWVRQKPGQGLEWIG | 425 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSSGGGGSGGGGSGGGGSGGGG | |
| | | SDIQMTQSPSSLSASVGDRVTITCHASQNIYVWLNWYQQKPGKAPKLLI | |
| | | YKASNLHTGVPSRFSGSGSGTDFTLTISSLQPEDFATYYCQQGQTYPYT | |
| | | FGGGTKVEIK | |
| | 3^{rd} | EIVLTQSPATLSLSPGERATLSCRASSSVSYMHWYQQKPGQAPRPWIYA | 426 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2005 | 1^{st} | QVQLVQSGAEVKKPGASVKVSCKASGYTFTSYVMHWVRQKPGQGLEWIG | 427 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 428 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCHASQNIYVWLNWYQQKPG | |
| | | KAPKLLIYKASNLHTGVPSRFSGSGSGTDFTLTISSLQPEDFATYYCQQ | |
| | | GQTYPYTFGGGTKVEIKGGGGSGGGGSGGGGSGGGGSQVQLVQSGAEVK | |
| | | KPGASVKVSCKASGYTFTSYYIHWVRQAPGQGLEWIGCIYPGNVNTNYN | |
| | | EKFKDRATLTVDTSISTAYMELSRLRSDDTAVYFCTRSHYGLDWNFDVW | |
| | | GQGTTVTVSS | |
| | 3^{rd} | EIVLTQSPATLSLSPGERATLSCRASSSVSYMHWYQQKPGQAPRPWIYA | 429 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2006 | 1^{st} | QVQLVQSGAEVKKPGASVKVSCKASGYTFTSYVMHWVRQKPGQGLEWIG | 430 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 431 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSSGGGGSGGGGSGGGGSGGGG | |
| | | SDIQMTQSPSSLSASVGDRVTITCHASQNIYVWLNWYQQKPGKAPKLLI | |
| | | YKASNLHTGVPSRFSGSGSGTDFTLTISSLQPEDFATYYCQQGQTYPYT | |
| | | FGGGTKVEIK | |
| | 3^{rd} | EIVLTQSPATLSLSPGERATLSCRASSSVSYMHWYQQKPGQAPRPWIYA | 432 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2007 | 1^{st} | QVQLVQSGAEVKKPGASVKVSCKASGYTFTSYVMHWVRQKPGQGLEWIG | 433 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTESTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCHASQNIYVW | |
| | | LNWYQQKPGKAPKLLIYKASNLHTGVPSRFSGSGSGTDFTLTISSLQPE | |
| | | DFATYYCQQGQTYPYTFGGGTKVEIKGGGGSGGGGSGGGGSGGGGSQVQ | |
| | | LVQSGAEVKKPGASVKVSCKASGYTFTSYYIHWVRQAPGQGLEWIGCIY | |
| | | PGNVNTNYNEKFKDRATLTVDTSISTAYMELSRLRSDDTAVYFCTRSHY | |
| | | GLDWNFDVWGQGTTVTVSS | |
| | 2^{nd} | QVQLVQSGAEVKKPGASVKVSCKASGYTFTSYVMHWVRQKPGQGLEWIG | 434 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCHASQNIYVWLNWYQQKPGKAPKLLI | |
| | | YKASNLHTGVPSRFSGSGSGTDFTLTISSLQPEDFATYYCQQGQTYPYT | |
| | | FGGGTKVEIKGGGGSGGGGSGGGGSGGGGSQVQLVQSGAEVKKPGASVK | |
| | | VSCKASGYTFTSYYIHWVRQAPGQGLEWIGCIYPGNVNTNYNEKFKDRA | |
| | | TLTVDTSISTAYMELSRLRSDDTAVYFCTRSHYGLDWNFDVWGQGTTVT | |
| | | VSS | |
| | 3^{rd} | EIVLTQSPATLSLSPGERATLSCRASSSVSYMHWYQQKPGQAPRPWIYA | 435 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2008 | 1^{st} | QVQLVQSGAEVKKPGASVKVSCKASGYTFTSYVMHWVRQKPGQGLEWIG | 436 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFTSY | |
| | | YIHWVRQAPGQGLEWIGCIYPGNVNTNYNEKFKDRATLTVDTSISTAYM | |
| | | ELSRLRSDDTAVYFCTRSHYGLDWNFDVWGQGTTVTVSSGGGGSGGGGS | |
| | | GGGGSGGGGSDIQMTQSPSSLSASVGDRVTITCHASQNIYVWLNWYQQK | |
| | | PGKAPKLLIYKASNLHTGVPSRFSGSGSGTDFTLTISSLQPEDFATYYC | |
| | | QQGQTYPYTFGGGTKVEIK | |
| | 2^{nd} | QVQLVQSGAEVKKPGASVKVSCKASGYTFTSYVMHWVRQKPGQGLEWIG | 437 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVKKPGASVKVSCKASGYTFTSYYIHWVRQAPGQGLEWI | |
| | | GCIYPGNVNTNYNEKFKDRATLTVDTSISTAYMELSRLRSDDTAVYFCT | |
| | | RSHYGLDWNFDVWGQGTTVTVSSGGGGSGGGGSGGGGSGGGGSDIQMTQ | |
| | | SPSSLSASVGDRVTITCHASQNIYVWLNWYQQKPGKAPKLLIYKASNLH | |
| | | TGVPSRFSGSGSGTDFTLTISSLQPEDFATYYCQQGQTYPYTFGGGTKV | |
| | | EIK | |
| | 3^{rd} | EIVLTQSPATLSLSPGERATLSCRASSSVSYMHWYQQKPGQAPRPWIYA | 438 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2009 | 1^{st} | QVQLVQSGAEVKKPGASVKVSCKASGYTFTSYVMHWVRQKPGQGLEWIG | 439 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCHASQNIYVW | |
| | | LNWYQQKPGKAPKLLIYKASNLHTGVPSRFSGSGSGTDFTLTISSLQPE | |
| | | DFATYYCQQGQTYPYTFGGGTKVEIKGGGGSGGGGSGGGGSGGGGSQVQ | |
| | | LVQSGAEVKKPGASVKVSCKASGYTFTSYYIHWVRQAPGQGLEWIGCIY | |
| | | PGNVNTNYNEKFKDRATLTVDTSISTAYMELSRLRSDDTAVYFCTRSHY | |
| | | GLDWNFDVWGQGTTVTVSS | |
| | 2^{nd} | QVQLVQSGAEVKKPGASVKVSCKASGYTFTSYVMHWVRQKPGQGLEWIG | 440 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 441 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2010 | 1^{st} | QVQLVQSGAEVKKPGASVKVSCKASGYTFTSYVMHWVRQKPGQGLEWIG | 442 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFTSY | |
| | | YIHWVRQAPGQGLEWIGCIYPGNVNTNYNEKFKDRATLTVDTSISTAYM | |
| | | ELSRLRSDDTAVYFCTRSHYGLDWNFDVWGQGTTVTVSSGGGGSGGGGS | |
| | | GGGGSGGGGSDIQMTQSPSSLSASVGDRVTITCHASQNIYVWLNWYQQK | |
| | | PGKAPKLLIYKASNLHTGVPSRFSGSGSGTDFTLTISSLQPEDFATYYC | |
| | | QQGQTYPYTFGGGTKVEIK | |
| | 2^{nd} | QVQLVQSGAEVKKPGASVKVSCKASGYTFTSYVMHWVRQKPGQGLEWIG | 443 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLIVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 444 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2011 | 1^{st} | QVQLVQSGAEVKKPGASVKVSCKASGYTFTSYVMHWVRQKPGQGLEWIG | 445 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 446 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCHASQNIYVWLNWYQQKPGKAPKLLI | |
| | | YKASNLHTGVPSRFSGSGSGTDFTLTISSLQPEDFATYYCQQGQTYPYT | |
| | | FGGGTKVEIKGGGGSGGGGSGGGGSGGGGSQVQLVQSGAEVKKPGASVK | |
| | | VSCKASGYTFTSYYIHWVRQAPGQGLEWIGCIYPGNVNTNYNEKFKDRA | |
| | | TLTVDTSISTAYMELSRLRSDDTAVYFCTRSHYGLDWNFDVWGQGTTVT | |
| | | VSS | |
| | 3^{rd} | EIVLTQSPATLSLSPGERATLSCRASSSVSYMHWYQQKPGQAPRPWIYA | 447 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2012 | 1^{st} | QVQLVQSGAEVKKPGASVKVSCKASGYTFTSYVMHWVRQKPGQGLEWIG | 448 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 449 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLVQSGAEVKKPGASVKVSCKASGYTFTSYYIHWVRQAPGQGLEWI | |
| | | GCIYPGNVNTNYNEKFKDRATLTVDTSISTAYMELSRLRSDDTAVYFCT | |
| | | RSHYGLDWNFDVWGQGTTVTVSSGGGGSGGGGSGGGGSGGGGSDIQMTQ | |
| | | SPSSLSASVGDRVTITCHASQNIYVWLNWYQQKPGKAPKLLIYKASNLH | |
| | | TGVPSRFSGSGSGTDFTLTISSLQPEDFATYYCQQGQTYPYTFGGGTKV | |
| | | EIK | |
| | 3^{rd} | EIVLTQSPATLSLSPGERATLSCRASSSVSYMHWYQQKPGQAPRPWIYA | 450 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2013 | 1^{st} | QVQLVQSGAEVKKPGASVKVSCKASGYTFTSYVMHWVRQKPGQGLEWIG | 451 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLVQSGAEVKKPGASVKVSCKASGYTFTSY | |
| | | YIHWVRQAPGQGLEWIGCIYPGNVNTNYNEKFKDRATLTVDTSISTAYM | |
| | | ELSRLRSDDTAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 452 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCHASQNIYVWLNWYQQKPGKAPKLLI | |
| | | YKASNLHTGVPSRFSGSGSGTDFTLTISSLQPEDFATYYCQQGQTYPYT | |
| | | FGGGTKVEIK | |
| | 3^{rd} | EIVLTQSPATLSLSPGERATLSCRASSSVSYMHWYQQKPGQAPRPWIYA | 453 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2014 | 1^{st} | QVQLVQSGAEVKKPGASVKVSCKASGYTFTSYVMHWVRQKPGQGLEWIG | 454 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 455 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 456 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCHASQNIYVWLNWYQQKPGKAPKLLIYKASNLHTGVPSR | |
| | | FSGSGSGTDFTLTISSLQPEDFATYYCQQGQTYPYTFGGGTKVEIKGGG | |
| | | GSGGGGSGGGGSGGGGSQVQLVQSGAEVKKPGASVKVSCKASGYTFTSY | |
| | | YIHWVRQAPGQGLEWIGCIYPGNVNTNYNEKFKDRATLTVDTSISTAYM | |
| | | ELSRLRSDDTAVYFCTRSHYGLDWNFDVWGQGTTVTVSS | |
| Ly2015 | 1^{st} | QVQLVQSGAEVKKPGASVKVSCKASGYTFTSYVMHWVRQKPGQGLEWIG | 457 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 458 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 459 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLVQSGAEVK | |
| | | KPGASVKVSCKASGYTFTSYYIHWVRQAPGQGLEWIGCIYPGNVNTNYN | |
| | | EKFKDRATLTVDTSISTAYMELSRLRSDDTAVYFCTRSHYGLDWNFDVW | |
| | | GQGTTVTVSSGGGGSGGGGSGGGGSGGGGSDIQMTQSPSSLSASVGDRV | |
| | | TITCHASQNIYVWLNWYQQKPGKAPKLLIYKASNLHTGVPSRFSGSGSG | |
| | | TDFTLTISSLQPEDFATYYCQQGQTYPYTFGGGTKVEIK | |
| Ly2016 | 1^{st} | QVQLVQSGAEVKKPGASVKVSCKASGYTFTSYVMHWVRQKPGQGLEWIG | 460 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCHASQNIYVWLNWYQQKPGKAPKLLIYKASNLHTGVPSRFSGSGSGTD | |
| | | FTLTISSLQPEDFATYYCQQGQTYPYTFGGGTKVEIKGGGGSGGGGSGG | |
| | | GGSGGGGSQVQLVQSGAEVKKPGASVKVSCKASGYTFTSYYIHWVRQAP | |
| | | GQGLEWIGCIYPGNVNTNYNEKFKDRATLTVDTSISTAYMELSRLRSDD | |
| | | TAVYFCTRSHYGLDWNFDVWGQGTTVTVSS | |
| | 2^{nd} | QVQLVQSGAEVKKPGASVKVSCKASGYTFTSYVMHWVRQKPGQGLEWIG | 461 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRETQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 462 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2017 | 1^{st} | QVQLVQSGAEVKKPGASVKVSCKASGYTFTSYVMHWVRQKPGQGLEWIG | 463 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLVQSGAEVKKPGASVKVS | |
| | | CKASGYTFTSYYIHWVRQAPGQGLEWIGCIYPGNVNTNYNEKFKDRATL | |
| | | TVDTSISTAYMELSRLRSDDTAVYFCTRSHYGLDWNFDVWGQGTTVTVS | |
| | | SGGGGSGGGGSGGGGSGGGGSDIQMTQSPSSLSASVGDRVTITCHASQN | |
| | | IYVWLNWYQQKPGKAPKLLIYKASNLHTGVPSRFSGSGSGTDFTLTISS | |
| | | LQPEDFATYYCQQGQTYPYTFGGGTKVEIK | |
| | 2^{nd} | QVQLVQSGAEVKKPGASVKVSCKASGYTFTSYVMHWVRQKPGQGLEWIG | 464 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 465 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2018 | 1^{st} | QVQLVQSGAEVKKPGASVKVSCKASGYTFTSYVMHWVRQKPGQGLEWIG | 466 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLQESGPGLVKPSETLSLTCTVSGGSISSYYWSWIRQPPGKGLEWIG | 467 |
| | chain | YIYYSGITHYNPSLKSRVTISVDTSKIQFSLKLSSVTAADTAVYYCARW | |
| | | GVRRDYYYYGMDVWGQGTTVTVSSASVAAPSVFIFPPSDEQLKSGTASV | |
| | | VCLLNNFYPREAKVQWKVDNALQSGNSQESVTEQDSKDSTYSLSSTLTL | |
| | | SKADYEKHKVYACEVTHQGLSSPVTKSFNRGECGGGGSGGGGSGGGGSG | |
| | | GGGSQAVVTQEPSLTVSPGGTVTLTCRSSTGAVTTSNYANWVQQKPGQA | |
| | | PRGLIGGTNKRAPWTPARFSGSLLGGKAALTITGAQAEDEADYYCALWY | |
| | | SNLWVFGGGTKLTVLPAPELLGPSVFLFPPKPKDTLMISRTPEVTCVVV | |
| | | DVSHEDPEVKFNWYVDGVEVHNAKTKPREEQYNSTYRVVSVLTVLHQDW | |
| | | LNGKEYKCKVSNKALPAPIEKTISKAKGQPREPQVYTLPPSREEMTKNQ | |
| | | VSLSCAVKGFYPSDIAVEWESNGQPENNYKTTPPVLDSDGSFFLVSKLT | |
| | | VDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 468 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | EIVLTQSPGTLSLSPGERATLSCRASQSVSSSYLAWYQQKPGQAPRLLI | 469 |
| | chain | YGASSRATGIPDRFSGSGSGTDFTLTISRLEPEDFAVYYCQQYGSSPWT | |
| | | FGQGTKVEIKSSASTKGPSVFPLAPSSKSTSGGTAALGCLVKDYFPEPV | |
| | | TVSWNSGALTSGVHTFPAVLQSSGLYSLSSVVTVPSSSLGTQTYICNVN | |
| | | HKPSNTKVDKKVEPKSC | |
| Ly2019 | 1^{st} | QVQLVQSGAEVKKPGASVKVSCKASGYTFTSYVMHWVRQKPGQGLEWIG | 470 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPGTLSLSPGERATL | |
| | | SCRASQSVSSSYLAWYQQKPGQAPRLLIYGASSRATGIPDRFSGSGSGT | |
| | | DFTLTISRLEPEDFAVYYCQQYGSSPWTFGQGTKVEIKGGGGSGGGGSG | |
| | | GGGSGGGGSQVQLQESGPGLVKPSETLSLTCTVSGGSISSYYWSWIRQP | |
| | | PGKGLEWIGYIYYSGITHYNPSLKSRVTISVDTSKIQFSLKLSSVTAAD | |
| | | TAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 471 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSEIVLTQSPGTLSLS | |
| | | PGERATLSCRASQSVSSSYLAWYQQKPGQAPRLLIYGASSRATGIPDRF | |
| | | SGSGSGTDFTLTISRLEPEDFAVYYCQQYGSSPWTFGQGTKVEIKGGGG | |
| | | SGGGGSGGGGSGGGGSQVQLQESGPGLVKPSETLSLTCTVSGGSISSYY | |
| | | WSWIRQPPGKGLEWIGYIYYSGITHYNPSLKSRVTISVDTSKIQFSLKL | |
| | | SSVTAADTAVYYCARWGVRRDYYYYGMDVWGQGTTVTVSS | |
| | 3^{rd} | EIVLTQSPATLSLSPGERATLSCRASSSVSYMHWYQQKPGQAPRPWIYA | 472 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 473 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2020 | 1^{st} | QVQLVQSGAEVKKPGASVKVSCKASGYTFTSYVMHWVRQKPGQGLEWIG | 474 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSSGGGGSGGGGSGGGGSGGGGSEIVLTQSPGTLSLSPGERATLSCRAS | |
| | | QSVSSSYLAWYQQKPGQAPRLLIYGASSRATGIPDRFSGSGSGTDFTLT | |
| | | ISRLEPEDFAVYYCQQYGSSPWTFGQGTKVEIK | |
| | 2^{nd} | QVQLVQSGAEVKKPGASVKVSCKASGYTFTSYVMHWVRQKPGQGLEWIG | 475 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLQESGPGLVKP | |
| | | SETLSLTCTVSGGSISSYYWSWIRQPPGKGLEWIGYIYYSGITHYNPSL | |
| | | KSRVTISVDTSKIQFSLKLSSVTAADTAVYYCARWGVRRDYYYYGMDVW | |
| | | GQGTTVTVSSGGGGSGGGGSGGGGSGGGGSEIVLTQSPGTLSLSPGERA | |
| | | TLSCRASQSVSSSYLAWYQQKPGQAPRLLIYGASSRATGIPDRFSGSGS | |
| | | GTDFTLTISRLEPEDFAVYYCQQYGSSPWTFGQGTKVEIK | |
| | 3^{rd} | EIVLTQSPATLSLSPGERATLSCRASSSVSYMHWYQQKPGQAPRPWIYA | 476 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 477 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2021 | 1^{st} | QVQLVQSGAEVKKPGASVKVSCKASGYTFTSYVMHWVRQKPGQGLEWIG | 478 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 479 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSEIVLTQSPGTLSLS | |
| | | PGERATLSCRASQSVSSSYLAWYQQKPGQAPRLLIYGASSRATGIPDRF | |
| | | SGSGSGTDFTLTISRLEPEDFAVYYCQQYGSSPWTFGQGTKVEIKGGGG | |
| | | SGGGGSGGGGSGGGGSQVQLQESGPGLVKPSETLSLTCTVSGGSISSYY | |
| | | WSWIRQPPGKGLEWIGYIYYSGITHYNPSLKSRVTISVDTSKIQFSLKL | |
| | | SSVTAADTAVYYCARWGVRRDYYYYGMDVWGQGTTVTVSS | |
| | 3^{rd} | EIVLTQSPATLSLSPGERATLSCRASSSVSYMHWYQQKPGQAPRPWIYA | 480 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 481 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2022 | 1^{st} | QVQLVQSGAEVKKPGASVKVSCKASGYTFTSYVMHWVRQKPGQGLEWIG | 482 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 483 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSQVQLQESGPGLVKP | |
| | | SETLSLTCTVSGGSISSYYWSWIRQPPGKGLEWIGYIYYSGITHYNPSL | |
| | | KSRVTISVDTSKIQFSLKLSSVTAADTAVYYCARWGVRRDYYYYGMDVW | |
| | | GQGTTVTVSSGGGGSGGGGSGGGGSGGGGSEIVLTQSPGTLSLSPGERA | |
| | | TLSCRASQSVSSSYLAWYQQKPGQAPRLLIYGASSRATGIPDRFSGSGS | |
| | | GTDFTLTISRLEPEDFAVYYCQQYGSSPWTFGQGTKVEIK | |
| | 3^{rd} | EIVLTQSPATLSLSPGERATLSCRASSSVSYMHWYQQKPGQAPRPWIYA | 484 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 485 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2023 | 1^{st} | QVQLVQSGAEVKKPGASVKVSCKASGYTFTSYVMHWVRQKPGQGLEWIG | 486 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPGTLSLSPGERATL | |
| | | SCRASQSVSSSYLAWYQQKPGQAPRLLIYGASSRATGIPDRFSGSGSGT | |
| | | DFTLTISRLEPEDFAVYYCQQYGSSPWTFGQGTKVEIKGGGGSGGGGSG | |
| | | GGGSGGGGSQVQLQESGPGLVKPSETLSLTCTVSGGSISSYYWSWIRQP | |
| | | PGKGLEWIGYIYYSGITHYNPSLKSRVTISVDTSKIQFSLKLSSVTAAD | |
| | | TAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 487 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 488 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 489 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2024 | 1^{st} | QVQLVQSGAEVKKPGASVKVSCKASGYTFTSYVMHWVRQKPGQGLEWIG | 490 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSSGGGGSGGGGSGGGGSGGGGSEIVLTQSPGTLSLSPGERATLSCRAS | |
| | | QSVSSSYLAWYQQKPGQAPRLLIYGASSRATGIPDRFSGSGSGTDFTLT | |
| | | ISRLEPEDFAVYYCQQYGSSPWTFGQGTKVEIK | |
| | 2^{nd} | QVQLVQSGAEVKKPGASVKVSCKASGYTFTSYVMHWVRQKPGQGLEWIG | 491 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 492 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 493 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2025 | 1^{st} | QVQLVQSGAEVKKPGASVKVSCKASGYTFTSYVMHWVRQKPGQGLEWIG | 494 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSS | |
| | 2^{nd} | QVQLVQSGAEVKKPGASVKVSCKASGYTFTSYVMHWVRQKPGQGLEWIG | 495 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSEIVLTQSPGTLSLS | |
| | | PGERATLSCRASQSVSSSYLAWYQQKPGQAPRLLIYGASSRATGIPDRF | |
| | | SGSGSGTDFTLTISRLEPEDFAVYYCQQYGSSPWTFGQGTKVEIK | |
| | 3^{rd} | EIVLTQSPATLSLSPGERATLSCRASSSVSYMHWYQQKPGQAPRPWIYA | 496 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 497 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly2026 | 1^{st} | QVQLVQSGAEVKKPGASVKVSCKASGYTFTSYVMHWVRQKPGQGLEWIG | 498 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPGTLSLSPGERATL | |
| | | SCRASQSVSSSYLAWYQQKPGQAPRLLIYGASSRATGIPDRFSGSGSGT | |
| | | DFTLTISRLEPEDFAVYYCQQYGSSPWTFGQGTKVEIKGGGGSGGGGSG | |
| | | GGGSGGGGSQVQLQESGPGLVKPSETLSLTCTVSGGSISSYYWSWIRQP | |
| | | PGKGLEWIGYIYYSGITHYNPSLKSRVTISVDTSKIQFSLKLSSVTAAD | |
| | | TAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 499 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 500 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2027 | 1^{st} | QVQLVQSGAEVKKPGASVKVSCKASGYTFTSYVMHWVRQKPGQGLEWIG | 501 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSSGGGGSGGGGSGGGGSGGGGSEIVLTQSPGTLSLSPGERATLSCRAS | |
| | | QSVSSSYLAWYQQKPGQAPRLLIYGASSRATGIPDRFSGSGSGTDFTLT | |
| | | ISRLEPEDFAVYYCQQYGSSPWTFGQGTKVEIK | |
| | 2^{nd} | QVQLVQSGAEVKKPGASVKVSCKASGYTFTSYVMHWVRQKPGQGLEWIG | 502 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 503 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2028 | 1^{st} | QVQLVQSGAEVKKPGASVKVSCKASGYTFTSYVMHWVRQKPGQGLEWIG | 504 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPGTLSLSPGERATL | |
| | | SCRASQSVSSSYLAWYQQKPGQAPRLLIYGASSRATGIPDRFSGSGSGT | |
| | | DFTLTISRLEPEDFAVYYCQQYGSSPWTFGQGTKVEIKGGGGSGGGGSG | |
| | | GGGSGGGGSQVQLQESGPGLVKPSETLSLTCTVSGGSISSYYWSWIRQP | |
| | | PGKGLEWIGYIYYSGITHYNPSLKSRVTISVDTSKIQFSLKLSSVTAAD | |
| | | TAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 505 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSEIVLTQSPGTLSLSPGERATLSCRASQSVSSSYLAWYQQKP | |
| | | GQAPRLLIYGASSRATGIPDRFSGSGSGTDFTLTISRLEPEDFAVYYCQ | |
| | | QYGSSPWTFGQGTKVEIKGGGGSGGGGSGGGGSGGGGSQVQLQESGPGL | |
| | | VKPSETLSLTCTVSGGSISSYYWSWIRQPPGKGLEWIGYIYYSGITHYN | |
| | | PSLKSRVTISVDTSKIQFSLKLSSVTAADTAVYYCARWGVRRDYYYYGM | |
| | | DVWGQGTTVTVSS | |
| | 3^{rd} | EIVLTQSPATLSLSPGERATLSCRASSSVSYMHWYQQKPGQAPRPWIYA | 506 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2029 | 1^{st} | QVQLVQSGAEVKKPGASVKVSCKASGYTFTSYVMHWVRQKPGQGLEWIG | 507 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSSGGGGSGGGGSGGGGSGGGGSEIVLTQSPGTLSLSPGERATLSCRAS | |
| | | QSVSSSYLAWYQQKPGQAPRLLIYGASSRATGIPDRFSGSGSGTDFTLT | |
| | | ISRLEPEDFAVYYCQQYGSSPWTFGQGTKVEIK | |
| | 2^{nd} | QVQLVQSGAEVKKPGASVKVSCKASGYTFTSYVMHWVRQKPGQGLEWIG | 508 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLQESGPGLVKPSETLSLTCTVSGGSISSYYWSWIRQPP | |
| | | GKGLEWIGYIYYSGITHYNPSLKSRVTISVDTSKIQFSLKLSSVTAADT | |
| | | AVYYCARWGVRRDYYYYGMDVWGQGTTVTVSSGGGGSGGGGSGGGGSGG | |
| | | GGSEIVLTQSPGTLSLSPGERATLSCRASQSVSSSYLAWYQQKPGQAPR | |
| | | LLIYGASSRATGIPDRFSGSGSGTDFTLTISRLEPEDFAVYYCQQYGSS | |
| | | PWTFGQGTKVEIK | |
| | 3^{rd} | EIVLTQSPATLSLSPGERATLSCRASSSVSYMHWYQQKPGQAPRPWIYA | 509 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2030 | 1^{st} | QVQLVQSGAEVKKPGASVKVSCKASGYTFTSYVMHWVRQKPGQGLEWIG | 510 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 511 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSEIVLTQSPGTLSLSPGERATLSCRASQSVSSSYLAWYQQKP | |
| | | GQAPRLLIYGASSRATGIPDRFSGSGSGTDFTLTISRLEPEDFAVYYCQ | |
| | | QYGSSPWTFGQGTKVEIKGGGGSGGGGSGGGGSGGGGSQVQLQESGPGL | |
| | | VKPSETLSLTCTVSGGSISSYYWSWIRQPPGKGLEWIGYIYYSGITHYN | |
| | | PSLKSRVTISVDTSKIQFSLKLSSVTAADTAVYYCARWGVRRDYYYYGM | |
| | | DVWGQGTTVTVSS | |
| | 3^{rd} | EIVLTQSPATLSLSPGERATLSCRASSSVSYMHWYQQKPGQAPRPWIYA | 512 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2031 | 1^{st} | QVQLVQSGAEVKKPGASVKVSCKASGYTFTSYVMHWVRQKPGQGLEWIG | 513 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 514 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSQVQLQESGPGLVKPSETLSLTCTVSGGSISSYYWSWIRQPP | |
| | | GKGLEWIGYIYYSGITHYNPSLKSRVTISVDTSKIQFSLKLSSVTAADT | |
| | | AVYYCARWGVRRDYYYYGMDVWGQGTTVTVSSGGGGSGGGGSGGGGSGG | |
| | | GGSEIVLTQSPGTLSLSPGERATLSCRASQSVSSSYLAWYQQKPGQAPR | |
| | | LLIYGASSRATGIPDRFSGSGSGTDFTLTISRLEPEDFAVYYCQQYGSS | |
| | | PWTFGQGTKVEIK | |
| | 3^{rd} | EIVLTQSPATLSLSPGERATLSCRASSSVSYMHWYQQKPGQAPRPWIYA | 515 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2032 | 1^{st} | QVQLVQSGAEVKKPGASVKVSCKASGYTFTSYVMHWVRQKPGQGLEWIG | 516 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSEIVLTQSPGTLSLSPGERATLSCRASQSVSSS | |
| | | YLAWYQQKPGQAPRLLIYGASSRATGIPDRFSGSGSGTDFTLTISRLEP | |
| | | EDFAVYYCQQYGSSPWTFGQGTKVEIKGGGGSGGGGSGGGGSGGGGSQV | |
| | | QLQESGPGLVKPSETLSLTCTVSGGSISSYYWSWIRQPPGKGLEWIGYI | |
| | | YYSGITHYNPSLKSRVTISVDTSKIQFSLKLSSVTAADTAVYYCARWGV | |
| | | RRDYYYYGMDVWGQGTTVTVSS | |
| | 2^{nd} | QVQLVQSGAEVKKPGASVKVSCKASGYTFTSYVMHWVRQKPGQGLEWIG | 517 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SEIVLTQSPGTLSLSPGERATLSCRASQSVSSSYLAWYQQKPGQAPRLL | |
| | | IYGASSRATGIPDRFSGSGSGTDFTLTISRLEPEDFAVYYCQQYGSSPW | |
| | | TFGQGTKVEIKGGGGSGGGGSGGGGSGGGGSQVQLQESGPGLVKPSETL | |
| | | SLTCTVSGGSISSYYWSWIRQPPGKGLEWIGYIYYSGITHYNPSLKSRV | |
| | | TISVDTSKIQFSLKLSSVTAADTAVYYCARWGVRRDYYYYGMDVWGQGT | |
| | | TVTVSS | |
| | 3^{rd} | EIVLTQSPATLSLSPGERATLSCRASSSVSYMHWYQQKPGQAPRPWIYA | 518 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2033 | 1^{st} | QVQLVQSGAEVKKPGASVKVSCKASGYTFTSYVMHWVRQKPGQGLEWIG | 519 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLQESGPGLVKPSETLSLTCTVSGGSISSY | |
| | | YWSWIRQPPGKGLEWIGYIYYSGITHYNPSLKSRVTISVDTSKIQFSLK | |
| | | LSSVTAADTAVYYCARWGVRRDYYYYGMDVWGQGTTVTVSSGGGGSGGG | |
| | | GSGGGGSGGGGSEIVLTQSPGTLSLSPGERATLSCRASQSVSSSYLAWY | |
| | | QQKPGQAPRLLIYGASSRATGIPDRFSGSGSGTDFTLTISRLEPEDFAV | |
| | | YYCQQYGSSPWTFGQGTKVEIK | |
| | 2^{nd} | QVQLVQSGAEVKKPGASVKVSCKASGYTFTSYVMHWVRQKPGQGLEWIG | 520 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLQESGPGLVKPSETLSLTCTVSGGSISSYYWSWIRQPPGKGLEWI | |
| | | GYIYYSGITHYNPSLKSRVTISVDTSKIQFSLKLSSVTAADTAVYYCAR | |
| | | WGVRRDYYYYGMDVWGQGTTVTVSSGGGGSGGGGSGGGGSGGGGSEIVL | |
| | | TQSPGTLSLSPGERATLSCRASQSVSSSYLAWYQQKPGQAPRLLIYGAS | |
| | | SRATGIPDRFSGSGSGTDFTLTISRLEPEDFAVYYCQQYGSSPWTFGQG | |
| | | TKVEIK | |
| | 3^{rd} | EIVLTQSPATLSLSPGERATLSCRASSSVSYMHWYQQKPGQAPRPWIYA | 521 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2034 | 1^{st} | QVQLVQSGAEVKKPGASVKVSCKASGYTFTSYVMHWVRQKPGQGLEWIG | 522 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSEIVLTQSPGTLSLSPGERATLSCRASQSVSSS | |
| | | YLAWYQQKPGQAPRLLIYGASSRATGIPDRFSGSGSGTDFTLTISRLEP | |
| | | EDFAVYYCQQYGSSPWTFGQGTKVEIKGGGGSGGGGSGGGGSGGGGSQV | |
| | | QLQESGPGLVKPSETLSLTCTVSGGSISSYYWSWIRQPPGKGLEWIGYI | |
| | | YYSGITHYNPSLKSRVTISVDTSKIQFSLKLSSVTAADTAVYYCARWGV | |
| | | RRDYYYYGMDVWGQGTTVTVSS | |
| | 2^{nd} | QVQLVQSGAEVKKPGASVKVSCKASGYTFTSYVMHWVRQKPGQGLEWIG | 523 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 524 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2035 | 1^{st} | QVQLVQSGAEVKKPGASVKVSCKASGYTFTSYVMHWVRQKPGQGLEWIG | 525 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLQESGPGLVKPSETLSLTCTVSGGSISSY | |
| | | YWSWIRQPPGKGLEWIGYIYYSGITHYNPSLKSRVTISVDTSKIQFSLK | |
| | | LSSVTAADTAVYYCARWGVRRDYYYYGMDVWGQGTTVTVSSGGGGSGGG | |
| | | GSGGGGSGGGGSEIVLTQSPGTLSLSPGERATLSCRASQSVSSSYLAWY | |
| | | QQKPGQAPRLLIYGASSRATGIPDRFSGSGSGTDFTLTISRLEPEDFAV | |
| | | YYCQQYGSSPWTFGQGTKVEIK | |
| | 2^{nd} | QVQLVQSGAEVKKPGASVKVSCKASGYTFTSYVMHWVRQKPGQGLEWIG | 526 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 527 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2036 | 1^{st} | QVQLVQSGAEVKKPGASVKVSCKASGYTFTSYVMHWVRQKPGQGLEWIG | 528 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 529 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SEIVLTQSPGTLSLSPGERATLSCRASQSVSSSYLAWYQQKPGQAPRLL | |
| | | IYGASSRATGIPDRFSGSGSGTDFTLTISRLEPEDFAVYYCQQYGSSPW | |
| | | TFGQGTKVEIKGGGGSGGGGSGGGGSGGGGSQVQLQESGPGLVKPSETL | |
| | | SLTCTVSGGSISSYYWSWIRQPPGKGLEWIGYIYYSGITHYNPSLKSRV | |
| | | TISVDTSKIQFSLKLSSVTAADTAVYYCARWGVRRDYYYYGMDVWGQGT | |
| | | TVTVSS | |
| | 3^{rd} | EIVLTQSPATLSLSPGERATLSCRASSSVSYMHWYQQKPGQAPRPWIYA | 530 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2037 | 1^{st} | QVQLVQSGAEVKKPGASVKVSCKASGYTFTSYVMHWVRQKPGQGLEWIG | 531 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 532 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SQVQLQESGPGLVKPSETLSLTCTVSGGSISSYYWSWIRQPPGKGLEWI | |
| | | GYIYYSGITHYNPSLKSRVTISVDTSKIQFSLKLSSVTAADTAVYYCAR | |
| | | WGVRRDYYYYGMDVWGQGTTVTVSSGGGGSGGGGSGGGGSGGGGSEIVL | |
| | | TQSPGTLSLSPGERATLSCRASQSVSSSYLAWYQQKPGQAPRLLIYGAS | |
| | | SRATGIPDRFSGSGSGTDFTLTISRLEPEDFAVYYCQQYGSSPWTFGQG | |
| | | TKVEIK | |
| | 3^{rd} | EIVLTQSPATLSLSPGERATLSCRASSSVSYMHWYQQKPGQAPRPWIYA | 533 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2038 | 1^{st} | QVQLVQSGAEVKKPGASVKVSCKASGYTFTSYVMHWVRQKPGQGLEWIG | 534 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSQVQLQESGPGLVKPSETLSLTCTVSGGSISSY | |
| | | YWSWIRQPPGKGLEWIGYIYYSGITHYNPSLKSRVTISVDTSKIQFSLK | |
| | | LSSVTAADTAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 535 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SEIVLTQSPGTLSLSPGERATLSCRASQSVSSSYLAWYQQKPGQAPRLL | |
| | | IYGASSRATGIPDRFSGSGSGTDFTLTISRLEPEDFAVYYCQQYGSSPW | |
| | | TFGQGTKVEIK | |
| | 3^{rd} | EIVLTQSPATLSLSPGERATLSCRASSSVSYMHWYQQKPGQAPRPWIYA | 536 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2039 | 1^{st} | QVQLVQSGAEVKKPGASVKVSCKASGYTFTSYVMHWVRQKPGQGLEWIG | 537 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 538 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 539 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSEIVLTQSPGTLS | |
| | | LSPGERATLSCRASQSVSSSYLAWYQQKPGQAPRLLIYGASSRATGIPD | |
| | | RFSGSGSGTDFTLTISRLEPEDFAVYYCQQYGSSPWTFGQGTKVEIKGG | |
| | | GGSGGGGSGGGGSGGGGSQVQLQESGPGLVKPSETLSLTCTVSGGSISS | |
| | | YYWSWIRQPPGKGLEWIGYIYYSGITHYNPSLKSRVTISVDTSKIQFSL | |
| | | KLSSVTAADTAVYYCARWGVRRDYYYYGMDVWGQGTTVTVSS | |
| Ly2040 | 1^{st} | QVQLVQSGAEVKKPGASVKVSCKASGYTFTSYVMHWVRQKPGQGLEWIG | 540 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 541 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 542 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSQVQLQESGPGLV | |
| | | KPSETLSLTCTVSGGSISSYYWSWIRQPPGKGLEWIGYIYYSGITHYNP | |
| | | SLKSRVTISVDTSKIQFSLKLSSVTAADTAVYYCARWGVRRDYYYYGMD | |
| | | VWGQGTTVTVSSGGGGSGGGGSGGGGSGGGGSEIVLTQSPGTLSLSPGE | |
| | | RATLSCRASQSVSSSYLAWYQQKPGQAPRLLIYGASSRATGIPDRESGS | |
| | | GSGTDFTLTISRLEPEDFAVYYCQQYGSSPWTFGQGTKVEIK | |
| Ly2041 | 1^{st} | QVQLVQSGAEVKKPGASVKVSCKASGYTFTSYVMHWVRQKPGQGLEWIG | 543 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPGTLSLSPGERATL | |
| | | SCRASQSVSSSYLAWYQQKPGQAPRLLIYGASSRATGIPDRFSGSGSGT | |
| | | DFTLTISRLEPEDFAVYYCQQYGSSPWTFGQGTKVEIKGGGGSGGGGSG | |
| | | GGGSGGGGSQVQLQESGPGLVKPSETLSLTCTVSGGSISSYYWSWIRQP | |
| | | PGKGLEWIGYIYYSGITHYNPSLKSRVTISVDTSKIQFSLKLSSVTAAD | |
| | | TAVYYCARWGVRRDYYYYGMDVWGQGTTVTVSS | |
| | 2^{nd} | QVQLVQSGAEVKKPGASVKVSCKASGYTFTSYVMHWVRQKPGQGLEWIG | 544 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 545 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly2042 | 1^{st} | QVQLVQSGAEVKKPGASVKVSCKASGYTFTSYVMHWVRQKPGQGLEWIG | 546 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSQVQLQESGPGLVKPSETLSLT | |
| | | CTVSGGSISSYYWSWIRQPPGKGLEWIGYIYYSGITHYNPSLKSRVTIS | |
| | | VDTSKIQFSLKLSSVTAADTAVYYCARWGVRRDYYYYGMDVWGQGTTVT | |
| | | VSSGGGGSGGGGSGGGGSGGGGSEIVLTQSPGTLSLSPGERATLSCRAS | |
| | | QSVSSSYLAWYQQKPGQAPRLLIYGASSRATGIPDRFSGSGSGTDFTLT | |
| | | ISRLEPEDFAVYYCQQYGSSPWTFGQGTKVEIK | |
| | 2^{nd} | QVQLVQSGAEVKKPGASVKVSCKASGYTFTSYVMHWVRQKPGQGLEWIG | 547 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 548 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |

Characterization of anti-B7H3/CD3/CD28 tri-specific antibodies

These tri-specific antibodies are evaluated for their in vitro and in vivo activity, including binding to target antigen (B7H3, CD3 and CD28), agonistic activity in CD3 and CD28 reporter assay system, activation of immune cells, anti-tumor activity in mouse models.

### Example 11: Anti-B7H3/CD3/OX40 Tri-Specific Antibodies

Anti-B7H3/CD3/OX40 tri-specific were produced using the same parent anti-B7H3 and anti-CD3 antibodies disclosed above and the anti-OX40 parent antibody Ly598 (CD40 Ab1).

The sequences of the parent antibody clones are provided in Table 1 above. cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 11 below:

TABLE 11
Exemplary Anti-B7H3/CD3/OX40 Tri-Specific Antibodies

| | | | SED ID |
|---|---|---|---|
| Antibody Clones | | Amino acid sequence | NO: |
| Ly1919 | 1^{st} | QVQLVQSGAEVKKPGASVKVSCKASGYTFTSYVMHWVRQKPGQGLEWIG | 549 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | EVQLVQSGAEVKKPGASVKVSCKASGYTFTDSYMSWVRQAPGQGLEWIG | 550 |
| | chain | DMYPDNGDSSYNQKFRERVTITRDTSTSTAYLELSSLRSEDTAVYYCVL | |
| | | APRWYFSVWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 551 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 552 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1920 | 1^{st} | QVQLVQSGAEVKKPGASVKVSCKASGYTFTSYVMHWVRQKPGQGLEWIG | 553 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 554 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIKGGGGS | |
| | | GGGGSGGGGSGGGGSEVQLVQSGAEVKKPGASVKVSCKASGYTFTDSYM | |
| | | SWVRQAPGQGLEWIGDMYPDNGDSSYNQKFRERVTITRDTSTSTAYLEL | |
| | | SSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 555 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 556 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1921 | 1^{st} | QVQLVQSGAEVKKPGASVKVSCKASGYTFTSYVMHWVRQKPGQGLEWIG | 557 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 558 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIKGGGGS | |
| | | GGGGSGGGGSGGGGSEVQLVQSGAEVKKPGASVKVSCKASGYTFTDSYM | |
| | | SWVRQAPGQGLEWIGDMYPDNGDSSYNQKFRERVTITRDTSTSTAYLEL | |
| | | SSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 559 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 560 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1922 | 1^{st} | QVQLVQSGAEVKKPGASVKVSCKASGYTFTSYVMHWVRQKPGQGLEWIG | 561 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 562 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 563 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 564 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1923 | 1^{st} | QVQLVQSGAEVKKPGASVKVSCKASGYTFTSYVMHWVRQKPGQGLEWIG | 565 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEVQLVQSGAEVKKPGASVKVS | |
| | | CKASGYTFTDSYMSWVRQAPGQGLEWIGDMYPDNGDSSYNQKFRERVTI | |
| | | TRDTSTSTAYLELSSLRSEDTAVYYCVLAPRWYFSVWGQGTLVTVSS | |
| | 2^{nd} | QVQLVQSGAEVKKPGASVKVSCKASGYTFTSYVMHWVRQKPGQGLEWIG | 566 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIK | |
| | 3^{rd} | EIVLTQSPATLSLSPGERATLSCRASSSVSYMHWYQQKPGQAPRPWIYA | 567 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 568 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1924 | 1^{st} | QVQLVQSGAEVKKPGASVKVSCKASGYTFTSYVMHWVRQKPGQGLEWIG | 569 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPATLSLSPGERATL | |
| | | SCRASKSVRTGMHWYQQKPGQAPRLLIYGASNLESGIPARFSGSGSGTD | |
| | | FTLTISSLEPEDFAVYYCQQSWNHFTFGQGTKLEIKGGGGSGGGGSGGG | |
| | | GSGGGGSQVQLQESGPGLVKPSETLSLTCTVSGFSLTSYNVHWIRQPPG | |
| | | KGLEWIGVIWSGVRTDYNSVLKPRVTISVDTSKNQFSLKLSSVTAADTA | |
| | | VYYCARGTYDDNYHDVMDAWGQGTLVTVSS | |
| | 2^{nd} | QVQLVQSGAEVKKPGASVKVSCKASGYTFTSYVMHWVRQKPGQGLEWIG | 570 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 571 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1925 | 1^{st} | QVQLVQSGAEVKKPGASVKVSCKASGYTFTSYVMHWVRQKPGQGLEWIG | 572 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 573 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCRASQDISNYLNWYQQKPG | |
| | | KAPKLLIYYTSRLRSGVPSRFSGSGSGTDFTLTISSLQPEDFATYYCQQ | |
| | | GHTLPPTFGQGTKVEIKGGGGSGGGGSGGGGSGGGGSEVQLVQSGAEVK | |
| | | KPGASVKVSCKASGYTFTDSYMSWVRQAPGQGLEWIGDMYPDNGDSSYN | |
| | | QKFRERVTITRDTSTSTAYLELSSLRSEDTAVYYCVLAPRWYFSVWGQG | |
| | | TLVTVSS | |
| | 3^{rd} | EIVLTQSPATLSLSPGERATLSCRASSSVSYMHWYQQKPGQAPRPWIYA | 574 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1926 | 1^{st} | QVQLVQSGAEVKKPGASVKVSCKASGYTFTSYVMHWVRQKPGQGLEWIG | 575 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 576 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSEIVLTQSPATLSLSPGERATLSCRASKSVRTGMHWYQQKPG | |
| | | QAPRLLIYGASNLESGIPARFSGSGSGTDFTLTISSLEPEDFAVYYCQQ | |
| | | SWNHFTFGQGTKLEIKGGGGSGGGGSGGGGSGGGGSQVQLQESGPGLVK | |
| | | PSETLSLTCTVSGFSLTSYNVHWIRQPPGKGLEWIGVIWSGVRTDYNSV | |
| | | LKPRVTISVDTSKNQFSLKLSSVTAADTAVYYCARGTYDDNYHDVMDAW | |
| | | GQGTLVTVSS | |
| | 3rd | EIVLTQSPATLSLSPGERATLSCRASSSVSYMHWYQQKPGQAPRPWIYA | 577 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1927 | 1^{st} | QVQLVQSGAEVKKPGASVKVSCKASGYTFTSYVMHWVRQKPGQGLEWIG | 578 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCRASQDISNY | |
| | | LNWYQQKPGKAPKLLIYYTSRLRSGVPSRFSGSGSGTDFTLTISSLQPE | |
| | | DFATYYCQQGHTLPPTFGQGTKVEIKGGGGSGGGGSGGGGSGGGGSEVQ | |
| | | LVQSGAEVKKPGASVKVSCKASGYTFTDSYMSWVRQAPGQGLEWIGDMY | |
| | | PDNGDSSYNQKFRERVTITRDTSTSTAYLELSSLRSEDTAVYYCVLAPR | |
| | | WYFSVWGQGTLVTVSS | |
| | 2^{nd} | QVQLVQSGAEVKKPGASVKVSCKASGYTFTSYVMHWVRQKPGQGLEWIG | 579 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIKGGGGSGGGGSGGGGSGGGGSEVQLVQSGAEVKKPGASVK | |
| | | VSCKASGYTFTDSYMSWVRQAPGQGLEWIGDMYPDNGDSSYNQKFRERV | |
| | | TITRDTSTSTAYLELSSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 580 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1928 | 1^{st} | QVQLVQSGAEVKKPGASVKVSCKASGYTFTSYVMHWVRQKPGQGLEWIG | 581 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCRASQDISNY | |
| | | LNWYQQKPGKAPKLLIYYTSRLRSGVPSRFSGSGSGTDFTLTISSLQPE | |
| | | DFATYYCQQGHTLPPTFGQGTKVEIKGGGGSGGGGSGGGGSGGGGSEVQ | |
| | | LVQSGAEVKKPGASVKVSCKASGYTFTDSYMSWVRQAPGQGLEWIGDMY | |
| | | PDNGDSSYNQKFRERVTITRDTSTSTAYLELSSLRSEDTAVYYCVLAPR | |
| | | WYFSVWGQGTLVTVSS | |
| | 2^{nd} | QVQLVQSGAEVKKPGASVKVSCKASGYTFTSYVMHWVRQKPGQGLEWIG | 582 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 583 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1929 | 1^{st} | QVQLVQSGAEVKKPGASVKVSCKASGYTFTSYVMHWVRQKPGQGLEWIG | 584 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 585 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIKGGGGSGGGGSGGGGSGGGGSEVQLVQSGAEVKKPGASVK | |
| | | VSCKASGYTFTDSYMSWVRQAPGQGLEWIGDMYPDNGDSSYNQKFRERV | |
| | | TITRDTSTSTAYLELSSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 586 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1930 | 1^{st} | QVQLVQSGAEVKKPGASVKVSCKASGYTFTSYVMHWVRQKPGQGLEWIG | 587 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| | 2^{nd} | QVQLVQSGAEVKKPGASVKVSCKASGYTFTSYVMHWVRQKPGQGLEWIG | 588 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIK | |
| | 3^{rd} | EIVLTQSPATLSLSPGERATLSCRASSSVSYMHWYQQKPGQAPRPWIYA | 589 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1931 | 1^{st} | QVQLVQSGAEVKKPGASVKVSCKASGYTFTSYVMHWVRQKPGQGLEWIG | 590 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 591 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 592 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly1932 | 1^{st} | QVQLVQSGAEVKKPGASVKVSCKASGYTFTSYVMHWVRQKPGQGLEWIG | 593 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 594 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 595 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1934 | 1^{st} | QVQLVQSGAEVKKPGASVKVSCKASGYTFTSYVMHWVRQKPGQGLEWIG | 596 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 597 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVQSGAEVKKPGASVKVSCKASGYTFTDSYMSWVRQAPGQGLEWIGD | |
| | | MYPDNGDSSYNQKFRERVTITRDTSTSTAYLELSSLRSEDTAVYYCVLA | |
| | | PRWYFSVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECGGGGSGGGGSGGGGSGGGGSQA | |
| | | VVTQEPSLTVSPGGTVTLTCRSSTGAVTTSNYANWVQQKPGQAPRGLIG | |
| | | GTNKRAPWTPARFSGSLLGGKAALTITGAQAEDEADYYCALWYSNLWVE | |
| | | GGGTKLTVLPAPEAAGPSVFLFPPKPKDTLMISRTPEVTCVVVDVSHED | |
| | | PEVKFNWYVDGVEVHNAKTKPREEQYNSTYRVVSVLTVLHQDWLNGKEY | |
| | | KCKVSNKALPAPIEKTISKAKGQPREPQVYTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 598 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 599 |
| | chain | YTSRLRSGVPSRFSGSGSGTDFTLTISSLOPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLOSSGLYSLSSVVTVPSSSLGTQTYICNVNH | |
| | | KPSNTKVDKKVEPKSC | |

### Characterization of Anti-B7H3/CD3/OX40 Tri-Specific Antibodies

These tri-specific antibodies are evaluated for their in vitro and in vivo activity, including binding to target antigen (B37H3, CD3 and OX40), agonistic activity in CD3 and OX40 reporter assay system, activation of immune cells, anti-tumor activity in mouse models.

### Example 12: Anti-B7H3/CD3/GITR Tri-Specific Antibodies

Anti-B7H3/CD3/GITR tri-specific were produced using the same anti-B37H3 and anti-CD3 parent clones disclosed above and the anti-GITR parent antibody GITR Ab1. The sequences of the parent antibody clones are provided in Table 1 above.

cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 12 below:

TABLE 12
Exemplary Anti-B7H3/CD3/GITR Tri-Specific Antibodies

| | | | SED ID |
|---|---|---|---|
| Antibody Clones | | Amino acid sequence | NO: |
| Ly1919 | 1^{st} | QVQLVQSGAEVKKPGASVKVSCKASGYTFTSYVMHWVRQKPGQGLEWIG | 600 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPELLGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | EVQLVQSGAEVKKPGASVKVSCKASGYTFTDSYMSWVRQAPGQGLEWIG | 601 |
| | chain | DMYPDNGDSSYNQKFRERVTITRDTSTSTAYLELSSLRSEDTAVYYCVL | |
| | | APRWYFSVWGQGTLVTVSSASVAAPSVFIFPPSDEQLKSGTASVVCLLN | |
| | | NFYPREAKVQWKVDNALQSGNSQESVTEQDSKDSTYSLSSTLTLSKADY | |
| | | EKHKVYACEVTHQGLSSPVTKSFNRGECGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPELLGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 602 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 603 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |
| Ly1920 | 1^{st} | QVQLVQSGAEVKKPGASVKVSCKASGYTFTSYVMHWVRQKPGQGLEWIG | 604 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVEL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 605 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIKGGGGS | |
| | | GGGGSGGGGSGGGGSEVQLVQSGAEVKKPGASVKVSCKASGYTFTDSYM | |
| | | SWVRQAPGQGLEWIGDMYPDNGDSSYNQKFRERVTITRDTSTSTAYLEL | |
| | | SSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 606 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 607 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1921 | 1^{st} | QVQLVQSGAEVKKPGASVKVSCKASGYTFTSYVMHWVRQKPGQGLEWIG | 608 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 609 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIKGGGGS | |
| | | GGGGSGGGGSGGGGSEVQLVQSGAEVKKPGASVKVSCKASGYTFTDSYM | |
| | | SWVRQAPGQGLEWIGDMYPDNGDSSYNQKFRERVTITRDTSTSTAYLEL | |
| | | SSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 610 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 611 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1922 | 1^{st} | QVQLVQSGAEVKKPGASVKVSCKASGYTFTSYVMHWVRQKPGQGLEWIG | 612 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 613 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGK | |
| | 3^{rd} | EIVLTQSPATLSLSPGERATLSCRASSSVSYMHWYQQKPGQAPRPWIYA | 614 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 615 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1923 | 1^{st} | QVQLVQSGAEVKKPGASVKVSCKASGYTFTSYVMHWVRQKPGQGLEWIG | 616 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEVQLVQSGAEVKKPGASVKVS | |
| | | CKASGYTFTDSYMSWVRQAPGQGLEWIGDMYPDNGDSSYNQKFRERVTI | |
| | | TRDTSTSTAYLELSSLRSEDTAVYYCVLAPRWYFSVWGQGTLVTVSS | |
| | 2^{nd} | QVQLVQSGAEVKKPGASVKVSCKASGYTFTSYVMHWVRQKPGQGLEWIG | 617 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSASVAAPSVFIFPPSDEQLKSGTA | |
| | | SVVCLLNNFYPREAKVQWKVDNALQSGNSQESVTEQDSKDSTYSLSSTL | |
| | | TLSKADYEKHKVYACEVTHQGLSSPVTKSFNRGECDKTHTCPPCPAPEA | |
| | | AGPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPAPIE | |
| | | KTISKAKGQPREPQVYTLPPSREEMTKNQVSLSCAVKGFYPSDIAVEWE | |
| | | SNGQPENNYKTTPPVLDSDGSFFLVSKLTVDKSRWQQGNVFSCSVMHEA | |
| | | LHNRFTQKSLSLSPGGGGGSGGGGSGGGGSGGGGSDIQMTQSPSSLSAS | |
| | | VGDRVTITCRASQDISNYLNWYQQKPGKAPKLLIYYTSRLRSGVPSRFS | |
| | | GSGSGTDFTLTISSLQPEDFATYYCQQGHTLPPTFGQGTKVEIK | |
| | 3^{rd} | EIVLTQSPATLSLSPGERATLSCRASSSVSYMHWYQQKPGQAPRPWIYA | 618 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | QAVVTQEPSLTVSPGGTVTLTCRSSTGAVTTSNYANWVQQKPGQAPRGL | 619 |
| | chain | IGGTNKRAPWTPARFSGSLLGGKAALTITGAQAEDEADYYCALWYSNLW | |
| | | VFGGGTKLTVLSSASTKGPSVFPLAPSSKSTSGGTAALGCLVKDYFPEP | |
| | | VTVSWNSGALTSGVHTFPAVLQSSGLYSLSSVVTVPSSSLGTQTYICNV | |
| | | NHKPSNTKVDKKVEPKSC | |
| Ly1924 | 1^{st} | QVQLVQSGAEVKKPGASVKVSCKASGYTFTSYVMHWVRQKPGQGLEWIG | 620 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSEIVLTQSPATLSLSPGERATL | |
| | | SCRASKSVRTGMHWYQQKPGQAPRLLIYGASNLESGIPARFSGSGSGTD | |
| | | FTLTISSLEPEDFAVYYCQQSWNHFTFGQGTKLEIKGGGGSGGGGSGGG | |
| | | GSGGGGSQVQLQESGPGLVKPSETLSLTCTVSGFSLTSYNVHWIRQPPG | |
| | | KGLEWIGVIWSGVRTDYNSVLKPRVTISVDTSKNQFSLKLSSVTAADTA | |
| | | VYYCARGTYDDNYHDVMDAWGQGTLVTVSS | |
| | 2^{nd} | QVQLVQSGAEVKKPGASVKVSCKASGYTFTSYVMHWVRQKPGQGLEWIG | 621 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGK | |
| | 3^{rd} | EIVLTQSPATLSLSPGERATLSCRASSSVSYMHWYQQKPGQAPRPWIYA | 622 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1925 | 1^{st} | QVQLVQSGAEVKKPGASVKVSCKASGYTFTSYVMHWVRQKPGQGLEWIG | 623 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 624 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSDIQMTQSPSSLSASVGDRVTITCRASQDISNYLNWYQQKPG | |
| | | KAPKLLIYYTSRLRSGVPSRFSGSGSGTDFTLTISSLQPEDFATYYCQQ | |
| | | GHTLPPTFGQGTKVEIKGGGGSGGGGSGGGGSGGGGSEVQLVQSGAEVK | |
| | | KPGASVKVSCKASGYTFTDSYMSWVRQAPGQGLEWIGDMYPDNGDSSYN | |
| | | QKFRERVTITRDTSTSTAYLELSSLRSEDTAVYYCVLAPRWYFSVWGQG | |
| | | TLVTVSS | |
| | 3^{rd} | EIVLTQSPATLSLSPGERATLSCRASSSVSYMHWYQQKPGQAPRPWIYA | 625 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1926 | 1^{st} | QVQLVQSGAEVKKPGASVKVSCKASGYTFTSYVMHWVRQKPGQGLEWIG | 626 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGK | |
| | 2^{nd} | QVQLVQSGAEVKKPGASVKVSCKASGYTFTSYVMHWVRQKPGQGLEWIG | 627 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLGGGGSGGGGSGGGGSGGGGSEVQLVESGGGLVQPGGSLR | |
| | | LSCAASGFTFSTYAMNWVRQAPGKGLEWVGRIRSKYNNYATYYADSVKG | |
| | | RFTISRDDSKNSLYLQMNSLKTEDTAVYYCVRHGNFGNSYVSWFAYWGQ | |
| | | GTLVTVSSDKTHTCPPCPAPEAAGPSVFLFPPKPKDTLMISRTPEVTCV | |
| | | VVDVSHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEMTK | |
| | | NQVSLSCAVKGFYPSDIAVEWESNGQPENNYKTTPPVLDSDGSFFLVSK | |
| | | LTVDKSRWQQGNVFSCSVMHEALHNRFTQKSLSLSPGGGGGSGGGGSGG | |
| | | GGSGGGGSEIVLTQSPATLSLSPGERATLSCRASKSVRTGMHWYQQKPG | |
| | | QAPRLLIYGASNLESGIPARFSGSGSGTDFTLTISSLEPEDFAVYYCQQ | |
| | | SWNHFTFGQGTKLEIKGGGGSGGGGSGGGGSGGGGSQVQLQESGPGLVK | |
| | | PSETLSLTCTVSGFSLTSYNVHWIRQPPGKGLEWIGVIWSGVRTDYNSV | |
| | | LKPRVTISVDTSKNQFSLKLSSVTAADTAVYYCARGTYDDNYHDVMDAW | |
| | | GQGTLVTVSS | |
| | 3^{rd} | EIVLTQSPATLSLSPGERATLSCRASSSVSYMHWYQQKPGQAPRPWIYA | 628 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1927 | 1^{st} | QVQLVQSGAEVKKPGASVKVSCKASGYTFTSYVMHWVRQKPGQGLEWIG | 629 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCRASQDISNY | |
| | | LNWYQQKPGKAPKLLIYYTSRLRSGVPSRFSGSGSGTDFTLTISSLQPE | |
| | | DFATYYCQQGHTLPPTFGQGTKVEIKGGGGSGGGGSGGGGSGGGGSEVQ | |
| | | LVQSGAEVKKPGASVKVSCKASGYTFTDSYMSWVRQAPGQGLEWIGDMY | |
| | | PDNGDSSYNQKFRERVTITRDTSTSTAYLELSSLRSEDTAVYYCVLAPR | |
| | | WYFSVWGQGTLVTVSS | |
| | 2^{nd} | QVQLVQSGAEVKKPGASVKVSCKASGYTFTSYVMHWVRQKPGQGLEWIG | 630 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLIVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIKGGGGSGGGGSGGGGSGGGGSEVQLVQSGAEVKKPGASVK | |
| | | VSCKASGYTFTDSYMSWVRQAPGQGLEWIGDMYPDNGDSSYNQKFRERV | |
| | | TITRDTSTSTAYLELSSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 631 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1928 | 1^{st} | QVQLVQSGAEVKKPGASVKVSCKASGYTFTSYVMHWVRQKPGQGLEWIG | 632 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSDIQMTQSPSSLSASVGDRVTITCRASQDISNY | |
| | | LNWYQQKPGKAPKLLIYYTSRLRSGVPSRFSGSGSGTDFTLTISSLQPE | |
| | | DFATYYCQQGHTLPPTFGQGTKVEIKGGGGSGGGGSGGGGSGGGGSEVQ | |
| | | LVQSGAEVKKPGASVKVSCKASGYTFTDSYMSWVRQAPGQGLEWIGDMY | |
| | | PDNGDSSYNQKFRERVTITRDTSTSTAYLELSSLRSEDTAVYYCVLAPR | |
| | | WYFSVWGQGTLVTVSS | |
| | 2^{nd} | QVQLVQSGAEVKKPGASVKVSCKASGYTFTSYVMHWVRQKPGQGLEWIG | 633 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3rd | EIVLTQSPATLSLSPGERATLSCRASSSVSYMHWYQQKPGQAPRPWIYA | 634 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1929 | 1^{st} | QVQLVQSGAEVKKPGASVKVSCKASGYTFTSYVMHWVRQKPGQGLEWIG | 635 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 636 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIKGGGGSGGGGSGGGGSGGGGSEVQLVQSGAEVKKPGASVK | |
| | | VSCKASGYTFTDSYMSWVRQAPGQGLEWIGDMYPDNGDSSYNQKFRERV | |
| | | TITRDTSTSTAYLELSSLRSEDTAVYYCVLAPRWYFSVWGQGTLVTVSS | |
| | 3^{rd} | EIVLTQSPATLSLSPGERATLSCRASSSVSYMHWYQQKPGQAPRPWIYA | 637 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1930 | 1^{st} | QVQLVQSGAEVKKPGASVKVSCKASGYTFTSYVMHWVRQKPGQGLEWIG | 638 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| | 2^{nd} | QVQLVQSGAEVKKPGASVKVSCKASGYTFTSYVMHWVRQKPGQGLEWIG | 639 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGGGGGSGGGGSGGGGSGGGG | |
| | | SDIQMTQSPSSLSASVGDRVTITCRASQDISNYLNWYQQKPGKAPKLLI | |
| | | YYTSRLRSGVPSRFSGSGSGTDFTLTISSLQPEDFATYYCQQGHTLPPT | |
| | | FGQGTKVEIK | |
| | 3^{rd} | EIVLTQSPATLSLSPGERATLSCRASSSVSYMHWYQQKPGQAPRPWIYA | 640 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1931 | 1^{st} | QVQLVQSGAEVKKPGASVKVSCKASGYTFTSYVMHWVRQKPGQGLEWIG | 641 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 642 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSQ | |
| | | AVVTQEPSLTVSPGGTVTLTCRSSTGAVTTSNYANWVQQKPGQAPRGLI | |
| | | GGTNKRAPWTPARFSGSLLGGKAALTITGAQAEDEADYYCALWYSNLWV | |
| | | FGGGTKLTVLPAPEAAGPSVFLFPPKPKDTLMISRTPEVTCVVVDVSHE | |
| | | DPEVKFNWYVDGVEVHNAKTKPREEQYNSTYRVVSVLTVLHQDWLNGKE | |
| | | YKCKVSNKALPAPIEKTISKAKGQPREPQVYTLPPSREEMTKNQVSLSC | |
| | | AVKGFYPSDIAVEWESNGQPENNYKTTPPVLDSDGSFFLVSKLTVDKSR | |
| | | WQQGNVFSCSVMHEALHNRFTQKSLSLSPGK | |
| | 3^{rd} | EIVLTQSPATLSLSPGERATLSCRASSSVSYMHWYQQKPGQAPRPWIYA | 643 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly1932 | 1^{st} | QVQLVQSGAEVKKPGASVKVSCKASGYTFTSYVMHWVRQKPGQGLEWIG | 644 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 2^{nd} | QVQLVQSGAEVKKPGASVKVSCKASGYTFTSYVMHWVRQKPGQGLEWIG | 645 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPEAAGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSTLSASVGDRVTI | |
| | | TCSASSSVSYMNWYQQKPGKAPKLLIYDTSKLASGVPSRFSGSGSGTEF | |
| | | TLTISSLQPDDFATYYCQQWSSNPFTFGQGTKLEIKGGGGSGGGGSGGG | |
| | | GSGGGGSQVQLVQSGAEVKKPGASVKVSCKASGYTFTRYTMHWVRQAPG | |
| | | QGLEWMGYINPSRGYTNYNQKFKDRVTMTRDTSTSTVYMELSSLRSEDT | |
| | | AVYYCARYYDDHYCLDYWGQGTLVTVSS | |
| | 3^{rd} | EIVLTQSPATLSLSPGERATLSCRASSSVSYMHWYQQKPGQAPRPWIYA | 646 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| Ly1934 | 1^{st} | QVQLVQSGAEVKKPGASVKVSCKASGYTFTSYVMHWVRQKPGQGLEWIG | 647 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVESGGGLVQPGGSLRLSCAASGFTFSTYAMNWVRQAPGKGLEWVGR | |
| | | IRSKYNNYATYYADSVKGRFTISRDDSKNSLYLQMNSLKTEDTAVYYCV | |
| | | RHGNFGNSYVSWFAYWGQGTLVTVSSPAPEAAGPSVFLFPPKPKDTLMI | |
| | | SRTPEVTCVVVDVSHEDPEVKFNWYVDGVEVHNAKTKPREEQYNSTYRV | |
| | | VSVLTVLHQDWLNGKEYKCKVSNKALPAPIEKTISKAKGQPREPQVYTL | |
| | | PPSREEMTKNQVSLWCLVKGFYPSDIAVEWESNGQPENNYKTTPPVLDS | |
| | | DGSFFLYSKLTVDKSRWQQGNVFSCSVMHEALHNHYTQKSLSLSPGK | |
| | 2^{nd} | QVQLVQSGAEVKKPGASVKVSCKASGYTFTSYVMHWVRQKPGQGLEWIG | 648 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCGGGGSGGGGSGGGGSGGGGSE | |
| | | VQLVQSGAEVKKPGASVKVSCKASGYTFTDSYMSWVRQAPGQGLEWIGD | |
| | | MYPDNGDSSYNQKFRERVTITRDTSTSTAYLELSSLRSEDTAVYYCVLA | |
| | | PRWYFSVWGQGTLVTVSSASVAAPSVFIFPPSDEQLKSGTASVVCLLNN | |
| | | FYPREAKVQWKVDNALQSGNSQESVTEQDSKDSTYSLSSTLTLSKADYE | |
| | | KHKVYACEVTHQGLSSPVTKSFNRGECGGGGSGGGGSGGGGSGGGGSQA | |
| | | VVTQEPSLTVSPGGTVTLTCRSSTGAVTTSNYANWVQQKPGQAPRGLIG | |
| | | GTNKRAPWTPARFSGSLLGGKAALTITGAQAEDEADYYCALWYSNLWVF | |
| | | GGGTKLTVLPAPEAAGPSVFLFPPKPKDTLMISRTPEVTCVVVDVSHED | |
| | | PEVKFNWYVDGVEVHNAKTKPREEQYNSTYRVVSVLTVLHQDWLNGKEY | |
| | | KCKVSNKALPAPIEKTISKAKGQPREPQVYTLPPSREEMTKNQVSLSCA | |
| | | VKGFYPSDIAVEWESNGQPENNYKTTPPVLDSDGSFFLVSKLTVDKSRW | |
| | | QQGNVFSCSVMHEALHNRFTQKSLSLSPGK | |
| | 3^{rd} | EIVLTQSPATLSLSPGERATLSCRASSSVSYMHWYQQKPGQAPRPWIYA | 649 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |
| | 4^{th} | DIQMTQSPSSLSASVGDRVTITCRASQDISNYLNWYQQKPGKAPKLLIY | 650 |
| | chain | YTSRLRSGVPSRFSGSGSGTDFTLTISSLQPEDFATYYCQQGHTLPPTF | |
| | | GQGTKVEIKSSASTKGPSVFPLAPSSKSTSGGTAALGCLVKDYFPEPVT | |
| | | VSWNSGALTSGVHTFPAVLQSSGLYSLSSVVTVPSSSLGTQTYICNVNH | |
| | | KPSNTKVDKKVEPKSC | |

### Characterization of Anti-B7H3/CD3/GITR Tri-Specific Antibodies

These tri-specific antibodies are evaluated for their in vitro and in vivo activity, including binding to target antigen (B37H3, CD3 and GITR), agonistic activity in CD3 and GITR reporter assay system, activation of immune cells, anti-tumor activity in mouse models.

### Example 13: Anti-B7H3/CD137/OX40 Tri-Specific Antibodies

Anti-B7H3/CD137/OX40 tri-specific were produced using the same parent anti-B37H3 and anti-CD137 antibodies disclosed above and the anti-OX40 parent antibody Ly598 (OX40 Ab1). The sequences of the parent antibody clones are provided in Table 1 above.

cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 13 below:

TABLE 13
Exemplary Anti-B7H3/CD137/OX40 Tri-Specific Antibodies

| | | | SED ID |
|---|---|---|---|
| Antibody Clones | | Amino acid sequence | NO: |
| Ly2076 | 1^{st} | QVQLVQSGAEVKKPGASVKVSCKASGYTFTSYVMHWVRQKPGQGLEWIG | 651 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | EIVLTQSPATLSLSPGERATLSCRASSSVSYMHWYQQKPGQAPRPWIYA | 652 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly2077 | 1^{st} | QVQLVQSGAEVKKPGASVKVSCKASGYTFTSYVMHWVRQKPGQGLEWIG | 653 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | EIVLTQSPATLSLSPGERATLSCRASSSVSYMHWYQQKPGQAPRPWIYA | 654 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly2078 | 1^{st} | QVQLVQSGAEVKKPGASVKVSCKASGYTFTSYVMHWVRQKPGQGLEWIG | 655 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | QVQLVQSGAEVKKPGASVKVSCKASGYTFTSYVMHWVRQKPGQGLEWIG | 656 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSSLSASVGDRVTI | |
| | | TCRASQDISNYLNWYQQKPGKAPKLLIYYTSRLRSGVPSRFSGSGSGTD | |
| | | FTLTISSLQPEDFATYYCQQGHTLPPTFGQGTKVEIKGGGGSGGGGSGG | |
| | | GGSGGGGSEVQLVQSGAEVKKPGASVKVSCKASGYTFTDSYMSWVRQAP | |
| | | GQGLEWIGDMYPDNGDSSYNQKFRERVTITRDTSTSTAYLELSSLRSED | |
| | | TAVYYCVLAPRWYFSVWGQGTLVTVSS | |
| | 3^{rd} | EIVLTQSPATLSLSPGERATLSCRASSSVSYMHWYQQKPGQAPRPWIYA | 657 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYP | |
| | | REAKVQWKVDNALQSGNSQESVTEQDSKDSTYSLSSTLTLS | |
| | | KADYEKHKVYACEVTHQGLSSPVTKSFNRGEC | |

### Characterization of Anti-B7H3/CD137/OX40 Tri-Specific Antibodies

These tri-specific antibodies are evaluated for their in vitro and in vivo activity, including binding to target antigen (B7H3, CD137 and OX40), agonistic activity in CD137 and OX40 reporter assay system, activation of immune cells, anti-tumor activity in mouse models.

### Example 14: Anti-B7H3/CD137/GITR Tri-Specific Antibodies

Anti-B7H3/CD137/GITR tri-specific were produced using the same anti-B7H3 and anti-CD137 parent clones disclosed above and the anti-GITR parent antibody GITR Ab1. The sequences of the parent antibody clones are provided in Table 1 above.

cDNAs encoding the V_{H} and V_{L} chains of these parent antibodies were used as the starting materials for making the tri-specific antibodies. CHO-cell transient expression was carried out with plasmids configured for expressing polypeptide chains of the tri-specific antibodies. These antibodies were purified by protein A affinity chromatography.

The amino acid sequences of the polypeptide chains of the tri-specific antibodies are provided in Table 14 below:

TABLE 14
Exemplary Anti-B7H3/CD137/GITR Tri-Specific Antibodies

| | | | SED ID |
|---|---|---|---|
| Antibody Clones | | Amino acid sequence | NO: |
| Ly2079 | 1^{st} | QVQLVQSGAEVKKPGASVKVSCKASGYTFTSYVMHWVRQKPGQGLEWIG | 658 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | EIVLTQSPATLSLSPGERATLSCRASSSVSYMHWYQQKPGQAPRPWIYA | 659 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly2080 | 1^{st} | QVQLVQSGAEVKKPGASVKVSCKASGYTFTSYVMHWVRQKPGQGLEWIG | 660 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLTCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | EIVLTQSPATLSLSPGERATLSCRASSSVSYMHWYQQKPGQAPRPWIYA | 661 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGECGGGGSGGGGSGGGGSGGGGSDIQMTQSPSSLS | |
| | | ASVGDRVTITCRASQDISNYLNWYQQKPGKAPKLLIYYTSRLRSGVPSR | |
| | | FSGSGSGTDFTLTISSLQPEDFATYYCQQGHTLPPTFGQGTKVEIKGGG | |
| | | GSGGGGSGGGGSGGGGSEVQLVQSGAEVKKPGASVKVSCKASGYTFTDS | |
| | | YMSWVRQAPGQGLEWIGDMYPDNGDSSYNQKFRERVTITRDTSTSTAYL | |
| | | ELSSLRSEDTAVYYCVLAPRWYFSVWGQGTLVTVSS | |
| Ly2081 | 1^{st} | QVQLVQSGAEVKKPGASVKVSCKASGYTFTSYVMHWVRQKPGQGLEWIG | 662 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLWCLVKGFYPSDIAVEWESNGQPEN | |
| | | NYKTTPPVLDSDGSFFLYSKLTVDKSRWQQGNVFSCSVMHEALHNHYTQ | |
| | | KSLSLSPGGGGGSGGGGSGGGGSGGGGSDIQMTQSPSSLSASVGDRVTI | |
| | | TCRASQDIRSNLNWYQQKPGGAVKLLIYYTSRLHSGVPSRFSGSGSGTD | |
| | | YTLTISSLQPEDFATYFCQQSEKLPRTFGGGTKVEIRGGGGSGGGGSGG | |
| | | GGSGGGGSQVQLVQSGAEVKKPGASVKVSCKASGYTFAGFEMHWVRQAP | |
| | | GQGLEWMGAIDPKTGGTDYNQKFKDRVTMTRDTSISTAYMELSRLRSDD | |
| | | TAVYYCARDLGYFDVWGQGTLVTVSS | |
| | 2^{nd} | QVQLVQSGAEVKKPGASVKVSCKASGYTFTSYVMHWVRQKPGQGLEWIG | 663 |
| | chain | YINPYNEGTESTDKFKGRATMTSDKSTSTVYMELSSLRSEDTAVYYCAS | |
| | | IYYGYEGTYFGVWGQGTLVTVSSASTKGPSVFPLAPSSKSTSGGTAALG | |
| | | CLVKDYFPEPVTVSWNSGALTSGVHTFPAVLQSSGLYSLSSVVTVPSSS | |
| | | LGTQTYICNVNHKPSNTKVDKKVEPKSCDKTHTCPPCPAPELLGPSVFL | |
| | | FPPKPKDTLMISRTPEVTCVVVDVSHEDPEVKFNWYVDGVEVHNAKTKP | |
| | | REEQYNSTYRVVSVLTVLHQDWLNGKEYKCKVSNKALPAPIEKTISKAK | |
| | | GQPREPQVYTLPPSREEMTKNQVSLSCAVKGFYPSDIAVEWESNGQPEN | |
| | | NYKTTPPVLDSDGSFFLVSKLTVDKSRWQQGNVFSCSVMHEALHNRFTQ | |
| | | KSLSLSPGGGGGSGGGGSGGGGSGGGGSDIQMTQSPSSVSASVGDRVTI | |
| | | TCRASQGIYSWLAWYQQKPGKAPNLLIYTASTLQSGVPSRFSGSGSGTD | |
| | | FTLTISSLQPEDFATYYCQQANIFPLTFGGGTKVEIKGGGGSGGGGSGG | |
| | | GGSGGGGSQVQLVQSGAEVKKPGASVKVSCKASGYTFTGYYMHWVRQAP | |
| | | GQGLEWMGWINPDSGGTNYAQKFQGRVTMTRDTSISTAYMELNRLRSDD | |
| | | TAVYYCARDQPLGYCTNGVCSYFDYWGQGTLVTVSS | |
| | 3^{rd} | EIVLTQSPATLSLSPGERATLSCRASSSVSYMHWYQQKPGQAPRPWIYA | 664 |
| | chain | TSNLASGIPARFSGSRSGTDYTLTISSLEPEDFAVYYCQQWSSNTLTFG | |
| | | GGTKVEIKRTVAAPSVFIFPPSDEQLKSGTASVVCLLNNFYPREAKVQW | |
| | | KVDNALQSGNSQESVTEQDSKDSTYSLSSTLTLSKADYEKHKVYACEVT | |
| | | HQGLSSPVTKSFNRGEC | |

### Characterization of Anti-B7H3/CD137/GITR Tri-Specific Antibodies

These tri-specific antibodies are evaluated for their in vitro and in vivo activity, including binding to target antigen (B7H3, CD137 and GITR), agonistic activity in CD137 and GITR reporter assay system, activation of immune cells, anti-tumor activity in mouse models.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A multi-specific antibody, comprising:
(a) a first antigen binding moiety binding to a first antigen, wherein the first antigen binding moiety comprising a first heavy chain variable region (V_{H}) and a first light chain variable region (V_{L}); and
(b) a second antigen binding moiety binding to a second antigen, wherein the second antigen binding moiety comprising a second V_{H} and a second V_{L}; and optionally
(c) a third antigen binding moiety binding to a third antigen, wherein the third antigen binding moiety comprising a third V_{H} and a third V_{L};
wherein one of the first antigen and the second antigen is human B7H3 and the other one is selected from the group consisting of human CD40, human CD137, human Glucocorticoid-Induced TNFR-Related protein (GITR), human OX40, human CD3, human CD28, and human CD47; and
wherein the optional third antigen is selected from the group consisting of human CD40, human CD137, human Glucocorticoid-Induced TNFR-Related protein (GITR), human OX40, human CD3, human CD28, and human CD47; the optional third antigen being different from the first antigen and the second antigen.

2. The multi-specific antibody of claim 1, wherein the antigen binding moiety that binds B7H3 comprises (i) a heavy chain variable region (V_{H}) that comprises the same heavy chain complementary determining regions (CDRs) as antibody B7H3 Ab1 or B7H3 Ab2, and (ii) a light chain variable region (V_{L}) that comprises the same light chain CDRs as antibody B7H3 Ab1 or B7H3 Ab2.

3. The multi-specific antibody of claim 1, wherein the antigen binding moiety that binds B7H3 comprises (i) a heavy chain variable region (V_{H}) that comprises heavy chain complementary determining regions (CDRs), which collectively contain up to 5 amino acid residue variations relative to the heavy chain CDR3 of antibody B7H3 Ab1 or B7H3 Ab2, and (ii) a light chain variable region (V_{L}) that comprises light chain CDRs, which collectively contain up to 5 amino acid residue variations relative to the light chain CDRs of antibody B7H3 Ab1 or B7H3 Ab2.

4. The multi-specific antibody of claim 3, wherein the antigen binding moiety that binds B7H3 comprises (i) the V_{H} that comprises an amino acid sequence at least 80% identical to that of antibody B7H3 Ab1 or B7H3 Ab2; and (ii) the V_{L} that comprises an amino acid sequence at least 80% identical to that of antibody B7H3 Ab1 or B7H3 Ab2.

5. The multi-specific antibody of claim 4, wherein the antigen binding moiety that binds B7H3 comprises the same V_{H} and same V_{L} as antibody B7H3 Ab1 or B7H3 Ab2.

6. The multi-specific antibody of claim 1, wherein the antigen binding moieties that bind human CD40, human CD137, human Glucocorticoid-Induced TNFR-Related protein (GITR), human OX40, human CD3, human CD28, or human CD47 each comprise (i) a heavy chain variable region (V_{H}) that comprises the same heavy chain complementary determining regions (CDRs) as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and (ii) a light chain variable region (V_{L}) that comprises the same light chain CDRs as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

7. The multi-specific antibody of claim 1, wherein the antigen binding moieties that bind human CD40, human CD137, human Glucocorticoid-Induced TNFR-Related protein (GITR), human OX40, human CD3, human CD28, or human CD47 each comprise:
(i) a heavy chain variable region (V_{H}) that comprises heavy chain complementary determining regions (CDRs), which collectively contain up to 5 amino acid residue variations relative to the heavy chain CDR3 of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and
(ii) a light chain variable region (V_{L}) that comprises light chain CDRs, which collectively contain up to 5 amino acid residue variations relative to the light chain CDRs of CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

8. The multi-specific antibody of claim 7, wherein the antigen binding moieties that bind human CD40, human CD137, human Glucocorticoid-Induced TNFR-Related protein (GITR), human OX40, human CD3, human CD28, or human CD47 each comprise:
(i) the V_{H} that comprises an amino acid sequence at least 80% identical to that of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2; and
(ii) the V_{L} that comprises an amino acid sequence at least 80% identical to that of antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

9. The multi-specific antibody of claim 8, wherein the antigen binding moieties that bind human CD40, human CD137, human Glucocorticoid-Induced TNFR-Related protein (GITR), human OX40, human CD3, human CD28, or human CD47 each comprise the same V_{H} and same V_{L} as antibody CD40 Ab1, CD137 Ab1, GITR Ab1, OX40 Ab1, CD28 Ab1, CD28 Ab2, CD28 Ab3, CD47 Ab1, CD47 Ab2, CD3 Ab1, or CD3 Ab2.

10. The multi-specific antibody of claim 1, which is a bi-specific antibody that comprises the first antigen binding moiety and the second antigen binding moiety.

11. The multi-specific antibody of claim 10, wherein the bi-specific antibody is of a two-chain format.

12. The multi-specific antibody of claim 11, wherein (i) the first antigen binding moiety comprises a heavy chain that comprises the first V_{H} and a heavy chain constant region or a fragment thereof and a light chain that comprises the first V_{L} and a light chain constant region; and (ii) the second antigen binding moiety is a single chain variable fragment (scFv); and wherein the scFv is linked to either the heavy chain or the light chain of (i).

13. The multi-specific antibody of claim 12, wherein the scFv is linked to the N-terminus of the heavy chain of (i) or the C-terminus of the heavy chain.

14. The multi-specific antibody of claim 12, which comprises a first polypeptide that comprises the heavy chain of (i) fused to the scFv, and a second polypeptide that comprises the light chain of (i).

15. The multi-specific antibody of claim 12, which comprises a first polypeptide that comprise the heavy chain of (i); and a second polypeptide that comprises the light chain of (i) fused to the scFv.

16. The multi-specific antibody of claim 12, wherein the first antigen binding moiety binds B7H3.

17. The multi-specific antibody of claim 14, wherein the multi-specific antibody is a multi-chain complex comprising two copies of each of the first polypeptide and the second polypeptide.

18. The multi-specific antibody of claim 10, which is of a three-chain format.

19. The multi-specific antibody of claim 18, which comprises:
(i) a first polypeptide that comprise a first heavy chain of the first antigen binding moiety, wherein the first heavy chain comprises the first V_{H} and a first heavy chain constant region or a fragment comprising the CH3 domain therein,
(ii) a second polypeptide that comprises a second heavy chain of the first antigen binding moiety and the second antigen binding moiety, which is a single chain variable fragment (scFv) comprising the second V_{H} and second V_{L}, wherein the second heavy chain comprises the first V_{H} and a second heavy chain constant region or a fragment comprising the CH3 domain therein; and
(iii) a third polypeptide that comprises a light chain of the first antigen binding moiety, which comprises the first V_{L} and a light chain constant region;
optionally wherein the first and second heavy chain constant regions comprise mutations in the CH3 domains that enhances heterodimeration over homodimeration as relative to the wild-type counterpart and/or reduces protein A binding.

20. The multi-specific antibody of claim 19, wherein in (ii), the scFv is located between the first V_{H} and the second Fc fragment or the CH3 domain thereof.

21. The multi-specific antibody of claim 19, wherein in (ii), the scFv is located at the C-terminus of the second polypeptide.

22. The multi-specific antibody of claim 18, which comprises:
(i) a first polypeptide that comprise a first heavy chain of the first antigen binding moiety and one of the second V_{H} and the second V_{L} of the second antigen binding moiety; wherein the first heavy chain comprises the first V_{H} and a first heavy chain constant region or a fragment comprising the CH3 domain therein,
(ii) a second polypeptide that comprises a second heavy chain of the first antigen binding moiety and the other one of the second V_{H} and second V_{L} of the second antigen binding moiety, wherein the second heavy chain comprises the first V_{H} and a second heavy chain constant region or a fragment comprising the CH3 domain therein; and
(iii) a third polypeptide that comprises a light chain of the first antigen binding moiety, which comprises the first V_{L} and a light chain constant region;
optionally wherein the first and second heavy chain constant regions comprise mutations in the CH3 domains that enhances heterodimeration over homodimeration as relative to the wild-type counterpart.

23. The multi-specific antibody of claim 19, wherein the first and second heavy chain constant regions comprise the mutations, which are knob-in-hole mutations, charged mutations, or ZW1 mutations.

24. The multi-specific antibody of claim 19, wherein one of the first and second heavy chain constant regions comprises a mutation that reduces protein A binding activity relative to the wild-type counterpart.

25. The multi-specific antibody of claim 10, which is of a four-chain format.

26. The multi-specific antibody of claim 25, which comprises:
(i) a first polypeptide comprising a first heavy chain of the first antigen binding moiety, the first heavy chain comprising the first V_{H} and a first heavy chain constant region or a fragment comprising the CH3 domain therein;
(ii) a second polypeptide comprising a second heavy chain of the second antigen binding moiety, the second heavy chain comprising the second V_{H}, a light chain constant region, and a second heavy chain constant fragment comprising a CH3 domain;
(iii) a third polypeptide comprising a light chain of the first antigen moiety, which comprises the first V_{L} and a light chain constant region; and
(iv) a fourth polypeptide comprising a light chain of the second antigen moiety, the light chain comprising the second V_{L} linked to a CH1 domain of a heavy chain constant region;
optionally wherein the first and second heavy chain constant regions comprise mutations in the CH3 domains that enhances heterodimeration over homodimeration as relative to the wild-type counterpart.

27. The multi-specific antibody of claim 25, which comprises:
(i) a first polypeptide comprising a first heavy chain of the first antigen binding moiety, wherein the first heavy chain comprises the first V_{H} and a first heavy chain constant region or a fragment comprising the CH3 domain therein;
(ii) a second polypeptide comprising a second heavy chain, which comprises the first V_{H}, the second antibody binding moiety that is a scFv fragment comprising the second V_{H} and the second V_{L}, and a second heavy chain constant region or a fragment comprising the CH3 domain therein;
(iii) a third polypeptide comprising a light chain of the first antigen binding moiety, which comprises the first V_{L} and a light chain constant region; and
(iv) a fourth polypeptide comprising the scFv;
optionally wherein the first and second heavy chain constant regions comprise mutations in the CH3 domains that enhances heterodimeration over homodimeration as relative to the wild-type counterpart.

28. The multi-specific antibody of claim 25, which comprises:
(i) a first polypeptide comprising a first heavy chain of the first antigen binding moiety, wherein the first heavy chain comprises the first V_{H} and a first heavy chain constant region or a fragment comprising the CH3 domain therein;
(ii) a second polypeptide comprising a light chain of the first antigen binding moiety, which comprises the first V_{L} and a light chain constant region;
(iii) a third polypeptide comprising the second V_{H}, a first TCR fragment, and a second heavy chain constant fragment comprising a CH3 domain; and
(iv) a fourth polypeptide comprising the second V_{L} and a second TCR fragment;
wherein the first and second TCR fragments collectively are a TCR alpha chain fragment and a TCR beta chain fragment, which form a dimer; and
optionally wherein the first and second heavy chain constant regions comprise mutations in the CH3 domains that enhances heterodimeration over homodimeration as relative to the wild-type counterpart.

29. The multi-specific antibody of claim 28, wherein the third polypeptide further comprises the first V_{H} linked to a CH1 domain of a heavy chain constant region.

30. The multi-specific antibody of claim 25, wherein the first and second heavy chain constant regions comprise the mutations, which are knob-in-hole mutations, charged mutations, or ZW1 mutations.

31. The multi-specific antibody of claim 25, wherein one of the first and second heavy chain constant regions comprises a mutation that reduces protein A binding activity relative to the wild-type counterpart.

32. The multi-specific antibody of claim 11, which is a bi-specific antibody comprising the first antigen binding moiety that binds B7H3 and the second antigen binding moiety that binds CD40.

33. The multi-specific antibody of claim 32, which is selected from the group consisting of Ly1581, Ly1660, Ly1661, Ly1662, Ly1663, Ly1679, Ly1935, and Ly1936.

34. The multi-specific antibody of claim 11, which is a bi-specific antibody comprising the first antigen binding moiety that binds B7H3 and the second antigen binding moiety that binds CD137.

35. The multi-specific antibody of claim 34, which is selected from the group consisting of Ly1937, Ly1938, Ly1939, Ly1940, Ly1941, Ly1942, Ly1943, and Ly1944.

36. The multi-specific antibody of claim 11, which is a bi-specific antibody comprising the first antigen binding moiety that binds B7H3 and the second antigen binding moiety that binds GITR.

37. The multi-specific antibody of claim 36, which is selected from the group consisting of Ly1945, Ly1946, Ly1947, Ly1948, Ly1049, Ly1950, Ly1951, and Ly1952.

38. The multi-specific antibody of claim 11, which is a bi-specific antibody comprising the first antigen binding moiety that binds B7H3 and the second antigen binding moiety that binds OX40.

39. The multi-specific antibody of claim 38, which is selected from the group consisting of Ly1953, Ly1954, Ly1955, Ly1956, Ly1957, Ly1958, Ly1959, and Ly1960.

40. The multi-specific antibody of claim 11, which is a bi-specific antibody comprising the first antigen binding moiety that binds B7H3 and the second antigen binding moiety that binds CD47.

41. The multi-specific antibody of claim 40, which is selected from the group consisting of Ly2043, Ly2044, Ly2045, Ly2046, Ly2047, Ly2048, Ly2049, Ly2050, Ly2051, Ly2052, Ly2053, Ly2054, Ly2055, Ly2056, Ly2057, Ly2058, Ly2059, Ly2060, Ly2061, Ly2062, Ly2063, and Ly2064.

42. The multi-specific antibody of claim 11, which is a bi-specific antibody comprising the first antigen binding moiety that binds B7H3 and the second antigen binding moiety that binds CD3.

43. The multi-specific antibody of claim 42, which is selected from the group consisting of Ly1900, Ly1901, Ly1902, Ly1903, and Ly1904.

44. The multi-specific antibody of claim 1, which is a tri-specific antibody that comprises (i) the first antigen binding moiety, (ii) the second antigen binding moiety, and (iii) the third antigen binding moiety.

45. The multi-specific antibody of claim 44, which comprises:
(i) a first polypeptide comprising a first heavy chain that comprises the first V_{H} of the first antigen binding moiety and first heavy chain constant region or a fragment comprising the CH3 domain therein;
(ii) a second polypeptide comprising the first V_{H}, a second heavy chain comprising the second V_{H} of the second antigen binding moiety and a light chain constant region, and a second heavy chain constant region or a fragment comprising the CH3 domain therein;
(iii) a third polypeptide comprising a light chain of the first antigen binding moiety, which comprises the first V_{L} and a light chain constant region; and
(iv) a fourth polypeptide comprising the second V_{L} of the second antigen binding moiety and a CH1 fragment of a heavy chain constant region;
wherein the first polypeptide, the second polypeptide or both further comprise the third antigen binding moiety, which is a single chain variable fragment (scFv);
optionally wherein the first and second heavy chain constant regions comprise mutations in the CH3 domains that enhances heterodimeration over homodimeration as relative to the wild-type counterpart.

46. The multi-specific antibody of claim 44, which comprises:
(i) a first polypeptide comprising a first heavy chain that comprises the first V_{H} of the first antigen binding moiety and a first heavy chain constant region or a fragment comprising the CH3 domain therein;
(ii) a second polypeptide comprising the first V_{H}, a second heavy chain comprising the second V_{L} of the second antigen binding moiety and a CH1 domain of a heavy chain constant region, and a second heavy chain constant region or a fragment comprising the CH3 domain therein;
(iii) a third polypeptide comprising a light chain of the first antigen binding moiety; and
(iv) a fourth polypeptide comprising the second V_{H} of the second antigen binding moiety and a light chain constant region;
wherein the first polypeptide, the second polypeptide or both further comprise the third antigen binding moiety, which is a single chain variable fragment (scFv); and
optionally wherein the first and second heavy chain constant regions comprise mutations in the CH3 domains that enhances heterodimeration over homodimeration as relative to the wild-type counterpart.

47. The multi-specific antibody of claim 44, which comprises:
(i) a first polypeptide comprising a first heavy chain that comprises the first V_{H} of the first antigen binding moiety, first heavy chain constant region or a fragment comprising the CH3 domain therein, and one of the third V_{H} and third V_{L} of the third antigen binding moiety;
(ii) a second polypeptide comprising the first V_{H}, a second heavy chain comprising the second V_{H} of the second antigen binding moiety and a light chain constant region, a second heavy chain constant region or a fragment comprising the CH3 domain therein, and the other one of the third V_{H} and third V_{L};
(iii) a third polypeptide comprising a light chain of the first antigen binding moiety, which comprises the first V_{L} and a light chain constant region; and
(iv) a fourth polypeptide comprising the second V_{L} of the second antigen binding moiety and a CH1 domain of a heavy chain constant region;
optionally wherein the first and second heavy chain constant regions comprise mutations in the CH3 domains that enhances heterodimeration over homodimeration as relative to the wild-type counterpart.

48. The multi-specific antibody of claim 44, which comprises:
(i) a first polypeptide comprising a first heavy chain that comprises the first V_{H} of the first antigen binding moiety, a first heavy chain constant region or a fragment comprising the CH3 domain therein, and one of the third V_{H} and third V_{L} of the third antigen binding moiety;
(ii) a second polypeptide comprising the first V_{H}, a second heavy chain comprising the second V_{L} of the second antigen binding moiety and a CH1 domain of a heavy chain constant region, a second heavy chain constant region or a fragment comprising the CH3 domain therein, and the other one of the third V_{H} and third V_{L};
(iii) a third polypeptide comprising a light chain of the first antigen binding moiety, which comprises the first V_{L} and a light chain constant region; and
(iv) a fourth polypeptide comprising the second V_{H} of the second antigen binding moiety and a light chain constant region;
optionally wherein the first and second heavy chain constant regions comprise mutations in the CH3 domains that enhances heterodimeration over homodimeration as relative to the wild-type counterpart.

49. The multi-specific antibody of claim 44, which comprises:
(i) a first polypeptide comprising a first heavy chain that comprises the first V_{H} of the first antigen binding moiety and a first heavy chain constant region or a fragment comprising the CH3 domain therein;
(ii) a second polypeptide comprising the first V_{H}, the second antigen binding moiety, which is a scFv, and a second heavy chain constant region or a fragment comprising the CH3 domain therein; and
(iii) a third polypeptide comprising a light chain of the first antigen binding moiety, which comprises the first V_{L} and a light chain constant region;
wherein the first polypeptide, the second polypeptide, or both further comprise the third antigen binding moiety, which is a scFv; and
optionally wherein the first and second heavy chain constant regions comprise mutations in the CH3 domains that enhances heterodimeration over homodimeration as relative to the wild-type counterpart.

50. The multi-specific antibody of claim 44, which comprises:
(i) a first polypeptide comprising a first heavy chain that comprises the first V_{H} of the first antigen binding moiety, one of the second V_{H} and second V_{L} of the second antigen binding moiety, and a first heavy chain constant region or a fragment comprising the CH3 domain therein;
(ii) a second polypeptide comprising the first V_{H}, the other one of the second V_{H} and second V_{L}, and a second heavy chain constant region or a fragment comprising the CH3 domain therein; and
(iii) a third polypeptide comprising a light chain of the first antigen binding moiety, which comprises the first V_{L} and a light chain constant region;
wherein the first polypeptide, the second polypeptide, or both further comprise the third antigen binding moiety, which is a scFv, or wherein the third polypeptide further comprises the third antigen binding moiety; and
optionally wherein the first and second heavy chain constant regions comprise mutations in the CH3 domains that enhances heterodimeration over homodimeration as relative to the wild-type counterpart.

51. The multi-specific antibody of claim 44, which comprises:
(i) a first polypeptide comprising a first heavy chain that comprises the first V_{H} of the first antigen binding moiety, one of the second V_{H} and second V_{L} of the second antigen binding moiety, a first heavy chain constant region or a fragment comprising the CH3 domain therein, and one of the third V_{H} and third V_{L} of the third antigen binding moiety;
(ii) a second polypeptide comprising the first V_{H}, the other one of the second V_{H} and second V_{L}, and a second heavy chain constant region or a fragment comprising the CH3 domain therein, and the other one of the third V_{H} and third V_{L}; and
(iii) a third polypeptide comprising a light chain of the first antigen binding moiety, which comprises the first V_{L} and a light chain constant region;
optionally wherein the first and second heavy chain constant regions comprise mutations in the CH3 domains that enhances heterodimeration over homodimeration as relative to the wild-type counterpart.

52. The multi-specific antibody of claim 44, which comprises:
(i) a first polypeptide comprising a first heavy chain that comprises the first V_{H} of the first antigen binding moiety, a first heavy chain constant region or a fragment comprising the CH3 domain therein, and the second antigen binding moiety, which is a scFv;
(ii) a second polypeptide comprising the first V_{H}, a second heavy chain constant region or a fragment comprising the CH3 domain therein, and the third antigen binding moiety, which is a scFv; and
(iii) a third polypeptide comprising a light chain of the first antigen binding moiety, which comprises the first V_{L} and a light chain constant region;
optionally wherein the first and second heavy chain constant regions comprise mutations in the CH3 domains that enhances heterodimeration over homodimeration as relative to the wild-type counterpart.

53. The multi-specific antibody of claim 44, which comprises:
(i) a first polypeptide comprising a first heavy chain that comprises the first V_{H} of the first antigen binding moiety and a first heavy chain constant region or a fragment comprising the CH3 domain therein;
(ii) a second polypeptide comprising a second heavy chain that comprises the second V_{H} of the second antigen binding moiety and one of the third V_{H} and third V_{L} of the third antigen binding moiety, wherein the third V_{H} is fused with a light chain constant region or the third V_{L} is fused with a CH1 domain of a heavy chain constant region;
(iii) a third polypeptide comprising a light chain of the second antigen binding moiety, which comprises the second V_{L} and a light chain constant region, and the other one of the third V_{H} and third V_{L} of the third antigen binding moiety, wherein the third V_{H} is fused with a light chain constant region or the third V_{L} is fused with a CH1 domain of a heavy chain constant region; and
(iv) a fourth polypeptide comprising a light chain of the first antigen binding moiety, which comprises the first V_{L} and a light chain constant region;
wherein either the second polypeptide or the third polypeptide further comprise a second heavy chain constant region or a fragment comprising the CH3 domain therein; and
optionally wherein the first and second heavy chain constant regions comprise mutations in the CH3 domains that enhances heterodimeration over homodimeration as relative to the wild-type counterpart.

54. The multi-specific antibody of claim 44, which comprises:
(i) a first polypeptide comprising the first V_{H} of the first antigen binding moiety, the second V_{H} of the second antigen binding moiety, and a first heavy chain constant region or a fragment comprising the CH3 domain therein;
(ii) a second polypeptide comprising the third V_{H} of the third antigen binding moiety and a light chain constant domain;
(iii) a third polypeptide comprising the third V_{L} of the third antigen binding moiety and a CH1 domain of a heavy chain constant region; and
(iv) a fourth polypeptide comprising a light chain of the first antigen binding moiety, which comprises the first V_{L} and a light chain constant region;
wherein either the second polypeptide or the third polypeptide further comprise the second V_{L} of the second antigen binding moiety and a second heavy chain constant region or a fragment comprising the CH3 domain therein; and
optionally wherein the first and second heavy chain constant regions comprise mutations in the CH3 domains that enhances heterodimeration over homodimeration as relative to the wild-type counterpart.

55. The multi-specific antibody of claim 44, which comprises:
(i) a first polypeptide comprising the first V_{H} of the first antigen binding moiety, the second V_{L} of the second antigen binding moiety, and a first heavy chain constant region or a fragment comprising the CH3 domain therein;
(ii) a second polypeptide comprising the third V_{H} of the third antigen binding moiety and a light chain constant domain;
(iii) a third polypeptide comprising the third V_{L} of the third antigen binding moiety and a CH1 domain of a heavy chain constant region; and
(iv) a fourth polypeptide comprising a light chain of the first antigen binding moiety, which comprises the first V_{L} and a light chain constant region;
wherein either the second polypeptide or the third polypeptide further comprise the second V_{H} of the second antigen binding moiety and a second heavy chain constant region or a fragment comprising the CH3 domain therein;
optionally wherein the first and second heavy chain constant regions comprise mutations in the CH3 domains that enhances heterodimeration over homodimeration as relative to the wild-type counterpart.

56. The multi-specific antibody of claim 54, wherein either the second polypeptide or the third polypeptide further comprises the first V_{H} fused to a CH1 of a heavy chain constant region.

57. The multi-specific antibody of claim 44, which comprises:
(i) a first polypeptide comprising a heavy chain of the 1^{st} antigen binding moiety and the second antigen binding moiety, wherein the heavy chain of the 1^{st} antigen binding moiety comprises the first V_{H} and a heavy chain constant region, and wherein the second antigen binding moiety is an scFv fragment; and
(ii) a second polypeptide comprising a light chain of the 1^{st} antigen binding moiety and the third antigen binding moiety, wherein the light chain comprises the first V_{L} and a light chain constant region, and wherein the third antigen binding moiety is an scFv fragment.

58. The multi-specific antibody of claim 44, which comprises:
(i) a first polypeptide comprising a heavy chain of the 1^{st} antigen binding moiety and the second antigen binding moiety, wherein the heavy chain of the 1^{st} antigen binding moiety comprises the first V_{H} and a first heavy chain constant region, and wherein the second antigen binding moiety is an scFv fragment; and
(ii) a second polypeptide comprising a heavy chain of the 1^{st} antigen binding moiety and the third antigen binding moiety, wherein the heavy chain comprises the first V_{H} and a second heavy chain constant region, and wherein the third antigen binding moiety is an scFv fragment; and
(iii) a third polypeptide comprising a light chain of the first antigen binding moiety, the light chain comprising the first V_{L} and a light chain constant region;
optionally wherein the first and second heavy chain constant regions comprise mutations in the CH3 domains that enhances heterodimeration over homodimeration as relative to the wild-type counterpart.

59. The multi-specific antibody of claim 45, wherein the first and second heavy chain constant regions comprise the mutations, which are knob-in-hole mutations, charged mutations, or ZW1 mutations.

60. The multi-specific antibody of claim 45, wherein one of the first and second heavy chain constant regions comprises a mutation that reduces protein A binding activity relative to the wild-type counterpart.

61. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD3, and CD137.

62. The multi-specific antibody of claim 61, which is selected from the group consisting of Ly1785, Ly1793, Ly1794, Ly1795, Ly1796, Ly1797, Ly1798, Ly1799, Ly1800, Ly1801, Ly1802, Ly1803, Ly1804, Ly1805, and Ly1849.

63. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD3, and GITR.

64. The multi-specific antibody of claim 63, which is selected from the group consisting of Ly1905, Ly1906, Ly1907, Ly1908, Ly1909, Ly1910, Ly1911, Ly1912, Ly1913, Ly1914, Ly1915, Ly1916, Ly1917, Ly1918, and Ly1933.

65. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD3, and OX40.

66. The multi-specific antibody of claim 65, which is selected from the group consisting of Ly1919, Ly1920, Ly1921, Ly1922, Ly1923, Ly1924, Ly1925, Ly1926, Ly1927, Ly1928, Ly1929, Ly1930, Ly1931, Ly1932, and Ly1934.

67. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD3, and CD28.

68. The multi-specific antibody of claim 67, which is selected from the group consisting of Ly1968-Ly2042.

69. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD137, and OX40.

70. The multi-specific antibody of claim 69, which is Ly2076, Ly2077, or Ly2078.

71. The multi-specific antibody of claim 44, which is a tri-specific antibody comprising (i) the first antigen binding moiety, (ii) the second antigen binding moiety, and (iii) the third antigen binding moiety, wherein (i)-(iii) bind B7H3, CD137, and GITR.

72. The multi-specific antibody of claim 71, which is Ly2079, Ly2080, or Ly2081.

73. A humanized antibody specific to human B7H3, wherein the humanized antibody comprises a heavy chain variable region (V_{H}) and a light chain variable region (V_{L}), wherein:
(i) the V_{H} comprises a framework of IGHV1-46*01 and heavy chain complementary determining regions (CDRs) 1, 2, and 3, which are either identical to those of parent murine antibody Ly383 or collectively contain no more than 5 amino acid residue variations relative to the parent murine antibody Ly383; or
(ii) the V_{H} comprises a framework of IGHV1-2*02 and heavy chain CDRs 1, 2, and 3, which are either identical to those of parent murine antibody Ly387 or collectively contain no more than 5 amino acid residue variations relative to the parent murine antibody Ly387.

74. The humanized antibody of claim 73, wherein the V_{L} comprises a framework of IGKV3-11*01 and light chain CDRs 1, 2, and 3, which are either identical to those of the parent murine antibody Ly383 or Ly387 or collectively contain no more than 5 amino acid residue variations relative to the parent murine antibody Ly383 or Ly387.

75. The humanized antibody of claim 73, wherein the antibody comprises the same heavy chain CDRs 1, 2, and 3 as antibody Ly383, and/or the same light chain CDRs 1, 2, and 3 and antibody Ly383.

76. The humanized antibody of claim 73, wherein the antibody comprises the same heavy chain CDRs 1, 2, and 3 as antibody Ly387, and/or the same light chain CDRs 1, 2, and 3 and antibody Ly387.

77. The humanized antibody of claim 75, wherein the V_{H} comprises one or more mutations in the V_{H} framework.

78. The humanized antibody of claim 77, wherein the mutations in the V_{H} framework are back mutations based on amino acid residues in the parent murine antibody at corresponding positions.

79. The humanized antibody of claim 73, wherein the V_{H} comprises the amino acid sequence of SEQ ID NO: 35, 39, 47, or 49; and/or wherein the V_{L} comprises the amino acid sequence of SEQ ID NO: 37 or 41.

80. The humanized antibody of claim 73, wherein the V_{H} comprises the amino acid sequence of SEQ ID NO: 43; and/or wherein the V_{L} comprises the amino acid sequence of SEQ ID NO: 45.

81. The humanized antibody of claim 73, wherein the antibody is a full-length antibody.

82. The humanized antibody of claim 81, wherein the full-length antibody is an IgG/kappa molecule.

83. The humanized antibody of claim 82, wherein the full-length antibody comprises a heavy chain that is an IgG1, IgG2, or IgG4 chain.

84. The humanized antibody of claim 83, wherein the heavy chain comprises a mutated Fc region, which exhibits altered binding affinity or selectivity to an Fc receptor as relative to the wild-type counterpart.

85. The humanized antibody of claim 73, wherein the antibody is selected from the group consisting of Ly1426, Ly1562, Ly1612, Ly1614, Ly1616, Ly1618, and Ly1442.

86. A nucleic acid or a nucleic acid set, which collectively encodes an antibody of any one of the preceding claims.

87. The nucleic acid or nucleic acid set of claim 86, which is an expression vector or an expression vector set.

88. A host cell, comprising the nucleic acid or nucleic acid set of claim 86.

89. The host cell of claim 88, which is a mammalian host cell.

90. A method for producing an antibody set forth in claim 1:
(i) culturing the host cell of comprising a nucleic acid(s) encoding the antibody under conditions allowing for expression of the antibody; and
(ii) harvesting the antibody thus produced.

91. A pharmaceutical composition comprising an antibody of claim 1 and a pharmaceutically acceptable carrier.

92. A method for modulating immune responses, comprising administering an effective amount of claim 1 or the pharmaceutical composition thereof to a subject in need thereof.

93. The method of claim 92, wherein the subject is a human patient having or suspected of having cancer.
